(19) United States
(12) Patent Application Publication
(10) Pub. No.: US 2007/0161029 A1
(43) Pub. Date: Jul. 12, 2007

(54) High throughput profiling of methylation status of promoter regions of genes

(75) Inventors: Xianqiang Li, Palo Alto, CA (US); Xin Jiang, Saratoga, CA (US)

Correspondence Address: QUINE INTELLECTUAL PROPERTY LAW GROUP, P.C., P O BOX 458, ALAMEDA, CA, 94501, US

(73) Assignee: Panomics, Inc., Fremont, CA (US)

(21) Appl. No.: 11/634,359

(22) Filed: Dec. 4, 2006

Related U.S. Application Data
(60) Provisional application No. 60/742,775, filed on Dec. 5, 2005.

(51) Int. Cl. C12Q 1/68; C12P 19/34

(52) U.S. Cl. 435/6; 435/91.2

(57) ABSTRACT

Rapid, sensitive, reproducible high-throughput methods for detecting methylation patterns in samples of nucleic acid, especially in the promoter region of genes which are enriched with CpG islands, are provided. The methods include isolating complexes of methylated DNA and methylation binding protein, optionally amplifying the isolated methylated DNA, and detecting the methylated DNA or its amplification products in a multiplex and robust manner. By using the inventive methodology, methylated and unmethylated sequences present in the original sample of nucleic acid can be distinguished. By profiling and comparing the methylation status of genes in different samples, one can utilize the information for diagnosis and treatment of diseases or conditions associated with aberrant DNA hypermethylation or hypomethylation.

### CROSS-REFERENCE TO RELATED APPLICATIONS

This application is a non-provisional utility patent application claiming priority to and benefit of the following prior provisional patent application: U.S. Ser. No. 60/742,775, filed Dec. 5, 2005, entitled “HIGH THROUGHPUT PROFILING OF METHYLATION STATUS OF PROMOTER REGIONS OF GENES” by Xianqiang Li et al., which is incorporated herein by reference in its entirety for all purposes.

### FIELD OF THE INVENTION

The present invention relates to detection of the methylation status of nucleic acids. In particular, methods in which methylated nucleic acids are isolated from unmethylated nucleic acids and then identified are described. Related compositions and kits are also provided.

### BACKGROUND OF THE INVENTION

DNA methylation is a commonly occurring modification of human DNA. This modification involves the transfer of a methyl group to DNA, a reaction that is catalyzed by DNA methyltransferase (DNMT) enzymes. Typically, DNA methylation involves the addition of a methyl group to cytosine residues at CpG dinucleotides. CpG dinucleotides are gathered in clusters called CpG islands, which are unequally distributed across the human genome. While methylation at the carbon 5 position of cytosine residues in CpG dinucleotides is the most common type of methylation in humans and other eukaryotes, methylation can also occur, for example, at CpA and CpT dinucleotides, at the N4 position of cytosine, and at the N6 position of adenine.

The methylation reaction that results in methylation of cytosine at carbon 5 involves flipping a target cytosine out of an intact double helix to allow the transfer of a methyl group from S-adenosylmethionine in a cleft of the enzyme DNA (cytosine-5)-methyltransferase (Klimasauskas et al., Cell 76:357-369, 1994) to form 5-methylcytosine (5-mCyt). This enzymatic conversion is the most common epigenetic modification of DNA known to exist in vertebrates and is essential for normal embryonic development (Bird, Cell 70:5-8, 1992; Laird and Jaenisch, Human Mol. Genet. 3:1487-1495, 1994; and Li et al., Cell 69:915-926, 1992). The presence of 5-mCyt at CpG dinucleotides has resulted in a 5-fold depletion of this sequence in the genome during vertebrate evolution, presumably due at least in part to spontaneous deamination of 5-mCyt to T and the consequent hypermutability of such sequences (Schoreret et al., Proc. Natl. Acad. Sci. USA 89:957-961, 1992). Those areas of the genome that do not show such suppression are referred to as “CpG islands” (Bird, Nature 321:209-213, 1986; and Gardiner-Garden et al., J. Mol. Biol. 196:261-282, 1987). These CpG island regions comprise about 1% of vertebrate genomes and also account for about 15% of the total number of CpG dinucleotides (Bird, Nature 321:209-213, 1986). CpG islands are typically between 0.2 to about 1 kb in length and are located upstream of many housekeeping and tissue-specific genes, but may also extend into gene coding regions. Methylation of cytosine residues within CpG islands in somatic tissues is believed to affect gene function by altering transcription (Cedar, Cell 53:3-4, 1988).

Methylation of cytosine residues contained within CpG islands of certain genes has been inversely correlated with gene activity. Some studies have demonstrated an inverse correlation between methylation of CpG islands and gene expression, however, most CpG islands on autosomal genes remain unmethylated in the germline and methylation of these islands is usually independent of gene expression. Tissue-specific genes are usually unmethylated in the receptive target organs but are methylated in the germline and in non-expressing adult tissues. CpG islands of constitutively-expressed housekeeping genes are normally unmethylated in the germline and in somatic tissues. Methylation may lead to decreased gene expression by a variety of mechanisms including, for example, disruption of local chromatin structure, inhibition of transcription factor-DNA binding, or recruitment of proteins which interact specifically with methylated sequences indirectly preventing transcription factor binding. While there are several theories as to how methylation affects mRNA transcription and gene expression, the exact mechanism of action is not completely understood.

It is considered that an altered DNA methylation pattern, particularly methylation of cytosine residues, causes genome instability and is mutagenic. This, presumably, has led to an 80% suppression of CpG methyl acceptor sites in eukaryotic organisms which methylate their genomes. Cytosine methylation further contributes to generation of polymorphism and germ line mutations and to transition mutations that can inactivate tumor-suppressor genes (Jones, Cancer Res. 56:2463-2467, 1996). Abnormal methylation of CpG islands associated with tumor suppressor genes may also cause decreased gene expression. Increased methylation of such regions may lead to progressive reduction of normal gene expression resulting in the selection of a population of cells having a selective growth advantage (i.e., a malignancy). Ushijima et al. (Proc. Natl. Acad. Sci. USA 94:2284-2289, 1997) characterized and cloned DNA fragments that show methylation changes during murine hepatocarcinogenesis. Data from a group of studies of altered methylation sites in cancer cells show that it is not simply the overall levels of DNA methylation that are altered in cancer, but changes in the distribution of methyl groups.

Research shows that a family of proteins selectively recognize methylated CpGs. The binding of these proteins to DNA leads to an altered chromatin structure, which subsequently prevents the binding of transcription machinery, and thus precludes gene expression. The abnormal methylation causes transcriptional repression of numerous genes, leading to tumor growth and development.

These studies suggest that methylation at CpG-rich sequences, known as CpG islands, provide an alternative pathway for the inactivation of tumor suppressors. Methylation of CpG oligonucleotides in the promoters of tumor suppressor genes can lead to their inactivation. Other studies provide data that alterations in the normal methylation process are associated with genomic instability (Lengauer et al. Proc. Natl. Acad. Sci. USA 94:2545-2550, 1997). Such abnormal epigenetic changes may be found in many types of cancer and can serve as potential markers for oncogenic transformation, provided that there is a reliable means for rapidly determining such epigenetic changes.

There has been a delay in the appreciation of methylation as an important epigenetic event in cancer progression. This has been due to the difficulties associated with the analysis of DNA methylation, as standard molecular biology techniques do not preserve methylation of the genomic DNA.

There are a variety of genome scanning methods that have been used to identify altered methylation sites in cancer cells. For example, one method involves restriction landmark genomic scanning (Kawai et al., Mol. Cell. Biol. 14:7421-7427, 1994), and another example involves methylation-sensitive arbitrarily primed PCR (Gonzalgo et al., Cancer Res. 57:594-599, 1997). Changes in methylation patterns at specific CpG sites have been monitored by digestion of genomic DNA with methylation-sensitive restriction enzymes followed by Southern analysis of the regions of interest (digestion-Southern method). The digestion-Southern method is a straightforward method, but it has inherent disadvantages in that it is time consuming and requires a large amount of high molecular weight. DNA (at least 5 μg) and has a limited scope for analysis of CpG sites (as determined by the presence of recognition sites for methylation-sensitive restriction enzymes).

Another method for analyzing changes in methylation patterns involves a PCR-based process that involves digestion of genomic DNA with methylation-sensitive restriction enzymes prior to PCR amplification (Singer-Sam et al., Nucl. Acids Res. 18:687, 1990). However, this method has not been shown effective because of a high degree of false positive signals (methylation present) due to inefficient enzyme digestion or overamplification in a subsequent PCR reaction.

Genomic sequencing has been simplified for analysis of DNA methylation patterns and 5-methylcytosine distribution by using bisulfite treatment (Frommer et al., Proc. Natl. Acad. Sci. USA 89:1827-1831, 1992). Bisulfite treatment of DNA distinguishes methylated from unmethylated cytosines, but original bisulfite genomic sequencing requires large-scale sequencing of multiple plasmid clones to determine overall methylation patterns, which prevents this technique from being commercially useful for determining methylation patterns in any type of a routine diagnostic assay.

In addition, other techniques have been reported which utilize bisulfite treatment of DNA as a starting point for methylation analysis. These include methylation-specific PCR (MSP) (Herman et al. Proc. Natl. Acad. Sci. USA 93:9821-9826, 1992) and restriction enzyme digestion of PCR products amplified from bisulfite-converted DNA (Sadri and Hornsby, Nucl. Acids Res. 24:5058-5059, 1996; and Xiong and Laird, Nucl. Acids Res. 25:2532-2534, 1997).

PCR techniques have been developed for detection of gene mutations (Kuppuswamy et al., Proc. Natl. Acad. Sci. USA 88:1143-1147, 1991) and quantitation of allelic-specific expression (Szabo and Mann, Genes Dev. 9:3097-3108, 1995; and Singer-Sam et al., PCR Methods Appl. 1:160-163, 1992). Such techniques use internal primers, which anneal to a PCR-generated template and terminate immediately 5′ of the single nucleotide to be assayed. However, an allelic-specific expression technique has not been tried within the context of assaying for DNA methylation patterns.

Most molecular biological techniques used to analyze specific loci, such as CpG islands in complex genomic DNA, involve some form of sequence-specific amplification, whether it is biological amplification by cloning in E. coli, direct amplification by PCR, or signal amplification by hybridization with a probe that can be visualized. Since DNA methylation is added post-replicatively by a dedicated maintenance DNA methyltransferase that is not present in either E. coli or in the PCR reaction, such methylation information is lost during molecular cloning or PCR amplification. Moreover molecular hybridization does not discriminate between methylated and unmethylated DNA, since the methyl group on the cytosine does not participate in base pairing. The lack of a facile way to amplify the methylation information in complex genomic DNA has probably been a most important impediment to DNA methylation research. Therefore, there is a need in the art to improve upon methylation detection techniques, especially in a quantitative manner.

The indirect methods for DNA methylation pattern determinations at specific loci that have been developed rely on techniques that alter the genomic DNA in a methylation-dependent manner before the amplification event. There are two primary methods that have been utilized to achieve this methylation-dependent DNA alteration. The first is digestion by a restriction enzyme that is affected in its activity by 5-methylcytosine in a CpG sequence context. The cleavage, or lack of it, can subsequently be revealed by Southern blotting or by PCR. The other technique that has received recent widespread use is the treatment of genomic DNA with sodium bisulfite. Sodium bisulfite treatment converts all unmethylated cytosines in the DNA to uracil by deamination, but leaves the methylated cytosine residues intact. Subsequent PCR amplification replaces the uracil residues with thymines and the 5-methylcytosine residues with cytosines. The resulting sequence difference has been detected using standard DNA sequence detection techniques, primarily PCR.

Many DNA methylation detection techniques utilize bisulfite treatment. Currently, bisulfite treatment-based methods involve bisulfite treatment followed by a PCR reaction to analyze specific loci within the genome. There are two principally different ways in which the sequence difference generated by the sodium bisulfite treatment can be revealed. The first is to design PCR primers that uniquely anneal with either methylated or unmethylated converted DNA. This technique is referred to as “methylation specific PCR” or “MSP”. The method used by other bisulfite-based techniques (such as bisulfite genomic sequencing, COBRA and Ms-SNuPE) is to amplify the bisulfite-converted DNA using primers that anneal at locations that lack CpG dinucleotides in the original genomic sequence. In this way, the PCR primers can amplify the sequence in between the two primers, regardless of the DNA methylation status of that sequence in the original genomic DNA. This results in a pool of different PCR products, all with the same length and differing in their sequence only at the sites of potential DNA methylation at CpGs located in between the two primers. The difference between these methods of processing the bisulfite-converted sequence is that in MSP, the methylation information is derived from the occurrence or lack of occurrence of a PCR product, whereas in the other techniques a mix of products is generated and the mixture is subsequently analyzed to yield quantitative information on the relative occurrence of the different methylation states. This method is very tedious and inconsistent, and all of the conventional methods are time consuming and only allow the analysis of one promoter at a time.

Therefore, there is a need in the art for reliable and rapid (high-throughput) methods for determining the methylation status of nucleic acids, for example, the methylation status of genomic nucleic acids from organisms where methylation is the preferred epigenetic alteration.

### SUMMARY OF THE INVENTION

The present invention provides methods for determining the methylation status of nucleic acids, including, for example, the methylation status of CpG islands within a sample of genomic DNA. The methods are optionally multiplexed and used to determine the methylation status of multiple nucleic acids simultaneously. Methods for diagnosing and/or treating diseases or conditions associated with aberrant methylation are also described. Compositions, kits, and systems related to the methods are provided.

In one aspect of the invention, a method is provided for detecting methylation status of one or more nucleic acids. The method comprises contacting a sample of nucleic acid comprising or suspected of comprising one or more methylated nucleic acids with a methylation binding protein (MBP), forming one or more methylated nucleic acid-MBP complexes, isolating the methylated nucleic acid-MBP complexes, and detecting the presence of the one or more methylated nucleic acids in the isolated methylated nucleic acid-MBP complexes. The presence of the methylated nucleic acid(s) in the isolated methylated nucleic acid-MBP complexes is preferably determined by a technique other than nucleic acid sequencing or target-specific PCR amplification.

In a preferred embodiment, the sample of the nucleic acid contains multiple different nucleic acid molecules with different sequences and different methylation patterns. The sample optionally comprises a plurality of genomic DNA fragments, e.g., a plurality of genomic DNA fragments in which at least one fragment contains a methylated CpG island wherein at least one of the cytosine residues is methylated at the 5 position. For example, a sample containing methylated genomic DNA can be digested with a restriction enzyme to produce DNA fragments, some of which contain methylated base residues (such as methylated CpG islands or other methylated residues).

As noted, the sample of nucleic acid is contacted with an MBP, which forms complexes with methylated nucleic acids (e.g., methylated DNA fragments). The methylated nucleic acid-MBP complexes are isolated from other (unmethylated and uncomplexed with MBP) nucleic acids in the sample, for example, by using a filter column in which a membrane retains the nucleic acid-MBP complexes. In one class of embodiments, the methylated nucleic acid-MBP complexes are isolated from other nucleic acids in the sample by binding the methylated nucleic acid-MBP complexes to a nitrocellulose membrane and washing the other nucleic acids away from the membrane-bound methylated nucleic acid-MBP complexes; the nitrocellulose membrane is optionally the filter in a filter column, e.g., a spin column or multiwell filter plate. Exemplary MBPs include, but are not limited to, an MBP comprising a methyl-CpG binding domain from mouse or human methyl CpG binding protein 2 (MeCP2) or a homolog thereof.

The methylated nucleic acids in the isolated complexes are optionally amplified (e.g., by PCR) and are detected by various methods, preferably by using a hybridization array to simultaneously detect multiple different nucleic acids (e.g., multiple different DNA fragments) containing methylated base residues, by capturing the nucleic acids to particles and then detecting them, and/or by using a branched DNA assay.

Thus, in one class of embodiments, the presence of the methylated nucleic acids in the isolated methylated nucleic acid-MBP complexes is detected with a nucleic acid hybridization array on which different nucleic acid hybridization probes with predetermined sequences are immobilized in discrete, different positions. The methylated nucleic acids can be hybridized to the array, e.g., after being labeled, or they can be amplified and the resulting amplified products hybridized to the array. The method optionally includes simultaneously amplifying the one or more methylated nucleic acids from the isolated methylated nucleic acid-MBP complexes (for example, using universal primers complementary to adaptors added to each of the methylated nucleic acids) to provide one or more amplified nucleic acids. In one class of embodiments, the amplified nucleic acids are contacted with a nucleic acid hybridization array, on which array different nucleic acid hybridization probes with predetermined sequences are immobilized at discrete, different positions, and hybridized with complementary nucleic acid hybridization probes, thereby capturing different amplified nucleic acids at different positions on the array. Which position(s) on the array have an amplified nucleic acid hybridized thereto is then determined, thereby determining which methylated nucleic acid(s) were present in the sample. The amount of nucleic acid captured on the array is optionally quantitated and correlated with an amount of methylated nucleic acid present in the original sample. The amplified nucleic acids are optionally labeled, for example, during or after the amplification. In one embodiment, biotin is incorporated into the amplified nucleic acids during the amplifying step, and which positions on the array have an amplified nucleic acid hybridized thereto is detected by binding a streptavidin-conjugated horseradish peroxidase enzyme to the biotin and then detecting a luminescent product of the enzyme. It will be evident that other streptavidin-conjugated moieties (e.g., streptavidin-conjugated enzymes or fluorophores) can similarly be employed, and that fluorophores or other labels can be incorporated directly into the amplified nucleic acids during the amplifying step and then detected.

In the embodiments described above, different methylated nucleic acids are captured at different positions on an array by hybridization to different nucleic acid hybridization probes that are immobilized on the array. In another aspect, different methylated nucleic acids are captured to different, distinguishable sets of particles instead of to different positions on a spatially addressable solid support. Thus, in one class of embodiments, a pooled population of particles is provided. The population includes one or more subsets of particles (typically, one subset for each nucleic acid whose methylation state is to be detected). The particles in each subset are distinguishable from the particles in the other subsets, and the particles in different subsets have associated therewith different nucleic acid hybridization probes with predetermined sequences. The one or more methylated nucleic acids from the isolated methylated nucleic acid-MBP complexes (or complements or copies thereof, e.g., produced by amplification of the methylated nucleic acids) are contacted with the pooled population of particles. The one or more methylated nucleic acids (or the complements or copies thereof) are hybridized with complementary nucleic acid hybridization probes, thereby capturing different methylated nucleic acids (or complements or copies thereof) to different subsets of particles. Which subsets of particles have nucleic acid captured on the particles is then detected, thereby indicating which methylated nucleic acids were present in the sample.

In one class of embodiments, the particles are microspheres. The microspheres of each subset can be distinguishable from those of the other subsets, e.g., on the basis of their fluorescent emission spectrum, their diameter, or a combination thereof.

In one aspect, the presence of the methylated nucleic acids in the isolated methylated nucleic acid-MBP complexes is detected with a branched DNA (bDNA) assay. Thus, in one class of embodiments, the methylated nucleic acids from the isolated methylated nucleic acid-MBP complexes are captured on a solid support. One or more subsets of m label extenders are provided, wherein m is at least two, and wherein each subset of m label extenders is capable of hybridizing to one of the methylated nucleic acids. A label probe system comprising a label, wherein a component of the label probe system is capable of hybridizing to the label extenders, is also provided. Each methylated nucleic acid captured on the solid support is hybridized to its corresponding subset of m label extenders, and the label probe system is hybridized to the label extenders. The presence or absence of the label on the solid support is then detected.

The bDNA assay is optionally a singleplex assay, used to detect the presence or absence of a single methylated nucleic acid in the sample. Thus, in one embodiment, the methylation status of one nucleic acid is to be detected, and the methylated nucleic acid is captured on the solid support by hybridizing it to n capture extenders, wherein n is at least two, and then hybridizing the capture extenders with a capture probe that is bound to the solid support (covalently or noncovalently).

Alternatively, the bDNA assay is a multiplex assay, used to simultaneously detect the presence or absence of two or more methylated nucleic acids in the sample. For example, in one class of embodiments in which the methylation status of two or more nucleic acids is to be detected, the methylated nucleic acids are captured to different subsets of particles by providing a pooled population of particles which constitute the solid support, the population comprising two or more subsets of particles, the particles in each subset being distinguishable from the particles in the other subsets, and the particles in each subset having associated therewith a different capture probe; providing two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected subset of the particles; and hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the subset of particles with which the capture extenders are associated. At least a portion of the particles from each subset are identified and the presence or absence of the label on those particles is detected. Since a correlation exists between a particular subset of particles and a particular methylated nucleic acid, which subsets of particles have the label present indicates which of the methylated nucleic acids were present in the sample.

In another exemplary class of embodiments in which the methylation status of two or more nucleic acids is to be detected, the methylated nucleic acids are captured to different positions on a spatially addressable solid support. In this class of embodiments, the solid support is preferably substantially planar, and comprises two or more capture probes, each of which is provided at a selected position on the solid support. The methylated nucleic acids are captured on the solid support by providing two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected position on the solid support; and hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the solid support at the selected position with which the capture extenders are associated. The presence or absence of the label at the selected positions on the solid support is then detected. Since a correlation exists between a particular position on the support and a particular methylated nucleic acid, which positions have a label present indicates which of the methylated nucleic acids were present in the sample.

The label probe system optionally includes an amplification multimer and a plurality of label probes, wherein the amplification multimer is capable of hybridizing to a label extender and to a plurality of label probes. As another example, the label probe system optionally includes a preamplifier, an amplification multimer and a label probe, where the preamplifier is capable of hybridizing simultaneously to a label extender and to a plurality of amplification multimers, and where the amplification multimer is capable of hybridizing simultaneously to the preamplifier and to a plurality of label probes. In one class of embodiments, the label probe comprises the label. In one aspect, the label is a fluorescent label, and detecting the presence of the label (e.g., on the particles or the spatially addressable solid support) comprises detecting a fluorescent signal from the label. Optionally, detecting the presence of the label on the support comprises measuring an intensity of a signal from the label, and the method includes correlating the intensity of the signal with a quantity of the corresponding methylated nucleic acid present.

In one aspect of the invention, a method for detecting methylation status of a plurality of genomic DNA fragments is provided. In the method, a sample of nucleic acid comprising or suspected of comprising the plurality of genomic DNA fragments is contacted with a methylation binding protein (MBP), and methylated DNA-MBP complexes are formed and isolated. With a nucleic acid hybridization array on which different nucleic acid hybridization probes with predetermined sequences are immobilized in discrete, different positions, the presence of the methylated DNAs in the isolated methylated DNA-MBP complexes is detected, thereby indicating which of the genomic DNA fragments in the sample were methylated.

Essentially all of the features noted for the methods above apply to these embodiments as well, as relevant; for example, with respect to detection of cytosines methylated at the carbon 5 position and/or within CpG islands, type of MBP employed, isolation of the methylated DNA-MBP complexes using a nitrocellulose membrane and/or a filter column, and the like. For example, it is worth noting that the methylated DNAs from the isolated methylated DNA-MBP complexes are optionally amplified, preferably simultaneously, to provide one or more amplified DNAs, which are then contacted with the nucleic acid hybridization array and hybridized with complementary nucleic acid hybridization probes, thereby capturing different amplified DNAs at different positions on the array; which positions on the array have an amplified DNA hybridized thereto is then determined, thereby determining which methylated DNAs were present in the sample and therefore which of the genomic DNA fragments in the sample were methylated.

In another aspect of the invention, a method for detecting methylation status of one or more nucleic acids is provided. In the method, a sample comprising or suspected of comprising one or more methylated nucleic acids is contacted with an MBP, and one or more methylated nucleic acid-MBP complexes are formed and isolated. A pooled population of particles comprising one or more subsets of particles is provided. The particles in each subset are distinguishable from the particles in the other subsets, and the particles in different subsets have associated therewith different nucleic acid hybridization probes with predetermined sequences. The one or more methylated nucleic acids from the isolated methylated nucleic acid-MBP complexes (or complements or copies thereof) are contacted with the pooled population of particles, and the one or more methylated nucleic acids (or the complements or copies thereof) are hybridized with complementary nucleic acid hybridization probes, thereby capturing different methylated nucleic acids (or complements or copies thereof) to different subsets of particles. Which subsets of particles have nucleic acid captured on the particles is detected, thereby indicating which methylated nucleic acids were present in the sample.

Essentially all of the features noted for the methods above apply to these embodiments as well, as relevant; for example, with respect to optional amplification of the nucleic acids from the isolated nucleic acid-MBP complexes, detection of cytosines methylated at the carbon 5 position and/or within CpG islands, type of MBP employed, isolation of the methylated DNA-MBP complexes using a nitrocellulose membrane and/or a filter column, type of particles, and the like.

In one aspect of the invention, as noted, the presence of the methylated nucleic acids in the isolated methylated nucleic acid-MBP complexes is detected with a branched DNA (bDNA) assay. Accordingly, one general class of embodiments provides a method for detecting methylation status of one or more nucleic acids, in which a sample comprising or suspected of comprising one or more methylated nucleic acids is contacted with an MBP, one or more methylated nucleic acid-MBP complexes are formed and isolated, and the methylated nucleic acids from the isolated methylated nucleic acid-MBP complexes are captured on a solid support. One or more subsets of m label extenders, wherein m is at least two, and wherein each subset of m label extenders is capable of hybridizing to one of the methylated nucleic acids, is provided, as is a label probe system comprising a label, wherein a component of the label probe system is capable of hybridizing to the label extenders. Each methylated nucleic acid captured on the solid support is hybridized to its corresponding subset of m label extenders, and the label probe system is hybridized to the label extenders. The presence or absence of the label on the solid support is detected, and thereby the presence or absence of the methylated nucleic acids on the solid support and in the sample is detected.

The bDNA assay is optionally a singleplex assay, used to detect the presence or absence of a single methylated nucleic acid in the sample. Thus, in one embodiment, the methylation status of one nucleic acid is to be detected, and the methylated nucleic acid is captured on the solid support by hybridizing it to n capture extenders, wherein n is at least two, and then hybridizing the capture extenders with a capture probe that is bound to the solid support (covalently or noncovalently).

Alternatively, the bDNA assay is a multiplex assay, used to simultaneously detect the presence or absence of two or more methylated nucleic acids in the sample. For example, in one class of embodiments in which the methylation status of two or more nucleic acids is to be detected, the methylated nucleic acids are captured to different subsets of particles by providing a pooled population of particles which constitute the solid support, the population comprising two or more subsets of particles, the particles in each subset being distinguishable from the particles in the other subsets, and the particles in each subset having associated therewith a different capture probe; providing two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected subset of the particles; and hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the subset of particles with which the capture extenders are associated. At least a portion of the particles from each subset are identified and the presence or absence of the label on those particles is detected. Since a correlation exists between a particular subset of particles and a particular methylated nucleic acid, which subsets of particles have the label present indicates which of the methylated nucleic acids were present in the sample.

In another exemplary class of embodiments in which the methylation status of two or more nucleic acids is to be detected, the methylated nucleic acids are captured to different positions on a spatially addressable solid support. In this class of embodiments, the solid support is preferably substantially planar, and comprises two or more capture probes, each of which is provided at a selected position on the solid support. The methylated nucleic acids are captured on the solid support by providing two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected position on the solid support; and hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the solid support at the selected position with which the capture extenders are associated. The presence or absence of the label at the selected positions on the solid support is then detected. Since a correlation exists between a particular position on the support and a particular methylated nucleic acid, which positions have a label present indicates which of the methylated nucleic acids were present in the sample.

Essentially all of the features noted for the methods above apply to these embodiments as well, as relevant; for example, with respect to detection of cytosines methylated at the carbon 5 position and/or within CpG islands, type of MBP employed, isolation of the methylated DNA-MBP complexes using a nitrocellulose membrane and/or a filter column, type of particles, and the like. For example, it is worth noting that the label probe system optionally includes an amplification multimer and a plurality of label probes, wherein the amplification multimer is capable of hybridizing to a label extender and to a plurality of label probes. As another example, the label probe system optionally includes a preamplifier, an amplification multimer and a label probe, where the preamplifier is capable of hybridizing simultaneously to a label extender and to a plurality of amplification multimers and where the amplification multimer is capable of hybridizing simultaneously to the preamplifier and to a plurality of label probes. In one class of embodiments, the label probe comprises the label. In one aspect, the label is a fluorescent label, and detecting the presence of the label (e.g., on the particles or the spatially addressable solid support) comprises detecting a fluorescent signal from the label. Optionally, detecting the presence of the label on the support comprises measuring an intensity of a signal from the label, and the method includes correlating the intensity of the signal with a quantity of the corresponding methylated nucleic acid present.

In another aspect of the invention, a method is provided for diagnosing a disease or condition associated with aberrant hypermethylation or hypomethylation, such as cancer or a hematological disorder. The method comprises contacting a sample of nucleic acid containing methylated nucleic acid or suspected of containing methylated nucleic acid with an MBP, wherein the sample of nucleic acid is derived from a sample of cells from a patient having or suspected of having a disease or condition associated with aberrant hypermethylation or hypomethylation; forming a methylated nucleic acid-MBP complex; isolating the methylated nucleic acid-MBP complex; detecting levels of the methylated nucleic acid in the isolated methylated nucleic acid-MBP complex, preferably with a technique other than nucleic acid sequencing or target-specific PCR amplification; and comparing levels of methylated nucleic acid with that of a reference sample containing nucleic acid derived from normal or healthy cells or from cells from a different sample, wherein an increase in the levels of methylated nucleic acid indicates that the patient has a disease associated with aberrant hypermethylation or wherein a decrease in the levels of methylated nucleic acid indicates that the patient has a disease associated with aberrant hypomethylation. Essentially all of the features noted for the methods above apply to these embodiments as well, as relevant.

In yet another aspect of the invention, a method is provided for treating a disease or condition associated with aberrant hypermethylation, such as cancer or a hematological disorder. The method comprises contacting a sample of nucleic acid containing methylated nucleic acid or suspected of containing methylated nucleic acid with an MBP, wherein the sample of nucleic acid is derived from a sample of cells from a patient having a disease or condition associated with aberrant hypermethylation; forming a methylated nucleic acid-MBP complex; isolating the methylated nucleic acid-MBP complex; detecting the presence of the methylated nucleic acid in the isolated methylated nucleic acid-MBP complex, preferably with a technique other than nucleic acid sequencing or target-specific PCR amplification; comparing the pattern of methylated nucleic acid with that of a reference sample containing nucleic acid derived from normal or healthy cells or from cells from a different sample; and treating the patient with a therapeutic agent that inhibits hypermethylation of DNA in the cells, such as 5-azacytidine (or azacytidine) and 5-aza-2′-deoxycytidine (or decitabine). Essentially all of the features noted for the methods above apply to these embodiments as well, as relevant.

Compositions and kits are also provided for performing the methods described herein. For example, in one embodiment, a kit for detecting one or more methylated nucleic acids is provided which comprises a methylation binding protein (MBP), a separation column for separating MBP-nucleic acid complexes from non-complexed nucleic acid, and instructions for separating MP-nucleic acid complexes from non-complexed nucleic acid by the separation column (e.g., a column comprising a nitrocellulose membrane). The kit can also comprise an array of predetermined, different nucleic acid hybridization probes immobilized on a surface of a substrate such that the hybridization probes are positioned in different defined regions on the surface. Preferably, each of the different nucleic acid hybridization probes comprises a different nucleic acid probe capable of hybridizing to a different region or fragment of a gene, preferably a promoter region of a gene, more preferably a promoter region of a gene listed in Table 1 (i.e., hybridizing to one of SEQ ID NOs:1-82 or a complement thereof). Most preferably, the array of predetermined, different nucleic acid hybridization probes comprises at least two different nucleic acid probes which are capable of separately hybridizing to at least two promoter regions of the genes listed in Table 1 (i.e., to at least two of SEQ ID NOs:1-82 or a complement thereof). The kit can be used for performing the methods provided in the present invention, and the instructions can include instructions on how to perform the methods. The kit optionally includes buffered solutions (e.g., for washing the separation column, eluting nucleic acid from the separation column, washing the array, or the like), a restriction enzyme, oligonucleotide adaptors and/or primers, PCR reagents (e.g., a thermostable DNA polymerase, nucleoside triphosphates, and the like), detection reagents (e.g., streptavidin-conjugated horseradish peroxidase and a luminescent substrate), and/or the like. Essentially all of the features noted for the methods above apply to these embodiments as well, as relevant

In another embodiment, a kit for detecting one or more methylated nucleic acids is provided which comprises a methylation binding protein (MBP), a nitrocellulose membrane, one or more subsets of m label extenders, wherein m is at least two and wherein each subset of m label extenders is capable of hybridizing to one of the methylated nucleic acids, and a label probe system comprising a label, wherein a component of the label probe system is capable of hybridizing to the label extenders. The kit also includes i) 1) a solid support comprising a capture probe and 2) a subset of n capture extenders, wherein n is at least two, wherein the subset of n capture extenders is capable of hybridizing to a methylated nucleic acid and is capable of hybridizing to the capture probe and thereby associating the capture extenders with the solid support; ii) 1) a pooled population of particles, the population comprising two or more subsets of particles, a plurality of the particles in each subset being distinguishable from a plurality of the particles in every other subset, and the particles in each subset having associated therewith a different capture probe, and 2) two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected subset of the particles; or iii) 1) a solid support comprising two or more capture probes, wherein each capture probe is provided at a selected position on the solid support, and 2) two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected position on the solid support. The components of the kit are packaged in one or more containers. The kit optionally includes a filter column (e.g., a spin column or a multiwell plate) comprising the nitrocellulose membrane, buffered solutions (e.g., for washing the filter column, eluting nucleic acid from the filter column, washing the particles or other solid support, or the like), a restriction enzyme, and/or the like. Essentially all of the features noted for the embodiments above apply to these embodiments as well, as relevant, for example, with respect to composition of the label probe system.

### BRIEF DESCRIPTION OF THE DRAWINGS

FIG. 1 Panel A illustrates a method of isolating and detecting methylated nucleic acid fragments by using a methylation binding protein (MBP) according to the present invention. Panel B illustrates an embodiment of an inventive method for high throughput detection of the methylation status of multiple genes, for example, in the promoter regions of the genes, using a nucleic acid hybridization array.

FIG. 2 shows a diagram of a DNA array for 82 different promoter regions of genes, the sequences of which are listed in Table 1.

FIG. 3 shows results of detection of methylation status of genes in normal and breast cancer cell lines: Hs 578Bst (Panel A); Hs 578T (Panel B); and MCF7 (Panel C). The promoter regions of specific genes detected to be methylated are identified individually.

FIG. 4 schematically illustrates isolation and detection of methylated nucleic acid fragments, using a methylation binding protein and a singleplex branched DNA (bDNA) assay.

FIG. 5 Panels A-E schematically depict a multiplex bDNA assay, in which methylated nucleic acids are captured on distinguishable subsets of microspheres and then detected.

FIG. 6 Panels A-D schematically depict a multiplex bDNA assay, in which methylated nucleic acids are captured at selected positions on a solid support and then detected. Panel A shows a top view of the solid support, while Panels B-D show the support in cross-section.

FIG. 7 shows results of detection of methylation status of genes in MCF7, T47D, and 1806 cell lines using a bDNA assay.

FIG. 8 Panels A and B show results of detection of methylation status of genes in an MCF7 breast cancer cell line. Results of detection using a hybridization array are shown in Panel A, and results of detection using a bDNA assay are shown in Panel B.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a molecule” includes a plurality of such molecules, and the like.

The term “polynucleotide” (and the equivalent term “nucleic acid”) encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking, and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, N.Y.), as well as in Ausubel, infra.

A first polynucleotide that is “capable of hybridizing” (or “configured to hybridize”) to a second polynucleotide comprises a first polynucleotide sequence that is complementary to a second polynucleotide sequence in the second polynucleotide.

The term “complementary” refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A “capture extender” or “CE” is a polynucleotide that is capable of hybridizing to a nucleic acid of interest (e.g., a methylated nucleic acid) and that is preferably also capable of hybridizing to a capture probe. The capture extender typically has a first polynucleotide sequence C-1, which is complementary to the capture probe, and a second polynucleotide sequence C-3, which is complementary to a polynucleotide sequence of the nucleic acid of interest. Sequences C-1 and C-3 are typically not complementary to each other. The capture extender is preferably single-stranded.

A “capture probe” or “CP” is a polynucleotide that is capable of hybridizing to at least one capture extender and that is tightly bound (e.g., covalently or noncovalently, directly or through a linker, e.g., streptavidin-biotin or the like) to a solid support, a spatially addressable solid support, a slide, a particle, a microsphere, or the like. The capture probe typically comprises at least one polynucleotide sequence C-2 that is complementary to polynucleotide sequence C-1 of at least one capture extender. The capture probe is preferably single-stranded.

A “label extender” or “LE” is a polynucleotide that is capable of hybridizing to a nucleic acid of interest (e.g., a methylated nucleic acid) and to a label probe system. The label extender typically has a first polynucleotide sequence L-1, which is complementary to a polynucleotide sequence of the nucleic acid of interest, and a second polynucleotide sequence L-2, which is complementary to a polynucleotide sequence of the label probe system (e.g., L-2 can be complementary to a polynucleotide sequence of an amplification multimer, a preamplifier, a label probe, or the like). The label extender is preferably single-stranded.

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Many labels are commercially available and can be used in the context of the invention.

A “label probe system” comprises one or more polynucleotides that collectively comprise a label and a polynucleotide sequence M-1, which is capable of hybridizing to at least one label extender. The label provides a signal, directly or indirectly. Polynucleotide sequence M-1 is typically complementary to sequence L-2 in the label extenders. Typically, the label probe system includes a plurality of label probes (e.g., a plurality of identical label probes) and an amplification multimer; it optionally also includes a preamplifier or the like, or optionally includes only label probes, for example.

An “amplification multimer” is a polynucleotide comprising a plurality of polynucleotide sequences M-2, typically (but not necessarily) identical polynucleotide sequences M-2. Polynucleotide sequence M-2 is complementary to a polynucleotide sequence in the label probe. The amplification multimer also includes at least one polynucleotide sequence that is capable of hybridizing to a label extender or to a nucleic acid that hybridizes to the label extender, e.g., a preamplifier. For example, the amplification multimer optionally includes at least one polynucleotide sequence M-1; polynucleotide sequence M-1 is typically complementary to polynucleotide sequence L-2 of the label extenders. Similarly, the amplification multimer optionally includes at least one polynucleotide sequence that is complementary to a polynucleotide sequence in a preamplifier. The amplification multimer can be, e.g., a linear or a branched nucleic acid. As noted for all polynucleotides, the amplification multimer can include modified nucleotides and/or nonstandard internucleotide linkages as well as standard deoxyribonucleotides, ribonucleotides, and/or phosphodiester bonds. Suitable amplification multimers are described, for example, in U.S. Pat. No. 5,635,352, U.S. Pat. No. 5,124,246, U.S. Pat. No. 5,710,264, and U.S. Pat. No. 5,849,481.

A “label probe” or “LP” is a single-stranded polynucleotide that comprises a label (or optionally that is configured to bind to a label) that directly or indirectly provides a detectable signal. The label probe typically comprises a polynucleotide sequence that is complementary to the repeating polynucleotide sequence M-2 of the amplification multimer; however, if no amplification multimer is used in the bDNA assay, the label probe can, e.g., hybridize directly to a label extender.

A “preamplifier” is a nucleic acid that serves as an intermediate between at least one label extender and amplification multimer. Typically, the preamplifier is capable of hybridizing simultaneously to at least one label extender and to a plurality of amplification multimers.

A “microsphere” is a small spherical, or roughly spherical, particle. A microsphere typically has a diameter less than about 1000 micrometers (e.g., less than about 100 micrometers, optionally less than about 10 micrometers).

The term “gene” is used broadly to refer to any nucleic acid associated with a biological function. Genes typically include coding sequences and/or the regulatory sequences required for expression of such coding sequences. The term “gene” applies to a specific genomic sequence, as well as to a cDNA or an mRNA encoded by that genomic sequence. Genes also include non-expressed nucleic acid segments that, for example, form recognition sequences for other proteins. Non-expressed regulatory sequences include “promoters” and “enhancers,” to which regulatory proteins such as transcription factors bind, resulting in transcription of adjacent or nearby sequences.

A “peptide” or “polypeptide” is a polymer comprising two or more amino acid residues (e.g., a protein). The polymer can additionally comprise non-amino acid elements such as labels, quenchers, blocking groups, or the like and can optionally comprise modifications such as glycosylation or the like. The amino acid residues of the polypeptide can be natural or non-natural and can be unsubstituted, unmodified, substituted or modified.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, includes antibodies or fragments either produced by the modification of whole antibodies or synthesized de novo using recombinant DNA methodologies. Antibodies include multiple or single chain antibodies, including single chain Fv (sFv or scFv) antibodies in which a variable heavy and a variable light chain are joined together (directly or through a peptide linker) to form a continuous polypeptide, and humanized or chimeric antibodies, as well as polyclonal and monoclonal antibodies.

A variety of additional terms are defined or otherwise characterized herein.

### DETAILED DESCRIPTION

Among other benefits, the present invention provides rapid, sensitive, and reproducible high-throughput methods for detecting methylation patterns in samples of nucleic acid. For example, the invention provides methods for isolation of methylated DNA, optional amplification thereof, and detection of the methylated DNA or its amplification products in a multiplex and high throughput manner. By using the inventive methodology, methylated and unmethylated sequences present in the original samples of nucleic acid can be distinguished. Related compositions, systems, and kits are also described.

In a preferred aspect, the methods, compositions, and kits of the invention provide for determining the methylation status of CpG islands within samples of genomic DNA, especially in the promoter regions of genes where the DNA is enriched with CpG islands.

According to the methods, compositions, and kits of the present invention, methylated DNA (or other methylated nucleic acid), such as DNA fragments produced by enzymatic digestion of genomic DNA, can be isolated from unmethylated DNA by exploiting the specific binding affinity of methylated DNA to a methylation binding protein (MBP). By forming methylated DNA-MBP complexes, multiple different methylated DNA fragments can be separated from a mixture of DNAs through isolation of the DNA-protein complexes.

As used herein, a “methylation binding protein” or an “MBP” is a protein or peptide that specifically binds to a nucleic acid with one or more methylated base residues, preferably a protein or peptide that binds to methylated CpG islet(s) in a DNA (e.g., to a DNA containing one or more methylated CpG dinucleotides, in preference to a DNA of the same sequence which is not methylated). Examples of MBP include, but are not limited to, the methylated-CpG binding protein 2 (MeCP2) and the methyl-CpG-binding domain proteins MBD1, MBD2, MBD3, and MBD4, and their homologs (preferably with at least 80% sequence identity, more preferably at least 90% sequence identity, and most preferably at least 95% sequence identity, e.g., to human, mouse, or rat MeCP2, MBD1, MBD2, MBD3, or MBD4) that bind to methylated DNA. Exemplary MBPs include, e.g., the methylated DNA binding domains from such proteins (e.g., from MeCP2, MBD1, MBD2, MBD3, or MBD4) and other truncated and/or mutant versions of the proteins as well as the full length wild-type proteins. See review by Ballestar and Wolffe (2001) “Methyl-CpG-binding proteins” Eur. J. Biochem. 268:1-6; Chen et al. (2003) “Derepression of BDNF transcription involves calcium-dependent phosphorylation of MeCP2” Science 302:885-889 and supplemental materials S1-S13; Jorgensen et al. (2006) “Engineering a high-affinity methyl-CpG-binding protein” Nucl Acids Res 34:e96; Gebhard et al. (2006) “Rapid and sensitive detection of CpG-methylation using methyl-binding (MB)-PCR” Nucl Acids Res 34:e82; Gebhard et al. (2006) “Genome-wide profiling of CpG methylation identifies novel targets of aberrant hypermethylation in myeloid leukemia” Cancer Res 66:6118-6128; Cross et al. (1994) “Purification of CpG islands using a methylated DNA binding column” Nature Genetics 6:236-244; Nan et al. (1993) “Dissection of the methyl-CpG binding domain from the chromosomal protein MeCP2” Nucl Acids Res 21:4886-4892; and Brock et al. (2001) “A novel technique for the identification of CpG islands exhibiting altered methylation patterns (ICEAMP)” Nucl Acids Res 29:e123, all of which are herein incorporated by reference. Exemplary MBPs also include antibodies that bind specifically to methylated nucleic acid (see, e.g., Sano et al. (1980) “Identification of 5-methylcytosine in DNA fragments immobilized on nitrocellulose paper” Proc Natl Acad Sci USA 77:3581-3585 and Storl et al. (1979) “Immunochemical detection of N6-methyladenine in DNA” Biochem Biophys Acta 564:23-30), or the MBP can be a polypeptide other than an antibody. Additional MBP sequences can be found, e.g., in Genbank and in the literature.

Methods for Detecting Methylation Status

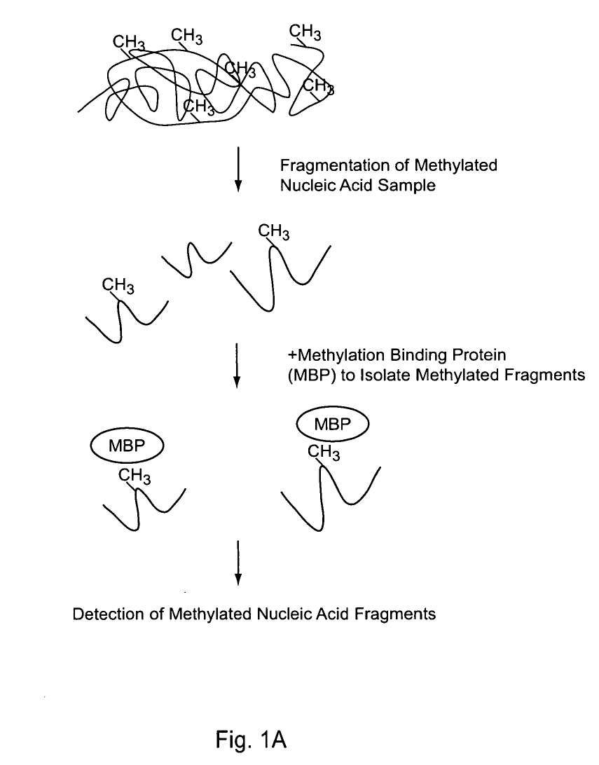

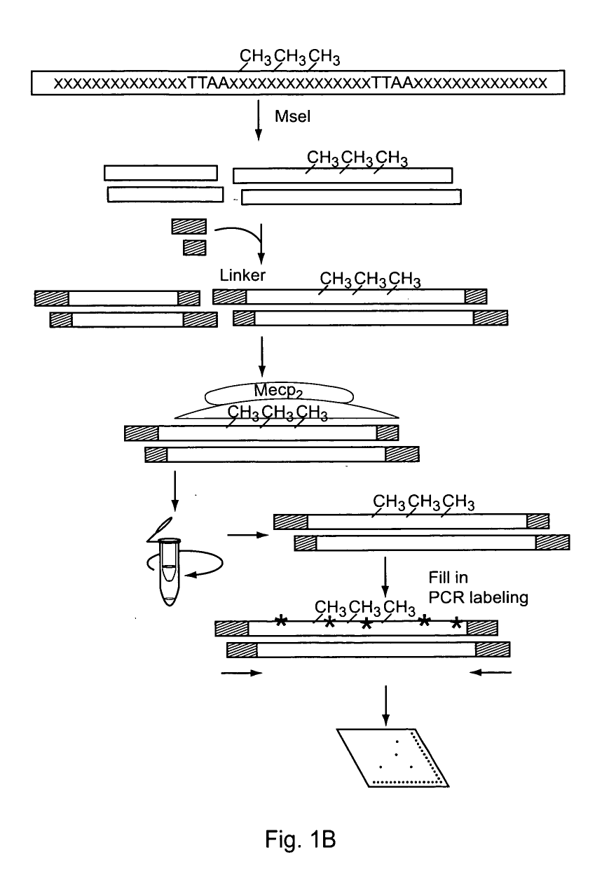

In one aspect of the invention, a method is provided for detecting methylation status of a nucleic acid. The method comprises: contacting a sample of nucleic acid containing methylated nucleic acid or suspected of containing methylated nucleic acid with an MBP; forming a methylated nucleic acid-MBP complex; isolating the methylated nucleic acid-MBP complex; and detecting the presence of the methylated nucleic acid in the isolated methylated nucleic acid-MBP complex. An exemplary embodiment of the method is illustrated in FIG. 1 Panel A, in which the sample of nucleic acid containing methylated nucleic acid or suspected of containing methylated nucleic acid is subjected to fragmentation of the nucleic acid to generate a mixture of nucleic acid fragments with or without methylated base residue(s).

In a preferred embodiment, the sample of nucleic acid contains multiple different nucleic acid molecules with different sequences and different methylation patterns. FIG. 1 Panel B illustrates an exemplary variant of this embodiment. As illustrated in FIG. 1 Panel B, a sample containing methylated genomic DNA is digested with a restriction enzyme (MseI, in the figure) to produce DNA fragments, some of which contain methylated base residues (such as methylated CpG islands in which at least one cytosine residue is methylated at the carbon 5 position). The mixture of DNA fragments is contacted with an MBP such as MeCP2, wherein the MBP forms complexes with methylated DNA fragments. The methylated DNA-MBP complexes are isolated from the mixture of DNA fragments, for example, by using a filter column in which a membrane retains the DNA-protein complexes. To PCR amplify the methylated DNA fragments, the DNA fragments generated from restriction digestion are linked with amplification linkers (also called adapters) and subsequently amplified by PCR to generate fragments with the same sequences as the templates but without methylated residues. Optionally a detectable label such as biotin is added to the amplification products to facilitate downstream detection of the DNA fragments by using various methods. As illustrated in FIG. 1 Panel B, the amplification products can be detected by using a hybridization array to simultaneously detect multiple different DNA fragments containing methylated base residues.

As exemplified in FIG. 1 Panel B, the methylated DNA fragments in the complexes can be amplified, for example, by PCR to produce a larger amount of the DNA fragments which share the same sequences as the templates but which no longer contain methylated residues due to the inability of the DNA polymerase to distinguish between a methylated and unmethylated residue. The sequences (i.e., identities) of the amplification products can then be determined by various methods, such as sequencing or more rapid techniques that do not involve sequencing, such as polynucleotide hybridization arrays and bDNA assays. As will be described in more detail below, a polynucleotide hybridization array can be constructed by spotting a library of polynucleotides (e.g., in the form of oligonucleotides or plasmids) onto specific, discrete positions on a hybridization membrane. The library of polynucleotides can be, e.g., a plurality of different sequences comprising the full length or a portion of the promoter regions of different genes. Examples of such promoter sequences (or their complements) that are incorporated into plasmids (e.g., by amplifying genomic regions by PCR and cloning the PCR products directly into a plasmid such as the TA cloning vector pCR® 2.1-TOPO from Invitrogen) spotted on the membrane are listed in Table 1. As demonstrated in Example 1 below, by using an embodiment of the present invention, methylation status of the promoter regions of multiple different genes can be determined simultaneously in a high throughput manner. In addition, methylation profiles of the genes in different cells or cell lines can be compared, such as those in cancer cells as compared to those in normal cells.

In contrast to previous methods for determining methylation patterns by using bisulfite treatment, detection of the methylated nucleic acid using the inventive method is relatively rapid and is based on binding of methylated nucleic acid to an MBP, optionally coupled with amplification of the isolated methylated nucleic acid (e.g., DNA), and multiplex detection. By exploiting the molecular interactions between methylated nucleic acid and methylation binding protein, methylated and unmethylated nucleic acid molecules (such as genomic DNA fragments containing CpG sites) in a mixture can be specifically distinguished and separated efficiently without going through bisulfite modification. Thus the present invention greatly reduces the amount of labor involved in the analysis of methylation status as compared to methods using bisulfite-treated DNA.

The present invention provides for significant advantages over previous PCR-based and other methods (e.g., Southern analysis) used for determining methylation patterns. The present invention is substantially more sensitive than Southern analysis, and facilitates the detection of a low number (percentage) of methylated alleles in very small nucleic acid samples, as well as from paraffin-embedded samples. Moreover, in the case of genomic DNA, analysis is not limited to DNA sequences recognized by methylation-sensitive restriction endonucleases, thus allowing for fine mapping of methylation patterns across broader CpG-rich or other regions. The present invention also eliminates the false-positive results due to incomplete digestion by methylation-sensitive restriction enzymes that are inherent in previous PCR-based methylation detection methods.

The present invention also offers significant advantages over MSP technology. For example, the method can be applied as a quantitative process for measuring methylation amounts, and it is substantially more rapid. One important advance over MSP technology is that the gel electrophoresis step in MSP, which is a time-consuming manual task that limits high throughput capabilities, can be avoided.

Further, one embodiment of the present invention provides for the unbiased amplification of all possible methylation states using primers that do not cover any CpG sequences in the original, unmodified DNA sequence (e.g., amplification using universal primers complementary to adaptors added to the original DNA molecules, as opposed to target-specific PCR amplification using a different pair of primers for each different sequence to be amplified). To the extent that all methylation patterns are amplified equally, quantitative information about DNA methylation patterns can then be distilled from the resulting PCR pool by any technique capable of detecting sequence differences (e.g., by fluorescence-based PCR, bDNA assays, and/or nucleic acid hybridization arrays).

The present invention provides, in fact, a method for simultaneously determining the complete methylation pattern present in the original unmodified sample of genomic DNA. This is accomplished in a fraction of the time and expense required for direct sequencing of the sample of genomic DNA, and the results are substantially more sensitive. Moreover, one embodiment of the present invention provides for a quantitative assessment of such a methylation pattern by determining the amount of methylated DNA fragment(s) that bind to an MBP.

To further enhance the efficiency and throughput of the isolation, especially when a large number of samples are involved, a robust membrane-based process is used for isolating the methylated DNA-MBP complexes from the mixture of digested genomic DNA containing methylated or non-methylated fragments (or other nucleic acid-MBP complexes from mixtures of methylated and non-methylated nucleic acids). Preferably, the membrane-based process is in a form of membrane-based filtration process. As exemplified in Examples 1 and 2 below, a protein-binding membrane, e.g., a nitrocellulose membrane, is used to retain the methylated DNA-MBP complexes while allowing those non-methylated DNA fragments not bound to protein to pass through (or be washed off) the membrane. The membrane-bound methylated DNA-MBP complexes are then eluted from the membrane, and the DNA fragments in the complexes are then isolated and/or characterized. For ease of handling, the membrane is optionally part of a device such as a spin column or multiwell filter plate, for example.

The protein-binding membrane can be incorporated to a filter column of any size, depending on the volume of the samples to be filtered. The protein-binding membrane is preferred not to bind to nucleic acid substantially, more preferably binds to less than 10% of free nucleic acid under the identical condition for binding to protein, and most preferably binds to less than 2% of free nucleic acid under the identical condition for binding to protein. The pore size of the membrane is preferably 0.01-10 μm, optionally 0.05-5 μm, optionally 0.2-1.0 μm, or optionally 0.2-0.5 μm. The membrane is most preferably a nitrocellulose membrane with pore size of about 0.45 μm (e.g., Hybond-ECL nitrocellulose membrane, Amersham). To reduce background noise, the mixture of methylated DNA and MBP can be incubated with the membrane at about 0-4° C. for about 20-30 min, more preferably for about 10-30 min, and most preferably for about 15-25 min.

By using the methods provided in the present invention, a library of diverse methylated DNA fragments bound to MBP can be efficiently and conveniently isolated. As described below, under suitable conditions, the isolated methylated DNA fragments can be sensitively and specifically detected by various nucleic acid arrays provided in the present invention with superb signal-to-noise ratios.

The methylated DNA fragments bound to MBP are separated from MBP by eluting with a protein denaturing buffer such as SDS.

While the discussion is couched largely in terms of detection of 5-mCyt methylated DNA, it will be evident that similar considerations apply to detection of other methylated nucleic acids. Such a methylated nucleic acid can be a nucleic acid other than DNA and/or a nucleic acid (including DNA) methylated at other base(s) and/or position(s), e.g., N6-methyladenine or N4-methylcytosine. See, e.g., Vanyushin (2005) “Adenine methylation in eukaryotic DNA” Molecular Biology 39:473-481 and Ratel et al. (2006) “N6-methyladenine: the other methylated base of DNA” BioEssays 28:309-315.

As noted above, a variety of different methods may be used to identify which methylated DNA fragments are present in the isolated methylated DNA-MBP complexes. By identifying which methylated DNA fragments are present in the sample of genomic DNA after restriction digestion, one is able to determine which region of a gene is methylated.

One method that may be used to identify which gene fragments are methylated and present in the isolated methylated DNA-MBP complexes is based on sequencing of the DNA fragments forming DNA-protein complexes with MBP. By identifying which DNA fragments are present based on the sequence information, one can determine which genes are methylated and can also quantify the extent of methylation of each identified gene. As noted above, however, such sequencing can be time consuming and limit multiplexing. Thus, in one aspect, detection is by a technique other than nucleic acid sequencing.

Another method for identifying which methylated gene fragments formed complexes with MBP involves hybridization of the methylated gene fragments or their amplified products with a hybridization probe comprising a complement to the sequence of the gene fragments prior to the methylation. Multiple gene fragments can be detected simultaneously, e.g., using a hybridization array or a particle-based assay.

Hybridization Assays and Arrays

A wide variety of assays have been developed for performing hybridization assays and detecting the formation of duplexes that may be used in the present invention. For example, hybridization probes with a fluorescent dye and a quencher where the fluorescent dye is quenched when the probe is not hybridized to a target and is not quenched when hybridized to a target oligonucleotide may be used. Such fluorescer-quencher probes are described in, for example, U.S. Pat. No. 6,070,787 and S. Tyagi et al., “Molecular Beacons: Probes that Fluoresce upon Hybridization”, Dept. of Molecular Genetics, Public Health Research Institute, New York, N.Y., Aug. 25, 1995, each of which are incorporated herein by reference. By attaching different fluorescent dyes to different hybridization probes, it is possible to determine which methylated gene fragments formed complexes with MBP based on which fluorescent dyes are present (e.g., using configurations with fluorescent dye and quencher on the hybridization probe or fluorescent dye on the hybridization probe and quencher on the methylated transcription factor probe). Different fluorescent dyes can also be attached to different methylated gene fragments or their amplified products and a change in fluorescence due to hybridization to a hybridization probe used to determine which methylated gene fragments or their amplified products are present (e.g., fluorescent dye on the methylated gene fragments or their amplified products, and quencher on hybridization probe).

A preferred assay for detecting the formation of duplexes between the methylated gene fragments or their amplified products and hybridization probes comprising their complements involves the use of an array of hybridization probes immobilized on a solid support. The hybridization probes comprise sequences that are complementary to at least a portion of the recognition sequences of the transcription factor probes (the methylated gene fragments or their amplified products) and thus are able to hybridize to the different probes in a transcription factor probe library.

In order to enhance the sensitivity of the hybridization array, the immobilized hybridization probes preferably provide at least 2, 3, 4 or more copies of a promoter region of a gene, preferably incorporated into a plasmid immobilized on a solid support, such as a nylon hybridization membrane or a glass-based hybridization array.

According to one embodiment of the present invention, the hybridization probes immobilized on the array preferably are at least 25 nucleotides in length, more preferably at least 50, 100, 200 or 500 nucleotides in length.

By immobilizing on a solid support hybridization probes which comprise one or more copies of a complement to at least a portion of the gene fragment, the hybridization probes serve as immobilizing agents for the gene fragments, each different hybridization probe being designed to selectively immobilize a different gene fragment, e.g., to a predetermined position on the array.

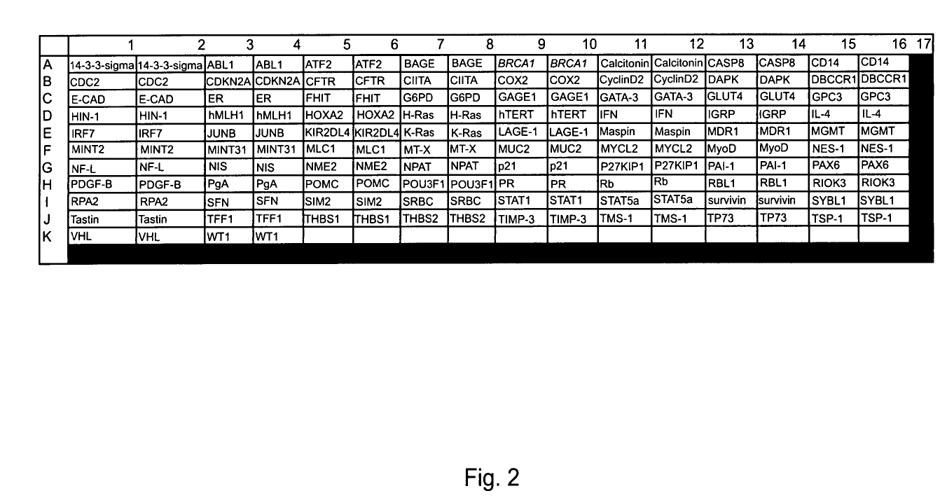

FIG. 2 illustrates an example of an array of hybridization probes attached to a solid support where different hybridization probes are attached to discrete, different regions of the array. Each different region of the array comprises one or more copies of a same hybridization probe which incorporates a sequence that is complementary to a promoter region of a specific gene. The sequences of the promoter regions of genes in the array are listed in Table 1. As a result, the hybridization probes in a given region of the array can selectively hybridize to and immobilize a different gene fragment with a methylated promoter sequence that is complementary to the promoter sequence in the hybridization probe.

By detecting which gene fragments hybridize to hybridization probes on the array, one can determine which genes are methylated and can also quantify the amount of each methylated gene fragment.

These arrays can be designed and used to profile methylation status of genes in a variety of biological processes, including cell proliferation, differentiation, transformation, apoptosis, drug treatment, and others described herein.

Numerous methods have been developed for attaching hybridization probes to solid supports in order to perform immobilized hybridization assays and detect target oligonucleotides in a sample. Numerous methods and devices are also known in the art for detecting the hybridization of a target oligonucleotide to a hybridization probe immobilized in a region of the array. Examples of such methods and device for forming arrays and detecting hybridization include, but are not limited to, those described in U.S. Pat. Nos. 6,197,506, 6,045,996, 6,040,138, 5,424,186, 5,384,261, each of which is incorporated herein by reference.

Provided below is a description of a procedure that is optionally used to hybridize isolated transcription factor probes (methylated gene fragments or their amplified products) to a hybridization array. It is noted that the below procedure may be varied and modified without departing from other aspects of the invention.

An array membrane having hybridization probes attached for the transcription factor probes is first placed into a hybridization bottle. The membrane is then wet by filling the bottle with deionized H₂O. After wetting the membrane, the water is decanted. Membranes that may be used as array membranes include any membrane to which a hybridization probe may be attached. Specific examples of membranes that may be used as array membranes include, but are not limited to NYTRAN membrane (Schleicher & Schuell), BIODYNE membrane (Pall), and NYLON membrane (Roche Molecular Biochemicals).

5 ml of prewarmed hybridization buffer is then added to each hybridization bottle containing an array membrane. The bottle is then placed in a hybridization oven at 42° C. for 2 hr. An example of a hybridization buffer that may be used is EXPHYP by Clonetech.

After incubating the hybridization bottle, a thermal cycler may be used to denature the hybridization probes by heating the probes at 90° C. for 3 min, followed by immediately chilling the hybridization probes on ice.

The isolated DNA fragments from their complex with MBP or their PCR amplified products are then added to the hybridization bottle. Hybridization is preferably performed at 42° C. overnight.

After hybridization, the hybridization mixture is decanted from the hybridization bottle. The membrane is then washed repeatedly.

In one embodiment, washing includes using 60 ml of a prewarmed first hybridization wash which preferably comprises 2×SSC/0.5% SDS. The membrane is incubated in the presence of the first hybridization wash at 42° C. for 20 min with shaking. The first hybridization wash solution is then decanted and the membrane washed a second time. A second hybridization wash, preferably comprising 0.1×SSC/0.5% SDS, is then used to wash the membrane further. The membrane is incubated in the presence of the second hybridization wash at 42° C. for 20 min with shaking. The second hybridization wash solution is then decanted and the membrane washed a second time.

The following describes a procedure that is optionally used to detect methylated gene fragments isolated on the hybridization array. It is noted that each membrane should be separately hybridized, washed, and detected in separate containers in order to prevent cross contamination between samples. It is also noted that it is preferred that the membrane is not allowed to dry during detection. As noted above, the procedure may be varied and modified without departing from other aspects of the invention.

According to the procedure, the membrane is carefully removed from the hybridization bottle and transferred to a new container containing 30 ml of 1× blocking buffer. The dimensions of each container are, e.g., about 4.5″×3.5″, equivalent in size to a 200 μL pipette-tip container. Table 2 provides an embodiment of a blocking buffer that may be used.

TABLE 2
1× Blocking Buffer:

Blocking reagent: 1%
0.1M Maleic acid
0.15M NaCl
Adjusted with NaOH to pH 7.5.

It is noted that the array membrane may tend to curl adjacent to its edges. It is desirable to keep the array membrane flush with the bottom of the container.

The array membrane is incubated at room temperature for 30 min with gentle shaking. 1 ml of blocking buffer is then transferred from each membrane container to a fresh 1.5 ml tube. In an embodiment in which the isolated DNA fragments or their amplified products are labeled with biotin, 3 μl of Streptavidin-AP conjugate is then added to the 1.5 ml tube and is mixed well. The contents of the 1.5 ml tube is then returned to the container and the container is incubated at room temperature for 30 min.

The membrane is then washed three times at room temperature with 40 ml of IX detection wash buffer, each 10 min. Table 3 provides an embodiment of a 1× detection wash buffer that may be used.

TABLE 3
1× Detection wash buffer:

10 mM Tris-HCl, pH 8.0
150 mM NaCl
0.05% Tween-20

30 ml of 1× detection equilibrate buffer is then added to each membrane and the combination is incubated at room temperature for 5 min. Table 4 provides an embodiment of a 1× detection equilibrate buffer that may be used.

TABLE 4
1× Detection equilibrate buffer:

0.1 M Tris-HCl pH 9.5
0.1 M NaCl

The resulting membrane is then transferred onto a transparency film. 3 ml of CPD-Star substrate, produced by Applera, Applied Biosystems Division, is then pipetted onto the membrane.

A second transparency film is then placed over the first transparency. It is important to ensure that substrate is evenly distributed over the membrane with no air bubbles. The sandwich of transparency films is then incubated at room temperature for 5 min.

The CPD-Star substrate is then shaken off and the films are wiped. The membrane is then exposed to Hyperfilm ECL, available from Amersham-Pharmacia. Alternatively, a chemiluminescence imaging system may be used, such as the ones produced by ALPHA INNOTECH. It may be desirable to try different exposures of varying lengths of time (e.g., 2-10 min).

The hybridization array may be used to obtain a quantitative analysis of the methylated gene fragments present. For example, if a chemiluminescence imaging system is being used, the instructions that come with that system's software should be followed. If Hyperfilm ECL is used, it may be necessary to scan the film to obtain numerical data for comparison.

One of the advantages provided by array hybridization for detecting methylated gene fragments is the ability to simultaneously analyze whether multiple different methylated gene fragments are present.

A further advantage provided is that the system allows one to compare a quantification of multiple different methylated gene fragments between two or more samples. When two or more arrays from multiple samples are compared, it is desirable to normalize them.

In order to facilitate normalization of the arrays, an internal standard may be used so that the intensity of detectable marker signals between arrays can be normalized. In certain instances, the internal standard may also be used to control the time used to develop the detectable marker.

In one embodiment, the internal standard for normalization is biotinylated DNA which is spotted on a portion of the array, preferably adjacent one or more sides of the array. For example, biotin-labeled ubiquitin DNA may be positioned on the bottom line and last column of the array. In order to normalize two or more arrays for comparison of results, the exposure time for each array can be adjusted so that the signal intensity in the region of the biotinylated DNA is approximately equivalent on both arrays.

Another preferred assay for detecting the formation of duplexes between the methylated gene fragments or their amplified products and hybridization probes complementary to them involves the use of hybridization probes immobilized on particles, where different hybridization probes complementary to at least a portion of different fragments or products are immobilized on different, distinguishable and identifiable subsets of particles (e.g., microspheres).

Thus, in one class of embodiments, a pooled population of particles is provided. The population includes one or more subsets of particles (typically, one subset for each nucleic acid whose methylation state is to be detected). The particles in each subset are distinguishable from the particles in the other subsets, and the particles in different subsets have associated therewith different nucleic acid hybridization probes with predetermined sequences. The one or more methylated nucleic acids from the isolated methylated nucleic acid-MBP complexes (or complements or copies thereof, e.g., produced by amplification of the methylated nucleic acids) are contacted with the pooled population of particles. The one or more methylated nucleic acids (or the complements or copies thereof) are hybridized with complementary nucleic acid hybridization probes, thereby capturing different methylated nucleic acids (or complements or copies thereof) to different subsets of particles. Which subsets of particles have nucleic acid captured on the particles is then detected, thereby indicating which methylated nucleic acids were present in the sample.

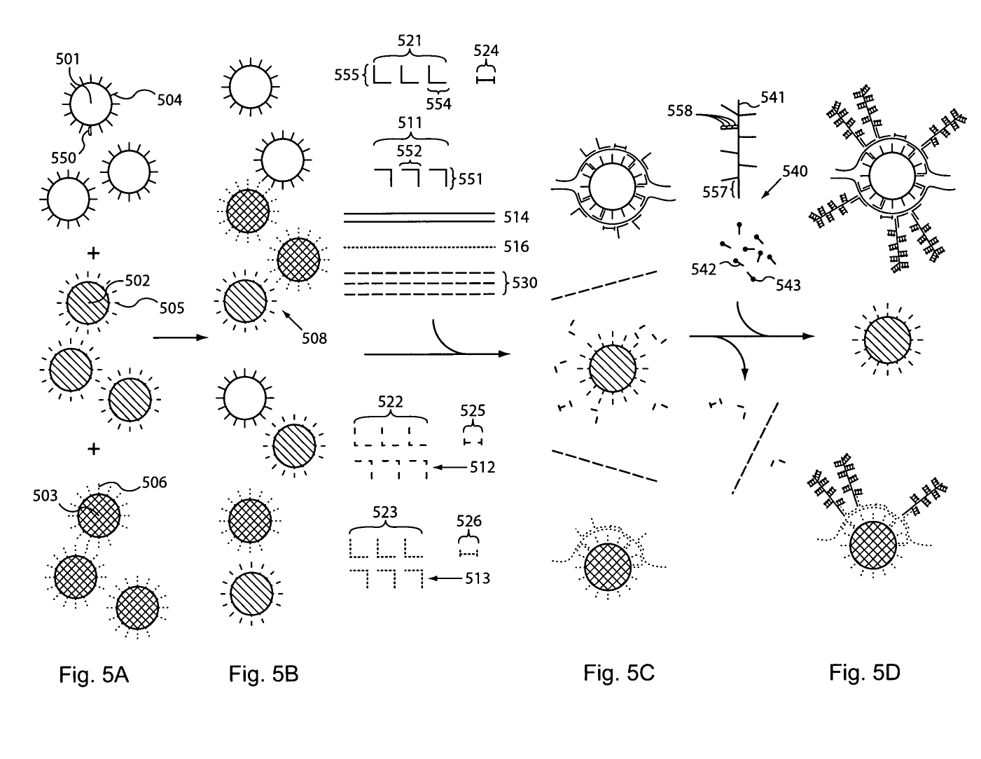

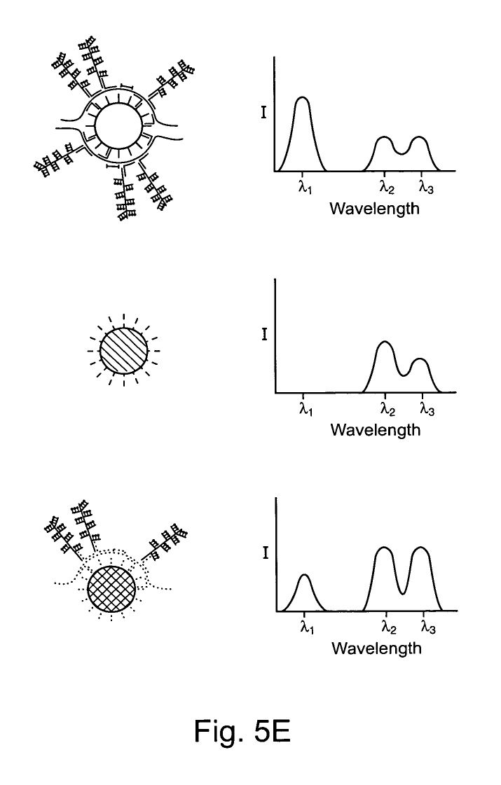

As for arrays of probes on spatially addressable solid supports, the hybridization probes can be bound to the particles directly or indirectly, e.g., covalently or noncovalently. For example, the hybridization probes can be immobilized on the particles through a linker, such as biotinylated probes binding to streptavidin-conjugated particles, or through hybridization to other nucleic acids which are bound to the particles (see, e.g., the embodiment illustrated in FIG. 5). Detection of which subsets of particles have nucleic acid captured on the particles can be performed using any convenient technique; for example, using labeled probes complementary to the nucleic acids or direct labeling of the nucleic acids themselves. In one embodiment, detection involves a bDNA assay, as described in greater detail below.

Branched DNA

In one aspect of the invention, the presence of the methylated nucleic acids in the isolated methylated nucleic acid-MBP complexes is detected with a branched DNA (bDNA) assay. In this aspect, the methylated nucleic acids from the isolated methylated nucleic acid-MBP complexes are captured on a solid support. One or more subsets of m label extenders, wherein m is at least one (and preferably at least two), and wherein each subset of m label extenders is capable of hybridizing to one of the methylated nucleic acids is provided, as is a label probe system comprising a label, wherein a component of the label probe system is capable of hybridizing to the label extenders. Each methylated nucleic acid captured on the solid support is hybridized to its corresponding subset of m label extenders, and the label probe system is hybridized to the label extenders. The presence or absence of the label on the solid support is detected, and thereby the presence or absence of the methylated nucleic acids on the solid support and in the sample is detected. The assay is optionally singleplex or multiplex, and, in multiplex embodiments, different methylated nucleic acids are optionally captured to different positions on an array or to different subsets of particles.

In a typical singleplex bDNA assay, used to detect the presence or absence of a single methylated nucleic acid in the sample, the methylated nucleic acid is captured on the solid support by hybridizing it to n capture extenders (where n is at least one and preferably at least two) and then hybridizing the capture extenders with a capture probe that is bound to the solid support (covalently or noncovalently).

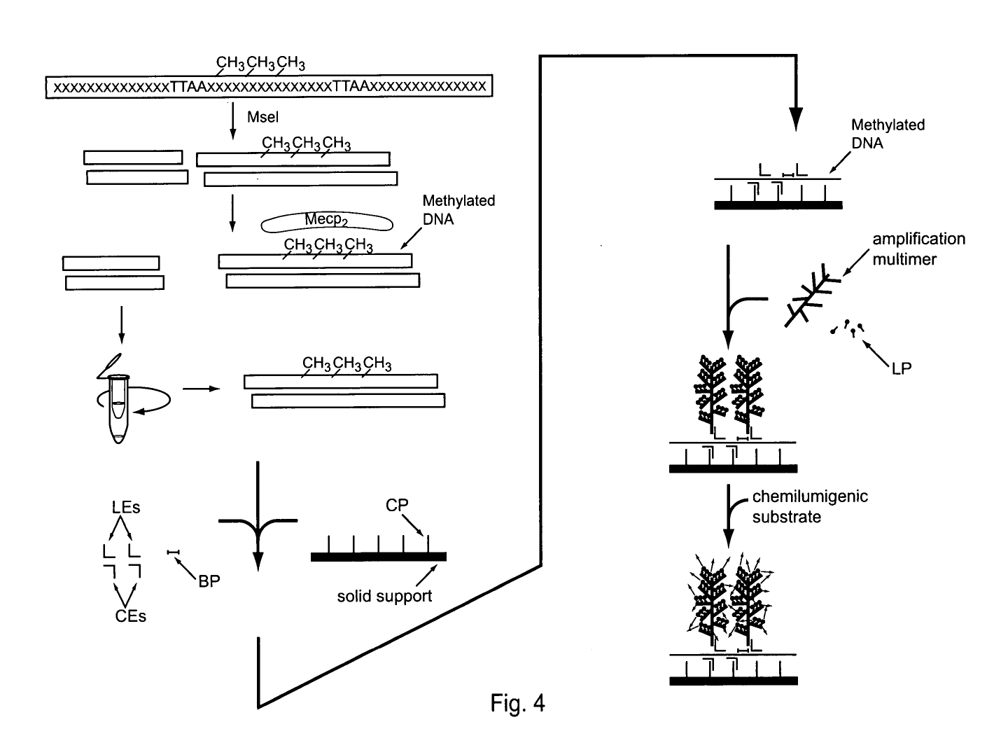

An exemplary singleplex bDNA assay for a methylated DNA fragment is schematically illustrated in FIG. 4. Genomic DNA is digested and the methylated fragment is isolated by formation and isolation of a DNA-MBP complex as described above. The methylated DNA from the isolated DNA-MBP complex is then captured by a Capture Probe (CP) on a solid surface (e.g., a well of a microtiter plate) through synthetic oligonucleotide probes called Capture Extenders (CEs). Each capture extender has a first polynucleotide sequence that can hybridize to the methylated DNA and a second polynucleotide sequence that can hybridize to the capture probe. Typically, two or more capture extenders are used. Probes of another type, called Label Extenders (LEs), hybridize to different sequences on the methylated DNA and to sequences on an amplification multimer. Additionally, Blocking Probes (BPs) are optionally used to reduce non-specific target probe binding. A probe set for a given methylated DNA thus consists of CEs, LEs, and optionally BPs for the methylated DNA. The CEs, LEs, and BPs are complementary to nonoverlapping sequences in the DNA, and are typically, but not necessarily, contiguous.

Signal amplification begins with the binding of the LEs to the methylated DNA. An amplification multimer is then typically hybridized to the LEs. The amplification multimer has multiple copies of a sequence that is complementary to a label probe (it is worth noting that the amplification multimer is typically, but not necessarily, a branched-chain nucleic acid; for example, the amplification multimer can be a branched, forked, or comb-like nucleic acid or a linear nucleic acid). A label, for example, alkaline phosphatase, is covalently attached to each label probe. (Alternatively, the label can be noncovalently bound to the label probes.) In the final step, labeled complexes are detected, e.g., by the alkaline phosphatase-mediated degradation of a chemilumigenic substrate, e.g., dioxetane. Luminescence is reported as relative light unit (RLUs) on a microplate reader. The amount of chemiluminescence is proportional to the level of methylated DNA captured on the support and thus the amount present in the original sample.

In the preceding example, the amplification multimer and the label probes comprise a label probe system. In another example, the label probe system also comprises a preamplifier, e.g., as described in U.S. Pat. No. 5,635,352 and U.S. Pat. No. 5,681,697, which further amplifies the signal from a single methylated DNA. In yet another example, the label extenders hybridize directly to the label probes and no amplification multimer or preamplifier is used, so the signal from a single target methylated DNA molecule is only amplified by the number of distinct label extenders that hybridize to that methylated DNA.

Basic bDNA assays have been well described. See, e.g., U.S. Pat. No. 4,868,105 to Urdea et al. entitled “Solution phase nucleic acid sandwich assay”; U.S. Pat. No. 5,635,352 to Urdea et al. entitled “Solution phase nucleic acid sandwich assays having reduced background noise”; U.S. Pat. No. 5,681,697 to Urdea et al. entitled “Solution phase nucleic acid sandwich assays having reduced background noise and kits therefor”; U.S. Pat. No. 5,124,246 to Urdea et al. entitled “Nucleic acid multimers and amplified nucleic acid hybridization assays using same”; U.S. Pat. No. 5,624,802 to Urdea et al. entitled “Nucleic acid multimers and amplified nucleic acid hybridization assays using same”; U.S. Pat. No. 5,849,481 to Urdea et al. entitled “Nucleic acid hybridization assays employing large comb-type branched polynucleotides”; U.S. Pat. No. 5,710,264 to Urdea et al. entitled “Large comb type branched polynucleotides”; U.S. Pat. No. 5,594,118 to Urdea and Horn entitled “Modified N-4 nucleotides for use in amplified nucleic acid hybridization assays”; U.S. Pat. No. 5,093,232 to Urdea and Horn entitled “Nucleic acid probes”; U.S. Pat. No. 4,910,300 to Urdea and Horn entitled “Method for making nucleic acid probes”; U.S. Pat. No. 5,359,100; U.S. Pat. No. 5,571,670; U.S. Pat. No. 5,614,362; U.S. Pat. No. 6,235,465; U.S. Pat. No. 5,712,383; U.S. Pat. No. 5,747,244; U.S. Pat. No. 6,232,462; U.S. Pat. No. 5,681,702; U.S. Pat. No. 5,780,610; U.S. Pat. No. 5,780,227 to Sheridan et al. entitled “Oligonucleotide probe conjugated to a purified hydrophilic alkaline phosphatase and uses thereof”; U.S. patent application Publication No. US2002172950 by Kenny et al. entitled “Highly sensitive gene detection and localization using in situ branched-DNA hybridization”; Wang et al. (1997) “Regulation of insulin preRNA splicing by glucose” Proc Nat Acad Sci USA 94:4360-4365; Collins et al. (1998) “Branched DNA (bDNA) technology for direct quantification of nucleic acids: Design and performance” in Gene Quantification, F Ferre, ed.; and Wilber and Urdea (1998) “Quantification of HCV RNA in clinical specimens by branched DNA (bDNA) technology” Methods in Molecular Medicine: Hepatitis C 19:71-78. In addition, kits for performing basic bDNA assays (QuantiGene® kits, comprising instructions and reagents such as amplification multimers, alkaline phosphatase labeled label probes, chemilumigenic substrate, capture probes immobilized on a solid support, and the like) are commercially available, e.g., from Panomics, Inc. (on the world wide web at www(dot)panomics(dot)com). Software for designing probe sets for a given nucleic acid target (i.e., for designing the regions of the CEs, LEs, and optionally BPs that are complementary to the target) is also commercially available (e.g., ProbeDesigner™ from Panomics, Inc.; see also Bushnell et al. (1999) “ProbeDesigner: for the design of probe sets for branched DNA (bDNA) signal amplification assays Bioinformatics 15:348-55).

Alternatively, the bDNA assay can be a multiplex assay, used to simultaneously detect the presence or absence of two or more methylated nucleic acids in the sample. Multiplex bDNA assays are described briefly herein, and additional details (for example, on configuration and design of capture extenders, label extenders, and/or the label probe system) can be found in U.S. patent application Ser. No. 11/433,081 filed May 11, 2006 entitled “Multiplex branched-chain DNA assays” by Luo et al and U.S. patent application Ser. No. 11/471,025 filed Jun. 19, 2006 entitled “Multiplex detection of nucleic acids” by Yuling Luo et al, each of which is herein incorporated by reference.

For example, in one class of embodiments in which the methylation status of two or more nucleic acids (e.g., five or more, 10 or more, 20 or more, 30 or more, 40 or more, 50 or more, or even 100 or more nucleic acids) is to be detected, the methylated nucleic acids from the isolated methylated nucleic acid-MBP complexes are captured to different subsets of particles by providing a pooled population of particles which constitute the solid support, the population comprising two or more subsets of particles, the particles in each subset being distinguishable from the particles in the other subsets, and the particles in each subset having associated therewith a different capture probe; providing two or more subsets of n capture extenders, wherein n is at least one (and preferably at least two), wherein each subset of n capture extenders is capable of hybridizing to one of the methylated nucleic acids, and wherein the capture extenders in each subset are capable of hybridizing to one of the capture probes and thereby associating each subset of n capture extenders with a selected subset of the particles; and hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the subset of particles with which the capture extenders are associated. At least a portion of the particles from each subset are identified and the presence or absence of the label on those particles is detected. Since a correlation exists between a particular subset of particles and a particular methylated nucleic acid, which subsets of particles have the label present indicates which of the methylated nucleic acids were present in the sample.

Essentially any suitable particles, e.g., particles having distinguishable characteristics and to which capture probes can be attached, can be used. For example, in one preferred class of embodiments, the particles are microspheres. The microspheres of each subset can be distinguishable from those of the other subsets, e.g., on the basis of their fluorescent emission spectrum, their diameter, or a combination thereof. For example, the microspheres of each subset can be labeled with a unique fluorescent dye or mixture of such dyes, quantum dots with distinguishable emission spectra, and/or the like. As another example, the particles of each subset can be identified by an optical barcode, unique to that subset, present on the particles.

The particles optionally have additional desirable characteristics. For example, the particles can be magnetic or paramagnetic, which provides a convenient means for separating the particles from solution, e.g., to simplify separation of the particles from any materials not bound to the particles.

An exemplary embodiment in which the methylated nucleic acids from the isolated methylated nucleic acid-MBP complexes are detected is schematically illustrated in FIG. 5. Panel A illustrates three distinguishable subsets of microspheres 501, 502, and 503, which have associated therewith capture probes 504, 505, and 506, respectively. Each capture probe includes a sequence C-2 (550), which is different from subset to subset of microspheres. The three subsets of microspheres are combined to form pooled population 508 (Panel B). A subset of three capture extenders is provided for each methylated nucleic acid; subset 511 for methylated nucleic acid 514, subset 512 for methylated nucleic acid 515 which is not present (e.g., in embodiments in which this nucleic acid was unmethylated in the original sample), and subset 513 for methylated nucleic acid 516. Each capture extender includes sequences C-1 (551, complementary to the respective capture probe's sequence C-2) and C-3 (552, complementary to a sequence in the corresponding methylated nucleic acid). Three subsets of label extenders (521, 522, and 523 for nucleic acids 514, 515, and 516, respectively) and three subsets of blocking probes (524, 525, and 526 for nucleic acids 514, 515, and 516, respectively) are also provided. Each label extender includes sequences L-1 (554, complementary to a sequence in the corresponding methylated nucleic acid) and L-2 (555, complementary to M-1). Non-target methylated nucleic acids 530 are also present in the mixture of nucleic acids from the isolated methylated nucleic acid-MBP complexes.

Nucleic acids 514 and 516 are hybridized to their corresponding subset of capture extenders (511 and 513, respectively), and the capture extenders are hybridized to the corresponding capture probes (504 and 506, respectively), capturing nucleic acids 514 and 516 on microspheres 501 and 503, respectively (Panel C). Materials not bound to the microspheres (e.g., capture extenders 512, nucleic acids 530, etc.) are separated from the microspheres by washing. Label probe system 540 including amplification multimer 541 (which includes sequences M-1 557 and M-2 558) and label probe 542 (which contains label 543) is hybridized to label extenders 521 and 523, which are hybridized to nucleic acids 514 and 516, respectively (Panel D). Materials not captured on the microspheres are optionally removed by washing the microspheres. Microspheres from each subset are identified, e.g., by their fluorescent emission spectrum (λ₂ and λ₃, Panel E), and the presence or absence of the label on each subset of microspheres is detected (λ₁, Panel E). Since each methylated nucleic acid is associated with a distinct subset of microspheres, the presence of the label on a given subset of microspheres correlates with the presence of the methylated nucleic acid in the original sample.

As depicted in FIG. 5, all of the label extenders in all of the subsets typically include an identical sequence L-2. Optionally, however, different label extenders (e.g., label extenders in different subsets) can include different sequences L-2. Also as depicted in FIG. 5, each capture probe typically includes a single sequence C-2 and thus hybridizes to a single capture extender. Optionally, however, a capture probe can include two or more sequences C-2 and hybridize to two or more capture extenders. Similarly, as depicted, each of the capture extenders in a particular subset typically includes an identical sequence C-1, and thus only a single capture probe is needed for each subset of particles; however, different capture extenders within a subset optionally include different sequences C-1 (and thus hybridize to different sequences C-2, within a single capture probe or different capture probes on the surface of the corresponding subset of particles).

In another exemplary class of embodiments in which the methylation status of two or more nucleic acids (e.g., five or more, 10 or more, 20 or more, 30 or more, 40 or more, 50 or more, or even 100 or more nucleic acids) is to be detected, the methylated nucleic acids from the isolated methylated nucleic acid-MBP complexes are captured to different positions on a spatially addressable solid support. In this class of embodiments, the solid support is preferably substantially planar, and it comprises two or more capture probes, each of which is provided at a selected position on the solid support. The methylated nucleic acids are captured on the solid support by providing two or more subsets of n capture extenders, wherein n is at least one (and preferably at least two), wherein each subset of n capture extenders is capable of hybridizing to one of the methylated nucleic acids, and wherein the capture extenders in each subset are capable of hybridizing to one of the capture probes and thereby associating each subset of n capture extenders with a selected position on the solid support; and hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the solid support at the selected position with which the capture extenders are associated. The presence or absence of the label at the selected positions on the solid support is then detected. Since a correlation exists between a particular position on the support and a particular methylated nucleic acid, which positions have a label present indicates which of the methylated nucleic acids were present in the sample.

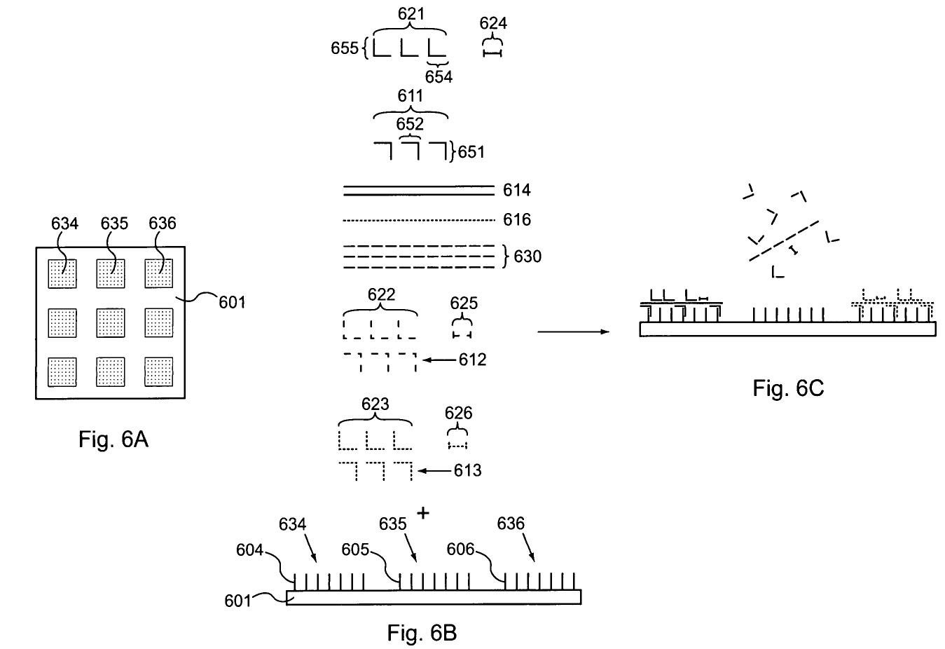

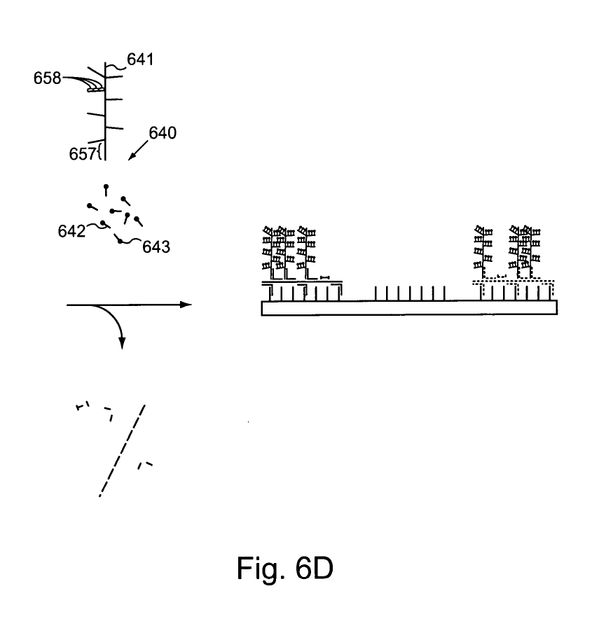

An exemplary embodiment in which the methylated nucleic acids from the isolated methylated nucleic acid-MBP complexes are detected is schematically illustrated in FIG. 6. Panel A depicts solid support 601 having nine capture probes provided on it at nine selected positions (e.g., 634-636). Panel B depicts a cross section of solid support 601, with distinct capture probes 604, 605, and 606 at different selected positions on the support (634, 635, and 636, respectively). A subset of capture extenders is provided for each methylated nucleic acid. Only three subsets are depicted; subset 611 for methylated nucleic acid 614, subset 612 for methylated nucleic acid 615 which is not present, and subset 613 for methylated nucleic acid 616. Each capture extender includes sequences C-1 (651, complementary to the respective capture probe's sequence C-2) and C-3 (652, complementary to a sequence in the corresponding methylated nucleic acid). Three subsets of label extenders (621, 622, and 623 for nucleic acids 614, 615, and 616, respectively) and three subsets of blocking probes (624, 625, and 626 for nucleic acids 614, 615, and 616, respectively) are also depicted (although nine would typically be provided, one for each methylated nucleic acid). Each label extender includes sequences L-1 (654, complementary to a sequence in the corresponding methylated nucleic acid) and L-2 (655, complementary to M-1). Non-target methylated nucleic acids 630 are also present in the mixture of nucleic acids from the isolated methylated nucleic acid-MBP complexes.

Methylated nucleic acids 614 and 616 are hybridized to their corresponding subset of capture extenders (611 and 613, respectively), and the capture extenders are hybridized to the corresponding capture probes (604 and 606, respectively), capturing nucleic acids 614 and 616 at selected positions 634 and 636, respectively (Panel C). Materials not bound to the solid support (e.g., capture extenders 612, nucleic acids 630, etc.) are separated from the support by washing. Label probe system 640 including amplification multimer 641 (which includes sequences M-1 657 and M-2 658) and label probe 642 (which contains label 643) is hybridized to label extenders 621 and 623, which are hybridized to nucleic acids 614 and 616, respectively (Panel D). Materials not captured on the solid support are optionally removed by washing the support, and the presence or absence of the label at each position on the solid support is detected. Since each methylated nucleic acid is associated with a distinct position on the support, the presence of the label at a given position on the support correlates with the presence of the corresponding methylated nucleic acid in the original sample.

The methods can optionally be used to quantitate the amounts of the methylated nucleic acids present in the sample. For example, in one class of embodiments, an intensity of a signal from the label is measured, e.g., for each subset of particles or selected position on the solid support, and correlated with a quantity of the corresponding methylated nucleic acid present.

For the multiplex embodiments, as for the singleplex embodiments above, it is worth noting that the label probe system optionally includes an amplification multimer and a plurality of label probes, wherein the amplification multimer is capable of hybridizing to a label extender and to a plurality of label probes. As another example, the label probe system optionally includes a preamplifier, an amplification multimer and a label probe, where the preamplifier is capable of hybridizing simultaneously to a label extender and to a plurality of amplification multimers and where the amplification multimer is capable of hybridizing simultaneously to the preamplifier and to a plurality of label probes. In one class of embodiments, the label probe comprises the label. In one aspect, the label is a fluorescent label, and detecting the presence of the label (e.g., on the particles or the spatially addressable solid support) comprises detecting a fluorescent signal from the label (and, as noted, optionally measuring its intensity and correlating it with a quantity of the corresponding methylated nucleic acid present).

Methods for Diagnosis or Treatment

The present invention has a wide variety of applications including genomic analysis, diagnostics and therapeutics. For example, the methods of the invention can be applied to high throughput analysis of genomic DNA containing or suspected of containing methylated base residues such as the CpG islands. In particular, the methods of the invention can be applied to analysis of aberrant methylation pattern (e.g., hypermethylation and/or hypomethylation pattern) of disease-related genes in a sample. Owing to the multiplex nature of sample processing and analysis, the methods can be used for robust and efficient determination of methylation patterns of a large number of samples and to analyze them in parallel with control/reference samples such as samples containing normal healthy cells in which the genes are relatively less or more methylated.

Accordingly, in one aspect of the invention, a method is provided for diagnosing a disease or condition associated with aberrant hypermethylation or hypomethylation, such as cancer or a hematological disorder. The method comprises contacting a sample of nucleic acid containing methylated nucleic acid or suspected of containing methylated nucleic acid with an MBP, wherein the sample of nucleic acid is derived from a sample of cells from a patient having or suspected of having a disease or condition associated with aberrant hypermethylation or hypomethylation; forming a methylated nucleic acid-MBP complex; isolating the methylated nucleic acid-MBP complex; detecting levels of the methylated nucleic acid in the isolated methylated nucleic acid-MBP complex; and comparing levels of methylated nucleic acid with that of a reference sample containing nucleic acid derived from normal or healthy cells or from cells from a different sample, wherein an increase in the levels of methylated nucleic acid indicates that the patient has a disease associated with aberrant hypermethylation or wherein a decrease in the levels of methylated nucleic acid indicates that the patient has a disease associated with aberrant hypomethylation.

In yet another aspect of the invention, a method is provided for treating a disease or condition associated with aberrant hypermethylation, such as cancer. The method comprises contacting a sample of nucleic acid containing methylated nucleic acid or suspected of containing methylated nucleic acid with an MBP, wherein the sample of nucleic acid is derived from a sample of cells from a patient having a disease or condition associated with aberrant hypermethylation; forming a methylated nucleic acid-MBP complex; isolating the methylated nucleic acid-MBP complex; detecting the presence of the methylated nucleic acid in the isolated methylated nucleic acid-MBP complex; comparing the pattern of methylated nucleic acid with that of a reference sample containing nucleic acid derived from normal or healthy cells or from cells from a different sample; and treating the patient with a therapeutic agent that inhibits hypermethylation of DNA in the cells, such as 5-azacytidine (or azacytidine) and 5-aza-2′-deoxycytidine (or decitabine).

In a particular application, the present invention can be used to determine aberrant hypermethylation of cancer-related genes.

In mammalian cells, approximately 3% to 5% of the cytosine residues in genomic DNA are present as 5-methylcytosine (Ehrlich et al (1982) Nucleic Acid Res. 10:2709-2721). This modification of cytosine takes place after DNA replication and is catalyzed by DNA methyltransferase using S-adenosyl-methionine as the methyl donor. Approximately 70% to 80% of 5-methylcytosine residues are found in the CpG sequence (Bird (1986) Nature 321:209-213). This sequence, when found at a high frequency in the genome, is referred to as CpG islands. Unmethylated CpG islands are associated with housekeeping genes, while the islands of many tissue-specific genes are methylated, except in the tissue where they are expressed (Yevin and Razin (1993) in DNA Methylation: Molecular Biology and Biological Significance. Basel: Birkhauser Verlag, p 523-568). This methylation of DNA has been proposed to play an important role in the control of expression of different genes in eukaryotic cells during embryonic development. Consistent with this hypothesis, inhibition of DNA methylation has been found to induce differentiation in mammalian cells (Jones and Taylor (1980) Cell 20:85-93).

Methylation of DNA in the regulatory region of a gene can inhibit transcription of the gene. Without limitation to any particular mechanism, this may be because 5-methylcytosine protrudes into the major groove of the DNA helix, which interferes with the binding of transcription factors.

The most commonly occurring methylated cytosine in DNA, 5-methylcytosine, can undergo spontaneous deamination to form thymine at a rate much higher than the deamination of cytosine to uracil (Shen et al. (1994) Nucleic Acid Res. 22:972-976). If the deamination of 5-methylcytosine is unrepaired, it will result in a C to T transition mutation. For example, many “hot spots” of DNA damage in the human p53 gene are associated with CpG to TpG transition mutations (Denissenko et al. (1997) Proc. Natl. Acad. Sci. USA 94:3893-1898).

Other than such transition mutations, many tumor suppressor genes can also be inactivated by aberrant methylation of the CpG islands in their promoter regions. Many tumor-suppressors and other cancer-related genes have been found to be hypermethylated in human cancer cells and primary tumors. Examples of genes that participate in suppressing tumor growth and are silenced by aberrant hypermethylation include, but are not limited to, tumor suppressors such as p15/INK4B (cyclin kinase inhibitor, p16/INK4A (cyclin kinase inhibitor), p73 (p53 homology), ARF/INK4A (regular level p53), Wilms tumor, von Hippel Lindau (VHL), retinoic acid receptor-β (RARβ), estrogen receptor, androgen receptor, mammary-derived growth inhibitor hypermethylated in cancer (HIC1), and retinoblastoma (Rb); invasion/metastasis suppressors such as E-cadherin, tissue inhibitor metalloproteinase-2 (TIMP-3), mts-1 and CD44; DNA repair/detoxify carcinogens such as methylguanine methyltransferase, hMLH1 (mismatch DNA repair), glutathione S-transferase, and BRCA-1; Angiogenesis inhibitors such as thrombospondin-1 (TSP-1) and TIMP3; and tumor antigens such as MAGE-1.

In particular, silencing of p16 is frequently associated with aberrant methylation in many different types of cancers. The p16/INK4A tumor suppressor gene codes for a constitutively expressed cyclin-dependent kinase inhibitor, which plays a vital role in the control of cell cycle by the cyclin D-Rb pathway (Hamel and Hanley-Hyde (1997) Cancer Invest. 15:143-152). P16 is located on chromosome 9p, a site that frequently undergoes loss of heterozygosity (LOH) in primary lung tumors. In these cancers, it is postulated that the mechanism responsible for the inactivation of the nondeleted allele is aberrant methylation. Indeed, for lung carcinoma cell lines that did not express p16, 48% showed signs of methylation of this gene (Otterson et al. (1995) Oncogene 11:1211-1216). About 26% of primary non-small cell lung tumors showed methylation of p16. Primary tumors of the breast and colon display 31% and 40% methylation of p16, respectively (Herman et al. (1995) Cancer Res. 55:4525-4530).

Aberrant methylation of retinoic acid receptors is also attributed to development of breast cancer, lung cancer, ovarian cancer, etc. Retinoic acid receptors are nuclear transcription factors that bind to retinoic acid responsive elements (RAREs) in DNA to activate gene expression. In particular, the putative tumor suppressor RARβ gene is located at chromosome 3p24, a site that shows frequent loss of heterozygosity in breast cancer (Deng et al. (1996) Science 274:2057-2059). Transfection of RARβcDNA into some tumor cells induced terminal differentiation and reduced their tumorigenicity in nude mice (Caliaro et al. (1994) Int. J. Cancer 56:743-748; and Houle et al. (1993) Proc. Natl. Acad. Sci. USA 90:985-989). Lack of expression of the RARβ gene has been reported for breast cancer and other types of cancer (Swisshelm et al. (1994) Cell Growth Differ. 5:133-141; and Crowe (1998) Cancer Res. 58:142-148). This reason for lack of expression of RARE gene is attributed to hypermethylation of RARE gene. Indeed, methylation of RARE was detected in 43% of primary colon carcinomas and in 30% of primary breast carcinoma (Cote et al. (1998) Anti-Cancer Drugs 9:743-750; and Bovenzi et al. (1999) Anticancer Drugs 10:471-476).

Hypermethylation of CpG islands in the 5′-region of the estrogen receptor gene has been found in multiple tumor types (Issa et al. (1994) J. Natl. Cancer Inst. 85:1235-1240). The lack of estrogen receptor expression is a common feature of hormone unresponsive breast cancers, even in the absent of gene mutation (Roodi et al. (1995) J. Natl. Cancer Inst. 87:446-451). About 25% of primary breast tumors that were estrogen receptor-negative displayed aberrant methylation at one site within this gene. Breast carcinoma cell lines that do not express the mRNA for the estrogen receptor displayed increased levels of DNA methyltransferase and extensive methylation of the promoter region for this gene (Ottaviano et al. (1994) 54:2552-2555).

Hypermethylation of human mismatch repair gene (hMLH-1) is also found in various tumors. Mismatch repair is used by the cell to increase the fidelity of DNA replication during cellular proliferation. Lack of this activity can result in mutation rates that are much higher than that observed in normal cells (Modrich and Lahue (1996) Annu. Rev. Biochem. 65:101-133). Methylation of the promoter region of the mismatch repair gene (hMLH-1) was shown to correlate with its lack of expression in primary colon tumors, whereas normal adjacent tissue and colon tumors the expressed this gene did not show signs of its methylation (Kane et al. (1997) Cancer Res. 57:808-811).

The molecular mechanisms by which aberrant methylation of DNA takes place during tumorigenesis are not clear. It is possible that the DNA methyltransferase makes mistakes by methylating CpG islands in the nascent strand of DNA without a complementary methylated CpG in the parental strand. It is also possible that aberrant methylation may be due to the removal of CpG binding proteins that “protect” these sites from being methylated. Whatever the mechanism, aberrant methylation is a rare event in normal mammalian cells.

Examples of genes that have been found to be aberrantly methylated include, but are not limited to, VHL (the Von Hippon Landau gene involved in renal cell carcinoma); P16/INK4A (involved in lymphoma); E-cadherin (involved in metastasis of breast, thyroid, gastric cancer); hMLH1 (involved in DNA repair in colon, gastric, and endometrial cancer); BRCA1 (involved in DNA repair in breast and ovarian cancer); LKB1 (involved in colon and breast cancer); P15/INK4B (involved in leukemia such as AML and ALL); ER (estrogen receptor, involved in breast, colon cancer and leukemia); O6-MGMT (involved in DNA repair in brain, colon, lung cancer and lymphoma); GST-pi (involved in breast, prostate, and renal cancer); TIMP-3 (tissue metalloprotease, involved in colon, renal, and brain cancer metastasis); DAPK1 (DAP kinase, involved in apoptosis of B-cell lymphoma cells); P73 (involved in apoptosis of lymphomas cells); AR (androgen receptor, involved in prostate cancer); RAR-beta (retinoic acid receptor-beta, involved in prostate cancer); Endothelin-B receptor (involved in prostate cancer); Rb (involved in cell cycle regulation of retinoblastoma); P14ARF (involved in cell cycle regulation); RASSF1 (involved in signal transduction); APC (involved in signal transduction); Caspase-8 (involved in apoptosis); TERT (involved in senescence); TERC (involved in senescence); TMS-1 (involved in apoptosis); SOCS-1 (involved in growth factor response of hepatocarcinoma); PITX2 (hepatocarcinoma breast cancer); MINT1; MINT2; GPR37; SDC4; MYOD1; MDR1; THBS1; PTC1; and pMDR1, as described in Santini et al. (2001) Ann. of Intern. Med. 134:573-586, which is herein incorporated by reference in its entirety.

The compositions, kits and methods of the present invention may be used in conjunction with diagnosis and/or treatment of a wide variety of indications such as hematological disorders and cancers that are associated with aberrant hypermethylation, as well as for diagnosis and/or treatment of diseases or conditions associated with hypomethylation (also recognized, e.g., as a cause of oncogenesis; see, e.g., Das and Singal (2004) “DNA methylation and cancer” J Clinical Oncology 22:4632-4642 and references therein).

Hematologic disorders include abnormal growth of blood cells which can lead to dysplastic changes in blood cells and hematological malignancies such as various leukemias. Examples of hematological disorders include but are not limited to acute myeloid leukemia, acute promyelocytic leukemia, acute lymphoblastic leukemia, chronic myelogenous leukemia, the myelodysplastic syndromes (MDS), thalassemia, and sickle cell anemia.

Examples of cancers include, but are not limited to, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, and kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, epidermoid carcinomas, and other carcinomas and sarcomas.

Examples of therapeutic agents for treating diseases associated with hypermethylation include, but are not limited to, azacytidine, decitabine, fazarabine (1-β-D-arabinofurasonyl-5-azacytosine), and dihydro-5-azacytidine as methylation inhibitors, and inhibitors of histone deacetylase (HDAC) including compounds such as hydroxamic acids, cyclic peptides, benzamides, and short-chain fatty acids.

Examples of hydroxamic acids and hydroxamic acid derivatives include, but are not limited to, trichostatin A (TSA), suberoylanilide hydroxamic acid (SAHA), oxamflatin, suberic bishydroxamic acid (SBHA), m-carboxy-cinnamic acid bishydroxamic acid (CBHA), and pyroxamide. TSA was isolated as an antifungi antibiotic (Tsuji et al (1976) J. Antibiot (Tokyo) 29:1-6) and found to be a potent inhibitor of mammalian HDAC (Yoshida et al. (1990) J. Biol. Chem. 265:17174-17179). The finding that TSA-resistant cell lines have an altered HDAC evidences that this enzyme is an important target for TSA. Other hydroxamic acid-based HDAC inhibitors, SAHA, SBHA, and CBHA are synthetic compounds that are able to inhibit HDAC at micromolar concentration or lower in vitro or in vivo. Glick et al. (1999) Cancer Res. 59:4392-4399. These hydroxamic acid-based HDAC inhibitors all possess an essential structural feature: a polar hydroxamic terminal linked through a hydrophobic methylene spacer (e.g. 6 carbon at length) to another polar site which is attached to a terminal hydrophobic moiety (e.g., benzene ring). Compounds developed having such essential features also fall within the scope of the hydroxamic acids that may be used as HDAC inhibitors.

Cyclic peptides used as HDAC inhibitors are mainly cyclic tetrapeptides. Examples of cyclic peptides include, but are not limited to, trapoxin A, apicidin and FR901228. Trapoxin A is a cyclic tetrapeptide that contains a 2-amino-8-oxo-9,10-epoxy-decanoyl (AOE) moiety (Kijima et al. (1993) J. Biol. Chem. 268:22429-22435). Apicidin is a fungal metabolite that exhibits potent, broad-spectrum antiprotozoal activity and inhibits HDAC activity at nanomolar concentrations (Darkin-Rattray et al. (1996) Proc. Natl. Acad. Sci. USA. 93:13143-13147). FR901228 is a depsipeptide that is isolated from Chromobacterium violaceum and has been shown to inhibit HDAC activity at micromolar concentrations.

Examples of benzamides include, but are not limited to, MS-27-275 (Saito et al. (1990) Proc. Natl. Acad. Sci. USA. 96:4592-4597). Examples of short-chain fatty acids include, but are not limited to, butyrates (e.g., butyric acid, arginine butyrate and phenylbutyrate (PB); see Newmark et al. (1994) Cancer Lett. 78:1-5 and Carducci et al. (1997) Anticancer Res. 17:3972-3973). In addition, depudecin, which has been shown to inhibit HDAC at micromolar concentrations (Kwon et al. (1998) Proc. Natl. Acad. Sci. USA. 95:3356-3361), also falls within the scope of a histone deacetylase inhibitor of the present invention. Zebularine or antisense or small inhibitory RNAs (siRNAs) can also be administered as therapeutic agents.

In embodiments in which a disease or condition associated with aberrant hypermethylation is treated by administration to the patient of a therapeutic agent that inhibits hypermethylation of DNA, a therapeutically effective amount of the agent (an amount that is effective for preventing, ameliorating, or treating the condition or disease) is typically administered to the patient. In one class of embodiments, after initiation of treatment, the patient displays decreased hypermethylation.

As will be understood by those of ordinary skill in the art, the appropriate doses of therapeutic agents of the invention (e.g., methylation inhibitors, inhibitors of HDAC, etc.) will be generally around those already employed in clinical therapies wherein similar moieties are administered alone or in combination with other therapeutics. Variation in dosage will likely occur depending on the condition being treated. The physician administering treatment will be able to determine the appropriate dose for the individual subject. Preparation and dosing schedules may be used according to manufacturers' instructions or determined empirically by the skilled practitioner.

For the prevention or treatment of disease, the appropriate dosage of the therapeutic agent will depend on the type of disease or condition to be treated, as defined above, the severity and course of the disease, whether the therapeutic agent is administered for preventive or therapeutic purposes, previous therapy, the patient's clinical history and response to the agent, and the discretion of the attending physician. Typically, the clinician will administer a therapeutic agent of the invention (alone or in combination with a second compound) until a dosage is reached that provides the required biological effect. The progress of the therapy is conveniently monitored as described herein and/or by conventional techniques and assays.

The moiety can be administered by any suitable means, including, e.g., parenteral, topical, subcutaneous, intraperitoneal, intrapulmonary, intranasal, and/or intralesional administration. Parenteral infusions include intramuscular, intravenous, intraarterial, intraperitoneal, or subcutaneous administration.

Compositions, Systems, and Kits

Compositions, systems, and kits are also provided for performing the methods described herein, as are compositions formed while practicing the methods.

For example, in one embodiment, a kit is provided which comprises a methylation binding protein (MBP), a separation column for separating MBP-nucleic acid complexes from non-complexed nucleic acid, and instructions for separating MBP-nucleic acid complexes from non-complexed nucleic acid by the separation column (e.g., a column comprising a nitrocellulose membrane). The kit can also comprise an array of predetermined, different nucleic acid hybridization probes immobilized on a surface of a substrate such that the hybridization probes are positioned in different defined regions on the surface. In one embodiment, each of the different nucleic acid hybridization probes comprises a different nucleic acid probe capable of hybridizing to a different region or fragment of a gene, preferably a promoter region of a gene, more preferably a promoter region of a gene listed in Table 1. Most preferably, the array of predetermined, different nucleic acid hybridization probes comprises at least two different nucleic acid probes which are capable of separately hybridizing to at least two promoter regions of the genes listed in Table 1 (that is, to at least two of SEQ ID NOs:1-82 or a complement thereof). The kit can be used for performing the methods provided in the present invention, and the instructions can include instructions on how to perform the methods.

The kit optionally includes buffered solutions (e.g., for washing the separation column, eluting nucleic acid from the separation column, washing the array, or the like), a restriction enzyme, oligonucleotide adaptors and/or primers, PCR reagents (e.g., a thermostable DNA polymerase, nucleoside triphosphates, and the like), detection reagents (e.g., streptavidin-conjugated horseradish peroxidase and a luminescent substrate), and/or the like. Essentially all of the features noted for the methods above apply to these embodiments as well, as relevant.

In another exemplary embodiment, a kit for detecting one or more methylated nucleic acids is provided which comprises a methylation binding protein (MBP), a nitrocellulose membrane, one or more subsets of m label extenders, wherein m is at least one or two and wherein each subset of m label extenders is capable of hybridizing to one of the methylated nucleic acids, and a label probe system comprising a label, wherein a component of the label probe system is capable of hybridizing to the label extenders. The kit also includes i) 1) a solid support comprising a capture probe and 2) a subset of n capture extenders, wherein n is at least one or two, wherein the subset of n capture extenders is capable of hybridizing to a methylated nucleic acid and is capable of hybridizing to the capture probe and thereby associating the capture extenders with the solid support; ii) 1) a pooled population of particles, the population comprising two or more subsets of particles, a plurality of the particles in each subset being distinguishable from a plurality of the particles in every other subset, and the particles in each subset having associated therewith a different capture probe, and 2) two or more subsets of n capture extenders, wherein n is at least one or two, wherein each subset of n capture extenders is capable of hybridizing to one of the methylated nucleic acids, and wherein the capture extenders in each subset are capable of hybridizing to one of the capture probes and thereby associating each subset of n capture extenders with a selected subset of the particles; or iii) 1) a solid support comprising two or more capture probes, wherein each capture probe is provided at a selected position on the solid support, and 2) two or more subsets of n capture extenders, wherein n is at least one or two, wherein each subset of n capture extenders is capable of hybridizing to one of the methylated nucleic acids, and wherein the capture extenders in each subset are capable of hybridizing to one of the capture probes and thereby associating each subset of n capture extenders with a selected position on the solid support. The components of the kit are packaged in one or more containers.

The kit optionally includes a filter column (e.g., a spin column or a multiwell plate) comprising the nitrocellulose membrane, buffered solutions (e.g., for washing the filter column, eluting nucleic acid from the filter column, washing the particles or other solid support, or the like), a restriction enzyme, and/or the like. Essentially all of the features noted for the embodiments above apply to these embodiments as well, as relevant, for example, with respect to composition of the label probe system.

In one aspect, the invention includes systems, e.g., systems used to practice the methods herein. The system can include, e.g., a fluid and/or microsphere handling element, a fluid and/or microsphere containing element, a laser for exciting a fluorescent label and/or fluorescent microspheres, a detector for detecting light emissions from a chemiluminescent reaction or fluorescent emissions from a fluorescent label and/or fluorescent microspheres, and/or a robotic element that moves other components of the system from place to place as needed (e.g., a multiwell plate handling element). For example, in one class of embodiments, a composition of the invention is contained in a flow cytometer, a Luminex 100™ or HTS™ instrument, a microplate reader, a microarray reader, a luminometer, a colorimeter, or like instrument.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, robotic element and/or laser). The computer can also receive data from other components of the system, e.g., from a detector, and can interpret the data, provide it to a user in a human readable format, or use that data to initiate further operations, in accordance with any programming by the user.

Labels

A wide variety of labels are well known in the art and can be adapted to the practice of the present invention. For example, luminescent labels and light-scattering labels (e.g., colloidal gold particles) have been described. See, e.g., Csaki et al. (2002) “Gold nanoparticles as novel label for DNA diagnostics” Expert Rev Mol Diagn 2:187-93.

As another example, a number of fluorescent labels are well known in the art, including but not limited to, hydrophobic fluorophores (e.g., phycoerythrin, rhodamine, Alexa Fluor 488 and fluorescein), green fluorescent protein (GFP) and variants thereof (e.g., cyan fluorescent protein and yellow fluorescent protein), and quantum dots. See e.g., Haughland (2003) Handbook of Fluorescent Probes and Research Products, Ninth Edition or Web Edition, from Molecular Probes, Inc., or The Handbook: A Guide to Fluorescent Probes and Labeling Technologies, Tenth Edition or Web Edition (2006) from Invitrogen (available on the world wide web at probes(dot)invitrogen(dot)com/handbook) for descriptions of fluorophores emitting at various different wavelengths (including tandem conjugates of fluorophores that can facilitate simultaneous excitation and detection of multiple labeled species). For use of quantum dots as labels for biomolecules, see e.g., Dubertret et al. (2002) Science 298:1759; Nature Biotechnology (2003) 21:41-46; and Nature Biotechnology (2003) 21:47-51.

Labels can be introduced to molecules, e.g. polynucleotides, during synthesis or by postsynthetic reactions by techniques established in the art; for example, kits for fluorescently labeling polynucleotides with various fluorophores are available from Molecular Probes, Inc. ((www.)molecularprobes.com), and fluorophore-containing phosphoramidites for use in nucleic acid synthesis are commercially available. Similarly, signals from the labels (e.g., absorption by and/or fluorescent emission from a fluorescent label) can be detected by essentially any method known in the art. For example, multicolor detection, detection of FRET, fluorescence polarization, and the like, are well known in the art.

Microspheres

Microspheres are preferred particles in certain embodiments described herein since they are generally stable, are widely available in a range of materials, surface chemistries and uniform sizes, and can be fluorescently dyed. Microspheres can be distinguished from each other by identifying characteristics such as their size (diameter) and/or their fluorescent emission spectra, for example.

Luminex Corporation (www(dot)luminexcorp(dot)com), for example, offers 100 sets of uniform diameter polystyrene microspheres. The microspheres of each set are internally labeled with a distinct ratio of two fluorophores. A flow cytometer or other suitable instrument can thus be used to classify each individual microsphere according to its predefined fluorescent emission ratio. Fluorescently-coded microsphere sets are also available from a number of other suppliers, including Radix Biosolutions (www(dot)radixbiosolutions(dot)com) and Upstate Biotechnology (www(dot)upstatebiotech(dot)com). Alternatively, BD Biosciences (www(dot)bd(dot)com) and Bangs Laboratories, Inc. (www(dot)bangslabs(dot)com) offer microsphere sets distinguishable by a combination of fluorescence and size. As another example, microspheres can be distinguished on the basis of size alone, but fewer sets of such microspheres can be multiplexed in an assay because aggregates of smaller microspheres can be difficult to distinguish from larger microspheres.

Microspheres with a variety of surface chemistries are commercially available, from the above suppliers and others (e.g., see additional suppliers listed in Kellar and Iannone (2002) “Multiplexed microsphere-based flow cytometric assays” Experimental Hematology 30:1227-1237 and Fitzgerald (2001) “Assays by the score” The Scientist 15[11]:25). For example, microspheres with carboxyl, hydrazide or maleimide groups are available and permit covalent coupling of molecules (e.g., polynucleotide capture probes with free amine, carboxyl, aldehyde, sulfhydryl or other reactive groups) to the microspheres. As another example, microspheres with surface avidin or streptavidin are available and can bind biotinylated capture probes; similarly, microspheres coated with biotin are available for binding capture probes conjugated to avidin or streptavidin. In addition, services that couple a capture reagent of the customer's choice to microspheres are commercially available, e.g., from Radix Biosolutions (www(dot)radixbiosolutions(dot)com).

Protocols for using such commercially available microspheres (e.g., methods of covalently coupling polynucleotides to carboxylated microspheres for use as capture probes, methods of blocking reactive sites on the microsphere surface that are not occupied by the polynucleotides, methods of binding biotinylated polynucleotides to avidin-functionalized microspheres, and the like) are typically supplied with the microspheres and are readily utilized and/or adapted by one of skill. In addition, coupling of reagents to microspheres is well described in the literature. For example, see Yang et al. (2001) “BADGE, Beads Array for the Detection of Gene Expression, a high-throughput diagnostic bioassay” Genome Res. 11:1888-98; Fulton et al. (1997) “Advanced multiplexed analysis with the FlowMetrix™ system” Clinical Chemistry 43:1749-1756; Jones et al. (2002) “Multiplex assay for detection of strain-specific antibodies against the two variable regions of the G protein of respiratory syncytial virus” 9:633-638; Camilla et al. (2001) “Flow cytometric microsphere-based immunoassay: Analysis of secreted cytokines in whole-blood samples from asthmatics” Clinical and Diagnostic Laboratory Immunology 8:776-784; Martins (2002) “Development of internal controls for the Luminex instrument as part of a multiplexed seven-analyte viral respiratory antibody profile” Clinical and Diagnostic Laboratory Immunology 9:41-45; Kellar and Iannone (2002) “Multiplexed microsphere-based flow cytometric assays” Experimental Hematology 30:1227-1237; Oliver et al. (1998) “Multiplexed analysis of human cytokines by use of the FlowMetrix system” Clinical Chemistry 44:2057-2060; Gordon and McDade (1997) “Multiplexed quantification of human IgG, IgA, and IgM with the FlowMetrix™ system” Clinical Chemistry 43:1799-1801; U.S. Pat. No. 5,981,180 entitled “Multiplexed analysis of clinical specimens apparatus and methods” to Chandler et al. (Nov. 9, 1999); U.S. Pat. No. 6,449,562 entitled “Multiplexed analysis of clinical specimens apparatus and methods” to Chandler et al. (Sep. 10, 2002); and references therein.

Methods of analyzing microsphere populations (e.g. methods of identifying microsphere subsets by their size and/or fluorescence characteristics, methods of using size to distinguish microsphere aggregates from single uniformly sized microspheres and eliminate aggregates from the analysis, methods of detecting the presence or absence of a fluorescent label on the microsphere subset, and the like) are also well described in the literature. See, e.g., the above references.

Suitable instruments, software, and the like for analyzing microsphere populations to distinguish subsets of microspheres and to detect the presence or absence of a label (e.g., a fluorescently labeled label probe) on each subset are commercially available. For example, flow cytometers are widely available, e.g., from Becton-Dickinson (www(dot)bd(dot)com) and Beckman Coulter (www(dot)beckman(dot)com). Luminex 100™ and Luminex HTS™ systems (which use microfluidics to align the microspheres and two lasers to excite the microspheres and the label) are available from Luminex Corporation (www(dot)luminexcorp(dot)com); the similar Bio-Plex™ Protein Array System is available from Bio-Rad Laboratories, Inc. (www(dot)bio-rad(dot)com). A confocal microplate reader suitable for microsphere analysis, the FMAT™ System 8100, is available from Applied Biosystems (www(dot)appliedbiosystems(dot)com).

As another example of particles that can be adapted for use in the present invention, sets of microbeads that include optical barcodes are available from CyVera Corporation (www(dot)cyvera(dot)com). The optical barcodes are holographically inscribed digital codes that diffract a laser beam incident on the particles, producing an optical signature unique for each set of microbeads.

Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., Current Protocols, a joint venture between Greene Publishing Associates, Inc. and John Wiley & Sons, Inc., supplemented through 2006 (“Ausubel”). Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg N.Y.) and Atlas and Parks (Eds.) The Handbook of Microbiological Media (1993) CRC Press, Boca Raton, Fla.

Making Polynucleotides

Methods of making nucleic acids (e.g., by in vitro amplification, purification from cells, or chemical synthesis), methods for manipulating nucleic acids (e.g., by restriction enzyme digestion, ligation, etc.) and various vectors, cell lines and the like useful in manipulating and making nucleic acids are described in the above references. In addition, methods of making branched polynucleotides (e.g., amplification multimers) are described in U.S. Pat. No. 5,635,352, U.S. Pat. No. 5,124,246, U.S. Pat. No. 5,710,264, and U.S. Pat. No. 5,849,481, as well as in other references mentioned above.

In addition, essentially any polynucleotide (including, e.g., labeled or biotinylated polynucleotides) can be custom or standard ordered from any of a variety of commercial sources, such as The Midland Certified Reagent Company (www(dot)mcrc(dot)com), The Great American Gene Company (www(dot)genco(dot)com), ExpressGen Inc. (www(dot)expressgen(dot)com), Qiagen (oligos(dot)qiagen(dot)com) and many others.

A label, biotin, or other moiety can optionally be introduced to a polynucleotide, either during or after synthesis. For example, a biotin phosphoramidite can be incorporated during chemical or enzymatic synthesis of a polynucleotide. Alternatively, any nucleic acid can be biotinylated using techniques known in the art; suitable reagents are commercially available, e.g., from Pierce Biotechnology (www(dot)piercenet(dot)com). Similarly, any nucleic acid can be fluorescently labeled, for example, by using commercially available kits such as those from Molecular Probes, Inc. (www(dot)molecularprobes(dot)com) or Pierce Biotechnology (www(dot)piercenet(dot)com) or by incorporating a fluorescently labeled phosphoramidite during chemical synthesis of a polynucleotide.

Sequence Comparison, Identity, and Homology

The terms “identical” or “percent identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence identity over 50, 100, 150 or more residues (nucleotides or amino acids) is routinely used to establish homology (e.g., over the full length of the two sequences to be compared, e.g., over a methylated DNA binding domain or a polynucleotide encoding such a domain). Higher levels of sequence identity, e.g., 30%, 40%, 50%, 60%, 70%, 80%, 90%, 95%, or 99% or more, can also be used to establish homology. Methods for determining sequence identity or similarity percentages (e.g., BLASTP and BLASTN using default parameters) are described herein and are generally available.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Exemplary Promoter Regions

TABLE 1

DNA sequences of 82 different promoter regions of genes.
*First column presents SEQ ID NOs. The methylation
reference number for each sequence corresponds to the
sequence's SEQ ID NO.

| | | | | |
|---|---|---|---|---|
| | Gene | | | Accession |
| * | Name | Description | Sequence | Number |

| | | | | |
|---|---|---|---|---|
| 1 | 14-3-3- | The 14-3- | CTCTGAAAGCTGCCACCTGCGCATTCTGGGAG | AF029081 |
| | sigma | 3sigma gene | CTCAGAGGGGACCCTGAGGGGGAATGAGGCC | |
| | | (also called | TGGAGGATGGAACCATCTTCAGGTAGACTGA | |
| | | stratifin) was | GAAGGAGCCTGGATCTCACTTCCAAACACAG | |
| | | originally | TCTGGAGCTCATAGGTCAGAGGCCTCAATGG | |
| | | characterized | GAGAAAAGCTAAAGGAAGAGGGTGCAGAAA | |
| | | as the human | GGAgtttcagggaattggtggctatgtgact | |
| | | mammary | ttgagcaaatctcacccctctctgagactta | |
| | | epithelial- | gtgttcccatctctatggtcctgtgtgtgtc | |
| | | specific | acagagacatggtggggattaaattcgatcg | |
| | | marker, HME- | tgaatatgaaagtgcttgggaaactccatgg | |
| | | 1, and is | cCCTACCTAAACATGAGTTATCCTCACCTGA | |
| | | expressed in | ACCAAGGGGGGAAGTTACCTGGCAGGATTAGG | |
| | | keratinocytes | AACCCCATCCTCCTGAACCTTTATGGGCTCTG | |
| | | and epithelial | TCGAGGCTGAAGCAGCCAGGGGCTAAAGCCGT | |
| | | cells. | CCTTAGCCCCTGGAAGGGCACTGTGAAAGTGG | |
| | | | ATCTGATTTGAGAAGCCGTTTCCTGATGTGGG | |
| | | | CAGCCATGTGATGCCAGCCCCGAACAAGAGG | |
| | | | GGGCAGCCTGGAGCCTGGAAAGGTGCCAGTG | |
| | | | CAGGTGGGGCCCACGCCCAGATTTCTCCTGCT | |
| | | | GACTGTTCTGATGATTCACCCCCACATCCCAG | |
| | | | CCTTTTTACCTTTACTGCAGAGCCGGAAAGGG | |
| | | | TGTGGGGAAGAGAGGAGAGGGAGGCAGGTCT | |
| | | | TGGGCCCTGGTCCCGCCCCCTGCTCCTCCCCA | |
| | | | CCCTTCTCTGGGCCTGGCCACCCAGCCAAAAG | |
| | | | GCAGGCCAAGAGCAGGAGAGACACAGAGTCC | |
| | | | GGCATTGGTCCCAGGCAGCAGTTAGCCCGCC | |
| | | | GCCCGCCTGTGTGTCCCCAGAGCCATGGAGA | |
| | | | GAGCCAGTCTGATCCAGAAGGCCAAGCTGGC | |
| | | | AGAGCAGGCCGAACGCTATGAGGACATGGCA | |
| | | | GCCTTCATGAAAGGCGCCGTGGAGAAGGGCG | |
| | | | AGGAGCTCTCCTGCGAAGAGCGAAACCTG | |
| 2 | ABL1 | v-abl Abelson | CTCGGGAGATGTGACTGCCTGAGGGCGGTGG | M15055 |
| | | murine | TGGTGTCAGCGTCCGGGGCCGGGGGAGGGGG | |
| | | leukemia viral | TGTCTCGGGCAGAGACCCCCGGGCTTGGGGC | |
| | | oncogene | AGCTGAGGCGGCCGGGCCTCCTCTACACGGG | |
| | | homolog 1 | GCCCGCCTTCCGCTGTCTGGGCCGCGAGAGTC | |
| | | | CTTCGTCCCTTACAGCCCCGCCCCGGCTTTGG | |
| | | | GACACTGCGGGTGGTCTGTTTCCCCCAGCTTG | |
| | | | GGACACCCCGTTTTCTGAGGCGTGGAAGAGC | |
| | | | GTCGCCCCGGAGTAAGCTGCCCGTGCCGCGC | |
| | | | CCCGACAGCTTCCCTCAGCCCCAAGCCGCCCC | |
| | | | TTATTCCGGATCCCGGCCCCAACTTTGGCCAC | |
| | | | GGAGCCTCCCATTCAAATCCCTCCCTTGCTGT | |
| | | | CAAGGGGTCTCCCCTTCCCCCAAGGTGGCTCC | |
| | | | CGCGAGCCTCTAATGCCCTGACTTCTTCCAAT | |
| | | | GTCACCTACGGCCCCCTTAGTCTCAGCTCAGC | |
| | | | CAAAAACTTTAATGCAAAGGAAAAGTCTGGA | |
| | | | TTGGTTCCACAGGCCTTTTAAAAAGCGGACTT | |
| | | | AAAAGTTGCTGGCAATGCATTCCTTTTCGTCA | |
| | | | GAGTCGAGGGCAAACTCGCTGAAATCTGGGT | |
| | | | GACCCGTGTCCTTTTCCGGAGAGCAAAGCAG | |
| | | | AGAAGCGAGAGCGGCCACTAGTTCGGCAGGA | |
| | | | AATTTGTTGGAAGATGAAGAAGCTAAGATAG | |
| | | | GGGGTTGGTGACTTCCACAGGAAAAGTTCTG | |
| | | | GAGGAGTAGCCAAAGACCATCAGCGTTTCCT | |
| | | | TTATGTGTGAGAATTGAAATGACTAGCATTAT | |
| | | | TGACCCTTTTCAGCATCCCCTGTGAATATTTCT | |
| | | | GTTTAGGTTTTTCTTCTTGAAAAGAAATTGTT | |
| | | | ATTCAGCCCGTTTAAAACAAATCAAGAAACTT | |
| | | | TTGGGTAACATTGCAATTACATGAAATTGATA | |
| | | | ACCGCGAAAATAATTGGAACTCCTGCTTGCA | |
| | | | AGTGTCAACCTAAAAAAAGTGCTTCCTTTTGT | |
| | | | TATGGAAGATGTCTTTCTGTG | |
| 3 | ATF2 | activating | TCCNATAGGGCGATTGGGCCCTCTAGATGCAT | J05623 |
| | | transcription | GCTCGAGCGGCCGCCAGTGTGATGGATATCT | |
| | | factor 2 | GCAGAATTCGCCCTTGTTCTCGGATCCCGATC | |
| | | | ATGTAAATTCTCACAGAGGCCTCTGATCATAC | |
| | | | TTTTCAACTTGTGCCTATTTATTGAATAACCA | |
| | | | ACATCCTTACAGTTAATATTAAAATCTTTAAG | |
| | | | TTGTGTGGGGTTTTTTGGAGGGGAGGGATGG | |
| | | | GCAATTACCAGCAAACTCCGCCTCCCCCAAAC | |
| | | | CTCACCTAACCCGAAGCTCCCCGCCTCAGGCT | |
| | | | CCCGGGGAGCCAAGGGGTGGGCTGAGGAACG | |
| | | | CAGCCTACTTTTACCCACCTCCCTACCTAGTG | |
| | | | CTGGGAAGTGACGGAAACGGAGACACCCGGC | |
| | | | TCCTGGGGCTGGGCTCGGAGGACCCATCCTGC | |
| | | | TTTCCCTCTAGCAGCCTTTCCGGAGCTCACCT | |
| | | | TTCCTCCCCTCACACCGCCAAAGCCCTGCCTA | |
| | | | GCCCTTCACCGCCGCCTGCACCCGCGCCCTCC | |
| | | | TCCAGCCGACAGCCAATCACAGTCTTCCACAG | |
| | | | CTCCGGGTTTACAGAAGTAACGCTCCTTGGGC | |
| | | | CCTCTGGTCCCGCCCCCTCCAGAACTGCTTCC | |
| | | | CGCCCTTCGGGCTCCTTGTCCAATCATGAGCG | |
| | | | CCCGAGTGCTCTTTGATGCCCGTCCCCTCTAC | |
| | | | CCGCCCTGCCGAAGACCCGCCTTCTTCTCCTT | |
| | | | AAGCCTGACGGAATCACCTGACTCGGAGGCG | |
| | | | CTCCCTCANAAGGAAGGCAAGAAGGGGCGTG | |
| | | | TGGGTGAAGGGGAGGGGCGCCAGAANGAAN | |
| | | | GTGGGGGATGCCGGNAGCGGGGCGAGCGGGC | |
| | | | GGGGGTTGTCAGTCCGATCTCGCGAGAGANG | |
| | | | ANGGAAGCCTGTGGGGAGCCCGTGGNCTTTA | |
| | | | AAGTGCCGTTCACCCTTTCCTNCNNNGNNGCT | |
| | | | TTGTAAAACCCGGTTGTGCTCAGGGCTCGCGG | |
| | | | GTGANCGAAAAGGATCATGAANTANTGACCT | |
| | | | GGAAAAGNGAGNAAC | |
| 4 | BAGE | B melanoma | tcctcccacttgtcacccttttcccccctcca | NM_001187 |
| | | antigen | tcactcaaaatctttttacccacagtcttctt | |
| | | | tccctttcttctctccccaccatatttttgca | |
| | | | aaccttctctccttcctgctcatccccgttcc | |
| | | | cccctcacgaccctctcttacccccttccatc | |
| | | | tacccaaaaactttttccccaccatctttctg | |
| | | | tgaaaccttctctccctcctgtttaccacc | |
| | | | ctgtttttccccctccatctaccccccaattt | |
| | | | tttttcccaacatcttttcctcaccgtcttta | |
| | | | tgcaatgacttctccggctcgccatccttttt | |
| | | | tccttttggcactaaccaccctctttaccctt | |
| | | | ccatctatcccaaaactattttccccttccta | |
| | | | cctttccagccacactacagtgtctgtcgcca | |
| | | | ccaactgcagggaggccagccacggtgcagca | |
| | | | ggctacagcctccagtctgtcctggtcctcta | |
| | | | agccgggctcggagcagctcggtgagcagaca | |
| | | | cagaagaacctggaacagcctgactcttcttc | |
| | | | agccccatttatgtactgaagttatgcatatg | |
| | | | cggttcgtggactacactttccaggattggat | |
| | | | aagagaaagcccggaggcctactctgattgga | |
| | | | ctttgttatcatgttctgattggatgaaagtc | |
| | | | ttaggacaaccaattagagtatgaaaataaag | |
| | | | tccaatcagagaaggcctagagattttctctc | |
| | | | acccaatcagaacatgtagtccagaaaccatg | |
| | | | cgcgtaaccccatgtgcatgccgagcaggcct | |
| | | | cacgccagtttagggtctctggtatctcccgc | |
| | | | tgagctgctctgttcccggcttagaggaccag | |
| | | | gagaagggggagttggaggctggagcctgtaa | |
| | | | caccgtggctcgtctcgctctggatggtggtg | |
| | | | gcaacagagatggcagcgcagctggagtgtta | |
| | | | ggagggcggcctgagcggtaggagtggggctg | |
| | | | gagcagtaag | |
| 5 | BRCA1 | breast cancer | GGCGATTGGGCCCTCTAGATGCATGCTCGAGC | NM_007295 |
| | | 1, early onset | GGCCGCCAGTGTGATGGATATCTGCAGAATTC | |
| | | | GCCCTTGAAATCCACTCTCCCACGCCAGTACC | |
| | | | CCAGAGCATCACTTGGGCCCCCTGTCCCTTTC | |
| | | | CCGGGACTCTACTACCTTTACCCAGAGCAGAG | |
| | | | GGTGAAGGCCTCCTGAGCGCAGGGGCCCAGT | |
| | | | TATCTGAGAAACCCCACAGCCTGTCCCCCGTC | |
| | | | CAGGAAGTCTCAGCGAGCTCACGCCGCGCAG | |
| | | | TCGCAGTTTTAATTTATCTGTAATTCCCGCGCT | |
| | | | TTTCCGTTGCCACGGAAACCAAGGGGCTACC | |
| | | | GCTAAGCAGCAGCCTCTCAGAATACGAAATC | |
| | | | AAGGTACAATCAGAGGATGGGAGGGACAGA | |
| | | | AAGAGCCAAGCGTCTCTCGGGGCTCTGGATT | |
| | | | GGCCACCCAGTCTGCCCCCGGATGACGTAAA | |
| | | | AGGAAAGAGACGGAAGAGGAAGAATTCTACC | |
| | | | TGAGTTTGCCATAAAGTGCCTGCCCTCTAGCC | |
| | | | TCTACTCTTCCAGTTGCGGCTTATTGCATCAC | |
| | | | AGTAATTGCTGTACGAAGGTCAGAATCGCTA | |
| | | | CCTATTGTCCAAAGCAGTCGTAAGAAGAGGT | |
| | | | CCCAATCCCCCACTCTTTCCGCCCTAATGGAG | |
| | | | GTCTCCAGTTTCGGTAAATATGAGTAATAAGG | |
| | | | ATTGTTGGGGGGGTGGAGGGAAATAATTATT | |
| | | | TCCAGCATGCNTTGCGGAATGAAAGGTCTTCG | |
| | | | CCACAGTGTTCCTTAGAAACTGTAGTCTTATG | |
| | | | GANAGGAACATCCAATACCANAGCGGGCACA | |
| | | | ATTCTCACGGGAAATCCAGTGGATANATTGG | |
| | | | AGACCTGTGCNCGCTTGTACTTGTCAACAGTT | |
| | | | TATGGNACTGGAGTGTTATGTTNANGGGCNA | |
| | | | TTTCCANCACACTGGCGGGCCG | |
| 6 | Calcitonin | Calcitonin | GGAGTGGCGGCTGAAGAAGCCAGGGTCACAA | NM_001742 |
| | CGPR | | TGTCTCTGGGATAAGGTTCTTGTGGAAACTCA | |
| | | | CCTCCCTCCGGAATTTGCATTCTCCGGGGAGG | |
| | | | GGACAGGGCTCCCAGAAAGCTGTCTCCCAGT | |
| | | | CCAGACTGTCGCCCCCCTCTCCCTCCCTACTC | |
| | | | AAGGTCTAACTCGGGTCCCTCGCCTGCTTCCT | |
| | | | GTGTTTACGCGGCGCTTTAGTCTCCCGGACTC | |
| | | | GCAGGGTGAGCCCCAGCCCTGACTGGAGCGA | |
| | | | GACAGCAGCCGCGAGCGCAGCCCCACTCGCG | |
| | | | GGCCGGGGCGACTGGGGCTGGCGCGAGGCGC | |
| | | | ACGGAGCTCACCAGCTCGCCCCTCCCTCTCCT | |
| | | | GGGACAGGAGGGGGCTGACTGGGGTGGCGGG | |
| | | | GTCCGGGAAGGGGGGCTGGCTCTCATCAATT | |
| | | | CTGCTGCCACCTCCTCTGCCGCCTGTCGGGAG | |
| | | | GCGGGCGGGGGTGGGGCGGGAGCGCAGGCTA | |
| | | | GGATTGAGACTCTTAAGTCAGGAGAAGTTTG | |
| | | | CGCACAGCTTCACAGCTGGGAGAGCGCAGGA | |
| | | | AGGCGCCGGGAAGGTGAGCCTCCTGGACTCT | |
| | | | GGGGAGGTAGAAAGCAAGCCAGGGGAAAGA | |
| | | | ACAGTTGTCTTTTAGCTGATAATACAACCTAG | |
| | | | ACTTGGGTCTGAACCACCTAAGACAGATTTAA | |
| | | | AGTGTCAGAAAACCAGGAGAGGGGCGGAGA | |
| | | | GGGAGGACTGAGACTAACGCAGTTTGCTCTC | |
| | | | GCATCAAACTAGGAAAGCCAGCCCACCAGCG | |
| | | | TCTGGGTGGGCTGCGCCGCGCGGCTGGCGGA | |
| | | | CCTTCCCGGGTTGGAGAAGTGCGCACGTCCGC | |
| | | | ACCTCACCCTGCGGCTGACATCTCCTGCCCAG | |
| | | | GAGATGGGCGCTGAAGCTTGAGCGCCTGAGT | |
| | | | CCCTGGAGCCACACCTGCGAACACCCTTTGCT | |
| | | | TCTATTGAGCTGTGCCCAGCCGCCCAGTGACA | |
| | | | GAATTCCAGGTAAGGAGCGTTTGGAAATGAG | |
| | | | CGGGACTTAACGATTTGGGGTGTCCAAG | |
| 7 | CASP8 | caspase 8, | ttaaaaatacaaaaattagccgggggtggtggt | AF422925 |
| | (CASPASE | apoptosis- | gggtgcctgtagtcccagctactcgggaggctg | |
| | 8) | related | aggcaggagaatcacctgaactcaggaggtgga | |
| | | cysteine | ggttgcagtgagtcaagatcgcaccactgtact | |
| | | protease | gttgcctgggcaacgcaccgagactccgtctca | |
| | | | aaaaaaaaaaaaaaaTGAGAGAACAGGGGAGGG | |
| | | | TCTAGGGCTCAGAGCTTTGGAGAACA | |
| | | | GACCTCAGTAGCACCAACACTCCAGGATCAA | |
| | | | TGCTACAAAGACACGGGTTACAACTAAACTG | |
| | | | GAGAACATGGCCAAGGATGGGAACTCAGCctg | |
| | | | agcagggctgagccgagcagggctaagccaagt | |
| | | | agggctgagcCAGAACACTTCCTCCTTTTTTCT | |
| | | | GAACAATCTACCTACATTTCAGCTACAGGGCTG | |
| | | | GCTTTACCCAGTCCGGCGGGAGGGAGGAGAGGG | |
| | | | CTGGTCTGTGACTTCAGTGCTGAGGTTTGATCA | |
| | | | AGGCAAAGGGAAACTTCCTATTCCCAGACCCTT | |
| | | | TGCAAGAAAGAATGGCATATTACTTGCCACCGA | |
| | | | CAGGGGTTATTATTACTAAATGGAGTCAGTATA | |
| | | | AATGCTTTCCAATAAAGCATGTCCAGCGCTCGG | |
| | | | GCTTTAGTTTGCACGTCCATGAATTGTCTGCCA | |
| | | | CATCCCTCTTCTGAATGGTTGGAAATTGGGCAT | |
| | | | CTGTTCCTTTAAACAGGAAACATTTCTTGTTCG | |
| | | | AGTGAGTCATCTCTGTTCTGCTTTAGGAGTAA | |
| | | | AGTTTACCCTGCAGTTCCTTCTGTGGTGAAGT | |
| | | | TTTCTCTTTCTCTCGGAGACCAGATTCTGCCTT | |
| | | | TCTGCTGGAGGGAAGTGTTTTCACAGGTTCTC | |
| | | | CTCCTTTTATCTTTTGTGTTTTTTTTCAAGCCC | |
| | | | TGCTGAATTTGCTAGTCAACTCAACAGGAAGT | |
| | | | GAGGCCATGGAGGGAGGCAGAAGAGCCAGG | |
| | | | GTGGTTATTGAAAGTAAAAGAAACTTCTTCCT | |
| | | | GGGAGCCTTTCCCACCCCCTTCCCTGCTGA | |
| 8 | CD 14 | CD 14 antige | ggatagtgtaagtgacccagagacttggccaat | AF097335 |
| | | | gtgtctctgttaaatacatccacttttaagaaa | |
| | | | gttagtactgccaggcacagtggctcacgcctg | |
| | | | taatcccagcactttgggaggccgaggcgggtg | |
| | | | ggatcacaaggtcaggagttcaaaccagcctgg | |
| | | | ccaagatgatgaaaacctgtctctactaaaaaa | |
| | | | tacaaaaattagctgggtgtggtggtgggcact | |
| | | | tgtaatcccagctactcgggaggctgaggcaga | |
| | | | gaattgcttgaacccaggaggcggaggttgcag | |
| | | | tgagccgagatcatggcactctactccagcctg | |
| | | | agcaacagagcaagactctatctcaaaaaaaaa | |
| | | | aaaaaaaaagaaagaaagttattacttaatcaa | |
| | | | aggagcaaggaaaaaaaaaggaagggggaattt | |
| | | | ttctttagaccaacttccttttcttgaacctaa | |
| | | | ttctaccccccttggtgccaacagatgaggttc | |
| | | | acaatctcttccacaaaacatgcagttaaatat | |
| | | | ctgaggatattcagggacttggatttggtggca | |
| | | | ggagatcaacataaaccaagacaaggaagaagt | |
| | | | caaagaaatgaatcaagtagattctctgggata | |
| | | | taaggtagggggattggggggttggatagtgca | |
| | | | gagtatggtactggcctaaggcactgaggatca | |
| | | | tccttttcccacacccaccagagaaggcttagg | |
| | | | ctcccgagtcaacagggcattcaccgcctgggg | |
| | | | cgcctgagtcatcaggacactgccaggagacac | |
| | | | agaaccctagatgccctgcagaatccttcctgt | |
| | | | tacggtccccctccctgaaacatccttcattgc | |
| | | | aatatttccaggaaaggaagggggctggctcgg | |
| | | | aggaagagaggtggggaggtgatcagggttcac | |
| | | | agaggagggaactgaatgacatcccaggattac | |
| | | | ataaactgtcagaggcagccgaagagttcacaa | |
| | | | gtgtgaagcctggaagccggcgggtgccgctgt | |
| | | | gtaggaaagaagctaaagcacttccagagcctg | |
| | | | tccggagctcagaggttcggaagacttatcgac | |
| | | | c | |
| 9 | CDC2 | Homo sapiens | CCTCTAGATGCATGCTCGAGCGGCCGCCAGTG | AF512554 |
| | | cell division | TGATGGATATCTGCAGAATTCGCCCTTGTTCT | |
| | | cycle 2, G1 | CGGATCCCGATCAGATCCCTGACCTCCAGTCC | |
| | | to S and G2 to | GGCCTTCTTAGAGGACCCCGTTCCTCAATACT | |
| | | M (CDC2) | CGCCCTCCGAGGCCCTCGGCCGTCCCCTAGAC | |
| | | | ACGACCCTGACCCCAGCCACTGTACCCGGCTT | |
| | | | ATTATTCCGCGGCGGCCGCAGCGGCAGCTAC | |
| | | | AACAACCGCGTCGCTCTCCGCTCAATTTCCAA | |
| | | | GAGCCAGCTTTGAAGCCAAGTGCGAGCAGTT | |
| | | | TCAAACTCACCGCGCTAAAGGGCCCCGGATT | |
| | | | CACCAATCGGGTAGCCCGTAGACTTTCAAAG | |
| | | | CAGCCAATCAGAGCCCAGCTACGCTGGGCAG | |
| | | | GCCTTCCCGGGTGGCTAGAGCGCGAAAGAAA | |
| | | | GAGGAAAGGGCGGCTAGAGAAAAAGCAGGA | |
| | | | GGGCGGGCGCCAACTGAGTGCGAGCGCAAGC | |
| | | | GCTCTCCTCCAGTCGGGAGAGTGTCGTCCTAC | |
| | | | TGTTTCTAGTCAGCGGAGCAGGAAGCTACTGT | |
| | | | TCGCTCCGTTCTTCTTTTAAATTTTTTCTCCCA | |
| | | | GCATTGGCACAGTTCAAATTTATTATACTCAA | |
| | | | AATAGCTCATCAAAAAAGTGATATTGTGTTTA | |
| | | | CATCGAGATTCCATTACTTTCACTTCTAATAC | |
| | | | TTAGGGTTAGGAGTGNATAGTTATGTTTTTCT | |
| | | | AAATGCGTGATTCGCGGGCTGGCTCCNAGGA | |
| | | | GCACATTTCAGTGACCTTAAGAAGGAAATGG | |
| | | | AAAACTCAAAAGACCGCCTCAAAAATGTAAA | |
| | | | GGAAAATTTATTATTTATATCGCTGTGCTTTG | |
| | | | TTTCTACCTCATTTTTGAATTTAATATTAAATT | |
| | | | ATTTTATTATTTACATTTTGTTTATTATACAAT | |
| | | | TAAAAACATTTGAAATGTATTAAATTTTAAAA | |
| | | | TATTTTCACATCAGAATTTTAAATATATAGAG | |
| | | | AGAGGCATG | |
| 10 | CDKN2 | cyclin- | actcatattcccttccccctttataattacgaa | NM_058196 |
| | A | dependent | aaatgcaaggtattttcagtaggaaagagaaat | |
| | | kinase | gtgagaagtgtgaaggagacaggacagtatttg | |
| | | inhibitor 2A | aagctggtctttggatcactgtgcaactctgct | |
| | | (melanoma, | tctagaacactgagcactttttctggtctagga | |
| | | p16, inhibits | attatgactttgagaatggagtccgtccttcca | |
| | | CDK4) | atgactccctccccattttcctatctgcctaca | |
| | | | ggcagaattctcccccgtccgtattaaataaac | |
| | | | ctcatcttttcagagtctgctcttataccaggc | |
| | | | aatgtacacgtctgagaaacccttgccccagac | |
| | | | agccgttttacacgcaggaggggaaggggaggg | |
| | | | gaaggagagagcagtccgactctccaaaaggaa | |
| | | | tcctttgaactagggtttctgacttagtgaacc | |
| | | | ccgcgctcctgaaaatcaagggttgagggggta | |
| | | | gggggacactttctagtcgtacaggtgatttcg | |
| | | | attctcggtggggctctcacaactaggaaagaa | |
| | | | tagttttgctttttcttatgattaaaagaagaa | |
| | | | gccatactttccctatgacaccaaacaccccga | |
| | | | ttcaatttggcagttaggaaggttgtatcgcgg | |
| | | | aggaaggaaacggggcgggggcggatttctttt | |
| | | | taacagagtgaacgcactcaaacacgcctttgc | |
| | | | tggcaggcgggggagcgcggctgggagcaggga | |
| | | | ggccggagggcggtgtggggggcaggtggggag | |
| | | | gagcccagtcctccttccttgccaacgctggct | |
| | | | ctggcgagggctgcttccggctggtgcccccgg | |
| | | | gggagacccaacctggggcgacttcaggggtgc | |
| | | | cacattcgctaagtgctcggagttaatagcacc | |
| | | | tcctccgagcactcgctcacggcgtccccttgc | |
| | | | ctggaaagataccgcggtccctccagaggattt | |
| | | | gagggacagggtcggagggggctcttccgccag | |
| | | | caccggaggaagaaagaggaggggctggctggt | |
| | | | caccagagggtggggcggaccgcgtgcgctcgg | |
| | | | cggctgcggagagggggagagcaggcagcgggc | |
| | | | ggcggggagcagc | |
| 11 | CFTR | cystic fibrosis | ACAAGGAACACATCCTGGGCCGGTAATTACG | NM_000492 |
| | | transmembrane | CAAAGCATTATCTCCTCTTACCTCCTTGCAGA | |
| | | conductance | TTTTTTTTTCTCTTTCAGTACGTGTCCTAAGAT | |
| | | regulator, | TTCTGTGCCACCCTTGGAGTTCACTCACCTAA | |
| | | ATP-binding | ACCTGAAACTAATAAAGCTTGGTTCTTTTCTC | |
| | | cassette (sub- | CGACACGCAAAGGAAGCGCTAAGGTAAATGC | |
| | | family C, | ATCAGACCCACACTGCCGCGGAACTTTTCGGC | |
| | | member 7) | TCTCTAAGGCTGTATTTTGATATACGAAAGGC | |
| | | | ACATTTTCCTTCCCTTTTCAAAATGCACCTTGC | |
| | | | AAACGTAACAGGAACCCGACTAGGATCATCG | |
| | | | GGAAAAGGAGGAGGAGGAGGAAGGCAGGCT | |
| | | | CCGGGGAAGCTGGTGGCAGCGGGTCCTGGGT | |
| | | | CTGGCGGACCCTGACGCGAAGGAGGGTCTAG | |
| | | | GAAGCTCTCCGGGGAGCCGGTTCTCCCGCCG | |
| | | | GTGGCTTCTTCTGTCCTCCAGCGTTGCCAACT | |
| | | | GGACCTAAAGAGAGGCCGCGACTGTCGCCCA | |
| | | | CCTGCGGGATGGGCCTGGTGCTGGGCGGTAA | |
| | | | GGACACGGACCTGGAAGGAGCGCGCGCgaggga | |
| | | | gggaggctgggagtcagaatcgggaaagggagg | |
| | | | tgcggggcggcgagggagcgaaggaggagagga | |
| | | | ggaaggagcgggagggGTGCTGGCGGGGGTGCG | |
| | | | TAGTGGGTGGAGAAAGCCGCTAGAGCAAATTTG | |
| | | | GGGCCGGACCAGGCAGCACTCGGCTTTTAACCT | |
| | | | GGGCAGTGAAGGCGGGGGAAAGAGCAAAAGGAA | |
| | | | GGGGTGGTGTGCGGAGTAGGGGTGGGTGGGGGG | |
| | | | AATTGGAAGCAAATGACATCACAGCAGGTCAGA | |
| | | | GAAAAAGGGTTGAGCGGCAGGCACCCAGAGTAG | |
| | | | TAGGTCTTTGGCATTAGGAGCTTGAGCCCAGAC | |
| | | | GGCCCTAGCAGGGACCCCAGCGCCCGAGAGACC | |
| | | | ATGCAGAGGTCGCCTCTGGAAAAGGCCAGCGTT | |
| | | | GTCTCCAAACTTTTTTTCAGGTGAGAAGGTGGC | |
| | | | CA | |
| 12 | CIITA | class II | taaccatttaacaagaaagcagagtgatgttag | U67329 |
| | | transactivator | attatagcaagatactgttgactgtagaaggct | |
| | | | ctgaggctagagagctgctttctataaaacaga | |
| | | | gtgatcatatattagaagaggtgttaaagacat | |
| | | | gttcacaccaagctgagacttcctccttgatac | |
| | | | caccaggaggatgggcagagactggaaaagaca | |
| | | | ctaactttctccctatgggagtcagtattattt | |
| | | | agcatcactttggcgggtcaccccaaaccatct | |
| | | | gactacaagggtaccatatttgggttaacactC | |
| | | | TTTTGGTATAATTTATGTTTTAGTCCAATG | |
| | | | TCTTGGGATGAAAATGACAGGTGGGCCAC | |
| | | | TTATGATCTCCAGAGAAATTCAGGGCA | |
| | | | ATTTGGTGTGGGAGTAGGCATGGTAGAGGA | |
| | | | GAGCAGCATCTAAGAAGTCC | |
| | | | CCAGCAGAGGCTCTCAGCTTGTCTTGAGGCAT | |
| | | | CTGGGCGGAGGGCTATGATACTGGCCCCATC | |
| | | | CTGCAGAAGGTGGCAGATATTGGCAGCTGGC | |
| | | | ACCAGTGCGGTTCCATTGTGATCATCATTTCT | |
| | | | GAACGTCAGACTGTTGAAGGTTCCCCCAACA | |
| | | | GACTTTCTGTGCAACTTTCTGTCTTCACCAAA | |
| | | | TTCAGTCCACAGTAAGGAAGTGAAATTAATTT | |
| | | | CAGAGGTGTGGGGAGGGCTTAAGGGAGTGTG | |
| | | | GTAAAATTAGAGGGTGTTCAGAAACAGAAAT | |
| | | | CTGACCGCTTGGGGCCACCTTGCAGGGAGAG | |
| | | | TTTTTTTGATGATCCCTCACTTGTTTCTTTGCA | |
| | | | TGTTGGCTTAGCTTGGCGGGCTCCCAACTGGT | |
| | | | GACTGGTtagtgatgaggctagtgatgaggctG | |
| | | | TGTGCTTCTGAGCTGGGCATCCGAAGGCATCC | |
| | | | TTGGGGAAGCTGAGGGCACGAGGAGGGGCTGC | |
| | | | CAGACTCCGGGAGCTGCTGCCTGGCTGGGAT | |
| | | | TCCTACACAATGCGTTGCCTGGCTCCACGCCCT | |
| | | | GCTGGGTCCTACCTGTCAGAGCCCCAA | |
| 13 | COX2 | prostaglandin- | TAGGACCAGTATTATGAGGAGAATTTACCTTT | NM_000963 |
| | | endoperoxide | CCCGCCTCTCTTTCCAAGAAACAAGGAGGGG | |
| | | synthase 2 | GTGAAGGTACGGAGAACAGTATTTCTTCTGTT | |
| | | (prostaglandin | GAAAGCAACTTAGCTACAAAGATAAATTACA | |
| | | G/H synthase | GCTATGTACACTGAAGGTAGCTATTTCATTCC | |
| | | and | ACAAAATAAGAGTTTTTTAAAAAGCTATGTAT | |
| | | cyclooxygenase) | GTATGTGCTGCATATAGAGCAGATATACAGC | |
| | | COX-2, | CTATTAAGCGTCGTCACTAAAACATAAAACAT | |
| | | COX2, | GTCAGCCTTTCTTAACCTTACTCGCCCCAGTC | |
| | | PGG/HS, | TGTCCCGACGTGACTTCCTCGACCCTCTAAAG | |
| | | PGHS-2 | ACGTACAGACCAGACACGGCGGCGGCGGCGG | |
| | | | GAGAGGGGATTCCCTGCGCCCCCGGACCTCA | |
| | | | GGGCCGCTCAGATTCCTGGAGAGGAAGCCAA | |
| | | | GTGTCCTTCTGCCCTCCCCCGGTATCCCATCC | |
| | | | AAGGCGATCAGTCCAGAACTGGCTCTCGGAA | |
| | | | GCGCTCGGGCAAAGACTGCGAAGAAGAAAAG | |
| | | | ACATCTGGCGGAAACCTGTGCGCCTGGGGCG | |
| | | | GTGGAACTCGGGGAGGAGAGGGAGGGATCA | |
| | | | GACAGGAGAGTGGGGACTACCCCCTCTGCTC | |
| | | | CCAAATTGGGGCAGCTTCCTGGGTTTCCGATT | |
| | | | TTCTCATTTCCGTGGGTAAAAAACCCTGCCCC | |
| | | | CACCGGGCTTACGCAATTTTTTTAAGGGGAGA | |
| | | | GGAGGGAAAAATTTGTGGGGGGTACGAAAAG | |
| | | | GCGGAAAGAAACAGTCATTTCGTCACATGGG | |
| | | | CTTGGTTTTCAGTCTTATAAAAAGGAAGGTTC | |
| | | | TCTCGGTTAGCGACCAATTGTCATACGACTTG | |
| | | | CAGTGAGCGTCAGGAGCACGTCCAGGAACTC | |
| | | | CTCAGCAGCGCCTCCTTCAGCTCCACAGCCAG | |
| | | | ACGCCCTCAGACAGCAAAGCCTACCCCCGCG | |
| | | | CCGCGCCCTGCCCGCCGCTGCGATGCTCGCCC | |
| | | | GCGCCCTGCTGCTGTGCGCGGTCCTGGCGCTC | |
| | | | AGCCATACAGGTGAGTACCTGGCG | |
| 14 | Cyclin | Cyclin D2 | cacgatggtttctgctcgaggatcacattcta | NM_001759 |
| | D2 | | tccctccagagaagcaccccccttccttccta | |
| | | | atacccacctctccctccctcttcttcctctg | |
| | | | cacacactctgcaggggggggcagaagggacg | |
| | | | ttgttctggtccctttaatcggggctttcgaa | |
| | | | acagcttcgaagttatcaggaacacagacttc | |
| | | | agggacatgacctttatctctgggtatgcgag | |
| | | | gttgctattttctaaaatcaccccctccctta | |
| | | | tttttcacttaagggacctatttctaaattgt | |
| | | | ctgaggtcaccccatcttcagataatctaccc | |
| | | | tacattcctggatcttaaatacaagggcagga | |
| | | | ggattaggatccgttttgaagaagccaaagtt | |
| | | | ggagggtcgtattttggcgtgctacacctaca | |
| | | | gaatgagtgaaattagagggcagaaataggag | |
| | | | tcggtagttttttgtgggttgccctgtccggg | |
| | | | gcccctggcatgcagggctggatggagggaga | |
| | | | ggggtggggggtggcgggggaccgcgtttgaa | |
| | | | gttgggtcgggccagctgctgttctccttaat | |
| | | | aacgagaggggaaaaggagggagggagggaga | |
| | | | gattgaaaggaggaggggaggaccgggagggg | |
| | | | aggaaaggggaggaggaaccagagcggggagc | |
| | | | gcggggagagggaggagagctaactgcccagc | |
| | | | cagcttgcgtcaccgcttcagagcggagaaga | |
| | | | gcgagcaggggagagcgagaccagttttaagg | |
| | | | ggaggaccggtgcgagtgaggcagccccgagg | |
| | | | ctctgctcgcccaccacccaatcctcgcctcc | |
| | | | cttctgctccaccttctctctctgccctcacc | |
| | | | tctcccccgaaaaccccctatttagccaaagg | |
| | | | aaggaggtcaggggaacgctctcccctcccct | |
| | | | tccaaaaaacaaaaacagaaaaacctttttcc | |
| | | | aggccggggaaagcaggagggagaggggccgc | |
| | | | cgggctggcc | |
| 15 | DAPK | death- | ggactctaatgtgtattttacacttacagcac | NM_004938 |
| | | associated | aattaatttgggactagctacatttcagctca | |
| | | protein kinase | acaatagccaatagcatatgggatagcgcaAA | |
| | | 1 | TAAACTCTGCGTCTCTGTTGCTTCTTTGGGTC | |
| | | | TCGGAGACCTCAACCCTTTCTTCAGATTGCAA | |
| | | | ACCTTCTTGccttcaagcctcggctccaacacca | |
| | | | gtccggcagaggaacccagtctaatgaggtacgc | |
| | | | tcccttcctgccattctctattccattaacct | |
| | | | gtttcgtggtaaacgtaggactgatcctccaa | |
| | | | aattaccttattaattagcttacatatttatta | |
| | | | tctatctgtcccaccagaatgcaggtttccgga | |
| | | | aggcagggatttaaaaaaatctgttttgttct | |
| | | | atgtgattttcccataccaagcaccgtgcccg | |
| | | | gcacaagctgggatcccagtacacatctCGGG | |
| | | | ACGGAAGAACCGTGTTTCCCTAGAACCCAGT | |
| | | | CAGAGGGCAGCTTAGCAATGTGTCACAGGTG | |
| | | | GGGCGCCCGCGTTCCGGGCGGACGC | |
| | | | ACTGGCTCCCCGGCCGGCGTGGGTGTGG | |
| | | | GGCGAGTGGGTGTGTGCGGGGTGTGCG | |
| | | | CGGTAGAGCGCGCCAGCGAGCCCGGAGC | |
| | | | GCGGAGCTGGGAGGAGCAGCGAGCGCCGCGC | |
| | | | AGAACCCGCAGCGCCGGCCTGGCAGGGCAGC | |
| | | | TCGGAGGTGGGTGGGCCGCGCCGCCAGCCCG | |
| | | | CTTGCAGGGTCCCCATTGGCCGCCTGCCGGCC | |
| | | | GCCCTCCGCCCAAAAGGCGGCAAGGAGCCGA | |
| | | | GAGGCTGCTTCGGAGTGTGAGGAGGACAGCC | |
| | | | GGACCGAGCCAACGCCGGGGACTTTGTTCCCT | |
| | | | CCGCGGAGGGGACTCGGCAACTCGCAGCGGC | |
| | | | AGGGTCTGGGGCCGGCGCCTGGGAGGGATCT | |
| | | | GCGCCCCCCACTCACTCCCTAGCTGTGTTCCC | |
| | | | GCCGCCGCCCCGGCTAGTCTCCGGCGCTGGCG | |
| | | | CCTATGGTCGGCCTCCGACAGCGCTCCGGAG | |
| 16 | DBCCR | deleted in | ATCATACGAGGGCTTTATTTTCTGCTTCAGGA | NM_014618 |
| | 1 | bladder cancer | AGAGGCCCTATGTTAGCAGCCCCAGCCTGCAT | |
| | | 1 | TCAGGCTGATTGCAGAGTATTTTGCTTTTTATT | |
| | | | TTCATGTCTTAGTCCCTGTACCCTCGCCCCTTC | |
| | | | CCCGCCTCTGGTGGTCTCCAGAGAACTTCGTG | |
| | | | TCCCCTCAGCTTCTCCCTCCTACATCCTGCCTA | |
| | | | CGTAGAGAAGCTCTTGCTTCATTCTGGGAGGT | |
| | | | TACGTGGGCTCTCGCCTACACACCGAGAGAA | |
| | | | ACAAACAGTGTCAAACACTCACAGAGAGACG | |
| | | | CGCAGACACAAACGGACCCACACGGGCAACT | |
| | | | CCCGAGACAAAACCCACACTCGATGGATCCA | |
| | | | CGCGGCCGTGGAAACACCTGCCGCCCCAGAA | |
| | | | ACACTCAGGTACTCGCGACACACACAGTACA | |
| | | | GTCACGCTTAAGGGCACCAGGATTCCGGGTTT | |
| | | | GCGCGTATGCGCGGTCCCTTTGGATGCTCGTG | |
| | | | CGCATAGACACAACACCCTACACGCCCCAGA | |
| | | | CCCACGAAACTCCCTACGGCTCAGCCCCAGCC | |
| | | | CACCCGGGCCGCCCTTCCCTCGAGGCGGCCTC | |
| | | | CCGTCTCTCCTCCTCTCGCTTCTCCTCCTCCTC | |
| | | | CGCCTAAAGATGTACAAAACACTCCTCGGAA | |
| | | | GCAACCCCGGCGTTCAGCTCCTCCCTCCCCGC | |
| | | | CCCCCGGCCGCCGCTCCCCCATTCATTTTCGG | |
| | | | CCGTCGCCGGCTAAGTCCCTCCCCCGGCGTAG | |
| | | | CCCGGCCTCCGCCGCTCCCCGCCCGGAGACCG | |
| | | | CGGCGCACTTGGACTTCCCTCTCCATTCGCCA | |
| | | | GCCGCCTCGCTCCCGGACCCCACGGCTGCAA | |
| | | | ACTGATCTGGCGCGCGGGGAGGAGgagagcgca | |
| | | | ggcgagcgaacccgcgagagagggagagagcg | |
| | | | agcgagcaacagcgagagcgagagcgagagag | |
| | | | cCGGGAGGCAGAGGGAGTA | GTGACCGCCTTC |
| | | | CGGAGCCGGGATTCATGCCT | GTCCTCGGGAC |
| | | | CAGCGAAGGGGACT | |
| 17 | E- | ECAD (E- | ggagagtctcttgaacccggcaggcggaggttgc | L34545 |
| | CAD(500) | cadherin) | agtgagccgagatcgtgccactgcactccagcctg | |
| | | | ggcaagacagagcgagactccgtctcaaaa | |
| | | | aatacaaacaaaacaaacaaacaaaaAATTAGGC | |
| | | | TGCTAGC | TCAGTGGCTCAtggctcacacctgaa |
| | | | atcctagcactttgggaggccaaggcaggaggatc | |
| | | | gcttcagcccaggagttcgagaccaggctgggcaa | |
| | | | tacagggagacacagcgcccccactgcccctgtcc | |
| | | | gccccgacttgtctctctacaaaaaggcaaaagaa | |
| | | | aaaaaaattagcctggcgtggtggtgtgcacctgt | |
| | | | actcccagctactagagaggctggggccagaggac | |
| | | | cgcttgagcccaggagttcgaggctgcagtgagct | |
| | | | gtgatcgcaccactgcactccagcttgggtgaaa | |
| | | | gagtgagaccccatctccaaaacgaacaaacaaaa | |
| | | | aatcccaaaaaacaaaAGAACTCAGCCAAGTGTAA | |
| | | | AAGCCCTTTCTGATCCCAGGTCTTAGTGAGCCACC | |
| | | | GGCGGGGCTGGGATTCGAACCCAGTGGAATCAGA | |
| | | | ACCGTGCAGGTCCCATAACCCACCTAGACCCT | |
| | | | AGCAACTCCAGGCTAGAGGGTCACCGCGTCT | |
| | | | ATGCGAGGCCGGGTGGGCGGGCCGTCAGCTC | |
| | | | CGCCCTGGGGAGGGGTCCGCGCTGCTGATTG | |
| | | | GCTGTGGCCGGCAGGTGAACCCTCAGCCAAT | |
| | | | CAGCGGTACGGGGGGCGGTGCCTCCGGGGCT | |
| | | | CACCTGGCTGCAGCCACGCACCCCCTCTCAGT | |
| | | | GGCGTCGGAACTGCAAAGCACCTGTGAGCTTGCG | |
| | | | GAAGTCAGTTCAGACTccagcccgctccagccc | |
| | | | ggcccgacccgaccgcacccggcgcctgccctcg | |
| | | | ctcggcGTCCCCG | GCCAGCCATGGGCCCTTGGA |
| | | | GCCGCAGCCTCTCGGCGCTGCTGCTGCTGCTG | |
| | | | CAGGTACCCCGGATCCCCTGACTTGCGAGGG | |
| 18 | ER | Estrogen | CCGACAATGTAACATAATTGCCAAAGCTTTGG | X62462 |
| | | receptor alpha | TTCGTGACCTGAGGTTATGTTTGGTATGAAAA | |
| | | | GGTCACATTTTATATTCAGTTTTCTGAAGTTTT | |
| | | | GGTTGCATAACCAACCTGTGGAAGGCATGAA | |
| | | | CACCCATGTGCGCCCTAACCAAAGGTTTTTCT | |
| | | | GAATCATCCTTCACATGAGAATTCCTAATGGG | |
| | | | ACCAAGTACAGTACTGTGGTCCAACATAAAC | |
| | | | ACACAAGTCAGGCTGAGAGAATCTCAGAAGG | |
| | | | TTGTGGAAGGGTCTATCTACTTTGGGAGcatt | |
| | | | ttgcagaggaagaaactgaggtcctggcaggtT | |
| | | | GCATTCTCCTGATGGCAAAATGCAGCTCTTCCT | |
| | | | ATATGTATACCCTGAATCTCCGCCCCCTTCCC | |
| | | | CTCAGATGCCCCCTGTCAGTTCCCCCAGCTGC | |
| | | | TAAATATAGCTGTCTGTGGCTGGCTGCGTATG | |
| | | | CAACCGCACACCCCATTCTATCTGCCCTATCTC | |
| | | | GGTTACAGTGTAGTCCTCCCCAGGGTCATCCT | |
| | | | ATGTACACACTACGTATTTCTAGCCAACGAGG | |
| | | | AGGGGGAATCAAACAGAAAGAGAGACAAACAGA | |
| | | | GATATATCGGAGTCTGGCACGGGGCACATAAG | |
| | | | GCAGCACATTAGAGAAAGCCGGCCCCTGGATCC | |
| | | | GTCTTTCGCGTTTATTTTAAGCCCAGTCTTCC | |
| | | | CTGGGCCACCTTTAGCAGATCCTCGTGCGCCC | |
| | | | CCGCCCCCTGGCCGTGAAACTCAGCCTCTATCC | |
| | | | AGCAGCGACGACAAGTAAAGTAAAGTTCAGGG | |
| | | | AAGCTGCTCTTTGGGATCGCTCCAAATCGAGTT | |
| | | | GTGCCTGGAGTGATGTTTAAGCCAATGTCAGGG | |
| | | | CAAGGCAACAGTCCCTGGCCGTCCTCCAGCA | |
| | | | CCTTTGTAATGCATATGAGCTCGGGAGACCAG | |
| | | | TACTTAAAGTTGGAGGCCCGGGAGCCCAGGA | |
| | | | GCTGGCGGAGGGCGTTCGTCCTGGGACTGCA | |
| | | | CTTGCTCCCGTCGGGTCGCCCGGCTTCACCGG | |
| | | | ACCCG | |
| 19 | FHIT | fragile | GAGAAAGGGAGACTAGGGGAGAAAGGTCAC | NM_002012 |
| | | histidine triad | TCTAGATTTCGTTCAATTATTGAAAATACGGT | |
| | | gene | gtatttactatgtgctgggcacttttctaggt | |
| | | | gctagaaagactacagtgaccaaaacaaaa | |
| | | | atccacatctgcagggatcttgcattctagtg | |
| | | | agaaagtaagatggtaaaaaagataaatacgt | |
| | | | aaattttatacaatgcttcgtaacgacaaatg | |
| | | | ctaaggagaaaaacagcacagaaaagacaga | |
| | | | aaggaaaagagaaggggcgcatgtggtgca | |
| | | | attttgttaggatgccagggagggcTGAGCGT | |
| | | | AGTCGTAAATGACCACATTATTTGATGGATCA | |
| | | | AGCCAGGGACTGCAAGTCTGTGTTTCTGAGA | |
| | | | GACACATAAAGAAAAGAAGGCTTAAGGAATC | |
| | | | CAGAAAGATCCAGAGTGGGGAAATGAAACGA | |
| | | | AAAGAAATCCAGCCAGTGGGAAGTCGTGAAG | |
| | | | GGATAGTTAAACGCGTTTTGGGAGGAAAGAA | |
| | | | AAAAGCAAAAGTGCGGTACAGCCTTTCGTTA | |
| | | | CACGTGAAAAGAATCATGTTTCTTTTTCTAGT | |
| | | | TAGAAAAAGCCAAAGATTGTGCGATTTATGC | |
| | | | CCCAAACCCCCTTGTAAGGGGATTCTCACCTC | |
| | | | AACTTGTCTTCTGTGGTCAGTGTTTCCCGCCC | |
| | | | CTGAATCAGGGTTACTGTCACTATGGCTTTCA | |
| | | | ATTGGCCCGGCGTAGGCGCATGCTCTGCGCGT | |
| | | | ATTGGCCTCCGCTCCTGTCCCCAGACAAGCGG | |
| | | | CCATCTTGGGTCCCGCCCCTACCGTGGGGTCT | |
| | | | TCTGGGAATTGCAGTCCCCGCTCTGCTCTGTC | |
| | | | CGGTCACAGGACTTTTTGCCCTCTGTTCCCGG | |
| | | | GTCCCTCAGGCGGCCACCCAGTGGGCACACT | |
| | | | CCCAGGCGGCGCTCCGGCCCCGCGCTCCCTCC | |
| | | | CTCTGCCTTTCATTCCCAGCTGTCAACATCCT | |
| | | | GGAAGGTAGGGGCGGGGAGGCAAGCCCAAG | |
| | | | TGGAATACTGTTTCTGGGGCGCGGG | |
| 20 | G6PD | glucose-6- | AATTTAACGACCTCGATAGAGCGCAGTCAAG | NM_000402 |
| | | phosphate | TTTGGTGAACAGAATATGTCTCTGAACTAGAG | |
| | | dehydrogenase | GAGTCCTCACACAAGGAGTAGGGTCAGACCC | |
| | | | CGCAGTGGAGGAGGAGGGAGGAGTAGAAAC | |
| | | | AGTCCAGCTCGCCGCCCAAGTAACCTGGGTCC | |
| | | | TGAATCGGCCCGCCTTGGCCAGTGCTCCAGAA | |
| | | | GCGCGGAGCAGGAACGGGCTGGGGCCCAAAA | |
| | | | AAGAGGGGGGAGCCTGAACGTCCGGGGGAA | |
| | | | GTTTCGGAGGCGGCGGAACGCCCACGGATGG | |
| | | | AACCCTGTCTTTGGGGAAAAGGACCACACCT | |
| | | | GTCAGCAGAGTCCGTCAGACGTGAGAAGGGT | |
| | | | GGGAGCGGCGGACTGTGAACGCTGGTAGGGC | |
| | | | CCCGGCGCTCCGAGAAAGTCCCAGTTTCGCG | |
| | | | GTCGCCCTTCCCTACCACGCTTCCGGCTTCCG | |
| | | | GTGTCATAGCTGTGGGATCCGGAAGTAAAAA | |
| | | | CACAAGCCCCGCCCCCGAGAACTCGGGAAGC | |
| | | | CGGCGAGAAGTGTGAGGCCGCGGTAGGGCCG | |
| | | | CATCCCGCTCCGGAGAGAAGTCTGAGTCCGC | |
| | | | CAGGCTCTGCAGGCCCGCGGAAGCTCGGTAA | |
| | | | TGATAAGCACGCCGGCCACTTTGCAGGGCGT | |
| | | | CACCGCCTACACGCCCCCTCGTCTCTCGGACG | |
| | | | GCGGCGTCTAGCCTCGGGGCGCTCGGCCGCC | |
| | | | CCGCCCTCTCCGGGGGAGGAATCAAGAAGAG | |
| | | | ACTGCCCAATAGGGCCGGCTTGACCCGCGAA | |
| | | | CAGGCGAGGGTTCCCGGGGGAGTGGCGCGGC | |
| | | | AGAAGGCCCCGCCCAGGAGCCGAGGGACAGC | |
| | | | CCAGAGGAGGCGTGGCCACGCTGCCGGCGGA | |
| | | | AGTGGAGCCCTCCGCGAGCGCGCGAGGCCGC | |
| | | | CGGGGCAGGCGGGGAAACCGGACAGTAGGG | |
| | | | GCGGGGCCTGGCCGGCGATGGGGATTCGGGA | |
| | | | GCACTACGCGGAGCTGCACCCGTGCCCGCCG | |
| | | | GAATTGGGGATGCAGAGCAGCGGCAGCGGGT | |
| | | | ATGGCA | |
| 21 | GAGE1 | G antigen 1 | attgatttaaagaaaactgtccttgactt | NM_001468 |
| | G | | accagtgtgtaagtccatgaaagcataattc | |
| | antigen 1 | | tgttgaaagcatatattgttaatgggtgttg | |
| | | | ggaaccgtgcactttccgctgctgtgggag | |
| | | | catgtccttggaggtacctttcatctgttt | |
| | | | tctcaactccaaacatcttaggaccatgggt | |
| | | | tgtgactggtaggactatgtatcttgctgct | |
| | | | ttcaagacggagtatattttcacgtggtgt | |
| | | | cactctggctgtcctgtttccctaataCTGT | |
| | | | CACTTCACcctctgcgattctgatgctacaa | |
| | | | atgatagatatcgttttagcattttcttacg | |
| | | | ggtcctagcgattctattcatttttctttc | |
| | | | agtctctttctctgacttgttcacattgaac | |
| | | | aatttcCTTTTGGGATAGGTTGCTATTTCT | |
| | | | GTTTTCGCAGGTGGTTTACCTGTCTTCC | |
| | | | CAGCCAGTCACAGTGGTCCTTGTCCCCAT | |
| | | | GGTGGGTCCGGGGCAAGAGAGGGCCCTGGGT | |
| | | | TGGGGGTGGGGTTCAGTTGAAGATGGGGTGA | |
| | | | GTTTTGAGGGGAGCACTACTTGAGTCCCAGA | |
| | | | GGCATAGGAAACAGCAGAGGGAGGTGGGATT | |
| | | | CCCTTATCCTCAATGAGGATGGGCATGGAGG | |
| | | | GTTTGGGGCGTGGCGCTGGGAACGGCAGCCC | |
| | | | TCCCCAGCCCACAGCCGCGCATGCTCCCTGGG | |
| | | | CTCCCGCCTCAGTGCGCATGTTCACTGGGCGT | |
| | | | CTTCTGCCCGGCCCCTTCGCCCACGTGAAGAA | |
| | | | CGCCAGGGAGCTGTGAGGCAGTGCTGTGTGG | |
| | | | TTCCTGCCGTCCGGACTCTTTTTCCTCTACTGA | |
| | | | GATTCATCTGGTAGGTGTGCAGGCCAGTCATC | |
| | | | CCGGGGGCTGAAGTGTGAGTGAGGGTGGAGA | |
| | | | GGGCCTCGGGTGGGTCAGGCGGGTCCCGCTT | |
| | | | CCTGGTCTGTGGCCTCCGAGGGAGAAGGGCC | |
| | | | ACGAGGTCGTCCTCCTTCCCTTCACAGGCTGC | |
| | | | GAGGCCACCGGCG | |
| 22 | GATA-3 | GATA3 | AATTCGCCCTTGGAAGATCTACCAGTACCAAC | NM_002051 |
| | | protein | CTGGGTAGCGAAGAGCAGAGAGGAGGAGGA | |
| | | | GGCGGCGGCGTACGACCTGCTCGGTCAGATT | |
| | | | GCGTTGCTCGCTCTGTCTCGCTCTCCCTCCGTC | |
| | | | TCTCTCTCTTCTTCTCTCTCTCTCTCCCTCTCT | |
| | | | CAGTATTTTTTTTTTTTTTTACAGGGAATGCA | |
| | | | TTCTTTCTGAAAGTATCAAGACGGCGCCAGGCA | |
| | | | GCTCAGTGTTCGCAGACAGCTGTGGCGCGAC | |
| | | | GCAACTTAAGGGGGTTCTAGTGTCATCCGCGC | |
| | | | CGGGGGGGAGGAGCCTGGCGCTGGCGAGTAG | |
| | | | GGGACAGGATCCCCGGCACAAGGAAACTGCA | |
| | | | ACCCAAACCCGCTCCAGGACTTCTCCCCCCGC | |
| | | | CCCGCGCACCCCCGCCCCTCCTCCCGCCCCTC | |
| | | | CACTGACCGGAAAGGGGNNCCGCAGAGGGCG | |
| | | | GCCGCCGGCGGAGGGGCGGCGGGCAGGGTGG | |
| | | | GCGAGGCCCGCGGGGCTTGGGGGCGGACGGG | |
| | | | AGGGAACGCGCGCTCTGGCCCTTTAAATGTG | |
| | | | GCCGCGGCTCCTGCCAATTCATTCGGGTCGGG | |
| | | | TGGACGATTCCGTCCCGGTGCAGCCAGCCTGC | |
| | | | CCCATTCATGAAGTTCATTTCGATGGGCAGAA | |
| | | | TTTTCTTTTTCAGACTTTTAAAAAATAGGCAC | |
| | | | GCATGGATCATTATTAGGATCCAATGCAGGGT | |
| | | | GTTTGGGAGAGCGCATCGATGTGGGGAAACG | |
| | | | TGCGCGTTAAATTGATCAGAAAAACNAAATG | |
| | | | TTTCATGTCAAGGTATTTTGAGATTTGCCTCTC | |
| | | | GGGCCGACTTCCTAAGAGGGTGAGTCATCGG | |
| | | | ATAAAGGGGAGANGCCTTTGACTGGAGCGTC | |
| | | | NGCGTCAATTTTGNTGTCATTGTCACCTCTTTC | |
| | | | CCNANCCTTCTGNNCTCTCAAGCCCCCA | |
| 23 | GLUT4 | solute carrier | GCTCCAGGAACCAACCTGGGGAATGTGTGTA | NM_001042 |
| | | family 2 | GGGGAAGGGCGGGATAGACAGTGCCCGGAGC | |
| | | (facilitated | AGGGAGGCGCTGAAAGACAGGACCAAGCAG | |
| | | glucose | CCCGGCCACCAGACCCGTTGTGGGAACGGAA | |
| | | transporter), | TTTCCTGGCCCCCAGGGCCACACTCGCGTGGG | |
| | | member 4 | AAGCATGTCGCGGACTCTTTAAGGCGTCATCT | |
| | | | CCCTGTCTCTCCGCCCCCGCCTGGGACAGGCC | |
| | | | GGGACGCCCGGGACCTGACATTTGGAGGCTC | |
| | | | CCAACGTGGGAGCTAAAAATAGCAGCCCCGG | |
| | | | GTTACTTTGGGGCATTGCTCCTCTCCCAACCC | |
| | | | GCGCGCCGGCTCGCGAGCCGTCTCAGGCCGC | |
| | | | TGGAGTTTCCCCGGGGCAAGTACACCTGGCCC | |
| | | | GTCCTCTCCTCTCAGACCCCACTGTCCAGACC | |
| | | | CGCAGAGTTTAAGATGCTTCTGCAGCCCGGG | |
| | | | ATCCTAGCTGGTGGGCGGAGTCCTAACACGT | |
| | | | GGGTGGGCGGGGCCTTTTGTTCCAGGGACTCT | |
| | | | TTTCTCAAAACTTCCCAGTCGGAGGCTGGCGG | |
| | | | GAACCCGAGAGGCGTGTCTCGCCAGCCACGC | |
| | | | GGAGGGGCGTGGCCTCATTGGCCCGCCCCAC | |
| | | | CAACTCCAGCCAAACTCTAAACCCCAGGCGG | |
| | | | AGGGGGCGTGGCCTTCTGGGGTGTGCGGGCT | |
| | | | CCTGGCCAATGGGTGCTGTGAAGGGCGTGGC | |
| | | | CCGCGGGGGCAGGAGCGAGGTGGCGGGGGCT | |
| | | | TCTCGCGTCTTTTCCCCCAGCCCCGCTCCACC | |
| | | | AGATCCGCGGGAGCCCCACTGCTCTCCGGGTC | |
| | | | CTTGGCTTGTGGCTGTGGGTCCCATCGGGCCC | |
| | | | GCCCTCGCACGTCACTCCGGGACCCCCGCGGC | |
| | | | CTCCGCAGGTTCTGCGCTCCAGGCCGGAGTCA | |
| | | | GAGACTCCAGGATCGGTTCTTTCATCTTCGCC | |
| | | | GCCCCTGCGCGTCCAGCTCTTCTAAGACGAGA | |
| | | | TGCCGTCGGGCTTCCAACAGATAGGCTCCGA | |
| | | | AGTAGGATTCATCATGAGGGGGCGG | |
| 24 | GPC3 | GPC3 | GGCGAANTGGGCCCTCTAGATGCATGCTCGA | NM_004484 |
| | | (Glypican 3) | GCGGCCGCCAGTGTGATGGATATCTGCAGAA | |
| | | | TTCGCCCTTGGAAGATCTTCCCGGCCATCCTG | |
| | | | CTTCGCAGGGAGCTAGGAGAGCGCGGGAGAG | |
| | | | TGGCAGCCGGAGCGAGAGCAGTCCCAGGACT | |
| | | | CGGCAAGCCTGGCAGTGGCCCTGAGGAGCAA | |
| | | | GAGACGTGCTGCTACCCAGCCGCTGCAAAAG | |
| | | | TTTCCTCGCAGCTACCTGGGCGCTGGGCGAGG | |
| | | | GCGGGAACCGCTTGGCGGCGCGGGGCAGGGC | |
| | | | GGGGCTGACTGGGGTGGGGCGGGGCGAGGAG | |
| | | | GGACGGGGCGGGGCGAGGCGAGCCGCGCGG | |
| | | | CCAGGGGGCGGTGGCGGTTGTGCGGCCGGTA | |
| | | | GCCGGCGGGGTGCGGGGGCGCGGCGTGGAGC | |
| | | | GCGGCGGGGGCCACTGGGGCACCGCGGCGCG | |
| | | | GGGACCGGGCGAAGGCAGTGCGAGAGGAGG | |
| | | | GTGCGGAGCCCGCGCGGTGGCTCCCGGCAGC | |
| | | | CGAGCCCAGCTGCCCGCTCGCAGCCGCTCTAC | |
| | | | ACAGGGCGCTCTGGCATAACTACTGCAGAGG | |
| | | | GGCTGCAGGCTCGAGCGCGCTGATTGGCTTCC | |
| | | | CAGCAGCCGTCCGCTCTGACTGGCTCTGGGAG | |
| | | | AAGTTCCCCAGCCTCACTCCTCCTTCCCGCCG | |
| | | | CTCATTGGCCTACAGCCTGGAGGGCTTTTCCC | |
| | | | TTTAGGATTTTTGTCTCCTTTTCATCCTTCCTG | |
| | | | GGGGCAGGTGGGGGTCCCTGACTTAGGTCCT | |
| | | | CCTCCGCTTCGCCACAGGCCTTCTTTCAGCTT | |
| | | | GTGCCAGCTCTTTCCTGGCCACCAGANCCACA | |
| | | | CAANGTGTTCCTTCACACAAAATCCACCTCCT | |
| | | | CATTCTACTCTCTGAGGAGCTTCCCGCGAACC | |
| | | | GTTTTCCTAACGCAGCTCTGACCGGGTTCTCA | |
| | | | AAGCCAACCTGCAAATAGTCGCGTTCNGGGA | |
| | | | CAGCCANGGACCGCTGGGCTTTTCACANCCTG | |
| | | | CCTCACCTTTGAATAT | |
| 25 | HIN-1 | HIV-1 induced | AGTTGTTTCAATTCACAGCTTTAGAATTTTGG | NM_199324 |
| | | protein | TAAAAGACCACATGCCAGTAAGTTATCTTTTT | |
| | | | GTTGAACTGGTTGTTTAATAGAAGAAAATGTA | |
| | | | AACTGCAGAGTGAGAGGATCTGGATCATACT | |
| | | | TTGTAGGTTGGTACTTTACAATTTAGGGCATA | |
| | | | AAAACAAACCCCAAACCTCTTGGGATGATAC | |
| | | | CACACAACATTTTTGCACCCCCTATGCTGCCT | |
| | | | ACTTGGATGTTCTTTCTTGTCTTATAAGCTTGA | |
| | | | TCACCAAGGAAAGAATGAGTGCCTTAATTTTT | |
| | | | CTGAAACCATAGTGGACTTAAATTTTTACACA | |
| | | | GAGCCTCTAAGTGGATTCAGAATTAATGGGA | |
| | | | AAAATAAATCGGCCTCTTACAGGCTGAAAGC | |
| | | | CTCAAAATACATTCCTACAGAAGTTGCCAGTT | |
| | | | TGTCTTTTTCAATATGTATAGGATGAAGTTGA | |
| | | | GCGTGGCGTAGCATGGATTTTGTTAGCTCTTC | |
| | | | TTTGTGAAGAGTAAAGTTATTGTGGAGGGAA | |
| | | | GGCCAAGGGAAGAGAGTGTCCTAAATTTACA | |
| | | | AAAATGTCCTAAAGGAGAAAGGCTAATAAAT | |
| | | | TCTTTACAAATTTGGCTTAAGAAGTAGTATTG | |
| | | | TTTGTATATGTCATGTCTTCGCTGTGCTTAGTT | |
| | | | AGAAGAAGAGGTAGGAATGAGTAAAGATATC | |
| | | | GAAATTATAGAAAGGGAAATGGAGAAAGACT | |
| | | | GATAATCTATTGGTTGTCAGATTATTTTGGGT | |
| | | | GTAAAAGAAGACATTAGGTTGTAACTTTTAAC | |
| | | | TAAATGCTTAATAGTGTGTTTGTTGCCTTTTCT | |
| | | | TTTTAGGTATTGCACTCTCAGTCTCGCCATGTT | |
| | | | GAAGTCAGAATGGCCTGTATTCACTATCTTCG | |
| | | | AGAGAACAGAGAGAAATTTGAAGCGGTAACT | |
| | | | TGTAATTTCAAACATGTAATGGTGTCTTGACT | |
| | | | TGGTTTTACATTTTGGCTTTTAGAAGTGTTCTA | |
| | | | GTAGAATTTCACAGGCTGGATCTTAATGCGGG | |
| | | | TTATGAAAATAAC | |
| 26 | hMLH1 | mutL homolog | TCTCAGCAACACCTCCATGCACTGGTATACAA | AB017806 |
| | | 1, colon | AGTCCCCCTCACCCCAGCCGCGACCCTTCAAG | |
| | | cancer, | GCCAAGAGGCGGCAGAGCCCGAGGCCTGCAC | |
| | | nonpolyposis | GAGCAGCTCTCTCTTCAGGAGTGAAGGAGGC | |
| | | type 2 | CACGGGCAAGTCGCCCTGACGCAGACGCTCC | |
| | | | ACCAGGGCCGCGCGCTCGCCGTCCGCCACAT | |
| | | | ACCGCTCGTAGTATTCGTGCTCAGCCTCGTAG | |
| | | | TGGCGCCTGACGTCGCGTTCGCGGGTAGCTAC | |
| | | | GATGAGGCGGCGACAGACCAGGCACAGGGCC | |
| | | | CCATCGCCCTCCGGAGGCTCCACCACCAAATA | |
| | | | ACGCTGGGTCCACTCGGGCCGGAAAACTAGA | |
| | | | GCCTCGTCGACTTCCATCTTGCTTCTTTTGGGC | |
| | | | GTCATCCACATTCTGCGGGAGGCCACAAGAG | |
| | | | CAGGGCCAACGTTAGAAAGGCCGCAAGGGGA | |
| | | | GAGGAGGAGCCTGAGAAGCGCCAAGCACCTC | |
| | | | CTCCGCTCTGCGCCAGATCACCTCAGCAGAGG | |
| | | | CACACAAGCCCGGTTCCGGCATCTCTGCTCCT | |
| | | | ATTGGCTGGATATTTCGTATTCCCCGAGCTCC | |
| | | | TAAAAACGAACCAATAGGAAGAGCGGACAGC | |
| | | | GATCTCTAACGCGCAAGCGCATATCCTTCTAG | |
| | | | GTAGCGGGCAGTAGCCGCTTCAGGGAGGGAC | |
| | | | GAAGAGACCCAGCAACCCACAGAGTTGAGAA | |
| | | | ATTTGACTGGCATTCAAGCTGTCCAATCAATA | |
| | | | GCTGCCGCTGAAGGGTGGGGCTGGATGGCGT | |
| | | | AAGCTACAGCTGAAGGAAGAACGTGAGCACG | |
| | | | AGGCACTGAGGTGATTGGCTGAAGGCACTTC | |
| | | | CGTTGAGCATCTAGACGTTTCCTTGGCTCTTC | |
| | | | TGGCGCCAAAATGTCGTTCGTGGCAGGGGTT | |
| | | | ATTCGGCGGCTGGACGAGACAGTGGTGAACC | |
| | | | GCATCGCGGCGGGGGAAGTTATCCAGCGGCC | |
| | | | AGCTAATGCTATCAAAGAGATGATTGAGAAC | |
| | | | TGGTACGGAGGGAGTCGAGCCGGGCT | |
| 27 | HOXA2 | homeo box A2 | TGGGCCCGGGGCGCAGACTCTGGGCTGGACA | NM_006735 |
| | | | CTgggaggggggcgagaggctgaggggagaag | |
| | | | gggaggcggacagaagagagagggagggag | |
| | | | aaagggggagaagaggaaaaagagggaaa | |
| | | | gggacagacaggaaggaaaacagaccgagaga | |
| | | | gaTCAGTTTTGAGATCCAGGAACTGCTTTTA | |
| | | | GGAAAAGTGAAGGAGGAAAAGGGAAAGAAAAG | |
| | | | GAAGACCCCTTCCCAACCAAAATCTTTCCTT | |
| | | | TCTtctctcttttctgtcttctctttctccat | |
| | | | ctctcaaactctctcttcttccctctctctt | |
| | | | tattctccctctctcatctcctctcttcctc | |
| | | | tGCTCCTTTCTCCTGCTTTAACAGAACTTA | |
| | | | TGTGGCTGGGACGCAGGGCCCTCGGGTGT | |
| | | | CAAAACTTTGAAGATTAATGGATTACTT | |
| | | | TGTTAATGACTGCAGGCGTCAGACTGAGGTG | |
| | | | CTTAAATGATTTGTGAGGTGCGAGGCGTCTTC | |
| | | | CCGACAGTCCCAAACAATGCGCGGAGTGTGC | |
| | | | GGGGGAGGCAGAGGGCAGCCACCGGCGGGA | |
| | | | CCGACAGCAGGGCTTacactcgcgcacatt | |
| | | | cacacacacacacacacactcccaggcacac | |
| | | | acacTAGATAGATCCTTGCAGATCAGGAGGCA | |
| | | | CGCAGGCACCCTCGCCCCCACGTACTCCGGGA | |
| | | | CATCCCCACCCACACCAACATATATGTATT | |
| | | | TTTGCTCTGAAAAAAGTGTAAATAAAGCCTCG | |
| | | | CTGGCCCCCAATGAGGCGTTCCTTCCCGAC | |
| | | | TTTTTTGGATCAATCAAACAGACAGTGGCTT | |
| | | | CTTTTGATTAAAGCCCAAATTGTCATTGGGCA | |
| | | | GAAGCAATCATGTGACAGCCAATTCGGTCC | |
| | | | AATTTCAACCTTGTCTCCATGAATTCAATAGT | |
| | | | TTAATAGTAGCGCGGTCCCCATACGGCTGTAA | |
| | | | TCAGTGAATTAGAAAAAAAACACCCTAGCAGC | |
| | | | GATATTCTATGATAGATTTTTTTTCCTCT | |
| | | | GCGCTCGCCTTT | |
| 28 | H-Ras | v-Ha-ras | TGTGGCAACTTGTGGGTACGGTTTAACTGGAC | NM_176795 |
| | | Harvey rat | CACGCTGAGCTTCTGCAGCGTTGGAACCTCAA | |
| | | sarcoma viral | GTTTGGGGGGACTGGGCGGGCAGGGTCGCCT | |
| | | oncogene | GCCACGCAGGCCCGAGAAAGAGGAGAGTGGT | |
| | | homolog | GGAGGGGGCGTTCTCACGCCTGGCCCCAGGG | |
| | | | CACACGGCTGCGCCCGCCGCCCGGAACCCCA | |
| | | | CCGGGGCTGCAAGCGTCCTCGGGGTGGGTTG | |
| | | | CGGTGGGAGTAGGGGAGCTGGGGTGCGTGGT | |
| | | | GGTAGGTGGGGTGCGCGGCCGCTCCACCTGC | |
| | | | GCGGAAGGGCAGCCGGGCAACCGGACCCCGC | |
| | | | GGCCACCCGGGGGCCCCCAGCTCCGAGCATC | |
| | | | CCGCCTTGGTCCCGGCGGATCCCAGCCTTTCC | |
| | | | CCAGCCCGTAGCCCCGGGACCTCCGCGGTGG | |
| | | | GCGGCGCCGCGCTGCCGGCGCAGGGAGGCC | |
| | | | TCTGGTGCACCGGCACCGCTGAGTCGGGTTCT | |
| | | | CTCGCCGGCCTGTTCCCGGGAGAGCCCGGGG | |
| | | | CCCTGCTCGGAGATGCCGCCCCGGGCCCCCA | |
| | | | GACACCGGCTCCCTGGCCTTCCTCGAGCAACC | |
| | | | CCGAGCTCGGCTCCGGTCTCCAGCCAAGCCCA | |
| | | | ACCCCGAGAGGCCGCGGCCCTACTGGCTCCG | |
| | | | CCTCCCGCGTTGCTCCCGGAAGCCCCGCCCGA | |
| | | | CCGCGGCTCCTGACAGACGGGCCGCTCAGCC | |
| | | | AACCGGGGTGGGGCGGGGCCCGATGGCGCGC | |
| | | | AGCCAATGGTAGGCCGCGCCTGGCAGACGGA | |
| | | | CGGGCGCGGGGCGGGGCGTGCGCAGGCCCGC | |
| | | | CCGAGTCTCCGCCGCCCGTGCCCTGCGCCCGC | |
| | | | AACCCGAGCCGCACCCGCCGCGGACGGAGCC | |
| | | | CATGCGCGGGGCGAACCGCGcgcccccgccc | |
| | | | ccgccccgccccggcctcggccccggccctg | |
| | | | gccccggGGGCAGTCGCGCCTGTGAACGGTGA | |
| | | | GTGCGGGCAGGGATCGGCCGGGCCGCGCGCC | |
| | | | CTCCTCGCCCCCAGGCGGCAGCAATAcgcg | |
| 29 | hTERT | telomerase | CGGCCAGCAGGAGCGCCTGGCTCCATTTCCCA | AF097365 |
| | | reverse | CCCTTTCTCGACGGGACCGCCCCGGTGGGTGA | |
| | | transcriptase | TTAACAGATTTGGGGTGGTTTGCTCATGGTGG | |
| | | | GGACCCCTCGCCGCCTGAGAACCTGCAAAGA | |
| | | | GAAATGACGGGCCTGTGTCAAGGAGCCCAAG | |
| | | | TCGCGGGGAAGTGTTGCAGGGAGGCACTCCG | |
| | | | GGAGGTCCCGCGTGCCCGTCCAGGGAGCAAT | |
| | | | GCGTCCTCGGGTTCGTCCCCAGCCGCGTCTAC | |
| | | | GCGCCTCCGTCCTCCCCTTCACGTCCGGCATT | |
| | | | CGTGGTGCCCGGAGCCCGACGCCCCGCGTCC | |
| | | | GGACCTGGAGGCAGCCCTGGGTCTCCGGATC | |
| | | | AGGCCAGCGGCCAAAGGGTCGCCGCACGCAC | |
| | | | CTGTTCCCAGGGCCTCCACATCATGGCCCCTC | |
| | | | CCTCGGGTTACCCCACAGCCTAGGCCGATTCG | |
| | | | ACCTCTCTCCGCTGGGGCCCTCGCTGGCGTCC | |
| | | | CTGCACCCTGGGAGCGCGAGCGGCGCGCGGG | |
| | | | CGGGGAAGCGCGGCCCAGACCCCCGGGTCCG | |
| | | | CCCGGAGCAGCTGCGCTGTCGGGGCCAGGCC | |
| | | | GGGCTCCCAGTGGATTCGCGGGCACAGACGC | |
| | | | CCAGGACCGCGCTTCCCACGTGGCGGAGGGA | |
| | | | CTGGGGACCCGGGCAcccgtcctgccccttca | |
| | | | ccttccagctccgcctcctccgcgcggaccc | |
| | | | cgccccgtcccgacccctcccgggtccccggc | |
| | | | ccagccccctccgggccctcccagcccctccc | |
| | | | cttcctttccgcggccccgcccTCTCCTCGC | |
| | | | GGCGCGAGTTTCAGGCAGCGCTGCGTCCTGC | |
| | | | TGCGCACGTGGGAAGCCCTGGCCCCGGCCACC | |
| | | | CCCGCGATGCCGCGCGCTCCCCGCTGCCGAGC | |
| | | | CGTGCGCTCCCTGCTGCGCAGCCACTACCGC | |
| | | | GAGGTGCTGCCGCTGGCCACGTTCGTGCGGCG | |
| | | | CCTGGGGCCCCAGGGCTGGCGGCTGGTGCAG | |
| | | | CGCGGGGACCCGGCGGCTTTCCGCG | |
| 30 | IFN | IFN gamma | ccctgggaatattctctacactgtatttcaag | NM_000619 |
| | | | gatttaatatgacaaaaagaatgtcaaatacc | |
| | | | ttattaacaatgtagtatattgatgcatact | |
| | | | gaagtactatttgggatatattggtttaaata | |
| | | | caatatattttaaaattatatttacctttta | |
| | | | aaaaaacttttattaatgaggctactagatca | |
| | | | tttaaatttacctgtgtggcttgtattgtatt | |
| | | | tctactgggcagtgctgATCTAGAGCAATTT | |
| | | | GAAACTTGTGGTAGATATTTTACTAACCAACT | |
| | | | CTGATGAAGGACTTCCTCACCAAATTGTTCT | |
| | | | TTTAACCGCATTCTTTCCTTGCTTTCTGGTC | |
| | | | ATTTGCAAGAAAAATTTTAAAAGGCTGCCCCT | |
| | | | TTGTAAAGGTTTGAGAGGCCCTAGAATTTCGT | |
| | | | TTTTCACTTGTTCCCAACCACAAGCAAATGA | |
| | | | TCAATGTGCTTTGTGAATGAAGAGTCAAC | |
| | | | ATTTTACCAGGGCGAAGTGGGGAGGTACAAAA | |
| | | | AAATTTCCAGTCCTTGAATGGTGTGAAGTAAA | |
| | | | AGTGCCTTCAAAGAATCCCACCAGAATGGCAC | |
| | | | AGGTGGGCATAATGGGTCTGTCTCATCGTCA | |
| | | | AAGGACCCAAGGAGTCTAAAGGAAACTCTAAC | |
| | | | TACAACACCCAAATGCCACAAAACCTTAGTTA | |
| | | | TTAATACAAACTATCATCCCTGCCTATCTGTC | |
| | | | ACCATCTCATCTTAAAAAACTTGTGAAAATAC | |
| | | | GTAATCCTCAGGAGACTTCAATTAGGTATAAA | |
| | | | TACCAGCAGCCAGAGGAGGTGCAGCACATTGTT | |
| | | | CTGATCATCTGAAGATCAGCTATTAGAAGAGA | |
| | | | AAGATCAGTTAAGTCCTTTGGACCTGATCAGCT | |
| | | | TGATACAAGAACTACTGATTTCAACTTCTTTG | |
| | | | GCTTAATTCTCTCGGAAACGATGAAATATACAA | |
| | | | GTTATATCTTGGCTTTTCAGCTCTGCATCGTT | |
| | | | TTGGGTTCTCTTGGCTGTTACTGCCAGGACCCA | |
| | | | TATGTAAAAGAAGC | |
| 31 | IGRP | glucose-6- | GCGGGTACGACTCCTATAGGGCGATTGGGCC | AF283575 |
| | | phosphatase, | CTCTAGATGCATGCTCGAGCGGCCGCCAGTGT | |
| | | catalytic, 2 | GATGGATATCTGCAGAATTCGCCCTTGGAAG | |
| | | | ATCTAACCAATCCCCAATGACTGCTACCCATA | |
| | | | TCATCTTGGTTCCAACTGTCTGATTAAATTGA | |
| | | | AAACAAAGTGGAAAATAAATGAAAAAGATAT | |
| | | | TCCTGGGGTCTCCAACATTGGACATAAAATTT | |
| | | | AGAAAAGTGTAGTAAGCTCGGTAGTCCTTCTG | |
| | | | CAAATGCTGAATTATGAGCACTCCATTCCTGT | |
| | | | GAAGGAAATCCATCTTGAAAAAGAGGCAATT | |
| | | | CTAAACATAGAGCAATTGGAGCTGAAGTGCT | |
| | | | CTGATTCCCACCGTTTTTATACTGTGCCTTTGT | |
| | | | GGCATGTCGAGCCATTACTGCAACATGTGATG | |
| | | | CTGACCATCTGTGGAGAGGGCACACCAGCCC | |
| | | | TCCTCTGCTGAATAGCTCATCTATTTATGATTT | |
| | | | TAATTGGTGGCAAAGAGTGAAGTACATGCTG | |
| | | | ATCTGTGGCAATTCGAGGGGGAAATTTGGAT | |
| | | | AGAAACACAATGAATTTCTTATGCAACCTCCC | |
| | | | TTTTGTGCGAACAGTTGGATCATGTTTGTTTG | |
| | | | AAATTTTTTGTACAGTTCATTTCCTCCAAGGT | |
| | | | CAGACATTAGCAATTTCTATGTTTGGTGAAAA | |
| | | | GACTTTGCAAATAATTATTGCATGTCAAATAG | |
| | | | CCCATAAAGCCCTGCATTTTAATTTAAGATAG | |
| | | | GCTGTGGCTCTCTATTTTATTGGGTCTTTGAG | |
| | | | GAAAATGGTTGAATAAATATCTGGGTATGAA | |
| | | | AAATATATGATATGACAGATTATGTTCTGATC | |
| | | | ACTGATTTAAAATAAGAATAGTTCAATTTTCT | |
| | | | TTATCCAAGAGAATGATAGAATATATATGGA | |
| | | | ACAGGGGAAAGAAATGTGTTGTTTTTTTGACT | |
| | | | ATAAGACAGAAAAGCAGAAATGAAAGTCTTT | |
| | | | TGGATAATTGAAATGTGTTAGGATCAAATCGT | |
| | | | ATCTTTATTACTAAAGA | |
| 32 | IL-4 | interleukin 4 | ATTCAATAAAAAACAAGCAGGGCGGGTGGTG | NM_000589 |
| | | | GGGCACTGACTAGGAGGGCTGATTTGTAAGT | |
| | | | TGGTAAGACTGTAGCTCTTTTTCCTAATTAGC | |
| | | | TGAGGATGTGTTTAGGTTCCATTCAAAAAGTG | |
| | | | GGCATTCCTggccaggcatggtggctcacacc | |
| | | | tgtaatctcagagctttgggagactgaggtag | |
| | | | gaggatcacttgagcccaggaatttgagatga | |
| | | | gcctaggcaacatagtgagactcttatctct | |
| | | | atcaaaaaataaaaataaaaatgagccaggca | |
| | | | tggtgcggtggcacgcacctactgctaggggg | |
| | | | gctgaggtgggaggatcacttgagcctgggag | |
| | | | gttgaggctgcagtgatccctgatcacaacat | |
| | | | tgcatttcagcctgggtgacagagtgagacc | |
| | | | ctgtctcagaaaaaaaaaaaaaaaaGTCATTC | |
| | | | CTGAAACCTCAGAATAGACCTACCTTGCCAAG | |
| | | | GGCTTCCTTATGGGTAAGGACCTTATGGACCT | |
| | | | GCTGGGACCCAAACTAGGCCTCACCTGATACG | |
| | | | ACCTGTCCTTCTCAAAACACCTAAAC | |
| | | | TTGGGAGAACATTGTCCCCCAGTGCTG | |
| | | | GGGTAGGAGAGTCTGCCTGTTATTCT | |
| | | | GCCTCTATGCAGAGAAGGAGCCCCAGATCAG | |
| | | | CTTTTCCATGACAGGACAGTTTCCAAGATGCC | |
| | | | ACCTGTACTTGGAAGAAGCCAGGTTAAAATA | |
| | | | CTTTTCAAGTAAAACTTTCTTGATATTACTCTA | |
| | | | TCTTTCCCCAGGAGGACTGCATTACAACAAAT | |
| | | | TCGGACACCTGTGGCCTCTCCCTTCTATGCAA | |
| | | | AGCAAAAAGCCAGCAGCAGCCCCAAGCTGAT | |
| | | | AAGATTAATCTAAAGAGCAAATTATGGTGTA | |
| | | | ATTTCCTATGCTGAAACTTTGTAGTTAATTTT | |
| | | | TTAAAAAGGTTTCATTTTCCTATTGGTCTGAT | |
| | | | TTCACAGGAACATTTTACCTGTTTGTGAGGCA | |
| | | | TTTTTTCTCCTGGAAGAGAGGTGCTGATTGGC | |
| 33 | IRF7 | interferon | AGGCCTAGGGGTGAGAGACACATTCCCCTCG | NM_004030 |
| | | regulatory | CTGCTCCCAAAGCCAGAGCCCAGGCTGGGCG | |
| | | factor 7 | CCCATGCCCAGAACCATCAAGGGATCCCTTGC | |
| | | | GGCTTGTCAGCACTTTCCCTAATGGAAATACA | |
| | | | CCATTAATTCCTTTCCAAATGTTTTAATTGTGA | |
| | | | GAGTATCTGATATTCTTGACTGAACAATGTAA | |
| | | | AAAACCCAAAGGGggctgcgcacggcggctct | |
| | | | cgcctaaatcccagcactttgggaggccgaggt | |
| | | | gggcagatcacctgaggtcgggagttcgacacc | |
| | | | agcctgaccaacatagagaaaccccgtctcta | |
| | | | ctaaaaatacaaaattagccgggcgtggtggtt | |
| | | | catgcctgtaatcccagctactcgggaggctt | |
| | | | aggtaggagaatcacttgaacccgggaggcgga | |
| | | | ggttgtggtgggccaagattgtgccaccgcact | |
| | | | ccagcctgggtaacaaaagcgaaactccatctc | |
| | | | aaaaaaagaaaCGCAAACGGTGCAGCTGCCCC | |
| | | | TTTTTCGAGGCACGTCCACCTCCCATTACCCAC | |
| | | | ttccttttttttttgagactgagtcttgctctg | |
| | | | tcccctgggctgtagtggagtggctccatctcg | |
| | | | gctcactgcagccACTCCCAACGCCCTCCACT | |
| | | | CCTCCCTACTCCGCGCTGGCCGGGGCGGGGTTC | |
| | | | CGCTGGTCGCATCCAATAATAAGAACAGGCGG | |
| | | | CGCGCGCCCTTCCCGGAAACTCCCGCCTGGCC | |
| | | | ACCATAAAAGCGCCGGCCCTCCGCTTCCCCGC | |
| | | | GAGACGAAACTTCCCGTCCCGGCGGCTCTGG | |
| | | | CACCCAGGTACTGGGGACCCCAGACCCACGC | |
| | | | GGTGCAGGCCGGGAGCGAGAGCCTCCGTGGG | |
| | | | GGCTCCGTGACCCCGGAGGGGTAGAGCCAAG | |
| | | | AGCTGGGGGAGCCTGAGAGATGAGGGTCgggc | |
| | | | ggggagggaggcggaggcggaggcggaggcg | |
| | | | gggTTCCGCGGAGCTGAGAACCGGACGGGG | |
| | | | TGGGAT | |
| 34 | JUNB | jun B proto- | AATTTCTGGCAGACATGTCTCCATCTTCTACC | U20734 |
| | | oncogene | TGGCATATTTTACCTGCCTCAGTGTACCCCAG | |
| | | | GCCGCTTACTAGCTTTCTGCATATCTAGACTT | |
| | | | CCCCTAATGCCTCCTTCCCGCTTACGGGAGAG | |
| | | | CCTCAGACTCTGGACTCAGCTCCCATGAGCTC | |
| | | | CTGGACCCCTACTCATTTCTTGCAATTTAATG | |
| | | | GGTCATGCAGCTCCACCCACTCACCCCTTTTG | |
| | | | ATCTCTCCCCTCCTCCGTCCTGTGAAAATTCC | |
| | | | AGTCCCGCATCCTTCTGAGCCCGGGACCCCCA | |
| | | | GTCAATTCCTGGGTCAGGTGTCTCCTTAACCC | |
| | | | TCCCGATTTACAGTGCTTAACCCTCATTTCTG | |
| | | | CTTTTTGGGGTCTCCCAATGGATTGTCAGTCC | |
| | | | TCCTACCCCTCTCGTATTCTGGGTACCTCAGG | |
| | | | GGTTTCTTCGCACATACTGGGACCCTCACCCC | |
| | | | ACTTGCTGCGTACCAGGTCCTGGTATTTGTCC | |
| | | | CAGTGGACTCCAGGGAAATCATCCTCCTCCCT | |
| | | | GAAACCCCTCACTCATGTGCCTGGGCCCCCCA | |
| | | | GCACCTCCTTCCATGCGTACCCCGAGGTCCTT | |
| | | | TGAGCCCCTCCCCCTGCAGCCCCGCCGAGCCA | |
| | | | CCCGGCCCGTGGCCGCTGTTTACAAGGACAC | |
| | | | GCGCTTCCTGACAGTGACGCGAGCCGCCTCCT | |
| | | | CCCCTTCCCCACGCTCGAGGAGGGGGGCGCG | |
| | | | GGGGCCCGGCTCCGGCGACGGCCAATCGGAG | |
| | | | CGCACTTCCGTGGCTGACTAGCGCGGTATAAA | |
| | | | GGCGTGTGGCTCAGGCTGAGCGGCTGGGACC | |
| | | | TTGAGAGCGGCCAGGCCAGCCTCGGAGCCAG | |
| | | | CAGGGAGCTGGGAGCTGGGGGAAACGACGCC | |
| | | | AGGAAAGCTATCGCGCCAGAGAGGGCGACGG | |
| | | | GGGCTCGGGAAGCCTGACAGGGCTTTTGCGC | |
| | | | ACAGCTGCCGGCTGGCTGCTACCCGCCCGCGC | |
| | | | CAGCCCCCGAGAACGCGCGACCAGGCACCCA | |
| | | | GTCCGGTCACCGCAGCG | |
| 35 | KIR2DL | killer cell | TTCTTACAAACTCCAGAAAGGTAGGTGTAAAT | AF110032 |
| | 4 | immunoglobulin- | AAGAGACATTTGTAAGAATGACAGCACATTA | |
| | | like | AATGTGTAGATTTCAACCTTCAGTTATTGCAA | |
| | | receptor, two | TATTCCAGTATCAAGTTGGAGGATGTTATCAG | |
| | | domains, long | TCTGATATTTTTTCCTCAAATGAGAGAGAGAA | |
| | | cytoplasmic | AGAAAGACACACAAACAACACAGGGAGAAA | |
| | | tail, 4 | AAAAGCACACGTTACAGAGAGACAAAAAGG | |
| | | | GAGACAGGGAACTGTGAATTTGGACTCTTGT | |
| | | | GTCATAAGACAAATTCTAGATAACACGACCA | |
| | | | GACCTTCAATTGACATATTGTGTTTTTGCTAA | |
| | | | TAAGGTGGAATTCTATGATGCGAAATAACTAT | |
| | | | ATAGTCTTTTCTACTGGGATTTAAATCATTTTA | |
| | | | TCTGTTTCTGGCTTAACAGGAAAAATACAACC | |
| | | | ATGGAAAATTATGATGATTTATTTAATACGAT | |
| | | | TGCTCTATAGTGTTAATAAAACCTATTAGGTA | |
| | | | TTTTGCATATTACATATCAAGGAGAGTTTGAA | |
| | | | TCTCAGGTAGAAACAAAAAAAAATACATCAA | |
| | | | AAGTTCCTCATGTGAGTGCAGAATTCAATCGT | |
| | | | CCCGTGCAGGGGTAAGTGAGTCTGAGATGTG | |
| | | | TTTTGAGCCTGGCCGTTGCGCATGATGTGAAG | |
| | | | TGACAAGTCTAGTCTGCAGTTTTCAGAAACCC | |
| | | | TCATTCCTCCCTTGACTGATTCACCACTTGAA | |
| | | | CCTCATATGACGTAGAAGAAGCCTACCTATGT | |
| | | | CCCCTTCACATGTTGTGGTCAATGTGTCAACT | |
| | | | GCACGATCCGGGCCCCTCACCACATCCTCTGC | |
| | | | ACCGGTCAGTCGAGCCGAGTCACTGCGTCCTG | |
| | | | GCAGCAGAAGCTGCACCATGTCCATGTCACC | |
| | | | CACGGTCATCATCCTGGCATGTCTTGGTGAGT | |
| | | | CCTGGAAGGGAAGGAGCACCAGGGTTACACT | |
| | | | ATGGGCCTGCAGATTGGGTGTCTCCCCAGCAG | |
| | | | AGAGCCATGTTCTGAAGCAAGTGAGTGGTGA | |
| | | | GGATGAGTTAATTTTCAGT | |
| 36 | K-Ras | v-Ha-ras | CTTGTGATGGGTTCAAAATATCAAGAAAGAT | NM_033360 |
| | | Harvey rat | AGCAAAATATCACAAGCCTCCTGACCCGAGA | |
| | | sarcoma viral | AGATTAGCGTTGAAAGGGTCTGTCGTGTTTGT | |
| | | oncogene | TTGGGCCTGGGGCTAAATTCCCAGCCCAAGTG | |
| | | homolog | CTGAGGCTGATAATAATCGGGGCGGCGATCA | |
| | | | GACAGCCCCGGTGTGGGAAATCGTCCGCCCG | |
| | | | GTCTCCCTAAGTCCCCGAAGTCGCCTCCCACT | |
| | | | TTTGGTGACTGCTTGTTTATTTACATGCAGTC | |
| | | | AATGATAGTAAATGGATGCGCGCCAGTATAG | |
| | | | GCCGACCCTGAGGGTGGCGGGGTGCTCTTCG | |
| | | | CAGCTTCTCTGTGGAGACCGGTCAGCGGGGC | |
| | | | GGCGTGGCCGCTCGCGGCGTCTCCCTGGTGGC | |
| | | | ATCCGCACAGCCCGCCGCGGTCCGGTCCCGCT | |
| | | | CCGGGTCAGAATTGGCGGCTGCGGGGACAGC | |
| | | | CTTGCGGCTAGGCAGGGGGCGGGCCGCCGCG | |
| | | | TGGGTCCGGCAGTCCCTCCTCCCGCCAAGgcg | |
| | | | ccgcccagacccgctctccagccggcccggct | |
| | | | cgccaccctagaccgccccagccaccccttc | |
| | | | ctccgccggcccggcccccgctcctcccccgc | |
| | | | cggcccggcccggccccctccttctccccgc | |
| | | | cggcgctcgctgcctccccctcttccctcttc | |
| | | | ccacaccgccctcagccgctccctctcgtacg | |
| | | | cccgtcTGAAGAAGAATCGAGCGCGGAACGC | |
| | | | ATCGATAGCTCTGCCCTCTGCGGCCGCCCGGC | |
| | | | CCCGAACTCATCGGTGTGCTCGGAGCTCGAT | |
| | | | TTTCCTAggcggcggccgcggcggcggaggca | |
| | | | gcagcggcggcggcagtggcggcggcgaagg | |
| | | | tggcggcggcTCGGCCAGTACTCCCGGCCCC | |
| | | | CGCCATTTCGGACTGGGAGCGAGCGCGGCGC | |
| | | | AGGCACTGAAGGCGGCGGCGGGGCCAGAGGC | |
| | | | TCAGCGGCTCCCAGGTGCGGGAGAGAGGTAC | |
| | | | GGAGCGGACCACCCCTCCTGGGCCCC | |
| 37 | LAGE-1 | LAGE-1a and | GCAGGGACTGATACTGCCGAACCCAGGAGCC | AJ275977 |
| | | LAGE-1b | AGGCCCGACCCAGCCTCAGGTCCAGCAGGTC | |
| | | proteins | CCGCCTGTCCACCTGGGCCAGGCCTAGAGCCC | |
| | | | GGGAGCCCCTGGCTGGTGGGAGGCCACCCGC | |
| | | | AACCCACCCCACACGCAGCTCCAGCTCCCCCA | |
| | | | CCAGGCGGGGCGACTAGGACAGGGACAGAAC | |
| | | | CCGTTGAACCCAGGAGTGAGATCCGGCCCCG | |
| | | | GGTCCCGCTGGGCCCTCCCGTCCACCTTGGCT | |
| | | | GGACCTGGCGCCTGGGAGACCTTGGCTGGCG | |
| | | | CGAGGCCACGCCCACCAGACATGCAGTTCCA | |
| | | | GCTACCCCACCAGCTGGGCGACCAGGACAGG | |
| | | | GACGGAGGCTGCTGAGCCCAGTTAGAGGCCT | |
| | | | GCCCCCCGGGGTCTGTCCTGGGCGCTCCCCCA | |
| | | | AGGACGGACAGGGCAGGCAGGGTCCGGGAC | |
| | | | GATGGCCGCACAGTCCCGGCCCCGTGTTCCCA | |
| | | | GGCCCGTCTTGCTCCTCGATGTGAGGGAGACC | |
| | | | CGGGGGATGGGACAGGCTGGGCCCCGCAGTG | |
| | | | CCTGACTCCCTGCAGGGCTCCCGGGACAGGG | |
| | | | GTCCGGCGGACAGCCGGCTGCTCACGGGTGA | |
| | | | GGGGTCCAAGCTGGCATTGCGGCCACCTTCCG | |
| | | | GCCCGGGCTCTCTTGGGGAGGGGCGGGGTTG | |
| | | | GTGAGAACCGGTCACGTGCTCCGGGGCTCAC | |
| | | | TCGGGGTCTCCCAGGGCCGGAAGTAGGGCCC | |
| | | | CTGTGCGCAGGCGCCCTGAGGATCCCGGGCT | |
| | | | GCCCATCTCACGCCAGGGGGCGGAACTTCCT | |
| | | | GCAGCCTCTCTGCCTCCGCATCCTCGTGGGCC | |
| | | | CTGACCTTCTCTCTGAGAGCCGGGCAGAGGCT | |
| | | | CCGGAGCCATGCAGGCCGAAGGCCAGGGCAC | |
| | | | AGGGGGTTCGACGGGCGATGCTGATGGCCCA | |
| | | | GGAGGCCCTGGCATTCCTGATGGCCCAGGGG | |
| | | | GCAATGCTGGCGGCCCAGGAGAGGCGGGTGC | |
| | | | CACGGGCGGCAGAGGTCCCCGGGGCGCAGGG | |
| 38 | Maspin | serpin | ctgggaccacaggcatgcatcaccacactag | NM_002639 |
| | | peptidase | gctattgttttacattttttgtagagatggg | |
| | | inhibitor, | gtctcaccatgttgcccaggttggtctcaaa | |
| | | clade B | ctcctgggctcaagcaatccgctcacgtca | |
| | | (ovalbumin), | acctccccaaatgctgggattacaggcgtga | |
| | | member 5 | gccaccgcgccaggccTGAGTAATCCTAAT | |
| | | [Homo | CACAGGATTTTAAAAAGAAACTTCCTGCGCCA | |
| | | sapiens] | CCCATTAAACAATATCTCCTACCAATTTGG | |
| | | | TAGTAAATATTTTGCTAATAGTACCTAATTT | |
| | | | TTAGGTAGGCACTGTGTTTATACATATATCCA | |
| | | | TTCCTTCTTTTTTGATTGTCTTTCTGTTT | |
| | | | AATGGGCAGCTACCTCTCTTGG | |
| | | | CATCTAGCAGAATGAGCTGCTGCAGT | |
| | | | TTACACAAAAAGAATGGAGATCAGAGTACTT | |
| | | | TTTGTGCCACCAACGTGTCTGAGAAATTTGTA | |
| | | | GTGTTACTATCATCACACATTACTTTTATTTCA | |
| | | | TCGAATATTTCACCTTCCGGTCCTGCGTGGGC | |
| | | | CGAGAGGATTGCCGTACGCATGTCTGTACGTA | |
| | | | TGCATGTAACTCACAGCCCCTTCCTGCCCGAA | |
| | | | CATGTTGGAGGCCTTTTGGAAGCTGTGCAGAC | |
| | | | AACAGTAACTTCAGCCTGAATCATTTCTTTCA | |
| | | | ATTGTGGACAAGCTGCCAAGAGGCTTGAGTA | |
| | | | GGAGAGGAGTGCCGCcgaggcggggcggggcg | |
| | | | gggcgtggagctgggctggcagtgggcgtgg | |
| | | | cggtgcTGCCCAGGTGAGCCACCGCTGCTTCT | |
| | | | GCCCAGACACGGTCGCCTCCACATCCAGGTCTT | |
| | | | TGTGCTCCTCGCTTGCCTGTTCCTTTTCCAC | |
| | | | GCATTTTCCAGGATAACTGTGACTCCAGGTAAG | |
| | | | CAAGGTGGGGTAGCAGGGCTGGTGACTTC | |
| | | | CTTTTTTCAGGGAAATTCATAAATATCG | |
| | | | TTATTTGAGCTGATTTGAGATGGTGAACA | |
| | | | AAATGGACTTAGGTCCATTTTGGGGC | |
| | | | TGTTTTCAAAGACGGGCTGTTGG | |
| 39 | MDR1 | Multidrug | TAGGGCGATTGGGCCCTCTAGATNGCATNGCT | M29422 |
| | | Resistance | CGAGCGGCCGCCAGTGTGATGGATATCTGCA | |
| | | Gene | GAATTCGCCCTTGGAAGATCTAGAAATTCTTA | |
| | | | ATTCTAATTAAATTTGATTGCAAACTTCTAGT | |
| | | | CAAGACAAATATATTCATAAGATTAGATTTGT | |
| | | | AAAATACAAACAATTAGAAAGAGTATTTGTA | |
| | | | CCTTACCTTTTATCTGGTTGCTTCCTGAAGTGA | |
| | | | GTACTCCTAGGAGAATGAGAAATGATCTCTA | |
| | | | ATCTTTAGGAATCTGGAGAATATCTGAATAAA | |
| | | | GTAGATTTCTTCATGTTCTACTCTTCACAGGT | |
| | | | AAAGAGTAATGATAGCCTTTAAAATGGTAAT | |
| | | | ACAAGTGTTTATCCCAGTACCAGAGGAGGAG | |
| | | | CTACATGAACTAAGGCAGGCAGGCTTGAAAG | |
| | | | CACTAATCAGTGAAAACCCAAGGATAAGTTT | |
| | | | GGGTGGAGGAAGGGTGGGAGTAGAGATAAA | |
| | | | ATAAATTTTGAGTACATGACTATGGCTCCAAA | |
| | | | GCATTGAAGAAATATGTGTGATCTTTTTGCTA | |
| | | | AGGTGTAGGACGCCTTAATGAGCAGTTGAAA | |
| | | | AAACAAACAAAAACCTCGAAGAGTTACATGG | |
| | | | CTTAGGGATTGGGGTATAATTGAAAAGTAGC | |
| | | | CAGAGTTGAGAAGTTTAGCCAGAATAGGCAG | |
| | | | AATGAAGATTAGAATCTAAGCTAAAAAAAAA | |
| | | | AAAAAAAAAGAGAGAGACTTCTTTTGGTAGG | |
| | | | TTACTGGGAAGACCTTCAAATGAGAAGTGAA | |
| | | | GTAAAAATTGAATTAATTTGTTCAAATTTTTA | |
| | | | ATTTCTCTTTATCCNCTGGCTAAAAAATAATT | |
| | | | AGTAAATTTCAATTTAAAATACCATATGATAT | |
| | | | TTCAAACAAAATTGAAAATGTAACAAGAATT | |
| | | | TGAAGTAATAAGTATGGAAAATATAAAGATA | |
| | | | AATTAGCTTTATGGAAATTCATTTGTTTACT | |
| | | | TTGCAATTATATCAGNNTTTAATTTATAATG | |
| | | | AAAAAGT | |
| 40 | MGMT | MGMT (O6 | cattgtgaggtactgggagttaggactccaa | NM_002412 |
| | | methyl | catagcttctctggtggacacaattcaactcc | |
| | | guanine | taataACGTCCACACAACCCCAAGCAGGGCC | |
| | | methyl | TGGCACCCTGTGTGCTCTCTGGAGAGCGGCTG | |
| | | transferase) | AGTCAGGCTCTGGCAGTGTCTAGGCCATC | |
| | | | GGTGACTGCAGCCCCTGGACGGCATCGCCCA | |
| | | | CCACAGGCCCTGGAGGCTGCCCCCACGGCC | |
| | | | CCCTGACAGGGTCTCTGCTGGTCTGGGGGTCC | |
| | | | CTGACTAGGGGAGCGGCACCAGGAGGGGAGA | |
| | | | GACTCGCGCTCCGGGCTCAGCGTAGCCGCCCC | |
| | | | GAGCAGGACCGGGATTCTCACTAAGCGGGCG | |
| | | | CCGTCCTACGACCCCCGCGCGCTTTCAGGACC | |
| | | | ACTCGGGCACGTGGCAGGTCGCTTGCACGCC | |
| | | | CGCGGACTATCCCTGTGACAGGAAAAGGTAC | |
| | | | GGGCCATTTGGCAAACTAAGGCACAGAGCCT | |
| | | | CAGGCGGAAGCTGGGAAGGCGCCGCCCGGCT | |
| | | | TGTACCGGCCGAAGGGCCATCCGGGTCAGGC | |
| | | | GCACAGGGCAGCGGCGCTGCCGGAGGACCAG | |
| | | | GGCCGGCGTGCCGGCGTCCAGCGAGGATGCG | |
| | | | CAGACTGCCTCAGGCCCGGCGCCGCCGCACA | |
| | | | GGGCATGCGCCGACCCGGTCGGGCGGGAACA | |
| | | | ccccgcccctcccgggctccgccccagctcc | |
| | | | gcccccgcgcgccccggccccgcccccg | |
| | | | cgcgctctcttgcttttctcaggtcctcgg | |
| | | | ctccgccccgctctagaccccgccccacgcc | |
| | | | gccatccccgtgcccctcggccccgccccc | |
| | | | gcgcccCGGATATGCTGGGACAGCCCGCGCCCC | |
| | | | TAGAACGCTTTGCGTCCCGACGCCCGCAGGTC | |
| | | | CTCGCGGTGCGCACCGTTTGCGACTTGGTGAG | |
| | | | TGTCTGGGTCGCCTCGCTCCCGGAAGAGTGCG | |
| | | | GAGCTCTCCCTCGGGACGGTGGCAGCCTCGA | |
| | | | GTGGTCCTGCAGGCGCCCTCACTTCGCCGTCG | |
| | | | GGTGT | |
| 41 | MINT2 | amyloid beta | ggcacaggcaggttacatagtcttctcaggat | NM_001163 |
| | | (A4) precursor | gtcagtggcagagctaggaCGTCTATCTCTGG | |
| | | protein- | CAGCTCAGTTCTGTGCGAATCCAGGCAGATGG | |
| | | binding, | TGCTGATCAGTAAGGGGTGCTGGCTGAGCGCT | |
| | | family A, | GATGGCCACCTGCATCTCAAGGAGAAACAG | |
| | | member 2 | TGTCACTGGCTAATCTGATGGCTTCTCT | |
| | | | GGGCACCAGCACGTGGGCACCATCACCCT | |
| | | | TTCTCTGCAGGGGGTTTGTTTAGTGT | |
| | | | ATTTGGTAGAACATCCCCCAGCCTACTAGGTG | |
| | | | TGGCATGCTCTATGCCACAAGCTCTGTATCTC | |
| | | | AGGCAGCATTTTGTACTTTGAAAAAACAAGTT | |
| | | | GGGAACAGAACCCTGATGAATGTGTTTCATTT | |
| | | | CCTGTCAGAGCAAATGAAACCTGAAATATTA | |
| | | | ATGGCACGAGATTTCCCTTATCTTCCTACAAA | |
| | | | ATCTTCCTACATTGAAAAATGTACTCCCCACA | |
| | | | AGCTTAGCATGCAGCTCTGCTACCTGTGGCCC | |
| | | | GAAATCATTAGTTGTCCATACTCACTGACCTT | |
| | | | TGGAAATAAACACGAAGGTTCACTTGAAGAC | |
| | | | TTGGGGGAGAATCACGGTCAACTTGTGACGC | |
| | | | TTGGTTTTTCAGATATTCAGCTGCTCTGGAGA | |
| | | | GCCTTGGAGTTCCAGCTGCTCTAGAGGTTCTG | |
| | | | GGGAGGGAGCTGTTAGCCTCCCATATGAGCG | |
| | | | TGTGGCCCATCGTTGCCATCCACACCTGCCCC | |
| | | | TCTGTGGGTGAATAAGTGGTTTCCTTTCTCAG | |
| | | | CTGGTTGACGCTTCATTTGTTTGTGTTCTTTTT | |
| | | | CTTTACAGTCTCCTGAATATTTACGCGTTGCT | |
| | | | GAATCTCCTGTGGACAAACCACCAATAGGCC | |
| | | | AGGACTGTCCTGTGGACAGACGGGGTGAGCC | |
| | | | TCTTCTTGTGTCTGGAGATTCTGAGTGAGTAG | |
| | | | AACCCGTTATGATCCCCACTGCACTTAATGTG | |
| | | | GCATTCATGAATGAGTCTGGGCTGATGTGCTA | |
| | | | ATTGGGGGCCGTAAGAAGAGTTATAGCC | |
| 42 | MINT31 | amyloid beta | CCGGGGCCTCTATCCTGGCGGGAAGGGCAGG | AF135531 |
| | | (A4) precursor | CCGACCCGGCAGACTGCGGCCTCTCGGGAGG | |
| | | protein- | GAAGAAGGTGTCAGACGCGCGGAGCAACCAT | |
| | | binding, | AAATAGCCCCCCTTTCCCAGAAGACGGCACG | |
| | | family A, | GGGTTCAAGACTCAGGCGCCGCATACTCAGA | |
| | | member 3 | ATGAGAGCAGAGACTCCCGCCAGGAAAAAAA | |
| | | | GGCACTTAGGGGATCTGCTCATTAGCATGAA | |
| | | | ATGCAAATGAGCCCGGCCGGCCTCATTTACAC | |
| | | | AACTCTGTGCATGGATTCGGCGAAAGGGCAA | |
| | | | CCAGGGAGACGACGGCGCAGCAGCCACTCTG | |
| | | | CCACTTCCCCCATCCCCTCCCCCCATCGGCCG | |
| | | | GGGCGGGAACTGAGACGACCCCAACCCTCTG | |
| | | | CGGTGGCGGGAGGTGCGCGGGGGCTGCGTGG | |
| | | | GTGGTGCAGCCTTAGGAGAGTGAACAACGCC | |
| | | | CAGGGGTGATGGCCTCAGCAAAGTGAGGGGT | |
| | | | GGTGATGGAGGTCATCCGACCCATCCCGCCG | |
| | | | CCTCTCCGCAGTGGCGCAAGCGCCCCAAAAT | |
| | | | CTCCGGAGAGGGAACTGACTGACCCACTAGG | |
| | | | TTCCGCCGTGTCTACCTCTCGCAGATGTTGGG | |
| | | | GAAGTGCTTCCCGGCGTCTAATCCTCGCTGTT | |
| | | | CCCCCCTCCACCGGCGCCCAGCACACCCGCG | |
| | | | GCGCTCCGCTCCCGGG | |
| 43 | MLC1 | megalencephalic | AGTGTTTTGAGCTGCATTTATGCGTACTTGAC | NM_015166 |
| | | leukoencephal- | ACTTACGCATTTTGATCGAGGTGATTTAGTGG | |
| | | opathy with | GCATTTTCACTGGGACAGGGATGCTTGTATGT | |
| | | subcortical | GTAATcttactaaaagctaataaaaacttac | |
| | | cysts 1 | taaaagctaataaaagcttactaaaagcttCT | |
| | | | TGCTTGATTGAAACGAAGACAACAGAACATC | |
| | | | CCATGGTCTGGAACCTGATGACTTTGCTC | |
| | | | AAGTTTTAATGTGGGTTCATGGTTTAA | |
| | | | GGAGCTGGTTTTTCAGAAACTTTAGTTTGAGC | |
| | | | CTTTTTACAATGTGCACAAAGAACCCGTTGCT | |
| | | | GTAGTTGTCAGGGTGCCAGTGTCTCTGGGCGA | |
| | | | CACACATTACTGTGGTTTTTCTCTGCTTGGTG | |
| | | | AGCAGAGATAAAGGGGGCAGCAGGACCGGG | |
| | | | CCCACCAGCCATCCGGGCTGCCCACGCAAAC | |
| | | | CACAGGGCCGAATCCGGAGCCGCCCAAGGCC | |
| | | | ACACAGCTAAGCCGAGTGCGTGAATGCTTAT | |
| | | | GTGACCGTGTGAAGGAGGTTCCCACCGTGTG | |
| | | | GCTGTGGGGGATGGAAAAAGGCTACTTGGAA | |
| | | | AGATGTAGAAGACCTTCGAGTAAACAGTTAC | |
| | | | GTTTCAGAAACAGAGCCTGCTCAGAATGTGT | |
| | | | ACTTGGTGGGATTCTATTCTTAGGGACGCTTC | |
| | | | TTTCTTCTGAGAGACCCGAGCTCTGTGGCGAG | |
| | | | TGGCACAGGCAGGGCCCCTTCCTTTCCTAGTT | |
| | | | GGGTTCTGACAGCTCCGAGGCAGTGGTTTACA | |
| | | | CAACCAACACGAAACATTTCTACGATCCACCC | |
| | | | GATTCCTCCCCTCATTGATATTCAGGAAGCAG | |
| | | | CTCTCCTTCCCCTGCCTTCAGCTCAAGTTTGCT | |
| | | | GAGCTTTTGTTTCATTTGTGAATACTTCTTGCT | |
| | | | GGAAGTCCCTCACCCAGAGACCAGTGCTCCC | |
| | | | AACGGCAGAGCAGCGGGGGAGGTAAGTGCTC | |
| | | | AGACATTAAGCCGTTGAGTAGAGGCATGTTTT | |
| | | | GCAATCTCTCGTTTAGCTACCAATTGG | |
| 44 | MT-X (I | metallothionein | ctaacacggattaaTGTTATGTAGAGTAATAGG | NM_005952 |
| | & II) | 1X | AATATGGAAGGAAAAATAACCCTGTTTCTTGCA | |
| | | | TTTTAATTTAATCCGGAATCCGCATATCACCTA | |
| | | | AAATGATCCCTTTTCTGGGAGCATTCCACATTT | |
| | | | TCCAAACTGTCATCCTGTGGTGGGGTGCCCGGC | |
| | | | TAGGCTATGGGGAGACCTGGAGAGTTTTATG | |
| | | | CAAAGGAGGACCTGGGCAAATGTGCCCATTC | |
| | | | AGCCTCTCAAGAGTGGAGAATGCAAGGACGG | |
| | | | GGGCAGAGCCCTGTGTCTGTTCTGTCCCTAGA | |
| | | | CATAAGAGAAACGTGGCCAACAGACCGAGGT | |
| | | | GGGGACGGGGACAGGGACCGGCAATGCAGG | |
| | | | AAATCCGAGTGTCACATCCTCTGCCTCTCATT | |
| | | | TGCACACTGCTCCCTCGCTATGCTCACCGCTC | |
| | | | CCGCCGATCCAGGGACGTGATCCAGGGACTC | |
| | | | TGGGAAATGCAAAGCTACACACAGTGGAGCG | |
| | | | GGGGCTGGGGGTGTGTAGACCGCCGGGATTC | |
| | | | CGAGTTTCCCGGCACGCCTAGGAGAGGGAGA | |
| | | | GGCAGGCAATGTCAGGGAAATTGGGCAGGCA | |
| | | | AGACGCCAGGGACGCCACGTACTGCCAGGTT | |
| | | | CTCAACGAGGTGGAGCCAAAGGGGCAGGCCC | |
| | | | CGCGGTGCGCCCGGCGCTGGGCTCACGGGTT | |
| | | | GCTGCACCCGGCCCAGGATCGCGGGCGGTGC | |
| | | | AGACTCAGCAGGGGCGGGTGCAAGGACGAGG | |
| | | | CGGGGCCTCTGCGCCCGGCCCTCTTCCCGGAC | |
| | | | TATAAAGAGAGCCGCCGGCTTCTGGGCTCCA | |
| | | | CCACGCTTTTCATCTGTCCCGCTGCGTGTTTTC | |
| | | | CTCTTGATCGGGAACTCCTGCTTCTCCTTGCCT | |
| | | | CGAAATGGACCCCAACTGCTCCTGCTCGCCTG | |
| | | | GTAAGGGACACCTAGCTCCGCGCCTTGGGAT | |
| | | | GCCCGTTTCCCAGCCACAGTACAGACTCTTCC | |
| | | | TGGGTTTGAAGAAGTCGCATTTAAAGTTCTGA | |
| | | | GCTGAAGGGGCTCCTTTAT | |
| 45 | MUC2 | Homo sapiens | CTGGGGAGCCTGGGCAGGCTGTCACCTCCTCA | NM_002457 |
| | | mucin 2, | GCTGTCAGGCCCGAGGTCCTCATGTGGTCCCC | |
| | | intestinal/ | AGGAGAAGGGGCAGACGGCCACTTCCGGCCA | |
| | | tracheal | CCAGCCAGCTCCCTGTGTGCCTGATTCCGTAA | |
| | | | CATGTCCCCTGGCTGGGCATGTACTCCCCAAG | |
| | | | TTCTAATTACATGTAACTGCAGAGAAGGGCTC | |
| | | | AGCCTGGGAAAAGGATGGGCATAGGGGGTGG | |
| | | | TTGGGGGCTGGGGCCTCTGACACAGCTCCATG | |
| | | | AGCCCGGCCAAGAGTCCCACACAAGTCAGTG | |
| | | | GCCCCCCCGGACCCTGAAGGATCCCACATCCT | |
| | | | CCCTGCCCTCGGGGAGGCCCCTTTCTGGGGTC | |
| | | | AGGCCTGGAAGCTGCCCCAGAGCTTGGGCCC | |
| | | | CAGGAATGGGTTGGTCCTCCCAGCGTAACGT | |
| | | | GAGCCTGATCAGGCCTGGGGACCTGCTCAGC | |
| | | | GGGTGTCTGGGGGCCCATGGCGGGCTAAGGA | |
| | | | GCCTGACCAGACTTGCTTCTGGCAGGACACCC | |
| | | | CTCCCCCGGCCACCCTGGGCTCGCCCCTCTAG | |
| | | | TAGCTGCATGTGTTCCCCGGGTGTGTGTTGGC | |
| | | | ATTCAGGCTACAGGGCTGCCTCATCCTGAAGA | |
| | | | AGGCTGCGTTTACCCAGGGAGCCATAAAGAG | |
| | | | ATGACCTCCGATAACCTGAATCAATATTTCCC | |
| | | | CATTGGGGCTCGGGCCCCCGCAGCTGTCTTCT | |
| | | | TGATCATCTGGCAGATGCCACACCCACCCTTG | |
| | | | GCCCTCCCCTGCCTTCCTGCCCTCCTACCCTCC | |
| | | | TGCCAGGACATATAAGGACCAGACCCCTGCC | |
| | | | CCCGGGCGCAACCCACACCGCCCCTGCCAGC | |
| | | | CACCATGGGGCTGCCACTAGCCCGCCTGGCG | |
| | | | GCTGTGTGCCTGGCCCTGTCTTTGGCAGGGGG | |
| | | | CTCGGAGCTCCAGACAGGTGAGAGAGCAGAC | |
| | | | ACAGGGGTCTGGGGCCTGGCAGAGTGTCCTG | |
| | | | GGGGCAGGGCGAGGCGGGCGGGCAAGTCGC | |
| | | | GTCTGGGAGGAGGAGCTGGTCC | |
| 46 | MYC L2 | v-myc | AGGGCGATTGGGCCCTCTAGATGCATGCTCG | J03069 |
| | (v-myc) | myclocytoma- | AGCGGCCGCCAGTGTGATGGATATCTGCAGA | |
| | | tosis viral | ATTCGCCCTTGTTCTCGGATCCCGATCATATC | |
| | | oncogene | CGCACTGCAGGTGTTCTCGGATCCCGATCATA | |
| | | homolog 2 | TCCACACTGCAGGTGGAGCTCATTGGCTCATG | |
| | | (avian) | CCTGTAATCCCAACACTTTAGGAGGCTGAGGC | |
| | | | ATACCGACCACTTGCGGTCAGGAATCAAGAC | |
| | | | CAGCCTGGCCAACATGGCGAAACCTCGTCTCT | |
| | | | ACTAGAAATACAAAAAATAAAAATAAAAATA | |
| | | | AATTAACCAGGCGTGGTGGCCCACGCGCCCC | |
| | | | TGTAGTCGTAGCTACTTTGGAGGCTGAGGTGG | |
| | | | GAGAATCACTTGAACTCGGGAGGCGGAGGTC | |
| | | | GCAGCGAGCAGAGATTGAGCCACTGCACTCC | |
| | | | AACCTGGGTGACACAAGAAAGAAAGAAAATG | |
| | | | AAGGAAAGAAGAAGGAAGGAAAGAAAGAAG | |
| | | | GAAAGAAGGAAGGAAGGAAGAAAGGAAGGA | |
| | | | AGGAAGGAAAAAAATAGCTGGACATGATGGA | |
| | | | GGACTAGCATTTCTCAATTTCAAAACGTACTA | |
| | | | CAAACCACACTAATCAAAACAATGTGGTACT | |
| | | | GGCATAAGGATAGACATATAGATCAATGGAG | |
| | | | TAGAATTGAGAGTCAGAAACCCATACATCTA | |
| | | | AGGTCAACTGATTTTCAAAGAGATGTCAAGA | |
| | | | CCATGCAATTGGAAAAGAATAATCTCTTCAAC | |
| | | | AAATGGTGCTGGAATACTTGGATACTCACATG | |
| | | | CAAAAGAATGAAGCTAGGCCCTTACCTCACG | |
| | | | CCATTTACAAAAAATAACTCAAAATGAACCA | |
| | | | AAGGCCTAAATATAAGAGCTAAAATTGTAAG | |
| | | | CCTCTTAGAAATAAACAGAGGGCGGGTCGCG | |
| | | | CGCTCGGTGGGCGCGTTGTGCGCGTGTGTGGA | |
| | | | GTGCCCTGCTGCCCCCAGC | |
| 47 | MyoD | myogenic | GTTTGGAGAGATTGGCGCGAAGCTTTAGCAG | NM_002478 |
| | | factor 3 | CAATCTCCGATTCCTGTACAACCATAGCTGGG | |
| | | | TTTCTAAGCGTCTAGGGAAGAAGGACTGGGC | |
| | | | CCACGACCTGCTGAGCAACTCCCAGGTCGGG | |
| | | | GACTGGCGGAATATCAGAGCCTCTACGACCC | |
| | | | GTTTGTCTCGGGCTCGCCCACTTCAACTCTCG | |
| | | | GGGTCTCTCCGCCTGTTGTTGCACTCGTGCGT | |
| | | | TTCTCTGCCCCTGACGCTCTAAGCTTTCTGCTT | |
| | | | TCTGCGTGTCTCTCAGCCTCTTTCGGTCCCTCT | |
| | | | TTCACGGTCTCACTCCTCAGCTCTGTGCCCCC | |
| | | | AATGCCTTGCCTCTCTCCAAATCTCTCACGAC | |
| | | | CTGATTTCTACAGCCGCTCTACCCATGGGTCC | |
| | | | CCCACAAATCAGGGGACAGAGGAGTATTGAA | |
| | | | AGTCAGCTCAGAGGTGAGCGCGCGCAGCCAG | |
| | | | CGTTTCCCGCGGATACAGCAGTCGGGTGTTGG | |
| | | | AGAGGTTTGGAAAGGGCGTGCCGGAGAGCCA | |
| | | | AGTGCAGCCGCCTAGGGCTGCCGGTCGCTCCC | |
| | | | TCCCTCCCTGCCCGGTAGGGGACCTAGCGCGC | |
| | | | ACGCCAGTGTGGAGGGGCGGGCTGGCTGGCC | |
| | | | AGTCTGCGGGCCCCTGCGGCCACCCCGGGGA | |
| | | | CCCCCCCAAGCCCCGCCCCGCAGTGTTCCTAT | |
| | | | TGGCCTCGGACTCCCCCTCCCCCAGCTGCCCG | |
| | | | CCTGGGCTCCGGGGCGTTTAGGCTACTACGGA | |
| | | | TAAATAGCCCAGGGCGCCTGGCGAGAAGCTA | |
| | | | GGGGTGAGGAAGCCCTGGGGCGCTGCCGCCG | |
| | | | CTTTCCTTAACCACAAATCAGGCCGGACAGG | |
| | | | AGAGGGAGGGGTGGGGGACAGTGGGTGGGC | |
| | | | ATTCAGACTGCCAGCACTTTGCTATCTACAGC | |
| | | | CGGGGCTCCCGAGCGGCAGAAAGTTCCGGCC | |
| | | | ACTCTCTGCCGCTTGGGTTGGGCGAAGCCAGG | |
| | | | ACCGTGCCGCGCCACCGCCAGGATATGGAGC | |
| | | | TACTGTCGCCACCGCTCCGCGA | |
| 48 | NES-1 | solute carrier | TTCCCTGGCAGGGGGTGCGGGAGAAGGGGCC | NM_024609 |
| | | family 5 | CTTCCCCAAGAACAGAACTTCCTAAAGCGGA | [ |
| | | (sodium iodide | TGTTTGAACCTCGCAGTTATACAGAAGACTTG | |
| | | symporter), | TAGGAAGGATGGACAAACGTTCTTAAGCCCA | |
| | | member 5 | TGACGGCCCTTAACCTGGTCGCTCCCTTTTCT | |
| | | | GATGGAGACTCAGGCAATAGCgtgtgtgcgtg | |
| | | | tgtgtgtgtgtgtgtgtgtgtgtgtgtatcc | |
| | | | gtgtgtCCTAATATCAGACATTTGTTCTTGTT | |
| | | | TTCCAGGCAGCGTCTCTCTAGCTTCTTTCTG | |
| | | | CAATGCTGTAGTACTCTCTCCAGTATTTCAGG | |
| | | | AGGAGGAGCATTTGCTATTTCAAAAACGAAA | |
| | | | AACAAAAACCTGGCCAcatccatttttttcag | |
| | | | cagccatgcgatttccatcattgctcacattt | |
| | | | tatggatgaggaaactgagtcttagaggaatt | |
| | | | cagtaaGTGATACCTCTCTCGGATGTGTTG | |
| | | | AGTAACTGAGACTGCACTCCCTCCCAGGC | |
| | | | TGGAACGTCCTGGTACTCCCACCCCCACAGGC | |
| | | | TCAGTTCTGTGCATTATCTGCCTTTTTCGGGG | |
| | | | ATTGTGACCCTTCTTCACAGCCTCCTCCCTCA | |
| | | | GAAAGCCACCACCATCAGATCCGATTCTCCAT | |
| | | | GGTACAGCTTCTTCTTTGGTTCCACTCTCCAG | |
| | | | CACCCTGGGGAAGCAGGAACAGAGGCTGCTG | |
| | | | CCACTCTCTGACCTCTAAGGGGTTAAGGCCTG | |
| | | | GGTCCCGCCCCTCTTCCCGCCCGCCTGGCGGG | |
| | | | AGTATGAATAGCCTCGCTCCCACTCCCGACTC | |
| | | | TCAGTCGCTCAGGCTACTCCCACCCCGCCCCG | |
| | | | CCCCGTCATTGTCCCCGTCGGTCTCTTTTCTCT | |
| | | | TCCGTCCTAAAAGCTCTGCGAGCCGCTCCCTT | |
| | | | CTCCCGGTGCCCCGCGTCTGTCCATCCTCAGT | |
| | | | GGGTCAGACGAGCAGGATGGAGGGCTGCATG | |
| | | | GGGGAGGAGTCGTTTCAGATGTGGGAGCTCA | |
| | | | ATCGGCGCCTGGAGGCCTACC | |
| 49 | NF-L | neurofilament, | ATACCTGCAGTAGTGCCGCAGTTTCACGAgtgtg | L04147 |
| | | light | tgtgtgtgtgtgtgtgtgtgtgcgcgcgcgcgc | |
| | | polypeptide | gcgcactcgcgcgcACATTCCCTATGTGTTAAG | |
| | | 68 kDa | CAGCTCATTAAAGAAAAAGAAAAATAATCAGGA | |
| | | | GAAAGGAAGATGAATTGCAGAAAGTGCCAGAA | |
| | | | AGCTAGAAAGAAATTAAAACTCTTCTCCATACA | |
| | | | TACTGCATACACATAACCTAGCCTATTTATTTG | |
| | | | TATCTAAAATTCCCTAGCCGCACCATCACCGTA | |
| | | | AACACCAAGGGAAAAAATTAAGGAGGTTCCTGG | |
| | | | TGGGAAAAGGGCGAGTTGGGGGGACAGGGTGTC | |
| | | | TGCGAGGTGACGGGATACAGAAAACTAGGGTGT | |
| | | | CAAAAGGGAGCAAGAACCTGTTTTGGGGGCAAC | |
| | | | TTAAGGATCCAAGTGTCACGGGGTCTGGGCA | |
| | | | ATGCAGGACGGGAGGGGCTGCGTGAGTGAGT | |
| | | | ACAGAAGGGAAATGAGTGAGGGGGCATGGG | |
| | | | ATCTCAGAGAAAATCAGGGCCCTCTGAGCAA | |
| | | | AGTGGAAAGGACGACCGCCGCAGCTCCTCGG | |
| | | | GCCGTAGCTCGACCCCGCCTTCCCTTTTGCGC | |
| | | | AGAATCCTCGCCTTGGCTGCAGCAGCGCGCTG | |
| | | | CCCCCACTGGCCGGCGTGCCGTGATCGATCGC | |
| | | | AGGCTGCGTCAGGAGCCTCCCGGCGTATAAA | |
| | | | TAGGGGTGGCAGAACGGCGCCGAGCCGCACA | |
| | | | CAGCCATCCATCCTCCCCCTTCCCTCTCTCCCC | |
| | | | TGTCCTCTCTCTCCGGGCTCCCACCGCCGCCG | |
| | | | CGGGCCGGGGAGCCACCGGCCGCCACCATGA | |
| | | | GTTCCTTCAGCTACGAGCCGTACTACTCGACC | |
| | | | TCCTACAAGCGGCGCTACGTGGAGACGCCCC | |
| | | | GGGTGCACATCTCCAGCGTGCGCAGCGGCTA | |
| | | | CAGCACCGCACGCTCAGCTTACTCCAGCTACT | |
| | | | CGGCGCCGGTGTCTTCCTCGCTGTCCGTGCGC | |
| | | | CGCAGCTACTCCTCCAGCTCTGGATCGTTGAT | |
| | | | GCCCAG | |
| 50 | NIS | solute carrier | CTGGCACAGGGCCAACTCTCAGTGCATATCTG | AF059566 |
| | | family 5 | CAAAGGAACCAATGAATGAATGAATGAAGTG | |
| | | (sodium iodide | ACAAATGaataaaggaataaatgaatgaggca | |
| | | symporter), | cttatcatgtaccaggctttcgttaccacgtc | |
| | | member 5 | ccatttattcctctgaggcagggtctatttta | |
| | | | tccttgttacagatggggaaactaaggcccag | |
| | | | ggaggagcaaagtcttccccaagTATGTACC | |
| | | | CACTCAGAACTTGAGCTCTGAATGTCTCCC | |
| | | | ACCCAGCTTAGCCCAAGAGCGGGGTTCAGTG | |
| | | | ATGCCCACCCCCTAAGGCTCTAGAGAAAGG | |
| | | | GGGTAGGCCCACATGCCAGTTTGGGGGTGG | |
| | | | TAAAGCCAGGTAAGTTTTCTTTATGGGTCC | |
| | | | CCTGAAACCCTGAAAGTGAACCCCAGTCCTG | |
| | | | CATGAAagtgagctccccatagctcaaggtat | |
| | | | tcaagcaCAATACGGCTTTGAGTGCTGAAGC | |
| | | | AGgctgtgcaggcttggatagtgacatgccct | |
| | | | ctctgagcctcaatttccccacctgtcaacag | |
| | | | cagacagtgacagctGTGATCAGGGGATCA | |
| | | | CAGTGCATGGGGATGGGTGGGTGCATGGGGAT | |
| | | | GGAGGGGCATTTGGGAGCCCTCCCCGATACCA | |
| | | | CCCCCTGCAGCCACCCAGATAGCCTGTCCTGG | |
| | | | CCTGTCTGTCCCAGTCCAGGGCTGAAAGGGTG | |
| | | | CGGGTCCTGCCCGCCCCTAGGTCTGGAGGCGG | |
| | | | AGTCGCGGTGACCCGGGAGCCCAATAAATCTG | |
| | | | CAACCCACAATCACGAGCTGCTCCCGTAAGCC | |
| | | | CCAAGGCGACCTCCAGCTGTCAGCGCTGAGCA | |
| | | | CAGCGCCCAGGGAGAGGGACAGACAGCCGGCT | |
| | | | GCATGGGACAGCGGAACCCAGAGTGAGAGGG | |
| | | | GAGGTGGCAGGACAGACAGACAGCAGGGGCGG | |
| | | | ACGCAGAGACAGACAGCGGGGACAGGGAGGC | |
| | | | CGACACGGACATCGACAGCCCATAGATTCCT | |
| | | | AACCCAGGGAGCCCCGGCCCCTCTCG | |
| 51 | NME2 | c-myc | ACTGGAAAACTCGACCGCACTTTAGTGCCAG | NM_002512 |
| | | transcription | GTGGGCAGGGATCCCCATGTCAGGGTGGGAG | |
| | | factor; non- | TGGGGCGGCTGATTGGGGCTGGAAATGTAGG | |
| | | metastatic | TGGGGAGGCGGCAGCCAGGGAGCAGGGCATC | |
| | | cells 2, pro- | CTGCGAGAAGAGCATCCCGCTAAGGAGTCTG | |
| | | tein (NM23) | AACGCCATCCTGTAGGCGGGGGAGTCATCAA | |
| | | expressed in; | GGCAGGGCAGAGGCAGGACCAGATGGCCGTT | |
| | | nucleoside- | TGAGGTGCTGAGCAAAGCTCCCGGTTTGCGC | |
| | | diphosphate | GGAGAGGTGAGATCGAGGCCCCTTGGGAGGC | |
| | | kinase 2 | CGAGGCTTAACCAGGGCTCAAGCAGAGGGGA | |
| | | | GGGAAGGCTGGATTTCAGAGGTAGGGAGGAT | |
| | | | AAGGACCGTGGGTGCACGACGGGGAGGGAG | |
| | | | AGCCAAGTCAAGGTTAATGCCGGTGCTCGGG | |
| | | | CGGATGGTGAAAGCAGCAGATGGCCTTGACC | |
| | | | GGGGTAGAGAACTCGAGCACAGGAGCAGGTT | |
| | | | Ctgtgtgtgtgtgtgtgtgtgtgtgtgtgt | |
| | | | AGGAGCTTTTGGGGTCACGGGAAGTACTGAG | |
| | | | AGGTGAGGAGTGGGATTTGGGACGTGCGTAG | |
| | | | TTGAACTCATAGGACGTCCAGGTGGAGAAGG | |
| | | | AATCACTTCCTGTCTCTGGATCCGTCTCGATC | |
| | | | TCTGCCTGGCGAGGGCGCGCCCCGGCTGGGCG | |
| | | | TGGACACTGTTCTCCGGCCGCGTCGGGCCGGG | |
| | | | CGGGTGGGGCGTTCCTGCGGGTTGGGCGGCTG | |
| | | | GGCCCTCCGGGGTGTGGCCACCCCGCGCT | |
| | | | CCGCCCTGCGCCCCTCCTCCGCCGCCGGCT | |
| | | | CCCGGGTGTGGTGGTCGCACCAGCTCTCTGC | |
| | | | TCTCCCAGCGCAGCGCCGCCGCCCGGCCC | |
| | | | CTCCAGCTTCCCGGTAAGGCGGTGGGGG | |
| | | | CGCATCCCCTGGCGACTCCTCCCGTTCCCT | |
| | | | CTTCCGCTTGCGCTGCCGCAGGTGGGCCC | |
| | | | GGTCTGTGGGCGCCCCCCGATTTCCCGCAGGT | |
| | | | CCCGCGCGGCGTCGGAGCGGGAGATTCCCTT | |
| | | | GCAGCTTGCGCCCCGC | |
| 52 | NPAT | nuclear | TACATACAAAGAGGCTTAAACTGCCCAGAAC | AY220758 |
| | | protein, | CTCCGAATGACGAAGAATCACCGCCAGTCTC | |
| | | antaxia- | AACTCGTAAGCTGGGAGGCAAAACCCCAAAG | |
| | | telangiectasia | CTTCCCTACCAAGGGAAAACCTTTGGCCTCAA | |
| | | locus | AGGTCCTTCTGTCCAGCATAGCCGGGTCCAAT | |
| | | | AACCCTCCATCCCGCGTCCGCGCTTACCCAAT | |
| | | | ACAAGCCGGGCTACGTCCGAGGGTAACAACA | |
| | | | TGATCAAAACCACAGCAGGAACCACAATAAG | |
| | | | GAACAAGACTCAGGTTAAAGCAAACACAGCG | |
| | | | ACAGCTCCTGCGCCGCATCTCCTGGTTCCAGT | |
| | | | GGCGGCACTGAACTCGCGGCAATTTGTCCCGC | |
| | | | CTCTTTCGCTTCACGGCAGCCAATCGCTTCCG | |
| | | | CCAGAGAAAGAAAGGCGCCGAAATGAAACCC | |
| | | | GCCTCCGTTCGCCTTCGGAACTGTCGTCACTT | |
| | | | CCGTCCTCAGACTTGGAGGGGCGGGGATGAG | |
| | | | GAGGGCGGGGAGGACGACGAGGGCGAAGAG | |
| | | | GGTGGGTGAGAGCCCCGGAGCCCGAGCCGAA | |
| | | | GGGCGAGCCGCAAACGCTAAGTCGCTGGCCA | |
| | | | TTGGTGGACATGGCGCAGGCGCGTTTGCTCCG | |
| | | | ACGGGCCGAATGTTTTGGGGCAGTGTTTTGAG | |
| | | | CGCGGAGACCGCGTGATACTGGATGCGCATG | |
| | | | GGCATACCGTGCTCTGCGGCTGCTTGGCGTTG | |
| | | | CTTCTTCCTCCAGAAGTGGGCGCTGGGCAGTC | |
| | | | ACGCAGGGTTTGAACCGGAAGCGGGAGTAGG | |
| | | | TAGCTGCGTGGCTAACGGAGAAAAGAAGCCG | |
| | | | TGGCCGCGGGAGGAGGCGAGAGGAGTCGGG | |
| | | | ATCTGCGCTGCAGCCACCGCCGCGGTTGATAC | |
| | | | TACTTTGACCTTCCGAGTGCAGTGGTAGGGGC | |
| | | | GCGGAGGCAACGCAGCGGCTTCTGCGCTGGG | |
| | | | AAATTCAGTCGTGTGCGACCCAGTCTGTCCTC | |
| | | | TCCCCAGACCGCCAATCTCATGCACCCCTCCA | |
| | | | GAGTGGCCCTTGACTCCTCCCTCTCC | |
| 53 | p21 | p21 protein | GGGTAACCGACTCCTATAGGGCGAATTGGGC | U24170 |
| | | | CCTCTAGATGCATGCTCGAGCGGCCGCCAGTG | |
| | | | TGATGGATATCTGCAGAATTCGCCCTTCTAGC | |
| | | | TAGCACCACAGGGATTTCTTCTGTTCAGGTGA | |
| | | | GTGTAGGGTGTAGGGAGATTGGTTCAATGTCC | |
| | | | AATTCTTCTGTTTCCCTGGAGATCAGGTTGCC | |
| | | | CTTTTTTGGTAGTCTCTCCAATTCCCTCCTTCC | |
| | | | CGGAAGCATGTGACAATCAACAACTTTGTAT | |
| | | | ACTTAAGTTCAGTGGACCTCAATTTCCTCATC | |
| | | | TGTGAAATAAACGGGACTGAAAAATCATTCT | |
| | | | GGCCTCAAGATGCTTTGTTGGGGTGTCTAGGT | |
| | | | GCTCCAGGTGCTTCTGGGAGAGGTGACCTAGT | |
| | | | GAGGGATCAGTGGGAATAGAGGTGATATTGT | |
| | | | GGGGCTTTTCTGGAAATTGCAGAGAGGTGCA | |
| | | | TCGTTTTTATAATTTATGAATTTTTATGTATTA | |
| | | | ATGTCATCCTCCTGATCTTTTCAGCTGCATTG | |
| | | | GGTAAATCCTTGCCTGCCAGAGTGGGTCAGC | |
| | | | GGTGAGCCAGAAAGGGGGCTCATTCTAACAG | |
| | | | TGCTGTGTCCTCCTGGAGAGTGCCAACTCATT | |
| | | | CTCCAAGTAAAAAAAGCCAGATTTGTGGCTC | |
| | | | ACTTCGTGGGGAAATGTGTCCAGCGCACCAA | |
| | | | CGCAGGCGAGGGACTGGGGGAGGGAAGGAA | |
| | | | GTGCCCTCCTGCAGCACGCGAGGTTCCGGGA | |
| | | | CCGGCTGGCCTGCTGGAACTCGGCCAGGCTC | |
| | | | AGCTGGCTCGGCGCTGGGCAGCCAGGAGCCT | |
| | | | GGGCCCCGGGGGAGGGCGGTCCCGGGCGGCG | |
| | | | CGGTGGGCCGAGCGCGGGTCCCGCCTCCTTG | |
| | | | AGGCGGGCCCGGGCGGGGCGGTTGTATATCA | |
| | | | GGGCCGCGCTGAGCTGCGCCAGCTGAGGTGT | |
| | | | GAGCAGCTGCCGAAGTCAGTTCCTTGTGGAG | |
| | | | CCGGAGCTGGGCGCGGATTCGCCGAGGCACC | |
| | | | GAGGCACTCAGAGGAGTGAGAGAGCGCGGCA | |
| | | | GACAACAGGGGACCCCGGGCCGGCGGCCCAG | |
| | | | AGCCGAGCCAAGCGTGCCCGCGTGTGTCCCT | |
| | | | GCTTGTCCGGAGATGCGTGTCCCGGTGTAAAT | |
| | | | CATCAAGGCGATCAGCCACCTGGCAGCCGTT | |
| | | | ATATGGATCCGACTCGGTACCAAGCTGGCGT | |
| | | | AATCAGGGT | |
| 56 | PAX6 | paired box | GGAGAAAGGAGAGAAGAAAGGGCGGGGAGA | U63833 |
| | | gene 6 | GCGGGGTGGAGGATTTGGACAGGCCCTGGAG | |
| | | | GCTTGGGCTGGGGAGGCCTCTGGCCTCGTTTA | |
| | | | GTTCTCGGCCCGGCAACCTCCTCTCGGCCTAG | |
| | | | GCTTCGCCGCGGCCTCCGCAGCTGGAATGGA | |
| | | | GCTGCCAGGACCCAGTGACGCTCCCGCCCCTT | |
| | | | TCCTCTTCTTCCAAGGGGCCAGGTGGGCTGGG | |
| | | | GTGCGGCCGCCGCTGTGCTCTGTGTCTTGGGG | |
| | | | CCCCGGCTGGGATGGGGTgggggcgggcggg | |
| | | | ggcggggcggcAGGCCACGCTGTCCTGGAGTT | |
| | | | GGCAAGAAAGGACAGCACAGAAACTTGCACCC | |
| | | | TCCGAGGACTGGGAGTCCCGAGTCCAGCTTAG | |
| | | | GGGGAGTGGGGGCGCGACCCCCAACCCAGAA | |
| | | | ACCTTCACTTGACCGCTCAAGTTCGCGGCAGC | |
| | | | AGGGCGGGCCGCGCCGAATCTCGGCGTGCGCG | |
| | | | GAGCGGGGAGATGCAGGCGAGCGCCAGAGCCC | |
| | | | GGGCTCGGGGGCCCTGCGCCGGGGAGAGGAGC | |
| | | | CGGGACCCACCGGCGGAGCCGAAAACAAGTGT | |
| | | | ATTCATATTCAAACAAACGGACCAATTGCACC | |
| | | | AGGCGGGGAGAGGGAGCATCCAATCGGCTGG | |
| | | | CGCGAGGCCCCGGCGCTGCTTTGCATAAAGC | |
| | | | AATATTTTGTGTGAGAGCGAGCGGTGCATTTG | |
| | | | CATGTTGCGGAGTGATTAGTGGGTTTGAAAA | |
| | | | GGGAACCGTGGCTCGGCCTCATTTCCCGCTCT | |
| | | | GGTTCAGGCGCAGGAGGAAGTGTTTTGCTGG | |
| | | | AGGATGATGACAGAGGTCAGGCTTCGCTAAT | |
| | | | GGGCCAGTGAGGAGCGGTGGAGGCGAGGCCG | |
| | | | GGCGCCGGCACACACACATTAACACACTTGA | |
| | | | GCCATCACCAATCAGCATAGGTGTGCTGGCTG | |
| | | | CAGCCACTTCCCTCACCCACACTCTTTATCT | |
| | | | CTCACTCTCCAGCCGCTGACAGCCCATTTTA | |
| | | | TTGTCAATCTCTGTCTCCTTCCC | |
| 54 | P27KIP1 | cyclin- | CAAAGTTTATTAAGGGACTTGAGAGACTAGAG | AB003688 |
| | | dependent | TTTTTTGTTTTTTTTTTTTAATCTTGAGTTCC | |
| | | kinase | TTTCTTATTTTCATTGAGGGAGAGCTTGAGTTC | |
| | | inhibitor 1B | ATGATAAGTGCCGCGTCTACTCCTGGCTAATT | |
| | | (p27, Kip1) | TCTAAAAGAAAGACGTTCGCTTTGGCTTCTTC | |
| | | | CCTAGGCCCCCAGCCTCCCCAGGGATGGCAG | |
| | | | AAACTTCTGGGTTAAGGCTGAGCGAACCATT | |
| | | | GCCCACTGCCTCCACCAGCCCCCAGCAAAGG | |
| | | | CAcgccggcgggggggcgcccagcccccccT | |
| | | | AGCAAACGCCCGCGGCCTCCCCCGCAGACCAC | |
| | | | GAGGTGGGGGCCGCTGGGGAGGGCCGAGCTGG | |
| | | | GGGCAGCTCGCCACCCCGGCTCCTAGCGAGCTG | |
| | | | CCGGCGACCTTCGCGGTCCTCTGGTCCAGGTCC | |
| | | | CGGCTTCCCGGGCGAGGAGCGGGAGGGAGGTCG | |
| | | | GGGCTTAGGCGCCGCGGCGAACCCGCCAACGCA | |
| | | | GCGCCGGGCCCCGAACCTCAGGCCCCGCCCCA | |
| | | | GGTTCCCGGCCGTTTGGCTAGTTTGTTTGTCTT | |
| | | | AATTTTAATTTCTCCGAGGCCAGCCAGAGCAG | |
| | | | GTTTGTTGGCAGCAGTACCCCTCCAGCAGTCA | |
| | | | CGCGACCAGCCAATCTCCCGGCGGCGCTCGG | |
| | | | GGAGGCGGCGCGCTCGGGAACGAGGGGAGGT | |
| | | | GGCGGAACCGCGCCGGGGCCACCTTAAGGCC | |
| | | | GCGCTCGCCAGCCTCGGCGGGGCGGCTCCCG | |
| | | | CCGCCGCAACCAATGGATCTCCTCCTCTGTTT | |
| | | | AAATAGACTCGCCGTGTCAATCATTTTCTTCT | |
| | | | TCGTCAGCCTCCCTTCCACCGCCATATTGGGC | |
| | | | CACTAAAAAAAGGGGGCTCGTCTTTTCGGGG | |
| | | | TGTTTTTCTCCCCCTCCCCTGTCCCCGCTTGCT | |
| | | | CACGGCTCTGCGACTCCGACGCCGGCAAGGT | |
| | | | TTGGAGAGCGGCTGGGTTCGCGGGACCCGCG | |
| | | | GGCTTGCACCCGCCCAGACTCGGACGGGCTTT | |
| | | | GCCACCCTCTCC | |
| 55 | PAI-1/ | serpin | aggacaagctgccccaagtcctagcgggcagct | AF386492 |
| | SERPINE1 | peptidase | cgaagaagtgaaacttacacgttggtctcctgt | |
| | | inhibitor, | ttccttaccaagcttttaccatggtaacccctg | |
| | | clade E | gtcccgttcagccaccaccaccccacccagcac | |
| | | (nexin, | acctccaacctcagccagacaaggttgttgaca | |
| | | plasminogen | caagagagccctcaggggcacagagagagtctg | |
| | | activator | gacacgtggggagtcagccgtgtatcatcggag | |
| | | inhibitor type | gcggccgggcacatggcagggatgagggaaaga | |
| | | 1), member 1 | ccaagagtcctctgttgggcccaagtcctagac | |
| | | | agacaaaacctagacaatcacgtggctggctgc | |
| | | | atgccctgtggctgttgggctgggcccaggagg | |
| | | | agggaggggcgctctttcctggaggtggtccag | |
| | | | agcaccgggtggacagccctgggggaaaacttc | |
| | | | cacgttttgatggaggttatctttgataactcc | |
| | | | acagtgacctggttcgccaaaggaaaagcaggc | |
| | | | aacgtgagctgttttttttttctccaagctgaa | |
| | | | cactaggggtcctaggctttttgggtcacccgg | |
| | | | catggcagacagtcaacctggcaggacatccgg | |
| | | | gagagacagacacaggcagagggcagaaaggtc | |
| | | | aagggaggttctcaggccaaggctattggggtt | |
| | | | tgctcaattgttcctgaatgctcttacacacgt | |
| | | | acacacacagagcagcacacacacacacacaca | |
| | | | catgcctcagcaagtcccagagagggaggtgtc | |
| | | | gagggggacccgctggctgttcagacggactcc | |
| | | | cagagccagtgagtgggtggggctggaacatga | |
| | | | gttcatctatttcctgcccacatctggtataaa | |
| | | | aggaggcagtggcccacagaggagcacagctgt | |
| | | | gtttggctgcagggccaagagcgctgtcaagaa | |
| | | | gacccacacgcccccctccagcagctgaattcc | |
| | | | tgcagctcagcagccgccgccagagcaggacga | |
| | | | accgccaatcgcaaggcacctctgagaacttca | |
| | | | gg | |
| 57 | PDGF-B | platelet- | ACCCCTGGCTGTTGCATTCTCTTGGCTGATCC | X83706 |
| | | derived growth | CAGCGTGCCCCGGGGAGGCCGCTGACAGCTG | |
| | | factor beta | GATGTTTCCCCAGCCTCCCCTTACCATTTCCA | |
| | | polypeptide | GCTTCGTCCAGCACCTCCTCCTTCTTTCCCACA | |
| | | (simian | GCTCCACGGGCTCGTGTATCTGGGGTGGAGG | |
| | | sarcoma viral | CTGTGGCACAGAAACTGCCTTTCTCCTCACTT | |
| | | (v-sis) | TAGTCACAGCATTCTTGAACACATGGCCACAG | |
| | | oncogene | GCGCGATGTATGTGGCACTTTGCAGTTTATGA | |
| | | homolog) | AGCACTTTGCTGCTAAGCCTGAGTGAGCCTCA | |
| | | | GGCTGGCCCTGGGGGAGGGGACCTGCATGGG | |
| | | | GATGGAACCACGCAGGGGTCAGTCCAGGAAG | |
| | | | GAGCTGTAATGGCCAGTGctgggagagtcaggg | |
| | | | caggcctgctggtggaggtggccttggagctg | |
| | | | TCCACGTCCTGGTCGTGCTCGGACTAATCTTTC | |
| | | | AGCAGACGGCAGGCAGCCGTGAGGCAGGGCTGG | |
| | | | GTGGAGGGCCTGCCGAGGCCTCTGAGGTGCCAT | |
| | | | CTCCACCAGCTGAGCTGGCTTCCAGGAGGGCGA | |
| | | | GTCCCACTGTCACGTGACGCGTCTGGCCTCAGC | |
| | | | ACACTTCTTCCGGGAAAGAGTGAAGGGCCCCAC | |
| | | | TGCCCTTTGCCATCCAGCTTCCTCTGGCTTTGC | |
| | | | TAATGGCCCTAGGGGGCAGGAGACCAACTGCTG | |
| | | | GAATCCCAGAGCCCTGGAGGTGTGCAAGGGCAG | |
| | | | GTCAAACAGAATTTGGAGGATCTGGTGCAAGA | |
| | | | GCCAGGAAGAGAGAGAGAGAGAGAgtgtgtgtgtg | |
| | | | tgtgtgtgtgCGCATCTgagagagagagagagaga | |
| | | | gaCTGACTGAGCAGGAATGGTGAGATGTTTATCAT | |
| | | | GGGCCTCGTAAGTACCTCTCCACGTCTTGTCTTCC | |
| | | | CCTCCCCACATTGAGGAGCCTCTTCTGTGACAA | |
| | | | CTCTTCCTATGTTCTGGtttatttcattgtttatt | |
| | | | acctgctttctctactggagtgtcaaccccatta | |
| | | | gagagctttcctcct | |
| 58 | PgA | pepsinogen A | CCCCCCAATGTGCTGTGAATAAGCAGTGACC | NM_014224 |
| | (pepsi- | | ACAACCAGTACCACCTATGACTGAGTCGGGA | |
| | nogen A) | | GGCTGCTCTCTAAGAACCCCAGCTGCGTGACC | |
| | | | ACGGGGACAAATCAGGCCACCTGGGGCTCCT | |
| | | | TCACATCTGTCCATTGCTGTGTTAAAAGTACT | |
| | | | TTTAAACAACTTTGTCGAAATGCTCAGCTTGT | |
| | | | AAAGTTTTAATGTAGGCCCTTGTCAATGCTTC | |
| | | | AGAAATAAGCCTCTGGCGGCGCGACAGAGCA | |
| | | | AAACTCCctcaggaaagaaaggaaagaaatgg | |
| | | | agaaagggagaaagggagaaagagaggaaaag | |
| | | | aaagaaagaaagaaagaaagagagagagagag | |
| | | | agaaagagagaaagagaaagaaagaaaagaaa | |
| | | | gaaagaaaaagaaagaaagggaaagaaagaag | |
| | | | gaaaggaaggaaagaaaagaaaggaaagaaag | |
| | | | gaaaagaaaacaaataagcctccaggtcattg | |
| | | | cttagaaagaaaaagaaaaaagaaagaaagaa | |
| | | | aagaaagaaaaagaaaagaaaagaaaaTAG | |
| | | | CCTCCCGGTCATTGCTCCTCTCTCTCTCTG | |
| | | | CGGGTCCACCCCCATGGCACCCTCCCCCCTCC | |
| | | | CCATGGTGCAAGGTTACAATGGAAAGTGCCT | |
| | | | CAGCTGGAAAGGTCTCAGAATGTGGCTCAGG | |
| | | | GCAGCCACAATCTTATCAGGAGCTTCTCTGTT | |
| | | | TGGGATCAGGGGAACCGGTGACTTTCAGAGG | |
| | | | CCGATAAGGCGGGACCCAACTTGTATATAAG | |
| | | | GGGCAGCTCATGCTGCTGCTCTGCACCTTCCT | |
| | | | CCCATCTTGCCTTCTCCCTCGAGTTGGGACCC | |
| | | | GGGAAGAACCATGAAGTGGCTGGTGCTGCTG | |
| | | | GGTCTGGTGGCGCTCTCTGAGTGCATCATGTA | |
| | | | CAAGTGAGTCCGGGTGGTGTGGGTGTGAAGA | |
| | | | CGCTGCCTCCCACATCACCTTTCTTTCCTCCCG | |
| | | | TGTCTTCCTTCTTCCCTTTTTTTTCTCTCTCTCT | |
| | | | TCAGCTGTCTCCATCCCCC | |
| 59 | POMC | proopiomelano- | ACTAAAGCCAAGCCAGAACTCCAGGGCCAAG | NM_000939 |
| | | cortin | GGGGATGTTGAAAATTGTCTGAGTCCCCAGA | |
| | | | CCACCCTGCCAGCTCATGGCAAAGGGAGGGA | |
| | | | TCAGAGGCCACAGGGAAAGCACTTCAGCTGC | |
| | | | TCTTCACAGCATCACCCTCTCCCCATTTAATG | |
| | | | GTTTAGGTTAACAGGACTTTTTCCTTGAGGCT | |
| | | | TGGGACACGGAAGGGAGCCTCCCCTAAACCA | |
| | | | GGCCCTTGGAGAGCAGGCCCCAGGGGAGCAG | |
| | | | TGCAACTCACCTTCACACCCACAAGACGGCTC | |
| | | | CTGACTTCTGCTCCCTCCTCCCCTCCCCAAAG | |
| | | | TGGAACAGAGAGAATATGATTCCCCACGACT | |
| | | | TCCACATCACAGTTTCCAAACAATGGGGAAA | |
| | | | TCGGAGGCCTCCCCGTGTGCAGACGGTGATAT | |
| | | | TTACCGCCAAATGCGAACCAGGCAGATGCCA | |
| | | | GCCCCAGCACGCACGCAGGTAACTTCACCCTC | |
| | | | GCCTCAACGACCTCAGAGGCTGCCCGGCCTG | |
| | | | CCCCACACGGGGGTGCTAAGCGTCCCGCCCGT | |
| | | | TCTAAGCGGAGACCCAACGCCATCCATAATT | |
| | | | AAGTTCTTCCTGAGGGCGAGCGGCCAGGTGC | |
| | | | GCCTTCGGCAGGACAGTGCTAATTCCAGCCCC | |
| | | | TTTCCAGCGCGTCTCCCCGCGCTCGTCCCCCG | |
| | | | TCTGGAAGCCCCCCTCCCACGCCCCGCGGCCC | |
| | | | CCCTTCCCCTGGCCCGGGGAGCTGCTCCTTGT | |
| | | | GCTGCCGGGAAGGTCAAAGTCCCGCGCCCAC | |
| | | | CAGGAGAGCTCGGCAAGTATATAAGGACAGA | |
| | | | GGAGCGCGGGACCAAGCGGCGGCGAAGGAG | |
| | | | GGGAAGAAGAGCCGCGACCGAGAGAGGCCG | |
| | | | CCGAGCGTCCCCGCCCTCAGAGAGCAGCCTC | |
| | | | CCGAGACAGGTAAGGGCGCAGCGTGGGGGAC | |
| | | | CCGTGCTCTTTCCCCGGGATCCCCTGTCCCCG | |
| | | | TCCTCGCGATGCAGTCGGCCGGCTCCGGCTCC | |
| | | | GAAGGCGGACCTGGGCGCCTCTGGCTCT | |
| 60 | POU3F1 | POU domain, | GAGGCGTGAAGCCAGAGTCCGTCCGACTCCG | NM_002699 |
| | | class 3, | CCCGCACCGGACGCGCTCTCAGGGCAGAGGA | |
| | | transcription | GGTCGGCGGAGTTGTGACGCTGGGACTAGAG | |
| | | factor 1, | GAAGGAGAAGGAAAGCCGAGACGGGCCGGG | |
| | | octamer- | CAGACGCGCCGAGGAGCGCCCAGTGCACGCT | |
| | | binding | GGCAGCCGCGGGAGGCGAGGCGGGCGCGGTG | |
| | | transcription | AGCAGTCGCGCCGGAACCGAGCCGCGAATCC | |
| | | factor 6 | GCGCCGCCTCGCGCTCGCAGCCGCCAGGACC | |
| | | | CGCGGGAATCCTGGTCCGCCGGCAGCGGTAC | |
| | | | TGAGGAGGGAGGGGCGCGGGGCTGAGCCGCT | |
| | | | TCTCGGAGCCCGAGCGCCTCCCGGAGCCGGC | |
| | | | AATCCCTGCTGCCCGGGCGGGATGCGGGCGG | |
| | | | GAATTCAGCTCGCGTGGAATGTGGGACCGGC | |
| | | | CGGGCTCGGAGTCTCCAGCGCTGGGGGAAAG | |
| | | | CGGGGCCCACACAGCCAGGACGAGAGGGGGT | |
| | | | GCGGTCCCAGGGCCACCCCCGCGCCACTCCCC | |
| | | | ACGTggcggccgcgcccccggggcgTGAGTGT | |
| | | | GTACGCGGACGGTAGGGGGGCCGTGAATGAAG | |
| | | | CCCCAGCGGCCAATCAGCACGGCCGGCGCGCG | |
| | | | GGACCCCGGGAGCGACGCCCAATGGAGAGCTC | |
| | | | TGGGCggccgggcagggtggcgggcgggcgcg | |
| | | | cggggcgggggccgggcaggggaggcgggagg | |
| | | | cagctccgcgggcagccaatgggcggcgggcg | |
| | | | gggtggggctccggagcgccgagcgggtcgg | |
| | | | ggctttaagccggcggagcgaggcggcggggc | |
| | | | ccgcagacggagcggagcggcggcggcggcgc | |
| | | | ggcgcagggcgcggGGCGGCATGGCCACCACC | |
| | | | GCGCAGTACCTGCCGCGGGGCCCCGGTGGCG | |
| | | | GAGCCGGGGGCACCGGGCCGCTTATGCACCC | |
| | | | GGACGCCgcggcggcggcggcggcggcggcgg | |
| | | | ccgcggAGCGATTGCATGCAGGGGCCGCGTA | |
| | | | CCGCGAAGTGCAGAAGCTGATGCACCAC | |
| 61 | PR | progesterone | agatttaggcggaaatgtggaataactgctag | NM_000926 |
| | | receptor | tgggtattgagattttagagtcatactcatgt | |
| | | | tacaaaattaatagtgctgatggttgcacaac | |
| | | | tctgagtacatgaaaaatcaatgaactgatac | |
| | | | tttgagtgagctgtatgatactggaattacac | |
| | | | ctcaataaagcATGGTAACTGTTTTAAGATAG | |
| | | | GCTGGAAAGAGAAAGCCTGAAAACAACAATAA | |
| | | | TGATATTAATAAATTAGTttacttctctagtc | |
| | | | tcatatacttctgtgcccacacttgctcctgt | |
| | | | tctattcataatggtccccttgcagttgccat | |
| | | | attatatcctgccatttgatgcccggtgaaca | |
| | | | ttctatacctgcttcccagaattctctttacc | |
| | | | tttcctctatctgcctaacttccacATATCTA | |
| | | | AAattaatcagagtaaactatttactagaaca | |
| | | | accaactccaaatcctagtaacctaacatgat | |
| | | | aaaggtttgtttctcactcatatAGCCCCTCC | |
| | | | CCAGATGATCGAGGGGTCCAGGCTCCTTACCT | |
| | | | CTAGTGGCTCCCCCACCTTCTGGAGTCTTCTG | |
| | | | CATTCTTTATACATGGTTGAGATAAACTATG | |
| | | | AGTCATTAGCACAGCTAGACCTTGAGGTCC | |
| | | | TACAAGAAAATTTGCAAATCATTCACTCTGTT | |
| | | | TTGAACAAGGTATATTTAAGATGATGTTAAAA | |
| | | | TACCCAATGGTCTTGGGTCAAATACAGTTTAT | |
| | | | GACTGTGTATCTAAAATATATATTGCAATAT | |
| | | | TCTTCCCTTTTTCTACTGACTTCATGAATTTA | |
| | | | GCGGGGATCCATTTTATAAGCTCAAAGATA | |
| | | | ATTACTTTTCAGACTAAGAATATTTAGGGTAA | |
| | | | AAAGTACTGTTCAACATCTCTACTGAGGATG | |
| | | | TTATGATGTAGCACACTGTATAAGCTG | |
| | | | GAGCTAAAGGAAACTTTCCTTAAAGTGC | |
| | | | TATTTACTAAAAATTGGAACACATTCCTT | |
| | | | AAGACAAATCGAAGTGTGGCACACAAC | |
| 62 | Rb | retinoblastoma1 | agaaagaaaaagaaaaaaaaggctgtttctgg | NM_000321 |
| | | | ggattaaataagacaattatgtaaggtggccagc | |
| | | | acagttcctggtacatagtaaatgtcagGCCTG | |
| | | | CCTGACAGACTTCTATTCAGCAGCTACTGCTC | |
| | | | CCCTGAAAATCTTCCTCAGACGTTTCCACGGT | |
| | | | GCTTCCCGTTCTTACACCACTACAATCCTTTAT | |
| | | | TACACTACTATCCGTTCATTCCCCACAGCTCC | |
| | | | CTCCCTTCCTTTCCCTAACCAGTGATCCCAAA | |
| | | | AGGCCAGCAAGTGTCTAACATTTTCTATCTTC | |
| | | | TAAGTGACTGGTAAAGTTCCGCACCTATCAGC | |
| | | | GCTCCAAGTTTGTTTTTGTTTTGGCCGACTTTG | |
| | | | CAAAACGGATTGGGCGGGATGAGAGGTGGGG | |
| | | | GGCGCCGCCCAAGGAGGGAGAGTGGCGCTCC | |
| | | | CGCCGAGGGTGCACTAGCCAGATATTCCCTGC | |
| | | | GGGGCCCGAGAGTCTTCCCTATCAGACCCCG | |
| | | | GGATAGGGATGAGGCCCACAGTCACCCACCA | |
| | | | GACTCTTTGTATAGCCCCGTTAAGTGCACCCC | |
| | | | GGCCTGGAGGGGGTGGTTCTGGGTAGAAGCA | |
| | | | CGTCCGGGCCGCGCCGGATGCCTCCTGGAAG | |
| | | | GCGCCTGGACCCACGCCAGGTTTCCCAGTTTA | |
| | | | ATTCCTCATGACTTAGCGTCCCAGCCCGCGCA | |
| | | | CCGACCAGCGCCCCAGTTCCCCACAGACGCC | |
| | | | GGCGGGCCCGGGAGCCTCGCGGACGTGACGC | |
| | | | CGCGGGCGGAAGTGACGTTTTCCCGCGGTTG | |
| | | | GACGCGGCGCTCAGTTGCCGGGCGGGGGAGG | |
| | | | GCGCGTCCGGTTTTTCTCAGGGGACGTTGAAA | |
| | | | TTATTTTTGTAACGGGAGTCGGGAGAGGACG | |
| | | | GGGCGTGCCCCGACGTGCGCGCGCGTCGTCCT | |
| | | | CCCCGGCGCTCCTCCACAGCTCGCTGGCTCCC | |
| | | | GCCGCGGAAAGGCGTCATGCCGCCCAAAACC | |
| | | | CCCCGAAAAACGgccgccaccgccgccgctgccg | |
| | | | ccgcggaaccccc | |
| 63 | RBL1 | retinoblastoma- | AGGGCGATTGGGCCCTCTAGATGCATGCTCG | BC017557 |
| | (p107) | like 1 (p107) | AGCGGCCGCCAGTGTGATGGATATCTGCAGA | |
| | | | ATTCGCCCTTGTTCTCGGATCCCGATCATGCA | |
| | | | GAAAAGGTCCAAGGGAACAGCCTCTGGTTCT | |
| | | | TTTGTTACTTAGGCGTGGAAAGTTGGGGTTTT | |
| | | | CCTTTCAATTTAGTTCTAAGAAGTCACGTGAA | |
| | | | ACAGCCATAGGTTCCCTGCCTCCAGACCCTAT | |
| | | | TCTCCTGCCTCATTTACTGCAGTCTTCTCTGCC | |
| | | | TGCCTCTTTTAGCGACTAGCATGAGATGAGGA | |
| | | | TTCGTCTTCTAATATCCGTCACCAATCCTTCCC | |
| | | | CTCTGTCATTTAGCGAACCACTCACTGGGCAC | |
| | | | TAGGACTTTGGGGAGAGTCCCAAGAGGCCCC | |
| | | | TCTTCGTCCAGGGGCTACTTTTTTCTCTTCCAG | |
| | | | CCTCCATCTCCTAACTCAAGGGGTACAGCTCA | |
| | | | GATTATGTTTGGCGCCCAGGGACAGTGACAA | |
| | | | ACCCAGGGCCCGTGGATAGAGGAGGCATCTC | |
| | | | ACTACGCTGCACGAGGCCACCTCGCAGTAGG | |
| | | | CAGCCCAGCCCTGCCCCAAAACCCGAGAGCC | |
| | | | TAACCAGGAGGACAGGGGGAGGCCGCGGGCT | |
| | | | TCATCTCCCAAGAGATGGACTACACCTCCCAG | |
| | | | CAGGCTCTGCGCGCGGGCTGAGGATCCCTCC | |
| | | | GCTCTTTTTCTGTCCCGCCGGCTGGGCCCCCC | |
| | | | GCGACCAGCCAAGGGCCAAGGACAGGTCTTT | |
| | | | CAGAATCTGAGGTACATCTTCTTATCACATTT | |
| | | | CCGGGGAGGGACTGCTAGGAGCTCCGGAGGA | |
| | | | AAAACGGACTTTTTTTGAGGAGAAAAGCGGA | |
| | | | GGCAGACGGTGGATGACAACACGTCCCGCAG | |
| | | | CTGCAGATTTTCGCGCGCTTTGGCGCAGGTGG | |
| | | | GTTGTGGGTAGCGCGCCTGGGANGGANAA | |
| 64 | RIOK3 | RIO kinase 3 | AGGGCGATTGGGCCCTCTAGATGCATGCTCG | NT_086888 |
| | (sudD) | | AGCGGCCGCCAGTGTGATGGATATCTGCAGA | |
| | | | ATTCGCCCTTGTTTCGGATCCCGATCTCCTAC | |
| | | | CAGATCCATTCGGGAATGAAGGCAGAGACAA | |
| | | | GAACAGAGCAGAGAGGTGGCAGGACGGGCA | |
| | | | GCAGGCTCCGCCGAGGAGACAGGCGGGACAC | |
| | | | GGGCGACTGGCTGCTGATGCCGGAGTGGAGG | |
| | | | TGACAGATGGCGGCGACGGCGGCGGCCGCGT | |
| | | | CCGGAACTGGATCTCTCCTCTTCCGCCCTCTT | |
| | | | CGCTAGGACAGTCGCTTGCAATTGGCCGCAC | |
| | | | GCCCCTAGCTCCTCCTTAAGGCACCTTTCCCC | |
| | | | GCCCCCGGGCGGGCTACTTCCGGCTGCTGACC | |
| | | | GCCGGGCTCGGAGAAGCAAGCATCAGCTGGC | |
| | | | TGTCGCTTGGGGTCACGTTGCCTGTGTCGGGC | |
| | | | AGGGCAGGGCAAGAACTGGGTGTGGCTTCCT | |
| | | | TTGGCCCAGGCTCTGCCCTGTCCCCGCACTGC | |
| | | | CATCTCCTTCTTTCCTCCTTGGCACCCCAAAA | |
| | | | ATTGCCGCTGGATCTAAACTAGATTAGACTAG | |
| | | | TGGATTGTAAATAAATAAACAAACTAGGCTC | |
| | | | TCTCTGTTCATTCATTATTTCCTGGAGCAGTTC | |
| | | | TAAACTGGGATGACTTGGGAGACAGAAAACG | |
| | | | GCAGGTTTATAGAGGGAAAGGGCCTGGAAAG | |
| | | | GACGGTCGGAGTTTGTGGTTGTTGTTGTTGAA | |
| | | | GGGCGGGGCGTGGAATGCGGAAAATGTGTAA | |
| | | | AATGTGTTACGTAACAGTGAACAAAAATAAN | |
| | | | ACTGCATAATAAAACTTTTGTTTCTGTATTTTG | |
| | | | TAGANATTCTAATAAAATGACCANATNAAAG | |
| | | | ATAACTAAACATTGCATTTCACTTANNATATC | |
| | | | ANTGCACTGTTCAAATCTTTCATTAACTTTTTA | |
| | | | NTCCTCAAATTACCGTGANANCTAAATTCTGT | |
| | | | CNTTATCTCTATTTTACTGATATTG | |
| 65 | RPA2 | replication | cagtagctgggaccccaggcacttgccaccacacc | NM_002946 |
| | | protein A2, | cagactaatttttaaaaatattttttgagagaggg | |
| | | 32 kDa | ctcactatgttgtccaggctggtctcaaacttcca | |
| | | | gcctcaagcggtcctcctgcctcagaccccatttg | |
| | | | ctgggtttacaggcatgagccacagcacctgctaa | |
| | | | tttttcttaaatacataaatGAACATAAA | |
| | | | ATTCTAACAATGCATGAGTATTTTGAGGAAGG | |
| | | | AACTGACAAAATGTTCCACTCCCTATGGGAGGCAA | |
| | | | CGTTATATGAAGAATtatgaaaaatggtcgaaatg | |
| | | | actggagaggccaagcctggatgagactgggatgg | |
| | | | ggacaggtgcgggacgaggggcaccaccctcacat | |
| | | | ctttcacaagtctgtcataggcaagagggcgtagg | |
| | | | tttctcacagccccactggggagaatcggcacca | |
| | | | tggtggcattacacgaagagaatgtgacctccta | |
| | | | tgtaaaagaacAAGCAACTCCACGCGGTGCTGT | |
| | | | GAGGCTAGTGCTGCGAGTCCCTGAGGTGCG | |
| | | | CAATTCCCGCACGACCGTGGGTGGGAA | |
| | | | ACACCGAAGCCAAAACTCCGCTACAG | |
| | | | CCCTTTAGATGAAGGCGTCGTCTGATTGGTGA | |
| | | | TAGTTTGGCGCGAACCTGAGCACGCCGAACA | |
| | | | AAGGAAGTGACGGCAGAAGTCGCGCACTTGA | |
| | | | CGAGGGTGGGATCACACGGCGCTGCGTCGCG | |
| | | | GTAGTATTGTTCTGATTGGTTGATTTCTTGCG | |
| | | | ATACCGCTCTGCCAGCCCCTTGCTTCCGCTAG | |
| | | | TGCGGAGGGTTTTGCCCTTCGTAAAGATGGCC | |
| | | | GCGGAGGCTTTTGGAGCCAACTGGGAGCGCA | |
| | | | GTACGCGTTTTCTGGAGCATGGGCAGAGGAG | |
| | | | ACAGGAACAAGCGTAGCATCCGTGAGCACCG | |
| | | | ATTGGCTGAAGCGAGCACCCCGGGAGCTGAC | |
| | | | TGGCTCCGCCATTCGCGGGAAGGCGTTTGTGG | |
| | | | TGCCAGAGAAAAGTAGCCAGAGCGGCGC | |
| 66 | SFN | stratifin | CTCTGAAAGCTGCCACCTGCGCATTCTGGGAG | NM_006142 |
| | | | CTCAGAGGGGACCCTGAGGGGGAATGAGGCC | |
| | | | TGGAGGATGGAACCATCTTCAGGTAGACTGA | |
| | | | GAAGGAGCCTGGATCTCACTTCCAAACACAG | |
| | | | TCTGGAGCTCATAGGTCAGAGGCCTCAATGG | |
| | | | GAGAAAAGCTAAAGGAAGAGGGTGCAGAAA | |
| | | | GGAgtttcagggaattggtggctatgtgactt | |
| | | | tgagcaaatctcacccctctctgagacttagt | |
| | | | gttcccatctctatggtcctgtgtgtgtcaca | |
| | | | gagacatggtggggattaaattcgatcgtgaa | |
| | | | tatgaaagtgcttgggaaactccatggcc | |
| | | | CTACCTAAACATGAGTTATCCTCACCTGAACC | |
| | | | AAGGGGGGAAGTTACCTGGCAGGATTAGGAA | |
| | | | CCCCATCCTCCTGAACCTTTATGGGCTCTGTC | |
| | | | GAGGCTGAAGCAGCCAGGGGCTAAAGCCGTC | |
| | | | CTTAGCCCCTGGAAGGGCACTGTGAAAGTGG | |
| | | | ATCTGATTTGAGAAGCCGTTTCCTGATGTGGG | |
| | | | CAGCCATGTGATGCCAGCCCCGAACAAGAGG | |
| | | | GGGCAGCCTGGAGCCTGGAAAGGTGCCAGTG | |
| | | | CAGGTGGGGCCCACGCCCAGATTTCTCCTGCT | |
| | | | GACTGTTCTGATGATTCACCCCCACATCCCAG | |
| | | | CCTTTTTACCTTTACTGCAGAGCCGGAAAGGG | |
| | | | TGTGGGGAAGAGAGGAGAGGGAGGCAGGTCT | |
| | | | TGGGCCCTGGTCCCGCCCCCTGCTCCTCCCCA | |
| | | | CCCTTCTCTGGGCCTGGCCACCCAGCCAAAAG | |
| | | | GCAGGCCAAGAGCAGGAGAGACACAGAGTCC | |
| | | | GGCATTGGTCCCAGGCAGCAGTTAGCCCGCC | |
| | | | GCCCGCCTGTGTGTCCCCAGAGCCATGGAGA | |
| | | | GAGCCAGTCTGATCCAGAAGGCCAAGCTGGC | |
| | | | AGAGCAGGCCGAACGCTATGAGGACATGGCA | |
| | | | GCCTTCATGAAAGGCGCCGTGGAGAAGGGCG | |
| | | | AGGAGCTCTCCTGCGAAGAGCGAAACCTG | |
| 67 | SIM2 | single-minded | CGCGCCGTGTGCACTCACCGCGACTTCCCCGA | NM_005069 |
| | | homolog 2 | ACCCGGGAGCGCGCGGGTCTCTCCCGGGAGA | |
| | | | GTCCCTGGAGGCAGCGACGCGGAGGCGCGCC | |
| | | | TGTGACTCCAGGGCCGCGGCGGGGTCGGAGG | |
| | | | CAAGATTCGccgcccccgcccccgccgcggtc | |
| | | | cctcccccctcccgctcccccctccgGGAC | |
| | | | CCAGGCGGCCAGTGCTCCGCCCGAAGGCG | |
| | | | GGTCTGCCATAAACAAACGCGGCTCGGCCG | |
| | | | CACGTGGACAGCGGAGGTGCTGCGCCT | |
| | | | AGCCACACATCGCGGGCTCCGGCGCTGC | |
| | | | GTCTCCAGGCACAGGGAGCCGCCAGGAA | |
| | | | GGGCAGGAGAGCGCGCCCGGGCCAGGGCCCG | |
| | | | GCCCCAGCCGCCTGCGACTCGCTCCCCTCCGC | |
| | | | TGGGCTCCCGCTCCATGGCTCCGCGGCCACCG | |
| | | | CCGCCCCTGTCGCCCTCCGGTCCGGAGGGGCC | |
| | | | TTGCCGCAGCCGGTTCGAGCACTCGACGAAG | |
| | | | GAGTAAGCAGCGCCTCCGCCTCCGCGCCGGC | |
| | | | CGCCCCCACCCCCCAGGAAGGCCGAGGCAGG | |
| | | | AGAGGCAGGAGGGAGGAAACAGGAGCGAGC | |
| | | | AGGAACGGGGCTCCGGTTGCTGCAGGACGGT | |
| | | | CCAGCCCGGAGGAGGCTGCGCTCCGGGCAgcg | |
| | | | gcgggcggcgccgccgggTTGCTCGGAGCTCA | |
| | | | GGCCCGGCGGCTGCGGGGAGGCGTCTCGGAA | |
| | | | CCCCGGGAGGCCCCCCGCACCTGCCCGCGGCC | |
| | | | CACTCCGCGGACTCACCTGGCTCCCGGCTCCC | |
| | | | CCTTCCCCATCCCCGCCGCCGCAGCCCGAGCG | |
| | | | GGGCTCCGCGGGCCTGGAGCACGGCCGGGTCT | |
| | | | AATATGCCCGGAGCCGAGGCGCGATGAAGGAG | |
| | | | AAGTCCAAGAATGCGGCCAAGACCAGGAGGGA | |
| | | | GAAGGAAAATGGCGAGTTTTACGAGCTTGCC | |
| | | | AAGCTGCTCCCGCTGCCGTCGGCCATCACTTC | |
| | | | GCAGCTGGACAAAGCGTCCATCATCCGCCTC | |
| | | | ACCACGAGC | |
| 68 | SRBC | protein kinase | ATCAAAGCAAAGACCAGTGCCTAGTCTAACG | AF408198 |
| | | C, delta | CTTTTAAGGATTTTAAAAGAGGTGAAGGTGTC | |
| | | binding | CTGCTTATCCTCCAAGCTTGGGTGCTGGGGCC | |
| | | protein | GGGGCGGCTGAGATTTACCAGTGAAACCCAA | |
| | | | AGAAAGAGAGGGCAGAAAACTAGAGAAAAG | |
| | | | AAACCAGATAATGCTACCCAAGAGGACGAAA | |
| | | | TAAAGAAGCAGGAAACGAAGCCTGAGGCTAA | |
| | | | ACCCTGGAGATGACTATTAGGAAAACACCAG | |
| | | | AGGATGCCCCGCCCGCCAGCCCACAATGAGC | |
| | | | AGCCTGTCCAAGTCACAAAGCGGGGCCTCGG | |
| | | | GCCTTGACAGTTCGCGATCTGTAAGCAGAATG | |
| | | | TTCCAGGGCCTCCCTGTCGCCTGCATCCAGCC | |
| | | | TGGGGGCAATCTTCACTGGTGTGGGAGGCCG | |
| | | | AAAGTGGACGGCGACGGAGGCCCCTCTGGTT | |
| | | | ATCTCTTTGCCGTGCCAACACAGTCTCTGCGC | |
| | | | CCACTAAGATGCATGAAATAAAAATTTCCGT | |
| | | | GACTCGCCCTTTGCAGTGGAGAACTGAAACA | |
| | | | GGCACACCAGGGAATTGGAGCGGAGGAGGGT | |
| | | | AACTCAAACTCAGAGTGAGAGGGTTTGCAGG | |
| | | | GGGCCGATTTGGGGCCAACAGGCTTCCCAGC | |
| | | | AGGCCCCCGGCGCGGGACAGCGGAAGGCGAA | |
| | | | ACGCTTTCAAGAGACCCCGCTGCCAACATCCC | |
| | | | CACGCCCTCGCGCCCTCCCGCCGCCCCAGAAG | |
| | | | GCCAACTCCGCCTGCCTGAGTCACAGCTGGA | |
| | | | GCTGGGGAGGAGCCAGGGAAAGGAGGCCCCT | |
| | | | GACCGTAGTGCGGCCAGCAGTTGCAGGCAGA | |
| | | | CGGAGCAGAGCGGTCAGGGATCATGAGGGAG | |
| | | | AGTGCGTTGGAGCGGGGGCCTGTGCCCGAGG | |
| | | | CGCCGGCGGGGGGTCCCGTGCACGCCGTGAC | |
| | | | GGTGGTGACCCTGCTGGAGAAGCTGGCCTCC | |
| | | | ATGCTGGAGACTCTGCGGGAGCGGCAGGGAG | |
| | | | GCCTGGCTCGAAGGCAGGGAGGCCTGGCAGG | |
| | | | GT | |
| 69 | STAT1 | signal | GGGCGATTGGGCCCTCTAGATGCATGCTCGA | AY865620 |
| | | transducer and | GCGGCCGCCAGTGTGATGGATATCTGCAGAA | |
| | | activator of | TTCGCCCTTGTTCTCGGATCCCGATCGGTTCT | |
| | | transcription 1 | GAACATAGTTTGTAGAGCTCACTGCACATACA | |
| | | | AGTGGAGAGGCAAGTGGGAQTTGTAGGTGTG | |
| | | | AAGCCCAGAGGAGAGGTGTGGACGGGATAAG | |
| | | | CATTTAAGACTCCTCCATCTAGAAGGAAACTG | |
| | | | AAGCTGTGGGTAAGGTCATCACAGCACAGCG | |
| | | | TTTAGGAGAAGCCCAGGTAAAGAAGCTGACG | |
| | | | AATGTCTGGACCCTGACAACCTTAACATATAA | |
| | | | TGGTTTGATAGTGGAGGTGGAGGCAATGTAG | |
| | | | AAAGAATGCCAGAGGCAGGAAAAAGCAAGG | |
| | | | AGGATGTGTTATCATCATGACCAAGGAAGAA | |
| | | | ACGTGTTTCAAGAACAAAGGCGTCAACTCTG | |
| | | | CCCCATGCTTCCGAGCTGTCAAGTAAAGTGAG | |
| | | | AAAAACAGAAAAGCGTTCCCTGGGTTTAGCA | |
| | | | ACACGGAGGTCAGTTGCTAAAGGGAGCTTCT | |
| | | | AGAATGACGACGTCGCCAAATCTGTCCTCTGC | |
| | | | CTGGATTCTCGGCGATGAAACTACTACAGAG | |
| | | | ACCTCCAAGTTTGGGCTTCTGCAAACACAGCA | |
| | | | CGTCCTTCTGATCGTTCTCTAAGATATGTAAA | |
| | | | CAGAACGCCAGTTCCCAGCGTGGCAACACGG | |
| | | | GNACTGGGCTGCAGCTCACCCAGCCGGCGGC | |
| | | | CCCCGCCGGAAGCCGGCGGAAATACCCCAGT | |
| | | | GCGTGGGCGGAGCAGCGGCCCGCAGAGGGAG | |
| | | | GCGGTGGCGCCNCACGGAACAGCCCNCGTCT | |
| | | | AATTGGCTGAGCGCGGAGGC | |
| 70 | STAT5a | signal | AGGGCGATTGGGCCCTCTAGATGCATGCTCG | AJ412877 |
| | | transducer and | AGCGGCCGCCAGTGTGATGGATATCTGCAGA | |
| | | activator of | ATTCGCCCTTGTTCTCGGATCCCGATCCCTGC | |
| | | transcription | CTGAAGGGAACTGCTGGAGGGCACAGGTGCC | |
| | | 5A | AAGTGGGACCCACCCAAATGTGGCAATGGGT | |
| | | | TTGTATCCAGCCACCGACAGGCTGCATGACG | |
| | | | GTGGCAAAGTCACTTCCCCTCTCTGGCCTTTG | |
| | | | TTTTTCCACTTGTAAAATCATCTTTATGGTCAC | |
| | | | TTCCAGCTGTGGCACTTGGCTTTCATTCCAGT | |
| | | | TGACCCCCTAGCTCTGTGTCTGACCCTCCCCT | |
| | | | GCCAAATCCATTGCCCAGAGTGGGAAAGGAG | |
| | | | AGGAGAGGGACTATACTTCCTCCTCCCTGGGG | |
| | | | CCCCCTGCAGAGCATCTGGGAAGCAAGGCTT | |
| | | | CCCTACATCCTCCATGCACCCCCTTAGAGTTT | |
| | | | TCAATTCCTTTCCTCGTGATCCTGCCAACTAA | |
| | | | GACACTGTGACCACACAGAGAAGGTGGGGAG | |
| | | | AACGCAGACATTTTGGCTTCTGCAGCTTTGAA | |
| | | | GTTCTTTTTTTTTCCTCTGAAGTTAAAAGAATG | |
| | | | AAACTGGGAGAGGTAGTAAGGGGCAAGAAA | |
| | | | GGAGAGTGGAAATGGAGAGAAAAGGGCAGC | |
| | | | TCTGAGAAGCGGCTGGGGAGGGAGGCAGATG | |
| | | | AGAATGCACCCCCCCCAACAGAACATGCAGT | |
| | | | CTTGGCCCAGCTGTGCTGTGAGTGGGCAGCTG | |
| | | | GGCTGGCCCCTCCTCTGGTGCTGCCAACCCGC | |
| | | | TGCCAGGCAGAGGGGAGGNCCANAGGAGAG | |
| | | | GGAAGCTGGGCAAAGGGGATGGAAGGCGTCC | |
| | | | AGCCCNACCTTACCAAACCCCTTGGGCCTCGT | |
| | | | GGGAAGGGGCCTCTTGGAGAGGGGACTGAGG | |
| | | | CTCTAGACAGGATATTCACTGCTGCGGCAAG | |
| | | | GCCTGTANAGAGTTTCGAAGTTANGA | |
| 71 | survivin | Homo sapiens | TGCGAAGGGAAAGGAGGAGTTTGCCCTGAGC | NM_001168 |
| | | baculoviral | ACAGGCCCCCACCCTCCACTGGGCTTTCCCCA | |
| | | IAP repeat- | GCTCCCTTGTCTTCTTATCACGGTAGTGGCCC | |
| | | containing 5 | AGTCCCTGGCCCCTGACTCCAGAAGGTGGCCC | |
| | | (survivin) | TCCTGGAAACCCAGGTCGTGCAGTCAACGAT | |
| | | (BIRC5)/ | GTACTCGCCGGGACAGCGATGTCTGCTGCACT | |
| | | Homo sapiens | CCATCCCTCCCCTGTTCATTTGTCCTTCATGCC | |
| | | apoptosis | CGTCTGGAGTAGATGCTTTTTGCAGAGGTGGC | |
| | | inhibitor | ACCCTGTAAAGCTCTCCTGTCTGACtttttttt | |
| | | survivin gene | tttttttagactgagttttgctcttgttgccta | |
| | | | ggctggagtgcaatggcacaatctcagctcact | |
| | | | gcaccctctgcctcccgggttcaagcgattctc | |
| | | | ctgcctcagcctcccgagtagttgggattacag | |
| | | | gcatgcaccaccacgcccagctaatttttgtatt | |
| | | | tttagtagagacaaggtttcaccgtgatggccag | |
| | | | gctggtcttgaactccaggactcaagtgatgct | |
| | | | cctgcctaggcctctcaaagtgttgggattacag | |
| | | | gcgtgagccactgcacccggccTGCACGCGTTCT | |
| | | | TTGAAAGCAGTCGAGGGGGCGCTAGGTGTGGGCA | |
| | | | GGGACGAGCTGGCGCGGCGTCGCTGGGTGCACCG | |
| | | | CGACCACGGGCAGAGCCACGCGGCGGGAGGAC | |
| | | | TACAACTCCCGGCACACCCCGCGCCGCCCCGC | |
| | | | CTCTACTCCCAGAAGGCCGCGGGGGGTGGAC | |
| | | | CGCCTAAGAGGGCGTGCGCTCCCGACATGCC | |
| | | | CCGCGGCGCGCCATTAACCGCCAGATTTGAAT | |
| | | | CGCGGGACCCGTTGGCAGAGGTGGCGGCGGC | |
| | | | GGCATGGGTGCCCCGACGTTGCCCCCTGCCTG | |
| | | | GCAGCCCTTTCTCAAGGACCACCGCATCTCTA | |
| | | | CATTCAAGAACTGGCCCTTCTTGGAGGGCTGC | |
| | | | GCCTGCACCCCGGAGCGGGTGAGACTGCCCG | |
| | | | GCCTCCTGGGGTCCCCCACGCCCGCCT | |
| 72 | SYBL1 | synaptobrevin- | aaaagtatctatttgttttagcaacaCTGTTGA | AJ004799 |
| | | like 1 | GAATTCTGTCTGTAAAGGAGAGGTGAGAGAAAG | |
| | | | ACCACTAGCTTATCTGTGTTTGGTCTGTGTTTG | |
| | | | ATGAGGGGGCTTggggtatggggttaagaaagg | |
| | | | tgactttggaatgttttagatgagagaaatttt | |
| | | | gacagcctttaagtcctgatagtaaagagcga | |
| | | | gttagcagagagccgttgaggagtcatgcaacg | |
| | | | gaagggttcatcagaggagcttgactctgagt | |
| | | | cggcaacagggaatagagatggaagagggctgg | |
| | | | cttagatcaaaggagagtagtcgtttattatta | |
| | | | ttattattgcaaaaagaataggagaaaggattg | |
| | | | gtgaggggtacaagaaaattagaaaatttcatg | |
| | | | gcgaaagtagaggcagttcctgtcagatgaatt | |
| | | | ctattttgtctgtgaggaaacgggcgacgctgc | |
| | | | ctactgagactaagcaggagagacggGGCAAGC | |
| | | | TTGGCTCTTCATTTATGCCGCCTACTCATTGCT | |
| | | | GGTAGATTCTTTATCTAGCCTGCATCCTCTCAT | |
| | | | TTTCCTGGATCCCTATACGGCATTTGACGCTGT | |
| | | | TTACCACAAGAGCTGTCGAACGAACGTGAAACA | |
| | | | CTCAGTGATACTCCAACCGGAACTACTACTCCC | |
| | | | AGAATGCAGTACGGCTCCTGGGAAGTGCGGGGG | |
| | | | GCTGGGAACGCAGCAGGCCTAGCCGTGTCGCCT | |
| | | | GCTGCCATTGGAGGAGCGCTCCCACTCCCAAGA | |
| | | | GGCCACGCGTAGACGGGGCGCTTCATGCGGAA | |
| | | | GTCAGCGGCGTCCGGTCCCAGCCTCCTCTGGG | |
| | | | AGCGGGCAGTTGGCGACCCTGCACTGACCCG | |
| | | | CGTCCCTCCGTCCCGAGCCCGCGCGCCCTCAG | |
| | | | AGGGTGCCCGGACAGGTAAATGGAGTGGGGT | |
| | | | GCGCCTGCGGGAGGCGGGGAGAGAACTGCGG | |
| | | | AGGGAGGGCGGAGGTGTCGATGGAAAGGTGC | |
| | | | TGGGGTGGAGCGAGGAGGCAGTG | |
| 73 | Tastin | trophinin | CGGAGACAACGTACAGATGttctctctttccct | NM_005480 |
| | | associated | ctttattttttttaagacagggtctctgttgcc | |
| | | protein | caggctggagtgcagtggcgcgaccacagctca | |
| | | (tastin) | ctacagcctcaacctcctgggctcaacacgatc | |
| | | | ctcctgcctcagcctccagagcggctgggacta | |
| | | | caagcgcgcaccactgcacagggattattatta | |
| | | | ttattttattattttgtagagaaacgggtggga | |
| | | | gtggtctcgctatgttgcccaggctggtctc | |
| | | | aaactcagctcaagagatcctcccgcctcggcg | |
| | | | tcccaaagtgttgggattacaggcgcctgccac | |
| | | | cgcgcccggACGCAGATATTTTCTATGGG | |
| | | | CATCTGGAATGGCGTCCCCAAAGCTT | |
| | | | GGCGCCGTGCTATGGTCAAGCCGGGTCG | |
| | | | GGGGCTCGGGCCAGCCTTCAACACCGTTGGC | |
| | | | AGCAATCGGAACGATCAACTGTACCCTCAGT | |
| | | | ACCGCGACCTCGCCCGGTCCTGCCAATGGCCG | |
| | | | GCCCCTAGCCGGTCCTGAGGCCTCGCGAGAG | |
| | | | CTCCCGTGGCTACGCCTTCCCCGGCCTCGGAA | |
| | | | CGGCCCCATCCTTCCTCTTTCCCCGCCTCCCA | |
| | | | GCGGCGCTCCACTCTCGGATTGGCTGATTGAT | |
| | | | CCGAGTCAGTTTTTTTCCTCGCCAGAAAGCGG | |
| | | | TTCGACAATTGGTCCTTCTTTTGGCCCCTCCTG | |
| | | | CGATGCCCGCGGATTGGACGGCTGAGTCTGG | |
| | | | CTACGCGGGCCTCCGCGGGAGCGCGACCGGG | |
| | | | CCAATCAAGAGCTTGGCGTATTTTACAAACTG | |
| | | | AGAAAGTAGCTCCAGCAGCACCCGAGAGGGT | |
| | | | CAGGAGAAAAGCGGAGGAAGCTGGGTAGGC | |
| | | | CCTGAGGGGCCTCGGTAAGGTAAGGCACGGG | |
| | | | GGTCTTGAAGGGAACGAAGGCTGCTGGGTTC | |
| | | | ATAGGGAGGAGGGCAGTTTGGGGCCCGAGGG | |
| | | | CGAAAGAGTAGGCTCGGGGTGTCTGGAGATA | |
| | | | GCACCCATAAGAGCGGTCTTGCAG | |
| 74 | TFF1 | trefoil factor | CCCCCAGCCCCTCCCagaaggagacttaatc | NM_003225 |
| | | 1 (breast can- | tgtcgctcaggctggagtgcagtagggtgat | |
| | | cer, estrogen- | ctcgactcactgcaacctccgcctcccaggt | |
| | | inducible | tcaagtgattctcctgacttaacctccagagt | |
| | | sequence | agctaggattacaggcacccgccaccatgcct | |
| | | expressed in | ggctaatttttgtattttttttttttgtagag | |
| | | | acggggtttcgccatgttggccaggctagtc | |
| | | | tcaaactcctgactttaagtgatccgcctgct | |
| | | | ttggcctcccaaagtgttgggattacaggcgt | |
| | | | gagccactgcgccaggccTACAATTTCA | |
| | | | TTATTAAAACAATTCCACTGTAAAAGAATT | |
| | | | AGCTTAGGCCTAGACGGAATGGGCTTCAT | |
| | | | GAGCTCCTTCCCTTCCCCCTGCAAGGTC | |
| | | | ACGGTGGCCACCCCGTGAGCCACTGTTGTCAC | |
| | | | GGCCAAGCCTTTTTCCGGCCATCTCTCACTAT | |
| | | | GAATCACTTCTGCAGTGAGTACAGTATTTACC | |
| | | | CTGGCGGGAGGGCCTCTCAGATATGAGTAGG | |
| | | | ACCTGGATTAAGGTCAGGTTGGAGGAGACTC | |
| | | | CCATGGGAAAGAGGGACTTTCTGAATCTCAG | |
| | | | ATCCCTCAGCCAAGATGACCTCACCACATGTC | |
| | | | GTCTCTGTCTATCAGCAAATCCTTCCATGTAG | |
| | | | CTTGACCATGTCTAGGAAACACCTTTGATAAA | |
| | | | AATCAGTGGAGATTATTGTCTCAGAGGATCCC | |
| | | | CGGGCCTCCTTAGGCAAATGTTATCTAACGCT | |
| | | | CTTTAAGCAAACAGAGCCTGCCCTATAAAATC | |
| | | | CGGGGCTCGGGCGGCCTCTCATCCCTGACTCG | |
| | | | GGGTCGCCTTTGGAGCAGAGAGGAGGCAATG | |
| | | | GCCACCATGGAGAACAAGGTGATCTGCGCCC | |
| | | | TGGTCCTGGTGTCCATGCTGGCCCTCGGCACC | |
| | | | CTGGCCGAGGCCCAGACAGGTAAGGCGTGCT | |
| | | | TCTTCCTGCTCTGTGGGGCCACAGCCAGCTCT | |
| | | | GGCAGCCTCCGCCAGGAGCCACTGTTTTACa | |
| 75 | THBS1 | thrombospondin | AATTCGAGTAGAAAGCAGCTGTCCTCCCCGG | NM_003246 |
| | | 1 | GCCCCTTGATGAGAATACGCACACCGCCCCC | |
| | | | AAGCGGCCGGCCGAGGGAGCGCCGCGGCAGC | |
| | | | GGGAGAGGCGTCTCTGTGGGCCCCCTGGCAG | |
| | | | CCGCGGCAGGAAAGGGCCCGAAGGCAGCGA | |
| | | | AGGCGAACGCGGCGCACCAACCTGCCGGCCC | |
| | | | CGCCGACGCCGCGCTCACCTCCCTCCGGGGCG | |
| | | | GGCGTGGGGCCAGCTCAGGACAGGCGCTCGG | |
| | | | GGGACGCGTGTCCTCACCCCACGGGGACGGT | |
| | | | GGAGGAGAGTCAGCGAGGGCCCGAGGGGCA | |
| | | | GGTACTTTAACGAATGGCTCTCTTGGTGTCCC | |
| | | | CTGCGCCCCGTCGGCCCATTTTTCTTTTTACAA | |
| | | | AACGGGCCCAGTCTCTAGTATCCACCTCTCGC | |
| | | | CATCAACCAGGCATTCCGGGAGATCAGCTCG | |
| | | | CCCGAAAGCCCCTGCGCCACCCCGCGGGCCC | |
| | | | TCCTAGGTGGTCTCCCCAGCCCCGTCCCTTTT | |
| | | | CGGGATGCTTGCTGATCACCCCGAGCCCGCGT | |
| | | | GGCGCAAGAGTACGAGCGCCGAGCCCGTGCG | |
| | | | CGCCAAGGCTGCGTGGGCGGGCACCGACTTT | |
| | | | TCTGAGAAGTTCTAGTGCTCCCAAGCCCCGAC | |
| | | | CCCCGCCCCCTTCACTTTCTAGCTGGAAAGTT | |
| | | | GCGCGCCAGGCAGCGGGGGGCGGAGAGAGG | |
| | | | AGCCCAGACTGGCCCCCACCTCCCGCTTCCTG | |
| | | | CCCGGCCGCCGCCCATTGGCCGGAGGAATCC | |
| | | | CCAGGAATGCGAGCGCCCCTTTAAAAGCGCG | |
| | | | CGGCTCCTCCGCCTTGCCAGCCGCTGCGCCCG | |
| | | | AGCTGGCCTGCGAGTTCAGGGCTCCTGTCGCT | |
| | | | CTCCAGGAGCAACCTCTACTCCGGACGCACA | |
| | | | GGCATTCCCCGCGCCCCTCCAGCCCTCGCCGC | |
| | | | CCTCGCCACCGCTCCCGGCCGCCGCGCTCCGG | |
| | | | TACACACAGGTAAGTCGCCCCCGGCGGCCGC | |
| | | | CGAGGACCAAAGCTGCCCGGGACATCCA | |
| 76 | THBS2 | thrombospondin | CACCTTAGAGCAGCAGCTTCCCCTTTCCACTG | NM_003247 |
| | | 2 | TATACCCTGACCTGGGAGAAGCAGCCCCTCC | |
| | | | GCATCCATCGTCCACCCTGACCTCTGAGAAGC | |
| | | | GGTGCCCCCCACCCCCATGCAGAGTGCACCCT | |
| | | | GATTGCGGGTGATGCCTGAGGTGTGGGAGGG | |
| | | | GCGGGGGTTAGCTGCTGCCACTGCTTCTCGTT | |
| | | | CTCTCGAGTCCTTGCTCTGTGCCTGCACGTCA | |
| | | | GGTTGTTCCTGTGATGGGGCCACGTGCAAGTG | |
| | | | TGCACCAAGGGGACTTGGCCGGGTACTGTAC | |
| | | | GTCCACTGGGACACACCCTTCTACGGGTATTG | |
| | | | CACGTCCACTGGGAGACGTCCTTCTAGGGGAT | |
| | | | CCTCACTGAGCAAATGAAGCAGAATTTGGGT | |
| | | | AAAAATGAATTTTCCCAAAGCTGCAGTACAG | |
| | | | CTTTTCAGTCCTCTAACTGCCTGAGATAAATG | |
| | | | TTGGCAACTTCCTTTTATATTAAATTTCATTTT | |
| | | | TGTCACATAATACACTTGATTATTGACCATAA | |
| | | | TAACTTTATTAATATACAGACTGATTATTGAT | |
| | | | ACTCACCGATGTATTTCATGTGTTATTGAGAG | |
| | | | TCACTCATTTGGTTTAGAAAGACCAATATCAC | |
| | | | ATTGAGTAATTCGAAACATATTTAAGGCATAG | |
| | | | AACTTGCATTTTTTTCTCTTAAGCAAAATGAG | |
| | | | GAGTTCTAGCCAATCTTGCTAGTGTTATTTAT | |
| | | | AGCATCTTATTTCCTGAGAGAAGACAGGAAA | |
| | | | AGTGAGTCCCTGCCTTCCCTCTCTCCGTCTGG | |
| | | | CTCCTCCCAGGCCTGTCTGGCAGGGGCCGGG | |
| | | | GTGCAGGAGGAGGAGACGGCATCCAGTACAG | |
| | | | AGGGGCTGGACTTGGACCCCTGCAGCAGGTA | |
| | | | CTCGGAGCAAATGGTGAGATCAGAAGGGGGA | |
| | | | TGATGTCATTCCTTCGAAGGAATGAATTAAAC | |
| | | | GTGCTTCCTCGTGTGTCTGATTGACAGCCCTG | |
| | | | CACAGGAGAAGCGGCATATAAAGCCGCGCTG | |
| | | | CCCGGGAGCCGCTCGGCC | |
| 77 | TIMP-3 | tissue | GGGCGATTGGGCCCTCTAGATGCATGCTCGA | AF001361 |
| | | inhibitor of | GCGGCCGCCAGTGTGATGGATATCTGCAGAA | |
| | | metalloprotein- | TTCGCCCTTAGAGGAGGAGAAGCCGTCTGAG | |
| | | ases-3 | CGCCCGCCGCCTGCCTGCTGCCCGCTCTGCGC | |
| | | | CGCTGCCTGGGCGGCCGAGTGATATAGCGCT | |
| | | | GGGCCCCCGGGGACCCCGCCTCGGGCTGTTG | |
| | | | GGGCCCGCCCCCTCAGACCAATGGCAGAGCC | |
| | | | GCATTACCTCATCGGCCCTCCAAAAAGGGGG | |
| | | | CGGGGCCGGGGGCAAGGGGTAACGGGGCGG | |
| | | | GGCCGCCCCCGGATCGTTCAGATCCTTATAGG | |
| | | | GAATAATGCCGCCGTGGGCACGCGAG | |
| 78 | TMS-1 | methylation- | tgactacaaggaacagtgaTTGTTACAACCCAGA | AF184073 |
| | | induced | TGAGAGGGAAAAATAAAGGATTCCAAATATCCC | |
| | | silencing 1 | CCTTGGGAAgtagagtcaggattcaaacaaagaa | |
| | | (TMS1) | ctgtatggcttcaagttcatggtctttaatct | |
| | | | cctggaggctgtctctctTTCTTTTTTCTTT | |
| | | | TTTTTAATCAGTGTTGGGATCAAATTCTGGCT | |
| | | | CCCCTAGGAAGCATCTGGCAAGGTTTCGGGA | |
| | | | GCCATCGGGTTGGCCATGTTATGCTGGAATAT | |
| | | | TTATAAGCACCGGAGGGttatccccatgtcgt | |
| | | | agaaaatgaaactgaagctcagagagat | |
| | | | tTGCACTCTCTGCCCTTTTGTACAACTCATT | |
| | | | TTTCCCCAGTATGTGGAATTGAGGGAGCTT | |
| | | | CACGCTTCTAGCTGTCATGATTCCAAGA | |
| | | | TTCTACGACATGTGGGAGAGGATCCTA | |
| | | | AGGTTCGGGGAACCGCGGAGGTTTCGGGGTT | |
| | | | CTAGAAATCCGAGGTTCTAAGCCTAGGTGCTC | |
| | | | CAATAAACCCAGTGAGAGCCAGCCCAGGTTT | |
| | | | CCGGTCTGTACCCGCTGGTGCAAGCCCAGAG | |
| | | | ACAAGCAGGCGCCACCCATGAGCCCCTCTGC | |
| | | | GGCCCCCTCCCGGGTCCCACCTCGCAGGCCAG | |
| | | | CTGGAGGGCGCGATCCTGGCGTCCCCCGACG | |
| | | | GCCTGGGGCCCCAATCCAGAGGCCTGGGTGG | |
| | | | GAGGGGACCAAGGGTGTAGTAAGGAAGCGCC | |
| | | | TTTTGCTGGAGGGCAACGGACCGGGGCGGGG | |
| | | | AGTCGGGAGACCAGAGTGGGAGGAAGGCGG | |
| | | | GGAGTCCAGGTTCCGCCCCGGAGCCGACTTCC | |
| | | | TCCTGGTCGGCGGCTGCAGCGGGGTGAGCGG | |
| | | | CGGCAGCGGCCGGGGATCCTGGAGCCATGGG | |
| | | | GCGCGCGCGCGACGCCATCCTGGATGCGCTG | |
| | | | GAGAACCTGACCGCCGAGGAGCTCAAGAAGT | |
| | | | TCAAGCTGAAGCTGCTGTCGGTGCCGCTGCGC | |
| | | | GAGGGCTACGGGCGCATCCCGCGGGGCGCGC | |
| | | | TGCT | |
| 79 | TP73 | tumor protein | CCCGGGAGTGTTCGCGTCCTGGGTGACCCCTG | AB031234 |
| | | p73 | GAAGGACGTGGGGCCCAAACTCCGGCTGGGG | |
| | | | TTGGGAGAGCAGCCCCCAGAGGCTCTCCGCG | |
| | | | GGATCCTCTGCCGGGCGGGACCGTGGCTCCA | |
| | | | CAGGAGAAGTGGGTGGCAAGCCCTGCTTGGC | |
| | | | GGAAAGCAGCCGTTCCCCTCCTCCTGGGCCTG | |
| | | | GGGCGGCGCCCCTCACCCCTGTTCCCCGCCCC | |
| | | | TCACCCCTGTTCCCCGCCGGCCACATCCCCTG | |
| | | | CCCCTTGGATTCCAAGCGCCCCGCGCGCCGAG | |
| | | | GAGCCCAGCGCTAGTGGCGGCGGCCAGGAGA | |
| | | | GACCCGGGTGTCAGGAAAGATGGGCCGTCTG | |
| | | | GGGGACAGCAGGGAGTCCGGGGGAAACGCA | |
| | | | GGCGTCGGGCACAGAGTCGGCACCGGCGTCC | |
| | | | CCAGCTCTGCCGAAGATCGCGGTCGGGTCTG | |
| | | | GCCCGCGGGAGGGGCCCTGGCGCCGGACCTG | |
| | | | CTTCGGCCCTGCGTGGGCGGCCTCGCCGGGCT | |
| | | | CTGCAGGAGCGACGCGCGCCAAAAGGCGGCG | |
| | | | GGAAGGAGGCGGGGCAGAGCGCGCCCGGGA | |
| | | | CCCCGACTTGGACGCGGCCAGCTGGAGAGGC | |
| | | | GGAGCGCCGGGAGGAGACCTTGGCCCCGCCG | |
| | | | CGACTCGGTGGCCCGCGCTGCCTTCCCGCGCG | |
| | | | CCGGGCTAAAAAGGCGCTAAcgcccgcggccg | |
| | | | cctactccccgcggcgcctcccctccccgcgcc | |
| | | | catataacccgcctaggggccgggcagcccgcc | |
| | | | ctgcctccccgcccgcgcacccgcccggaggc | |
| | | | tcgcgcgcccgcGAAGGGGACGCAGCGAAACCG | |
| | | | GGGCCCGCGCCAGGCCAGCCGGGACGGACGCCG | |
| | | | ATGCCCGGGGCTGCGACGGCTGCAGGTAGGAGG | |
| | | | CCCAGGGCCGGGGGGCGGTTCGGCTCCGCGG | |
| | | | GCGGGGGCTGGAGCGCAGCGCTGGGCAGGCA | |
| | | | CCTGGGCTCGCAGCTCCGAAGCTGGGAGGTG | |
| | | | AGGGGAGAGCGATCGGGGACGA | |
| 80 | TSP-1 | thrombospondin | AATTCGAGTAGAAAGCAGCTGTCCTCCCCGG | NM_003246 |
| | | 1 | GCCCCTTGATGAGAATACGCACACCGCCCCC | |
| | | | AAGCGGCCGGCCGAGGGAGCGCCGCGGCAGC | |
| | | | GGGAGAGGCGTCTCTGTGGGCCCCCTGGCAG | |
| | | | CCGCGGCAGGAAAGGGCCCGAAGGCAGCGA | |
| | | | AGGCGAACGCGGCGCACCAACCTGCCGGCCC | |
| | | | CGCCGACGCCGCGCTCACCTCCCTCCGGGGCG | |
| | | | GGCGTGGGGCCAGCTCAGGACAGGCGCTCGG | |
| | | | GGGACGCGTGTCCTCACCCCACGGGGACGGT | |
| | | | GGAGGAGAGTCAGCGAGGGCCCGAGGGGCA | |
| | | | GGTACTTTAACGAATGGCTCTCTTGGTGTCCC | |
| | | | CTGCGCCCCGTCGGCCCATTTTTCTTTTTACAA | |
| | | | AACGGGCCCAGTCTCTAGTATCCACCTCTCGC | |
| | | | CATCAACCAGGCATTCCGGGAGATCAGCTCG | |
| | | | CCCGAAAGCCCCTGCGCCACCCCGCGGGCCC | |
| | | | TCCTAGGTGGTCTCCCCAGCCCCGTCCCTTTT | |
| | | | CGGGATGCTTGCTGATCACCCCGAGCCCGCGT | |
| | | | GGCGCAAGAGTACGAGCGCCGAGCCCGTGCG | |
| | | | CGCCAAGGCTGCGTGGGCGGGCACCGACTTT | |
| | | | TCTGAGAAGTTCTAGTGCTCCCAAGCCCCGAC | |
| | | | CCCCGCCCCCTTCACTTTCTAGCTGGAAAGTT | |
| | | | GCGCGCCAGGCAGCGGGGGGCGGAGAGAGG | |
| | | | AGCCCAGACTGGCCCCCACCTCCCGCTTCCTG | |
| | | | CCCGGCCGCCGCCCATTGGCCGGAGGAATCC | |
| | | | CCAGGAATGCGAGCGCCCCTTTAAAAGCGCG | |
| | | | CGGCTCCTCCGCCTTGCCAGCCGCTGCGCCCG | |
| | | | AGCTGGCCTGCGAGTTCAGGGCTCCTGTCGCT | |
| | | | CTCCAGGAGCAACCTCTACTCCGGACGCACA | |
| | | | GGCATTCCCCGCGCCCCTCCAGCCCTCGCCGC | |
| | | | CCTCGCCACCGCTCCCGGCCGCCGCGCTCCGG | |
| | | | TACACACAGGTAAGTCGCCCCCGGCGGCCGC | |
| | | | CGAGGACCAAAGCTGCCCGGGACATCCA | |
| 81 | VHL | von Hippel- | tgatgattgggtgttcccgtgtgagatgcgcca | NM_0005 |
| | | Lindau tumor | ccctcgaaccttgttacgacgtcggcacattg | |
| | | suppressor | cgcgtctgacatgaagaaaaaaaaaattcagtt | |
| | | | agtccaccaggcacagtggctaaggcctgtaa | |
| | | | tccctgcactttgagaggccaaggcaggaggatc | |
| | | | acttgaacccaggagttcgagaccagcctaggc | |
| | | | aacatagcgagactccgtttcaaacaacaaata | |
| | | | aaaataattagtcgggcatggtggtgcgcgcc | |
| | | | tacagtaccaactactcgggaggctgaggcgaga | |
| | | | cgatcgcttgagccagggaggtcaaggctgcag | |
| | | | tgagccaagctcgcgccactgcactccagcccggg | |
| | | | cgacagagtgagaccctgtctccaaaaaaaaaaa | |
| | | | aaaacaccaaaccttagaggggtgaaaaaaaattt | |
| | | | tatagtggaaatacagtaacgagttggcctagcc | |
| | | | tcgcctccgttacaacagcctacggtgctggagga | |
| | | | tccttctgcgcacgcgcacagcctccggccggct | |
| | | | atttccgcgagcgcgttccatcctctaccgagcgc | |
| | | | gcgcgaagactacggaggtcgactcgggagcgcg | |
| | | | cACGCAGCTCCGCCCCGCGTCCGACCCGCGGA | |
| | | | TCCCGCGGCGTCCGGCCCGGGTGGTCTGGATC | |
| | | | GCGGAGGGAatgCCCCGGAGGGCGGAGAACTG | |
| | | | GGACGAGGCCGAGGTAGGCGCGGAGGAGGC | |
| | | | AGGCGTCGAAGAGTACGGCCCTGAAGAAGAC | |
| | | | GGCGGGGAGGAGT | |
| 82 | WT1 | Wilms tumor | CTGTTTTCCCGGCTTAACCGTAGAAGAATTAG | X74840 |
| | | | ATATTCCTCACTGGAAAGGGAAACTAAGTGC | |
| | | | TGCTGACTCCAATTTTAGGTAGGCGGCAACCG | |
| | | | CCTTCCGCCTGGCGCAAACCTCACCAAGTAAA | |
| | | | CAACTACTAGCCGATCGAAATACGCCCGGCTT | |
| | | | ATAACTGGTGCAACTCCCGGCCACCCAACTG | |
| | | | AGGGACGTTCGCTTTCAGTCCCGACCTCTGGA | |
| | | | ACCCACAAAGGGCCACCTCTTTCCCCAGTGAC | |
| | | | CCCAAGATCATGGCCACTCCCCTACCCGACAG | |
| | | | TTCTAGAAGCAAGAGCCAGACTCAAGGGTGC | |
| | | | AAAGCAAGGGTATACGCTTCTTTGAAGCTTGA | |
| | | | CTGAGTTCTTTCTGCGCTTTCCTGAAGTTCCCG | |
| | | | CCCTCTTGGAGCCTACCTGCCCCTCCCTCCAA | |
| | | | ACCACTCTTTTAGATTAACAACCCCATCTCTA | |
| | | | CTCCCACCGCATTCGACCCTGCCCGGACTCAC | |
| | | | TGCTTACCTGAACGGACTCTCCAGTGAGACGA | |
| | | | GGCTCCCACACTGGCGAAGGCCAAGAAGGGG | |
| | | | AGGTGGGGGGAGGGTTGTGCCACACCGGCCA | |
| | | | GCTGAGAGCGCGTGTTGGGTTGAAGAGGAGG | |
| | | | GTGTCTCCGAGAGGGACGCTCCCTCGGACCC | |
| | | | GCCCTCACCCCAGCTGCGAGGGCGCCCCCAA | |
| | | | GGAGCAGCGCGCGCTGCCTGGCCGGGCTTGG | |
| | | | GCTGCTGAGTGAATGGAGCGGCCGAGCCTCC | |
| | | | TGGCTCCTCCTCTTCCCCGCGCCGCCGGCCCC | |
| | | | TCTTATTTGAGCTTTGGGAAGCTGAGGGCAGC | |
| | | | CAGGCAGCTGGGGTAAGGAGTTCAAGGCAGC | |
| | | | GCCCACACCCGGGGGCTCTCCGCAACCCGAC | |
| | | | CGCCTGTCCGCTCCCCCACTTcccgccctcc | |
| | | | ctcccacctactcattcacccacccacccacc | |
| | | | caGAGCCGGGACGGCAGCCCAGGCGCCCGGG | |
| | | | CCCCGCCGTCTCCTCGCCGCGATCCTGGACTT | |
| | | | CCTCTTGCTGCAGGACCCGGC | |

Methylation References
1 Ferguson et al, PNAS 2000, 97:6049-6054
2 Blood, 1999, 94:2452-2460
4 J Cell Biochem. 2003 Apr. 1; 88(5):899-910.
5 CpG methylation within the 5′ regulatory region of the BRCA1 gene is tumor specific and includes a putative CREB binding site. Oncogene 16:1161-1169, 1998.
6 Blood, 1991, 77: 2435-2440
7 Clin. Cancer Res., December 2003; 9: 6401-6409
8 Genes Chromosomes Cancer. 2003 July; 37(3):300-5.
9 PNAS Jun. 6, 2000 vol. 97 no. 12 6481-6486
10 Clin Cancer Res. 2002 Feb.; 8(2):464-70. Cancer Research, Vol 55, Issue 20 4525-4530
11 DNA Cell Biol. 1995 Sep.; 14(9):811-5.
12 J. Immunol. 2000 Apr. 15; 164(8):4143-9.
13 Cancer Res. 2000 Aug. 1; 60(15):4044-8
14 INTERNATIONAL JOURNAL OF ONCOLOGY 23: 1663-1670, 2003
15 Molecular Cancer 2003, 2:24 Cancer Research 62, 351-355,
16 Am. J. Pathol., March 1999; 154: 721-727
17 Molecular Cancer 2003, 2:24
18 Cancer Res. 1996 Aug. 15; 56(16):3655-8.
19 Cancer Res. 2001 Dec. 15; 61(24):8659-63.
20 Int J Cancer. 2001 Oct. 15; 94(2):212-7.
21 Clin. Cancer Res., February 1999; 5: 335-341.
22 Cell Res. 2003 Oct.; 13(5):319-33.
23 Diabetes, April 1999; 48: 685-690.
24 Cancer Res., February 1999; 59: 807-810
25 Krop, I. E. et al. HIN-1, a putative cytokine highly expressed in normal but not cancerous mammary epithelial cells. Proc. Natl. Acad. Sci. U.S. A 98, 9796-9801 (2001).
26 Clin. Cancer Res., September 2000; 6: 3607-3613.
27 Proc Natl Acad Sci USA. 1997 Apr. 29; 94(9):4342-7.
28 EMBO J., June 1985; 4: 1449-1454. Carcinogenesis, May 2002; 23: 777-785.
29 Br J. Cancer. 2003 Oct. 20; 89(8): 1473-8.
30 Mol. Cell. Biol., September 1998; 18: 5166-5177.
31 Diabetes 50:502-514, 2001
32 PNAS, August 2002; 99: 10623-10628.
33 J. Biol. Chem., October 2000; 275: 31805-31812
34 Blood, April 2003; 101: 3205-3211.
35 J. Immunol., October 2002; 169: 4253-4261.
36 Cancer Res., October 2003; 63: 6206-6211.
37 Mol. Cell. Biol., November 1999; 19: 7327-7335.
38 Am. J. Pathol., November 2003; 163: 1911-1919.
39 Mol. Cell. Biol., March 2002; 22: 1844-1857.
40 Carcinogenesis, October 2001; 22: 1715-1719
41 Journal of the National Cancer Institute, Vol. 94, No. 10, May 15, 2002
42 Clinical Cancer Research Vol. 8, 3164-3171, October 2002
43 Development 121, 2245-2253 (1995)
44 Am. J. Pathol., November 2003; 163: 2009-2019
45 Ann. N.Y. Acad. Sci., November 1998; 859: 180-183
46 Cancer Res., August 2003; 63: 4538-4546
47 Mol. Cell. Biol., September 1994; 14: 6143-6152.
48 Li, B. et al. CpG methylation as a basis for breast tumor-specific loss of NES1/kallikrein 10 expression. Cancer Res. 61, 8014-8021 (2001).
49 Cell Research (2003); 13(5):319-333
50 The Journal of Clinical Endocrinology & Metabolism Vol. 84, No. 7 2449-2457
51 Molecular Cancer 2002, 1:8 doi:10.1186/1476-4598-1-8
52 Lancet Oncol. 2004 Jan.; 5(1):27-36.
53 Mol Cell Biol. 2003 Jun.; 23(12):4056-65.
56 Int J Cancer. 2000 Jul. 15; 87(2):179-85.
54 Am. J. Pathol., November 1998; 153: 1475-1482
55 Br J Cancer. 2005 Jun. 20; 92(12):2171-80.
57 Nucleic Acids Res., April 1995; 23: 1119-1126.
58 Eur J Biochem. 1993 May 1; 213(3): 1283-96.
59 J Mol Endocrinol. 1991 Feb.; 6(1):53-61.
60 BMC Cancer 2004, 4:65
61 Mol Cell Endocrinol. 2003 Apr. 28; 202(1-2):201-7.
62 Blood, Vol. 94 No. 7 (Oct. 1), 1999: pp. 2445-2451
63 Molecular Carcinogenesis Volume 38, Issue 3, 2003. Pages 124-129
66 Leukemia & Lymphoma Volume 44, Number 11 Sep. 2003, 1855-1864
67 Cancer Research 65, 828-834, Feb. 1, 2005
68 Cancer Research 61, 7943-7949, Nov. 1, 2001
69 Cell & Developmental Biology 14 (2003) 161-168
70 FEBS Lett. 2004 Mar. 26; 562(1-3):27-34.
71 Cancer Lett. 2001 Aug. 28; 169(2):155-64.
72 THE LANCET Vol 361 May 17, 2003, 1693-1699
74 Gene, Volume 266, Number 1, 21 Mar. 2001, pp. 67-75(9)
75 Clin Cancer Res. 2002 Jul.; 8(7):2217-24.
76 Clin Cancer Res. 2002 Jul.; 8(7):2217-24.
77 Cancer Genetics and Cytogenetics 144 (2003) 134-142
78 Oncogene. 2003 May 29; 22(22):3475-88.
79 Molecular Cancer 2003, 2:24
80 Oncology. 2003; 64(4):423-9.
81 Cancer Res., July 2003; 63: 3724-3728
82 Loeb, D. M. et al. Wilms' tumor suppressor gene (WT1) is expressed in primary breast tumors despite tumor-specific promoter methylation. Cancer Res. 61, 921-925 (2001).

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Array Analysis of Promoter Methylation

In this example, an embodiment of the methodology provided in the present invention was used for the high throughput analysis of promoter methylation, which simultaneously profiles the methylation status of 82 different promoter regions, from one sample.

As illustrated in FIG. 1 Panel B, this embodiment includes 3 steps:

(1) Genomic DNA is digested with a restriction enzyme to isolate DNA with CpG islands. The digests are purified and adapted with linkers.

(2) The adapted DNA is incubated with the methylation binding protein (MBP), which forms a protein/DNA complex. These complexes are separated and methylated DNA is isolated.

(3) The methylated DNA is labeled with biotin-dCTP via PCR and these probes are hybridized to the methylation array.

The details of the above procedure are described below.

I. Fragmentation of Genomic DNA

We digested 2 μg of genomic DNA from cell samples such as Hs 578Bst, Hs 578T and MCF7 cells with MseI restriction enzyme, to produce small fragments of DNA (<200 bp) that retain the CpG islands.

1. Set up the following restriction digest:

| | |
|---|---|
| Genomic DNA (200 ng/μl) | 10 μl |
| 10× NE Buffer 2 with BSA | 2 μl |

(1× buffer2 + BSA = 50 mM NaCl, 10 mM Tris-HCl, 10 mM MgCl2,
1 mM DTT and 100 ug/ml BSA)

| | |
|---|---|
| MseI (New England) | 1 μl |
| dH₂O | 7 μl |
| Total Volume | 20 μl |

2. Mix well by pipetting and incubate at 37° C. for 2 hours.
3. Add 100 μl PB Buffer (Qiagene Cat# 1906)) to the digest reaction and transfer all solution to the DNA purification column (Qiagen)
4. Bind the DNA to the column, centrifuge at 10,000 g, for 30-60 s.
5. Discard flow through.
6. Add 750 μl PE Buffer (Qiagene Cat# 19065) centrifuge at 10,000 g, for 30-60 s.
7. Discard the flow-through and centrifuge the column at maximum speed, for 1 min.
8. Elute the DNA by adding 10 μl dH20 to the center of the column membrane and let the column stand for 5 min. Then centrifuge the column at maximum speed, for 1 min.
II. Ligation of PCR Adaptors to DNA Fragments

We added the adaptors for future PCR steps to the restricted ends of the DNA fragments.

1. Add the following components to a 0.5 ml microfuge tube.

| | |
|---|---|
| Digested DNA | 3 μl |
| 50 uM Linker (H12 + H24) | 1 μl |
| H-24 | |
| AGGCAACTGTGCTATCCGAGGGAT | SEQ ID NO:83 |
| H-12 | |
| TAATCCCTCGGA | SEQ ID NO:84 |
| 2X ligase buffer | 5 μl |
| (Roche Cat#1635379) | |
| Total Volume | 9 μl |

The linkers H24 and H12 were added to the end of MseI digested DNA fragments as illustrated below.

```
                          H24 H12
5′ AGGCAACTGTGCTATCCGAGGGATTAAxxxxxxxxxxxxTTAATCCC
TCGGA3′
3′ AGGCTCCCTAATTxxxxxxxxxxxxAATTAGGAGCCTATCCTGTCAA
CGGA5′
              H12 H24
```

Underlined nucleotides are sticky ends generated by MseI digestion (cut site for MseI is TTAA).
2. Heat the samples at 50° C. for 3 min and lower the temperature to 25° C. slowly in a PCR machine (ramp temperature at a rate of 0.1° C./sec).
3. Add 1 μl of ligase.
4. Mix components by pipetting and incubate at room temperature for 30 min.
5. Repeat steps 3-8 in section I above to purify the genomic DNA fragments adapted with linkers H12 and H24.
III. Isolation of Methylated DNA Fragments

We isolated the methylated DNA fragments from the non-methylated fragments. All centrifuge steps were carried out on a regular benchtop centrifuge at 7,000 rpm at 4° C.

1. Prepare methylation binding protein MeCP2/DNA complexes:

Add the following components to a 0.5 ml microfuge tube:

| | |
|---|---|
| Recombinant MeCP2 (50 ng) | 2 μl |
| Purified DNA fragment | 6 μl |
| 5× Binding Buffer | 4 μl |
| dH₂O | 8 μl |
| Total Volume | 20 μl |

Recombinant MeCP2 used in this experiment is a full length human MeCP2 or a His-tagged mouse MeCP2 (1-206 amino acids) expressed in E. coli and purified according to Chen et al. (2003) Science 302:885-889 and supplemental materials; and Nan et al. (1993) Nucleic Acid Res. 21:4886-92, which are herein incorporated by reference.
2. Mix components by pipetting and incubate at 15° C., for 30 min.
3. Meanwhile, wash the Separation Column (containing, e.g., a 0.45 μm pore size nitrocellulose membrane) by adding 500 μl chilled IX Column Incubation Buffer (0.5×TBE (45 mM Tris base, 45 mM boric acid, 1 mM EDTA pH 8.0)) and centrifuging at 7,000 rpm for 30 sec at the room temperature.
4. Add 20 μl 1× Column Incubation Buffer (0.5×TBE) to the MBP-DNA, and transfer all of this onto the membrane of the Separation Column.
5. Incubate the Separation Column on ice for 30 min.
6. Centrifuge column at 7,000 rpm for 30 sec at 4° C. and discard the flow-through.
7. Add 600 μl 1× Column Wash Buffer (0.5×TBE+0.01% Tween 20) to column and incubate for 10 min on ice.
8. Centrifuge column at 7,000 rpm for 30 sec at 4° C. and discard the flow through.
9. Wash the column by adding 600 μl 1× Column Wash Buffer to the Separation Column and centrifuging at 7,000 rpm for 30 sec at 4° C.
10. Repeat step 9 three times.
11. Remove residual Wash Buffer by an additional centrifugation at 7,000 rpm for 30 sec at 4° C.
12. Add 10 μl 1× Column Elution Buffer (0.01% SDS or 0.5% SDS) to the center of the Separation Column and incubate at room temperature for 5 min.
13. Place the Separation Column in a clean 1.5 ml microcentrifuge tube and centrifuge for 1 minute at 10,000 rpm at room temperature.
14. Place the microfuge tube containing the collected flow through on ice and use for further steps.
IV. Biotinylation of Methylated DNA Fragments

The purified methylated DNA fragments were then converted into biotinylated probes.

1. Mix the following components in a 0.5 ml microfuge tube:

Methylated DNA (from step 14 in section III above) 1 μl
biotin dCTP 5 μl
1×PCR buffer 50 μl (1×XL PCR reaction buffer (Perkin-Elmer); 1.1 mM Mg(OAc)₂, and 1 μl of 50 uM Linker mix (H12 and H24 primers))
Polymerase rtTh (Perkin-Elmer) 1 μl
   2. Mix well by pipetting and carry out the following PCR steps, for 30 cycles:

72° C. 3 min

30 cycles of the following steps:

94° C. 1 min

55° C. 1 min

72° C. 2 min

4° C. Forever

3. Denature at 98° C. for 5 min using PCR machine with heated lid and then quickly chill on ice for 2 min.

V. Hybridization

The probes amplified from the isolated methylated DNA fragments and labeled with biotin were hybridized to an array of DNA sequences corresponding to 82 different promoter regions of genes (Table 1).

1. Place each array membrane into a hybridization bottle. Wet the membrane by filling the bottle with deionized H₂O. Then, carefully decant the water. Be sure to place the membrane in the hybridization bottle such that the spotted oligos face the center of the tube (away from the walls).

2. To each hybridization bottle that contains an array membrane, add 3-5 ml of prewarmed Hybridization Buffer (20% sodium dodecyl sulfate (SDS), 1 mM EDTA, 250 mM sodium phosphate). Place each bottle in the hybridization oven at 50° C. for 2 hr.

3. Add half of the denatured probe to each hybridization bottle and hybridize at 50° C. overnight.

4. Decant the hybridization mixture from each hybridization bottle, and wash each membrane as follows.

5. Add 50 ml of prewarmed Hybridization Wash I (2×SSC (0.3M NaCl and 0.03M citric Acid)/0.5% SDS), incubate at 50° C. for 20 min in a rotating hybridization oven. Decant liquid and repeat wash.

6. Add 50 ml of prewarmed Hybridization Wash 11 (0.1×SSC (15 mM NaCl and 1.5 mM Citric Acid)/0.5% SDS), incubate at 50° C. for 20 min in a rotating hybridization oven. Decant liquid and repeat wash.

VI. Detection

The biotinylated probes amplified from the isolated methylated DNA fragments that were hybridized to the DNA array in section V above were detected as follows.

1. Using forceps, carefully remove each membrane from the hybridization bottle and transfer to a new container containing 20 ml of 1× Blocking Buffer. (Container was approx. 4.5″×3.5″).

1× Blocking Buffer:

1× SuperBlock Dry Blend (TBS) Block Buffer (Cat#37545, Pierce)

2. Block the membrane by incubating at room temperature for 15 minutes with gentle shaking.

3. Dilute 20 μl of Strepavidin-HRP (horseradish peroxidase) conjugate into 1 ml of Blocking Buffer and add to each membrane. Do not pipet diluted Strepavidin-HRP directly onto the membrane. Continue shaking the membrane for 15 minutes at room temperature.

4. Decant the Blocking Buffer and wash three times at room temperature with IX Wash Buffer (20 mM Tris pH 7.6, 140 mM NaCl), 8 minutes for each wash, shaking gently.

5. Add 20 ml of 1× Detection Buffer (0.1 M Tris-HCl pH 9.5, 0.1 M NaCl) to each membrane and incubate at room temperature for 5 minutes, shaking gently.

6. Combine equal amounts of Stable Peroxide Solution (Pierce, cat. #89880F) and Luminol/Enhancer Solution (Pierce, cat. #89880E). Place the membrane on a plastic sheet protector or overhead transparency. Overlay each membrane with 1 ml of substrate solution, ensuring that the substrate is evenly distributed over the membrane. Place another plastic sheet over the top of the membrane, without trapping air bubbles on the membrane. Incubate at room temperature for 5 minutes.

7. Remove excess substrate by pressing a paper towel over the plastic sheet. Expose the membranes using either Hyperfilm ECL nitrocellulose membrane for 2-10 min or a chemiluminescence imaging system (e.g., Fluor Chem imager from Alpha Innotech).

VII. Detection of Methylation Status of Promoter Regions of Genes in Breast Cell Lines

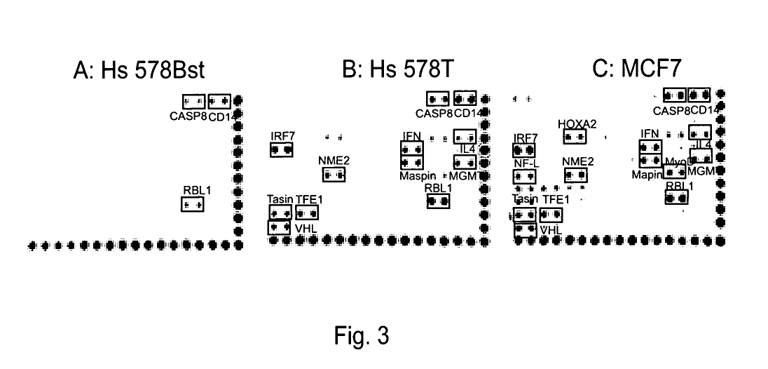

Methylation status of promoter regions of genes in normal and breast cancer cells was analyzed by using the embodiment of the inventive method described above. Briefly, 2 μg of genomic DNA from cells from each sample of breast cells was digested with MseI, and the methylated DNA was incubated with a methylation binding protein MeCP2 and separated by a spin column as described above. The methylated DNA was amplified and labeled with biotin by PCR. The denatured PCR product was hybridized with the methylation array shown in FIG. 2. The results of the hybridization array are shown in FIG. 3. Hs578Bst (Panel A) and Hs578T (Panel B) are cell lines established from breast tissue of the same patient: Hs578Bst is from normal breast tissue, Hs578T is from cancer breast tissue. MCF7 (Panel C) is a breast cancer cell line from adenocarcinoma.

As shown in FIG. 3, methylated DNA fragments that hybridized to the array are detected based on the spots on the membrane. As the hybridization membrane was spotted with a DNA plasmid containing a predetermined promoter sequence of a gene at a specific position in the array (FIG. 2), the DNA fragment that hybridized to the particular spot is the one containing the promoter sequence. Since such a DNA fragment was PCR amplified from the methylated genomic DNA fragment, the identity of the promoter region that has been methylated could thus be determined by correlating with the identity of the spot. As indicated in FIG. 3, in the normal breast cell line, Hs578Bst, few genes are methylated, except for moderate methylation in the promoter regions of CASP8, CD14 and RBL1. In contrast, there is extensive methylation in the promoter regions of genes in breast cancer cell lines: for Hs578T, CASP8, CD14, IRF7, IFN, IL4, NME2, Maspin, MGMT, RBL1, Tasin, TFE1, and VHL; and for MCF7, CASP8, CD14, IRF7, HOXA2, IFN, IL4, NF-L, NME2, Maspin, MyoD, MGMT, RBL1, Tasin, TFE1, and VHL. The density of the spot usually correlates with the quantity of the particular methylated DNA fragments that hybridized to the predetermined promoter sequence on that spot. Thus, this assay not only can profile methylation status of multiple genes, but can also distinguish the extent to which each gene is methylated, in a high throughput and quantitative manner.

### Example 2

### BDNA Analysis of Promoter Methylation

The following sets forth a series of experiments that demonstrate isolation and detection of methylated nucleic acids, using a nitrocellulose filter-based 96 well plate separation method to isolate methylated DNA-MBP complexes and a bDNA assay to detect the DNA from the isolated complexes. Use of the multiwell filter separation plate facilitates high throughput analysis of multiple samples, since large numbers of samples (e.g., up to 96, on a 96 well plate) can be processed simultaneously to separate methylated nucleic acid from unmethylated nucleic acid. Use of the bDNA detection technique shortens the procedure as compared to array detection, since the bDNA assay does not include linker ligation, PCR biotin labeling, or array hybridization steps. The procedure is schematically illustrated in FIG. 4.

Methylated DNA Preparation

1.5 μg genomic DNA prepared from MCF7, T47D, and 1806 breast cancer cell lines (American Type Culture Collection) is digested with MseI (New England Biolabs Cat# R0525S) for 2 hours at 37° C. The digested DNA fragments are purified with a QIAgene column, and eluted in 20 μl ddH₂O. 6 μl of purified DNA is incubated with 2 μl (100 ng) full length recombinant human MeCP2 protein (see, e.g., Hendrich and Bird (1998) Molecular and Cellular Biology 18(11):6538-6547) at 15° C. for 30 min in total volume of 20 μl of binding buffer (final concentration in the binding reaction is 20 mM HEPES, free acid, pH 7.6, 1 mM EDTA, 10 mM ammonium sulfate, 1 mM DTT, 30 mM KCl, 0.1 μg poly(dI-C), and 0.2% Tween-20) to form protein-DNA complexes. The 20 μl reaction is loaded on a nitrocellulose-based filter plate, e.g., a 96 well 0.45 μm cellulose nitrate plate from Whatman, catalog number 7700-3307 (or an individual spin column as described above), and incubated on ice for 20 min. The filter plate with bound protein-DNA complexes is washed with washing buffer (44.5 mM Tris, 44.5 mM Borate, 1 mM EDTA, and 0.02% NP-40) for 5 times at 4° C. The methylated DNA is eluted with 60 μl elution buffer (0.01% SDS) at 4° C. (or at room temperature).

bDNA Assay

Four gene promoters were targeted, including IRF7, BRCA1, VHL and BIRC5. Two CpG islands were selected from within −3000 bp to +1000 bp of each gene promoter region. Probe sets (LE, CE and BP) were designed based on the CpG island sequences. Island sequences are presented in Table 5, and the corresponding probe sets are presented in Table 6.

To denature the DNA, 20 μl of DNA eluted from the nitrocellulose plate (or spin column) is incubated with 2 μl of 2.5 N NaOH at 53° C. for 15 min, then mixed with 20 μl 12M Hepes acid. 20 μl denatured DNA is incubated with 80 μl lysis mixture and 10 μl probe set at 53° C. on a capture plate overnight. The final concentration of the probes is 1 nM for each LE, 0.23 nM for each CE, and 0.5 nM for each BP.

Detection is continued using reagents from Panomics's QuantiGene® Assay Kits (www(dot)panomics(dot)com) according to the manufacturer's instructions. In brief, the capture plate is washed three times with 200 μl/well washing buffer, followed by incubation with amplification multimer (100 μl/well amplifier working reagent) at 46° C. for 1 hour. After washing, 100 μl/well label probe is incubated on the plate at 46° C. for 1 hour. After washing, 100 μl/well substrate is incubated on the plate at 46° C. for 30 minutes. The plate is then read in a luminometer. Capture plate (a 96 well plate coated with capture probe), lysis mixture, bDNA amplifier, label probe, wash buffer, and substrate commercially available, e.g., in Panomics's QuantiGene® Assay Kits (www(dot)panomics(dot)com), were used in these experiments, but other suitable buffers and other reagents can be prepared by one of skill in the art (see, e.g., the references herein, including U.S. patent application Ser. No. 11/433,081, and U.S. patent application Ser. No. 11/543,752 filed Oct. 4, 2006 entitled “Detection of nucleic acids from whole blood” by Zhi Zheng et al.).

Analysis of Promoter Methylation

In an initial experiment, methylated DNA from each of two CpG islands was detected for each of the four target genes (IRF7, BRCA1, VHL and BIRC5) from each of three cell lines. As described above, 1.5 μg of genomic DNA from cell lines MCF7, T47D, and 1806 was digested with MseI. The digested DNA fragments were incubated with MeCP2, and the methylated DNA fragments were separated by spin filter column or plate. The eluted methylated DNA was subjected to bDNA detection with probe sets for the two CpG islands for each gene promoter.

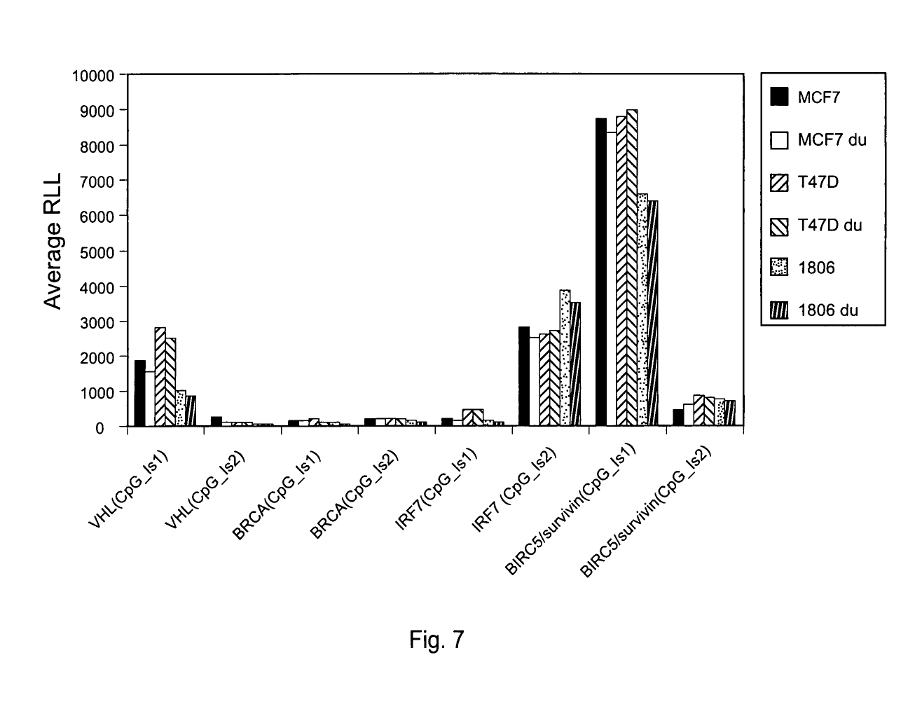

Results from two repetitions of the assay are depicted in FIG. 7 (du represents the independent repetition of the assay).

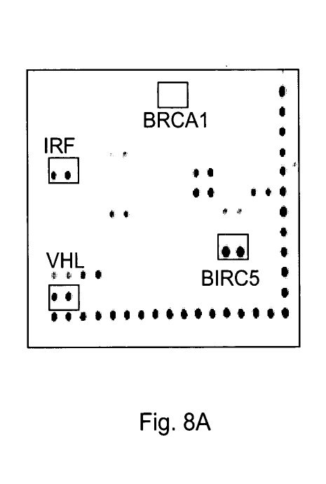

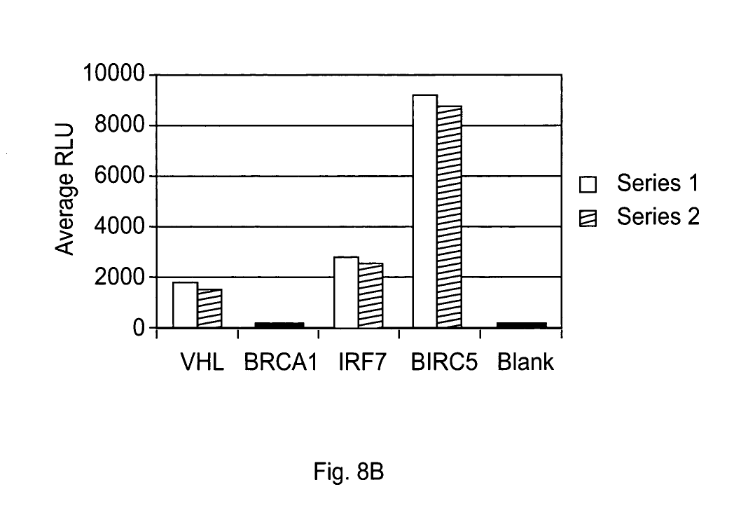

One island (and the corresponding probe set) was selected for each of the four genes, and results from the bDNA assay were compared to results for the same four promoters from the array assay (FIG. 8).

Results from the array assay are shown in FIG. 8 Panel A. For the array assay, performed as described herein, 1.5 μg of genomic DNA prepared from MCF7 cells was digested with MseI. The digested DNA was ligated with linker, then incubated with MeCP2. Methylated DNA binds with MeCP2 and was separated by filter spin column or plate. The eluted methylated DNA was labeled with biotin by PCR. The labeled PCR products were hybridized to the array. The spots in boxes are the four gene promoters for comparison with bDNA detection.

Results from the bDNA assay are shown in FIG. 8 Panel B. MseI-digested DNA from MCF7 cells was incubated with MeCP2. The methylated DNA was separated using a spin column or plate and subjected to bDNA detection, as described herein, with the following probe sets: VHL island1, BRCA island1, IRFI island2, and BIRC5/survivin island1. Two repetitions of the assay were performed (indicated as series 1 and series 2 in FIG. 8 Panel B). The bars labeled “Blank” represent results from controls with no added genomic DNA and the BIRC5 probe set.

The bDNA-based method can detect as little as 0.025 pg genomic DNA, and generates compatible results with those from the array assay.

TABLE 5
Target names and sequences.

| | Target | |
|---|---|---|
| SEQ | accession | |
| ID | number and | |
| NO | name | Sequence |
| 85 | NM_000551 | ataagcgtgatgattgggtgttcccgtgtgagatgcgccaccctcgaaccttgttacgacgtcggcacattgcg |
| | (VHL) CpG | cgtctgacatgaAGAAAAAAAAAATTCAGTTAGTCCAccaggcacagtggctaaggc |
| | island1 | ctgtaatccctgcactttgagaggccaaggcaggaggatcacttgaacccaggagttcgagaccagcctagg |
| | | caacatagcgagactccgtttcaaacaacaaataaaaataattagtcgggcatggtggtgcgcgcctacagtac |
| | | caactactcgggaggctgaggcgagacgatcgcttgagccagggaggtcaaggctgcagtgagccaagctc |
| | | gcgccactgcactccagcccgggcgacagagtgagaccc |
| 86 | NM_000551 | GGGCGGAGAACTGGGACGAGGCCGAGGTAGGCGCGGAGGAGGCA |
| | (VHL) CpG | GGCGTCGAAGAGTACGGCCCTGAAGAAGACGGCGGGGAGGAGTC |
| | island2 | GGGCGCCGAGGAGTCCGGCCCGGAAGAGTCCGGCCCGGAGGAAC |
| | | TGGGCGCCGAGGAGGAGATGGAGGCCGGGCGGCCGCGGCCCGTG |
| | | CTGCGCTCGGTGAACTCGCGCGAGCCCTCCCAGGTCATCTTCTGCA |
| | | ATCGCAGTCCGCGCGTCGTGCTGCCCGTATGGCTCAACTTCGACGG |
| | | CGAGCCGCAGCCCTACCCAACGCTGCCGCCTGGCACGGGCCGCCG |
| | | CATCCACAGCTACCGAGGTACGGGCCCGGCGCTTAGGCCCGACCC |
| | | AGCAGGGACGATAGCACGGTCTGAAGC |
| 87 | NM_001168 | GGAGTAGATGCTTTTTGCAGAGGTGGCACCCTGTAAAGCTCTCCTG |
| | (BIRC5) | TCTGACtttttttttttttttagactgagttttgctcttgttgcctaggctggagtgcaatggcacaatctcagctc |
| | CpG | actgcaccctctgcctcccgggttcaagcgattctcctgcctcagcctcccgagtagttgggattacaggcatgc |
| | island1 | accaccacgcccagctaatttttgtatttttagtagagacaaggtttcaccgtgatggccaggctggtcttgaactc |
| | | caggactcaagtgatgctcctgcctaggcctctcaaagtgttgggattacaggcgtgagccactgcacccggc |
| | | cTGCACGCGTTCTTTGAAAGCAGTCGAGGGGGCGCTAGGTGTGGG |
| | | CAGGGACGA |
| 88 | NM_001168 | CTGGGTGCACCGCGACCACGGGCAGAGCCACGCGGCGGGAGGAC |
| | (BIRC5) | TACAACTCCCGGCACACCCCGCGCCGCCCCGCCTCTACTCCCAGAA |
| | CpG | GGCCGCGGGGGGTGGACCGCCTAAGAGGGCGTGCGCTCCCGACAT |
| | island2 | GCCCCGCGGCGCGCCATTAACCGCCAGATTTGAATCGCGGGACCC |
| | | GTTGGCAGAGGTGGCGGCGGCGGCATGGGTGCCCCGACGTTGCCC |
| | | CCTGCCTGGCAGCCCTTTCTCAAGGACCACCGCATCTCTACATTCA |
| | | AGAACTGGCCCTTCTTGGAGGGCTGCGCCTGCACCCCGGAGCGGG |
| | | TGAGACTGCCCGGCCTCCTGGGGTCCCCCACGCCCGCCTTGCCCTG |
| | | TCCCTAGCGAGGCCAC |
| 89 | NM_001572 | GCTGGCGGAAGCCCCACGGCGGTGAGGTCCATCCTGACCAAGGAG |
| | (IRF7) | CGGCGGCCGGAGGGCGGGTACAAGGCTGTCTGGTTTGGCGAGGAC |
| | CpG | ATCGGGACGGAGGCAGACGTGGTCGTTCTCAACGCGCCCACCCTG |
| | island1 | GACGTGGATGGCGCCAGTGACTCCGGCAGCGGCGATGAGGGCGA |
| | | GGGCGCGGGGAGGGGTGGGGGTCCCTACGATGCGCCCGGTGGTGA |
| | | TGACTCCTACATCTAAGTGGCCCCTCCACCCTCTCCCCCAGCCGCA |
| | | CGGGCACTGGAGGTCTCGCTCCCCCAGCCTCCGACCCGAGGCAGA |
| | | ATAAAGCAAGGCTCCCGAAACC |
| 90 | NM_001572 | TGCCAAGAGATCCATACCGAGGCAGCGTCGGTGGCTACAAGCCCT |
| | (IRF7) | CAGTCCACACCTGTGGACACCTGTGACACCTGGCCACACGACCTG |
| | CpG | TGGCCGCGGCCTGGCGTCTGCTGCGACAGGAGCCCTTACCTCCCCT |
| | island2 | GTTATAACACCTGACCGCCACCTAACTGCCCCTGCAGAAGGAGCA |
| | | ATGGCCTTGGCTCCTGAGAGGTAAGAGCCCGGCCCACCCTCTCCA |
| | | GATGCCAGTCCCCGAGCGCCCTGCAGCCGGCCCTGACTCTCCGCG |
| | | GCCGGGCACCCGCAGGGCAGCCCCACGCGTGCTGTTCGGAGAGTG |
| | | GCTCCTTGGAGAGATCAGCAGCGGCTGCTATGAGGGG |
| 91 | NM_007294 | TTAGTGTGACGTGACCCCACCCCTAGCTAACCCAGGCTGCTTCCTT |
| | (BRCA1) | ACCAGCTTCCCGCCCCCTGGGGAGGCGGCAATGCAAAGACCGTCC |
| | CpG | GCTGCCAGCTCTGCCGCTATCTCTGTGGGGTGAATCTAACATGGCG |
| | island1 | GACAAAGACAGTAACTAGTCCCGTTTCTCCGCGTTTTCGCCAAGAA |
| | | GATTGGCTCTTACCACTTGTCCCTCAAAACGACCACCCCATTGACT |
| | | GGTGGCGATTGCGTCGACGGAGACGGGGCAAAAGCAAGCTGAAC |
| | | CCGAAAAATAACAAACACTGGGGCTGAGGGGTGGAACTACGAGT |
| | | GCGCAGACATGGGCCAGAGCGCATTTCCCCTGCCCCAGGCAAATT |
| | | CGGCGCTCACTGCGTCCCCGCAGGCCACTG |
| 92 | NM_007294 | TAAATTAAAACTGCGACTGCGCGGCGTGAGCTCGCTGAGACTTCC |
| | (BRCA1) | TGGACGGGGGACAGGCTGTGGGGTTTCTCAGATAACTGGGCCCCT |
| | CpG | GCGCTCAGGAGGCCTTCACCCTCTGCTCTGGGTAAAGGTAGTAGA |
| | island2 | GTCCCGGGAAAGGGACAGGGGGCCCAAGTGATGCTCTGGGGTACT |
| | | GGCGTGGGAGAGTGGATTTCCGAAGCTGACAGATGGGTATTCTTT |
| | | GACGGGGGGTAGGGGCGGAACCTGAGAGGCGTAAGGCGTTGTGA |
| | | ACCCTGGGGAGGGGGGCAGTTTGTAGGTCGCGAGGGAAGCGCTGA |
| | | GGATCAGGAAGGGGGCACTGAGTGTCCGTGGGGGA |

TABLE 6
Probe sets (CEs, LEs, and BPs).

| | | |
|---|---|---|
| | | SEQ ID |
| Target name | Sequence | NO |

| | | | |
|---|---|---|---|
| VHL island1 | CE | ctgaattttttttttcttcatgtcaTTTTTctcttggaaagaaa | 93 |
| | | gt | |
| VHL island1 | CE | gtgcagggattacaggccttagTTTTTctcttggaaagaaagt | 94 |
| VHL island1 | CE | gttgcctaggctggtctcgaTTTTTctcttggaaagaaagt | 95 |
| VHL island1 | CE | tgtttgaaacggagtctcgctatTTTTTctcttggaaagaaagt | 96 |
| VHL island1 | CE | gccttgacctccctggctcTTTTTctcttggaaagaaagt | 97 |
| VHL island1 | CE | gggctggagtgcagtggcTTTTTctcttggaaagaaagt | 98 |
| VHL island1 | LE | gaacacccaatcatcacgcttatTTTTTaggcataggacccgtg | 99 |
| | | tct | |
| VHL island1 | LE | tggcgcatctcacacggTTTTTaggcataggacccgtgtct | 100 |
| VHL island1 | LE | cgtcgtaacaaggttcgagggTTTTTaggcataggacccgtgtc | 101 |
| | | t | |
| VHL island1 | LE | gacgcgcaatgtgccgaTTTTTaggcataggacccgtgtct | 102 |
| VHL island1 | LE | ccactgtgcctggtggactaaTTTTTaggcataggacccgtgtc | 103 |
| | | t | |
| VHL island1 | LE | cctgccttggcctctcaaaTTTTTaggcataggacccgtgtct | 104 |
| VHL island1 | LE | tgcccgactaattatttttatttgtTTTTTaggcataggacccg | 105 |
| | | tgtct | |
| VHL island1 | LE | gcctcccgagtagttggtactgTTTTTaggcataggacccgtgt | 106 |
| | | ct | |
| VHL island1 | LE | aagcgatcgtctcgcctcaTTTTTaggcataggacccgtgtct | 107 |
| VHL island1 | LE | gcgagcttggctcactgcaTTTTTaggcataggacccgtgtct | 108 |
| VHL island1 | LE | gggtctcactctgtcgcccTTTTTaggcataggacccgtgtct | 109 |
| VHL island1 | BL | actcctgggttcaagtgatcct | 110 |
| VHL island1 | BL | taggcgcgcaccacca | 111 |
| VHL island2 | CE | gccggactcctcggcgTTTTTctcttggaaagaaagt | 112 |
| VHL island2 | CE | cgtcccagttctccgcccTTTTTctcttggaaagaaagt | 113 |
| VHL island2 | CE | cgcgcctacctcggcctTTTTTctcttggaaagaaagt | 114 |
| VHL island2 | CE | gggccggactcttccggTTTTTctcttggaaagaaagt | 115 |
| VHL island2 | CE | tgcgattgcagaagatgacctTTTTTctcttggaaagaaagt | 116 |
| VHL island2 | CE | gcggcagcgttgggtagTTTTTctcttggaaagaaagt | 117 |
| VHL island2 | CE | cctgctgggtcgggcctaTTTTTctcttggaaagaaagt | 118 |
| VHL island2 | LE | cttcgacgcctgcctcctcTTTTTaggcataggacccgtgtct | 119 |
| VHL island2 | LE | cgtcttcttcagggccgtactTTTTTaggcataggacccgtgtc | 120 |
| | | t | |
| VHL island2 | LE | cggcgcccagttcctccTTTTTaggcataggacccgtgtct | 121 |
| VHL island2 | LE | ccggcctccatctcctcctTTTTTaggcataggacccgtgtct | 122 |
| VHL island2 | LE | gttcaccgagcgcagcacTTTTTaggcataggacccgtgtct | 123 |
| VHL island2 | LE | gggagggctcgcgcgaTTTTTaggcataggacccgtgtct | 124 |
| VHL island2 | LE | agcacgacgcgcggacTTTTTaggcataggacccgtgtct | 125 |
| VHL island2 | LE | cgaagttgagccatacgggcTTTTTaggcataggacccgtgtct | 126 |
| VHL island2 | LE | ggctgcggctcgccgtTTTTTaggcataggacccgtgtct | 127 |
| VHL island2 | LE | acctcggtagctgtggatgcTTTTTaggcataggacccgtgtct | 128 |
| VHL island2 | LE | gcttcagaccgtgctatcgtcTTTTTaggcataggacccgtgtc | 129 |
| | | t | |
| VHL island2 | BL | cccgactcctccccgc | 130 |
| VHL island2 | BL | gggccgcggccgc | 131 |
| VHL island2 | BL | ggcggcccgtgccag | 132 |
| VHL island2 | BL | agcgccgggcccgt | 133 |
| BIRC5 island1 | CE | ggagagctttacagggtgccaTTTTTctcttggaaagaaagt | 134 |
| BIRC5 island1 | CE | ttgtgccattgcactccagcTTTTTctcttggaaagaaagt | 135 |
| BIRC5 island1 | CE | cagagggtgcagtgagctgagaTTTTTctcttggaaagaaagt | 136 |
| BIRC5 island1 | CE | tcgcttgaacccgggaggTTTTTctcttggaaagaaagt | 137 |
| BIRC5 island1 | CE | ccgggtgcagtggctcacTTTTTctcttggaaagaaagt | 138 |
| BIRC5 island1 | CE | ttcaaagaacgcgtgcaggTTTTTctcttggaaagaaagt | 139 |
| BIRC5 island1 | LE | cctctgcaaaaagcatctactccTTTTTaggcataggacccgtg | 140 |
| | | tct | |
| BIRC5 island1 | LE | ctaggcaacaagagcaaaactcaTTTTTaggcataggacccgtg | 141 |
| | | tct | |
| BIRC5 island1 | LE | ggaggctgaggcaggagaaTTTTTaggcataggacccgtgtct | 142 |
| BIRC5 island1 | LE | ggcctaggcaggagcatcacTTTTTaggcataggacccgtgtct | 143 |
| BIRC5 island1 | LE | tgcctgtaatcccaactactcgTTTTTaggcataggacccgtgt | 144 |
| | | ct | |
| BIRC5 island1 | LE | ctgggcgtggtggtgcaTTTTTaggcataggacccgtgtct | 145 |
| BIRC5 island1 | LE | ccttgtctctactaaaaatacaaaaattagTTTTTaggcatagg | 146 |
| | | acccgtgtct | |
| BIRC5 island1 | LE | ttgagtcctggagttcaagaccagTTTTTaggcataggacccgt | 147 |
| | | gtct | |
| BIRC5 island1 | LE | gcctgtaatcccaacactttgagaTTTTTaggcataggacccgt | 148 |
| | | gtct | |
| BIRC5 island1 | LE | gcgccccctcgactgctTTTTTaggcataggacccgtgtct | 149 |
| BIRC5 island1 | LE | tcgtccctgcccacacctaTTTTTaggcataggacccgtgtct | 150 |
| BIRC5 island1 | BL | gtctaaaaaaaaaaaaaaagtcagaca | 151 |
| BIRC5 island1 | BL | cctggccatcacggtgaaa | 152 |
| BIRC5 island2 | CE | ggagttgtagtcctcccgccTTTTTctcttggaaagaaagt | 153 |
| BIRC5 island2 | CE | gagtagaggcggggcggTTTTTctcttggaaagaaagt | 154 |
| BIRC5 island2 | CE | caaatctggcggttaatggcTTTTTctcttggaaagaaagt | 155 |
| BIRC5 island2 | CE | tccttgagaaagggctgccTTTTTctcttggaaagaaagt | 156 |
| BIRC5 island2 | CE | cggggtgcaggcgcagTTTTTctcttggaaagaaagt | 157 |
| BIRC5 island2 | CE | gtggcctcgctagggacagTTTTTctcttggaaagaaagt | 158 |
| BIRC5 island2 | LE | ggtcgcggtgcacccagTTTTTaggcataggacccgtgtct | 159 |
| BIRC5 island2 | LE | gcgtggctctgcccgtTTTTTaggcataggacccgtgtct | 160 |
| BIRC5 island2 | LE | cctcttaggcggtccacccTTTTTaggcataggacccgtgtct | 161 |
| BIRC5 island2 | LE | tgtcgggagcgcacgcTTTTTaggcataggacccgtgtct | 162 |
| BIRC5 island2 | LE | caacgggtcccgcgattTTTTTaggcataggacccgtgtct | 163 |
| BIRC5 island2 | LE | cttgaatgtagagatgcggtggTTTTTaggcataggacccgtgt | 164 |
| | | ct | |
| BIRC5 island2 | LE | ccctccaagaagggccagttTTTTTaggcataggacccgtgtct | 165 |
| BIRC5 island2 | LE | gggcagtctcacccgctcTTTTTaggcataggacccgtgtct | 166 |
| BIRC5 island2 | LE | ggggaccccaggaggccTTTTTaggcataggacccgtgtct | 167 |
| BIRC5 island2 | BL | cgcggggtgtgccg | 168 |
| BIRC5 island2 | BL | cccgcggccttctgg | 169 |
| BIRC5 island2 | BL | gcgccgcggggca | 170 |
| BIRC5 island2 | BL | ccgccgccacctctgc | 171 |
| BIRC5 island2 | BL | ggggcacccatgccg | 172 |
| BIRC5 island2 | BL | aggcagggggcaacgtc | 173 |
| BIRC5 island2 | BL | ggcaaggcgggcgtg | 174 |
| IRF7 island1 | CE | tggggcttccgccagcTTTTTctcttggaaagaaagt | 175 |
| IRF7 island1 | CE | gatggacctcaccgccgTTTTTctcttggaaagaaagt | 176 |
| IRF7 island1 | CE | cgccgctccttggtcagTTTTTctcttggaaagaaagt | 177 |
| IRF7 island1 | CE | cacgtccagggtgggcgTTTTTctcttggaaagaaagt | 178 |
| IRF7 island1 | CE | ttattctgcctcgggtcggTTTTTctcttggaaagaaagt | 179 |
| IRF7 island1 | CE | ggtttcgggagccttgctTTTTTctcttggaaagaaagt | 180 |
| IRF7 island1 | LE | gtacccgccctccggcTTTTTaggcataggacccgtgtct | 181 |
| IRF7 island1 | LE | cgccaaaccagacagccttTTTTTaggcataggacccgtgtct | 182 |
| IRF7 island1 | LE | cctccgtcccgatgtcctTTTTTaggcataggacccgtgtct | 183 |
| IRF7 island1 | LE | cgttgagaacgaccacgtctgTTTTTaggcataggacccgtgtc | 184 |
| | | t | |
| IRF7 island1 | LE | cggagtcactggcgccatcTTTTTaggcataggacccgtgtct | 185 |
| IRF7 island1 | LE | ccctcatcgccgctgcTTTTTaggcataggacccgtgtct | 186 |
| IRF7 island1 | LE | tcaccaccgggcgcatTTTTTaggcataggacccgtgtct | 187 |
| IRF7 island1 | LE | gggccacttagatgtaggagtcaTTTTTaggcataggacccgtg | 188 |
| | | tct | |
| IRF7 island1 | LE | cctccagtgcccgtgcgTTTTTaggcataggacccgtgtct | 189 |
| IRF7 island1 | BL | tccccgcgccctcg | 190 |
| IRF7 island1 | BL | cgtagggacccccacccc | 191 |
| IRF7 island1 | BL | gctgggggagagggtggag | 192 |
| IRF7 island1 | BL | aggctgggggagcgaga | 193 |
| IRF7 island2 | CE | ctcggtatggatctcttggcaTTTTTctcttggaaagaaagt | 194 |
| IRF7 island2 | CE | gcagcagacgccaggccTTTTTctcttggaaagaaagt | 195 |
| IRF7 island2 | CE | cttctgcaggggcagttaggTTTTTctcttggaaagaaagt | 196 |
| IRF7 island2 | CE | gccgggctcttacctctcaTTTTTctcttggaaagaaagt | 197 |
| IRF7 island2 | CE | cagggcgctcggggacTTTTTctcttggaaagaaagt | 198 |
| IRF7 island2 | CE | tctccgaacagcacgcgtTTTTTctcttggaaagaaagt | 199 |
| IRF7 island2 | LE | tgtagccaccgacgctgcTTTTTaggcataggacccgtgtct | 200 |
| IRF7 island2 | LE | cacaggtgtggactgagggctTTTTTaggcataggacccgtgtc | 201 |
| | | t | |
| IRF7 island2 | LE | ggccaggtgtcacaggtgtcTTTTTaggcataggacccgtgtct | 202 |
| IRF7 island2 | LE | gcggccacaggtcgtgtTTTTTaggcataggacccgtgtct | 203 |
| IRF7 island2 | LE | gggaggtaagggctcctgtcTTTTTaggcataggacccgtgtct | 204 |
| IRF7 island2 | LE | tggcggtcaggtgttataacagTTTTTaggcataggacccgtgt | 205 |
| | | ct | |
| IRF7 island2 | LE | ggagccaaggccattgctcTTTTTaggcataggacccgtgtct | 206 |
| IRF7 island2 | LE | tggcatctggagagggtggTTTTTaggcataggacccgtgtct | 207 |
| IRF7 island2 | LE | gagagtcagggccggctgTTTTTaggcataggacccgtgtct | 208 |
| IRF7 island2 | LE | gctgatctctccaaggagccacTTTTTaggcataggacccgtgt | 209 |
| | | ct | |
| IRF7 island2 | LE | cccctcatagcagccgctTTTTTaggcataggacccgtgtct | 210 |
| IRF7 island2 | BL | gtgcccggccgcg | 211 |
| IRF7 island2 | BL | ggggctgccctgcgg | 212 |
| BRCA1 island1 | CE | agcagcctgggttagctaggTTTTTctcttggaaagaaagt | 213 |
| BRCA1 island1 | CE | ggcgggaagctggtaaggaTTTTTctcttggaaagaaagt | 214 |
| BRCA1 island1 | CE | agcggacggtctttgcattTTTTTctcttggaaagaaagt | 215 |
| BRCA1 island1 | CE | agatagcggcagagctggcTTTTTctcttggaaagaaagt | 216 |
| BRCA1 island1 | CE | ttcgggttcagcttgcttttTTTTTctcttggaaagaaagt | 217 |
| BRCA1 island1 | CE | gcagtgagcgccgaatttgTTTTTctcttggaaagaaagt | 218 |
| BRCA1 island1 | LE | ggtggggtcacgtcacactaaTTTTTaggcataggacccgtgtc | 219 |
| | | t | |
| BRCA1 island1 | LE | ccatgttagattcaccccacagTTTTTaggcataggacccgtgt | 220 |
| | | ct | |
| BRCA1 island1 | LE | gggactagttactgtctttgtccgTTTTTaggcataggacccgt | 221 |
| | | gtct | |
| BRCA1 island1 | LE | gggtggtcgttttgagggaTTTTTaggcataggacccgtgtct | 222 |
| BRCA1 island1 | LE | gcaatcgccaccagtcaatgTTTTTaggcataggacccgtgtct | 223 |
| BRCA1 island1 | LE | gccccgtctccgtcgacTTTTTaggcataggacccgtgtct | 224 |
| BRCA1 island1 | LE | tcagccccagtgtttgttatttTTTTTaggcataggacccgtgt | 225 |
| | | ct | |
| BRCA1 island1 | LE | cgcactcgtagttccaccccTTTTTaggcataggacccgtgtct | 226 |
| BRCA1 island1 | LE | cgctctggcccatgtctgTTTTTaggcataggacccgtgtct | 227 |
| BRCA1 island1 | LE | cctggggcaggggaaatgTTTTTaggcataggacccgtgtct | 228 |
| BRCA1 island1 | LE | cagtggcctgcggggacTTTTTaggcataggacccgtgtct | 229 |
| BRCA1 island1 | BL | gccgcctccccaggg | 230 |
| BRCA1 island1 | BL | ggcgaaaacgcggagaaac | 231 |
| BRCA1 island1 | BL | caagtggtaagagccaatcttctt | 232 |
| BRCA1 island2 | CE | tctcagcgagctcacgccTTTTTctcttggaaagaaagt | 233 |
| BRCA1 island2 | CE | agcgcaggggcccagtTTTTTctcttggaaagaaagt | 234 |
| BRCA1 island2 | CE | cagagggtgaaggcctcctgTTTTTctcttggaaagaaagt | 235 |
| BRCA1 island2 | CE | gccccctgtccctttcccTTTTTctcttggaaagaaagt | 236 |
| BRCA1 island2 | CE | cgcctctcaggttccgccTTTTTctcttggaaagaaagt | 237 |
| BRCA1 island2 | CE | gcccccttcctgatcctcaTTTTTctcttggaaagaaagt | 238 |
| BRCA1 island2 | LE | gcgcagtcgcagttttaatttaTTTTTaggcataggacccgtgt | 239 |
| | | ct | |
| BRCA1 island2 | LE | tgtcccccgtccaggaagTTTTTaggcataggacccgtgtct | 240 |
| BRCA1 island2 | LE | tatctgagaaaccccacagccTTTTTaggcataggacccgtgtc | 241 |
| | | t | |
| BRCA1 island2 | LE | gggactctactacctttacccagagTTTTTaggcataggacccg | 242 |
| | | tgtct | |
| BRCA1 island2 | LE | gtaccccagagcatcacttggTTTTTaggcataggacccgtgtc | 243 |
| | | t | |
| BRCA1 island2 | LE | aatccactctcccacgccaTTTTTaggcataggacccgtgtct | 244 |
| BRCA1 island2 | LE | acccatctgtcagcttcggaTTTTTaggcataggacccgtgtct | 245 |
| BRCA1 island2 | LE | ccagggttcacaacgccttaTTTTTaggcataggacccgtgtct | 246 |
| BRCA1 island2 | LE | gcgcttccctcgcgacTTTTTaggcataggacccgtgtct | 247 |
| BRCA1 island2 | LE | tcccccacggacactcagtTTTTTaggcataggacccgtgtct | 248 |
| BRCA1 island2 | BL | cctaccccccgtcaaagaat | 249 |
| BRCA1 island2 | BL | ctacaaactgcccccctcc | 250 |

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method for detecting methylation status of one or more nucleic acids, comprising:
contacting a sample of nucleic acid comprising or suspected of comprising one or more methylated nucleic acids with a methylation binding protein (MBP);
forming one or more methylated nucleic acid-MBP complexes;
isolating the methylated nucleic acid-MBP complexes; and
detecting the presence of the one or more methylated nucleic acids in the isolated methylated nucleic acid-MBP complexes, by a technique other than nucleic acid sequencing or target-specific PCR amplification.

2. The method of claim 1, wherein the sample of nucleic acid comprises multiple different nucleic acid molecules with different sequences and different methylation patterns.

3. The method of claim 1, wherein the sample of nucleic acid comprises a plurality of genomic DNA fragments.

4. The method of claim 3, wherein at least one of the plurality of genomic DNA fragments contains a methylated CpG island wherein at least one of the cytosine residues is methylated at the 5 position.

5. The method of claim 1, wherein the methylated nucleic acid-MBP complexes are isolated from other nucleic acids in the sample by using a filter column in which a membrane retains the nucleic acid-MBP complexes.

6. The method of claim 1, wherein the methylated nucleic acid-MBP complexes are isolated from other nucleic acids in the sample by binding the methylated nucleic acid-MBP complexes to a nitrocellulose membrane and washing the other nucleic acids away from the membrane-bound methylated nucleic acid-MBP complexes.

7. The method of claim 1, wherein the MBP comprises a methyl-CpG binding domain from mouse or human methyl CpG binding protein 2 (MeCP2) or a homolog thereof.

8. The method of claim 1, wherein the presence of the methylated nucleic acids in the isolated methylated nucleic acid-MBP complexes is detected with a nucleic acid hybridization array on which different nucleic acid hybridization probes with predetermined sequences are immobilized in discrete, different positions.

9. The method of claim 1, comprising simultaneously amplifying the one or more methylated nucleic acids from the isolated methylated nucleic acid-MBP complexes to provide one or more amplified nucleic acids.

10. The method of claim 9, comprising:
contacting the amplified nucleic acids with a nucleic acid hybridization array, on which array different nucleic acid hybridization probes with predetermined sequences are immobilized at discrete, different positions;
hybridizing the amplified nucleic acids with complementary nucleic acid hybridization probes, thereby capturing different amplified nucleic acids at different positions on the array; and
determining which positions on the array have an amplified nucleic acid hybridized thereto, thereby determining which methylated nucleic acids were present in the sample.

11. The method of claim 10, comprising incorporating biotin into the amplified nucleic acids during the amplifying step; wherein detecting which positions on the array have an amplified nucleic acid hybridized thereto comprises binding a streptavidin-conjugated horseradish peroxidase enzyme to the biotin and then detecting a luminescent product of the enzyme.

12. The method of claim 1, wherein detecting the presence of the one or more methylated nucleic acids in the isolated methylated nucleic acid-MBP complexes comprises:
providing a pooled population of particles, the population comprising one or more subsets of particles, the particles in each subset being distinguishable from the particles in the other subsets, and the particles in different subsets having associated therewith different nucleic acid hybridization probes with predetermined sequences;
contacting the one or more methylated nucleic acids from the isolated methylated nucleic acid-MBP complexes, or complements or copies thereof, with the pooled population of particles;
hybridizing the one or more methylated nucleic acids, or the complements or copies thereof, with complementary nucleic acid hybridization probes, thereby capturing different methylated nucleic acids, or complements or copies thereof, to different subsets of particles; and
detecting which subsets of particles have nucleic acid captured on the particles, thereby indicating which methylated nucleic acids were present in the sample.

13. The method of claim 1, wherein detecting the presence of the one or more methylated nucleic acids in the isolated methylated nucleic acid-MBP complexes comprises:
a) capturing the methylated nucleic acids from the complexes on a solid support;
b) providing one or more subsets of m label extenders, wherein m is at least two, wherein each subset of m label extenders is capable of hybridizing to one of the methylated nucleic acids;
c) providing a label probe system comprising a label, wherein a component of the label probe system is capable of hybridizing to the label extenders;
d) hybridizing each methylated nucleic acid captured on the solid support to its corresponding subset of m label extenders;
e) hybridizing the label probe system to the label extenders; and
f) detecting the presence or absence of the label on the solid support.

14. The method of claim 13, wherein the methylation status of one nucleic acid is to be detected, wherein capturing the methylated nucleic acid on the solid support comprises hybridizing the methylated nucleic acid to n capture extenders, wherein n is at least two, and then hybridizing the capture extenders with a capture probe bound to the solid support.

15. The method of claim 13, wherein the methylation status of two or more nucleic acids is to be detected;
wherein capturing the methylated nucleic acids on the solid support comprises:
providing a pooled population of particles which constitute the solid support, the population comprising two or more subsets of particles, the particles in each subset being distinguishable from the particles in the other subsets, and the particles in each subset having associated therewith a different capture probe;
providing two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected subset of the particles; and
hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the subset of particles with which the capture extenders are associated;
and wherein detecting the presence or absence of the label on the solid support comprises identifying at least a portion of the particles from each subset and detecting the presence or absence of the label on those particles, thereby determining which subsets of particles have a methylated nucleic acid captured on the particles and indicating which of the methylated nucleic acids were present in the sample.

16. The method of claim 13, wherein the methylation status of two or more nucleic acids is to be detected;
wherein the solid support is a substantially planar solid support that comprises two or more capture probes, wherein each capture probe is provided at a selected position on the solid support;
wherein capturing the methylated nucleic acids on the solid support comprises:
providing two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected position on the solid support; and
hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the solid support at the selected position with which the capture extenders are associated; and
wherein detecting the presence or absence of the label on the solid support comprises detecting the presence or absence of the label at the selected positions on the solid support, thereby determining which selected positions have a methylated nucleic acid captured at that position and indicating which of the methylated nucleic acids were present in the sample.

17. The method of claim 13, wherein the label probe system comprises an amplification multimer and a plurality of label probes, wherein the amplification multimer is capable of hybridizing to a label extender and to a plurality of label probes, and wherein the label probe comprises the label.

18. The method of claim 13, wherein the label probe system comprises a preamplifier, an amplification multimer and a label probe; wherein the preamplifier is capable of hybridizing simultaneously to a label extender and to a plurality of amplification multimers; wherein the amplification multimer is capable of hybridizing simultaneously to the preamplifier and to a plurality of label probes; and wherein the label probe comprises the label.

19. A method for detecting methylation status of a plurality of genomic DNA fragments, the method comprising:
contacting a sample of nucleic acid comprising or suspected of comprising the plurality of genomic DNA fragments with a methylation binding protein (MBP);
forming methylated DNA-MBP complexes;
isolating the methylated DNA-MBP complexes; and
detecting, with a nucleic acid hybridization array on which different nucleic acid hybridization probes with predetermined sequences are immobilized in discrete, different positions, the presence of the methylated DNAs in the isolated methylated DNA-MBP complexes.

20. The method of claim 19, comprising simultaneously amplifying the methylated DNAs from the isolated methylated DNA-MBP complexes to provide one or more amplified DNAs; wherein detecting the presence of the methylated DNAs comprises:
contacting the amplified DNAs with the nucleic acid hybridization array;
hybridizing the amplified DNAs with complementary nucleic acid hybridization probes, thereby capturing different amplified DNAs at different positions on the array; and
determining which positions on the array have an amplified DNA hybridized thereto, thereby determining which methylated DNAs were present in the sample.

21. The method of claim 19, wherein the methylated DNA-MBP complexes are isolated from other nucleic acids in the sample by binding the methylated DNA-MBP complexes to a nitrocellulose membrane and then washing the other nucleic acids away from the membrane-bound methylated DNA-MBP complexes.

22. The method of claim 19, wherein the MBP comprises a methyl-CpG binding domain from mouse or human methyl CpG binding protein 2 (MeCP2) or a homolog thereof.

23. A kit for detecting one or more methylated nucleic acids, comprising:
a methylation binding protein (MBP);
a separation column for separating MBP-nucleic acid complexes from non-complexed nucleic acid; and
instructions for separating MBP-nucleic acid complexes from non-complexed nucleic acid by the separation column.

24. The kit of claim 23, comprising an array of predetermined, different nucleic acid hybridization probes immobilized on a surface of a substrate, wherein the hybridization probes are positioned in different defined regions on the surface.

25. The kit of claim 24, wherein each of the different nucleic acid hybridization probes comprises a different nucleic acid probe capable of hybridizing to a different region or fragment of a gene.

26. The kit of claim 25, wherein each of the different nucleic acid hybridization probes is capable of hybridizing to a different promoter region of a gene.

27. The kit of claim 24, wherein the array of predetermined, different nucleic acid hybridization probes comprises at least two different nucleic acid probes which are capable of separately hybridizing to at least two of SEQ ID NOs:1-82 or a complement thereof.

28. The kit of claim 23, wherein the separation column comprises a nitrocellulose membrane.

29. A method for detecting methylation status of one or more nucleic acids, the method comprising:
contacting a sample comprising or suspected of comprising one or more methylated nucleic acids with a methylation binding protein (MBP);
forming one or more methylated nucleic acid-MBP complexes;
isolating the methylated nucleic acid-MBP complexes;
providing a pooled population of particles, the population comprising one or more subsets of particles, the particles in each subset being distinguishable from the particles in the other subsets, and the particles in different subsets having associated therewith different nucleic acid hybridization probes with predetermined sequences;
contacting the one or more methylated nucleic acids from the isolated methylated nucleic acid-MBP complexes, or complements or copies thereof, with the pooled population of particles;
hybridizing the one or more methylated nucleic acids, or the complements or copies thereof, with complementary nucleic acid hybridization probes, thereby capturing different methylated nucleic acids, or complements or copies thereof, to different subsets of particles; and
detecting which subsets of particles have nucleic acid captured on the particles, thereby indicating which methylated nucleic acids were present in the sample.

30. A method for detecting methylation status of one or more nucleic acids, comprising:
contacting a sample comprising or suspected of comprising one or more methylated nucleic acids with a methylation binding protein (MBP);
forming one or more methylated nucleic acid-MBP complexes;
isolating the methylated nucleic acid-MBP complexes;
capturing the methylated nucleic acids from the isolated methylated nucleic acid-MBP complexes on a solid support;
providing one or more subsets of m label extenders, wherein m is at least two, wherein each subset of m label extenders is capable of hybridizing to one of the methylated nucleic acids;
providing a label probe system comprising a label, wherein a component of the label probe system is capable of hybridizing to the label extenders;
hybridizing each methylated nucleic acid captured on the solid support to its corresponding subset of m label extenders;
hybridizing the label probe system to the label extenders; and
detecting the presence or absence of the label on the solid support, thereby detecting the presence or absence of the methylated nucleic acids on the solid support and in the sample.

31. The method of claim 30, wherein the methylation status of one nucleic acid is to be detected, wherein capturing the methylated nucleic acid on the solid support comprises hybridizing the methylated nucleic acid to n capture extenders, wherein n is at least two, and hybridizing the capture extenders with a capture probe bound to the solid support.

32. The method of claim 30, wherein the methylation status of two or more nucleic acids is to be detected;
wherein capturing the methylated nucleic acids on the solid support comprises:
providing a pooled population of particles which constitute the solid support, the population comprising two or more subsets of particles, the particles in each subset being distinguishable from the particles in the other subsets, and the particles in each subset having associated therewith a different capture probe;
providing two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected subset of the particles; and
hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the subset of particles with which the capture extenders are associated;
and wherein detecting the presence or absence of the label on the solid support comprises identifying at least a portion of the particles from each subset and detecting the presence or absence of the label on those particles, thereby determining which subsets of particles have a methylated nucleic acid captured on the particles and indicating which of the methylated nucleic acids were present in the sample.

33. The method of claim 30, wherein the methylation status of two or more nucleic acids is to be detected;
wherein the solid support is a substantially planar solid support that comprises two or more capture probes, wherein each capture probe is provided at a selected position on the solid support;
wherein capturing the methylated nucleic acids on the solid support comprises:
providing two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected position on the solid support; and
hybridizing each of the methylated nucleic acids to its corresponding subset of n capture extenders and hybridizing the subset of n capture extenders to its corresponding capture probe, whereby the hybridizing the methylated nucleic acid to the n capture extenders and the n capture extenders to the corresponding capture probe captures the nucleic acid on the solid support at the selected position with which the capture extenders are associated; and
wherein detecting the presence or absence of the label on the solid support comprises detecting the presence or absence of the label at the selected positions on the solid support, thereby determining which selected positions have a methylated nucleic acid captured at that position and indicating which of the methylated nucleic acids were present in the sample.

34. The method of claim 30, wherein the label probe system comprises an amplification multimer and a plurality of label probes, wherein the amplification multimer is capable of hybridizing to a label extender and to a plurality of label probes, and wherein the label probe comprises the label.

35. The method of claim 30, wherein the label probe system comprises a preamplifier, an amplification multimer and a label probe; wherein the preamplifier is capable of hybridizing simultaneously to a label extender and to a plurality of amplification multimers; wherein the amplification multimer is capable of hybridizing simultaneously to the preamplifier and to a plurality of label probes; and wherein the label probe comprises the label.

36. A kit for detecting one or more methylated nucleic acids, comprising:
a) a methylation binding protein (MBP);
b) a nitrocellulose membrane;
c) i) 1) a solid support comprising a capture probe, and 2) a subset of n capture extenders, wherein n is at least two, wherein the subset of n capture extenders is capable of hybridizing to a methylated nucleic acid and is capable of hybridizing to the capture probe and thereby associating the capture extenders with the solid support;
ii) 1) a pooled population of particles, the population comprising two or more subsets of particles, a plurality of the particles in each subset being distinguishable from a plurality of the particles in every other subset, and the particles in each subset having associated therewith a different capture probe, and 2) two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected subset of the particles; or
iii) 1) a solid support comprising two or more capture probes, wherein each capture probe is provided at a selected position on the solid support, and 2) two or more subsets of n capture extenders, wherein n is at least two, wherein each subset of n capture extenders is capable of hybridizing to one of the methylated nucleic acids, and wherein the capture extenders in each subset are capable of hybridizing to one of the capture probes and thereby associating each subset of n capture extenders with a selected position on the solid support;
d) one or more subsets of m label extenders, wherein m is at least two, wherein each subset of m label extenders is capable of hybridizing to one of the methylated nucleic acids; and
e) a label probe system comprising a label, wherein a component of the label probe system is capable of hybridizing to the label extenders;
packaged in one or more containers.

37. The kit of claim 36, comprising a filter column comprising the nitrocellulose membrane.

38. A method for diagnosing a disease or condition associated with aberrant hypermethylation or aberrant hypomethylation, comprising:
contacting a sample of nucleic acid comprising methylated nucleic acid or suspected of comprising methylated nucleic acid with a methylation binding protein (MBP), wherein the sample of nucleic acid is derived from a sample of cells from a patient having or suspected of having a disease or condition associated with aberrant hypermethylation or aberrant hypomethylation;
forming a methylated nucleic acid-MBP complex;
isolating the methylated nucleic acid-MBP complex;
detecting levels of the methylated nucleic acid in the isolated methylated nucleic acid-MBP complex with a technique other than nucleic acid sequencing or target-specific PCR amplification; and
comparing levels of methylated nucleic acid with that of a reference sample containing nucleic acid derived from normal or healthy cells or from cells from a different sample, wherein an increase in the levels of methylated nucleic acid indicates that the patient has a disease or condition associated with aberrant hypermethylation or wherein a decrease in the levels of methylated nucleic acid indicates that the patient has a disease associated with aberrant hypomethylation.

39. The method of claim 38, wherein the patient has or is suspected of having a disease or condition associated with aberrant hypermethylation, wherein the disease or condition associated with aberrant hypermethylation is a hematological disorder or cancer.

40. A method for treating a disease or condition associated with aberrant hypermethylation, comprising:
contacting a sample of nucleic acid comprising methylated nucleic acid or suspected of comprising methylated nucleic acid with a methylation binding protein (MBP), wherein the sample of nucleic acid is derived from a sample of cells from a patient having a disease or condition associated with aberrant hypermethylation;
forming a methylated nucleic acid-MBP complex;
isolating the methylated nucleic acid-MBP complex;
detecting the presence of the methylated nucleic acid in the isolated methylated nucleic acid-MBP complex with a technique other than nucleic acid sequencing or target-specific PCR amplification;
comparing the pattern of methylated nucleic acid with that of a reference sample containing nucleic acid derived from normal or healthy cells or from cells from a different sample; and
treating the patient with a therapeutic agent that inhibits hypermethylation of DNA in the cells.


SEQUENCE LISTING (250 sequences)

SEQ ID NO: 1 - DNA, 1000 bp, Homo sapiens
ctctgaaagctgccacctgcgcattctgggagctcagaggggaccctgagggggaatgaggcctggaggatggaaccatcttcaggtagactgagaaggagcctggatctcacttccaaacacagtctggagctcataggtcagaggcctcaatgggagaaaagctaaaggaagagggtgcagaaaggagtttcagggaattggtggctatgtgactttgagcaaatctcacccctctctgagacttagtgttcccatctctatggtcctgtgtgtgtcacagagacatggtggggattaaattcgatcgtgaatatgaaagtgcttgggaaactccatggccctacctaaacatgagttatcctcacctgaaccaaggggggaagttacctggcaggattaggaaccccatcctcctgaacctttatgggctctgtcgaggctgaagcagccaggggctaaagccgtccttagcccctggaagggcactgtgaaagtggatctgatttgagaagccgtttcctgatgtgggcagccatgtgatgccagccccgaacaagagggggcagcctggagcctggaaaggtgccagtgcaggtggggcccacgcccagatttctcctgctgactgttctgatgattcacccccacatcccagcctttttacctttactgcagagccggaaagggtgtggggaagagaggagagggaggcaggtcttgggccctggtcccgccccctgctcctccccacccttctctgggcctggccacccagccaaaaggcaggccaagagcaggagagacacagagtccggcattggtcccaggcagcagttagcccgccgcccgcctgtgtgtccccagagccatggagagagccagtctgatccagaaggccaagctggcagagcaggccgaacgctatgaggacatggcagccttcatgaaaggcgccgtggagaagggcgaggagctctcctgcgaagagcgaaacctg

SEQ ID NO: 2 - DNA, 1000 bp, Homo sapiens
ctcgggagatgtgactgcctgagggcggtggtggtgtcagcgtccggggccgggggagggggtgtctcgggcagagacccccgggcttggggcagctgaggcggccgggcctcctctacacggggcccgccttccgctgtctgggccgcgagagtccttcgtcccttacagccccgccccggctttgggacactgcgggtggtctgtttcccccagcttgggacaccccgttttctgaggcgtggaagagcgtcgccccggagtaagctgcccgtgccgcgccccgacagcttccctcagccccaagccgccccttattccggatcccggccccaactttggccacggagcctcccattcaaatccctcccttgctgtcaaggggtctccccttcccccaaggtggctcccgcgagcctctaatgccctgacttcttccaatgtcacctacggcccccttagtctcagctcagccaaaaactttaatgcaaaggaaaagtctggattggttccacaggccttttaaaaagcggacttaaaagttgctggcaatgcattccttttcgtcagagtcgagggcaaactcgctgaaatctgggtgacccgtgtccttttccggagagcaaagcagagaagcgagagcggccactagttcggcaggaaatttgttggaagatgaagaagctaagatagggggttggtgacttccacaggaaaagttctggaggagtagccaaagaccatcagcgtttcctttatgtgtgagaattgaaatgactagcattattgacccttttcagcatcccctgtgaatatttctgtttaggtttttcttcttgaaaagaaattgttattcagcccgtttaaaacaaatcaagaaacttttgggtaacattgcaattacatgaaattgataaccgcgaaaataattggaactcctgcttgcaagtgtcaacctaaaaaaagtgcttccttttgttatggaagatgtctttctgtg

SEQ ID NO: 3 - DNA, 995 bp, Homo sapiens
  misc_feature (4)..(4): n is a, c, g, or t
  misc_feature (740)..(740): n is a, c, g, or t
  misc_feature (788)..(788): n is a, c, g, or t
  misc_feature (792)..(792): n is a, c, g, or t
  misc_feature (807)..(807): n is a, c, g, or t
  misc_feature (853)..(853): n is a, c, g, or t
  misc_feature (856)..(856): n is a, c, g, or t
  misc_feature (880)..(880): n is a, c, g, or t
  misc_feature (907)..(907): n is a, c, g, or t
  misc_feature (909)..(911): n is a, c, g, or t
  misc_feature (913)..(914): n is a, c, g, or t
  misc_feature (954)..(954): n is a, c, g, or t
  misc_feature (971)..(971): n is a, c, g, or t
  misc_feature (974)..(974): n is a, c, g, or t
  misc_feature (988)..(988): n is a, c, g, or t
  misc_feature (992)..(992): n is a, c, g, or t
tccnatagggcgattgggccctctagatgcatgctcgagcggccgccagtgtgatggatatctgcagaattcgcccttgttctcggatcccgatcatgtaaattctcacagaggcctctgatcatacttttcaacttgtgcctatttattgaataaccaacatccttacagttaatattaaaatctttaagttgtgtggggttttttggaggggagggatgggcaattaccagcaaactccgcctcccccaaacctcacctaacccgaagctccccgcctcaggctcccggggagccaaggggtgggctgaggaacgcagcctacttttacccacctccctacctagtgctgggaagtgacggaaacggagacacccggctcctggggctgggctcggaggacccatcctgctttccctctagcagcctttccggagctcacctttcctcccctcacaccgccaaagccctgcctagcccttcaccgccgcctgcacccgcgccctcctccagccgacagccaatcacagtcttccacagctccgggtttacagaagtaacgctccttgggccctctggtcccgccccctccagaactgcttcccgcccttcgggctccttgtccaatcatgagcgcccgagtgctctttgatgcccgtcccctctacccgccctgccgaagacccgccttcttctccttaagcctgacggaatcacctgactcggaggcgctccctcanaaggaaggcaagaaggggcgtgtgggtgaaggggaggggcgccagaangaangtgggggatgccggnagcggggcgagcgggcgggggttgtcagtccgatctcgcgagaganganggaagcctgtggggagcccgtggnctttaaagtgccgttcaccctttcctncnnngnngctttgtaaaacccggttgtgctcagggctcgcgggtgancgaaaaggatcatgaantantgacctggaaaagngagnaac

SEQ ID NO: 4 - DNA, 1000 bp, Homo sapiens
tcctcccacttgtcacccttttcccccctccatcactcaaaatctttttacccacagtcttctttccctttcttctctccccaccatatttttgcaaaccttctctccttcctgctcatccccgttcccccctcacgaccctctcttacccccttccatctacccaaaaactttttccccaccatctttctgtgaaaccttctctccctcctgtttaccaccctgtttttccccctccatctaccccccaattttttttcccaacatcttttcctcaccgtctttatgcaatgacttctccggctcgccatccttttttccttttggcactaaccaccctctttacccttccatctatcccaaaactattttccccttcctacctttccagccacactacagtgtctgtcgccaccaactgcagggaggccagccacggtgcagcaggctacagcctccagtctgtcctggtcctctaagccgggctcggagcagctcggtgagcagacacagaagaacctggaacagcctgactcttcttcagccccatttatgtactgaagttatgcatatgcggttcgtggactacactttccaggattggataagagaaagcccggaggcctactctgattggactttgttatcatgttctgattggatgaaagtcttaggacaaccaattagagtatgaaaataaagtccaatcagagaaggcctagagattttctctcacccaatcagaacatgtagtccagaaaccatgcgcgtaaccccatgtgcatgccgagcaggcctcacgccagtttagggtctctggtatctcccgctgagctgctctgttcccggcttagaggaccaggagaagggggagttggaggctggagcctgtaacaccgtggctcgtctcgctctggatggtggtggcaacagagatggcagcgcagctggagtgttaggagggcggcctgagcggtaggagtggggctggagcagtaag

SEQ ID NO: 5 - DNA, 904 bp, Homo sapiens
  misc_feature (704)..(704): n is a, c, g, or t
  misc_feature (760)..(760): n is a, c, g, or t
  misc_feature (778)..(778): n is a, c, g, or t
  misc_feature (814)..(814): n is a, c, g, or t
  misc_feature (830)..(830): n is a, c, g, or t
  misc_feature (857)..(857): n is a, c, g, or t
  misc_feature (874)..(874): n is a, c, g, or t
  misc_feature (876)..(876): n is a, c, g, or t
  misc_feature (881)..(881): n is a, c, g, or t
  misc_feature (889)..(889): n is a, c, g, or t
ggcgattgggccctctagatgcatgctcgagcggccgccagtgtgatggatatctgcagaattcgcccttgaaatccactctcccacgccagtaccccagagcatcacttgggccccctgtccctttcccgggactctactacctttacccagagcagagggtgaaggcctcctgagcgcaggggcccagttatctgagaaaccccacagcctgtcccccgtccaggaagtctcagcgagctcacgccgcgcagtcgcagttttaatttatctgtaattcccgcgcttttccgttgccacggaaaccaaggggctaccgctaagcagcagcctctcagaatacgaaatcaaggtacaatcagaggatgggagggacagaaagagccaagcgtctctcggggctctggattggccacccagtctgcccccggatgacgtaaaaggaaagagacggaagaggaagaattctacctgagtttgccataaagtgcctgccctctagcctctactcttccagttgcggcttattgcatcacagtaattgctgtacgaaggtcagaatcgctacctattgtccaaagcagtcgtaagaagaggtcccaatcccccactctttccgccctaatggaggtctccagtttcggtaaatatgagtaataaggattgttgggggggtggagggaaataattatttccagcatgcnttgcggaatgaaaggtcttcgccacagtgttccttagaaactgtagtcttatgganaggaacatccaataccanagcgggcacaattctcacgggaaatccagtggatanattggagacctgtgcncgcttgtacttgtcaacagtttatggnactggagtgttatgttnangggcnatttccancacactggcgggccg

SEQ ID NO: 6 - DNA, 1000 bp, Homo sapiens
ggagtggcggctgaagaagccagggtcacaatgtctctgggataaggttcttgtggaaactcacctccctccggaatttgcattctccggggaggggacagggctcccagaaagctgtctcccagtccagactgtcgcccccctctccctccctactcaaggtctaactcgggtccctcgcctgcttcctgtgtttacgcggcgctttagtctcccggactcgcagggtgagccccagccctgactggagcgagacagcagccgcgagcgcagccccactcgcgggccggggcgactggggctggcgcgaggcgcacggagctcaccagctcgcccctccctctcctgggacaggagggggctgactggggtggcggggtccgggaaggggggctggctctcatcaattctgctgccacctcctctgccgcctgtcgggaggcgggcgggggtggggcgggagcgcaggctaggattgagactcttaagtcaggagaagtttgcgcacagcttcacagctgggagagcgcaggaaggcgccgggaaggtgagcctcctggactctggggaggtagaaagcaagccaggggaaagaacagttgtcttttagctgataatacaacctagacttgggtctgaaccacctaagacagatttaaagtgtcagaaaaccaggagaggggcggagagggaggactgagactaacgcagtttgctctcgcatcaaactaggaaagccagcccaccagcgtctgggtgggctgcgccgcgcggctggcggaccttcccgggttggagaagtgcgcacgtccgcacctcaccctgcggctgacatctcctgcccaggagatgggcgctgaagcttgagcgcctgagtccctggagccacacctgcgaacaccctttgcttctattgagctgtgcccagccgcccagtgacagaattccaggtaaggagcgtttggaaatgagcgggacttaacgatttggggtgtccaag

SEQ ID NO: 7 - DNA, 1000 bp, Homo sapiens
ttaaaaatacaaaaattagccgggggtggtggtgggtgcctgtagtcccagctactcgggaggctgaggcaggagaatcacctgaactcaggaggtggaggttgcagtgagtcaagatcgcaccactgtactgttgcctgggcaacgcaccgagactccgtctcaaaaaaaaaaaaaaaatgagagaacaggggagggtctagggctcagagctttggagaacagacctcagtagcaccaacactccaggatcaatgctacaaagacacgggttacaactaaactggagaacatggccaaggatgggaactcagcctgagcagggctgagccgagcagggctaagccaagtagggctgagccagaacacttcctccttttttctgaacaatctacctacatttcagctacagggctggctttacccagtccggcgggagggaggagagggctggtctgtgacttcagtgctgaggtttgatcaaggcaaagggaaacttcctattcccagaccctttgcaagaaagaatggcatattacttgccaccgacaggggttattattactaaatggagtcagtataaatgctttccaataaagcatgtccagcgctcgggctttagtttgcacgtccatgaattgtctgccacatccctcttctgaatggttggaaattgggcatctgttcctttaaacaggaaacatttcttgttcgagtgagtcatctctgttctgctttaggagtaaagtttaccctgcagttccttctgtggtgaagttttctctttctctcggagaccagattctgcctttctgctggagggaagtgttttcacaggttctcctccttttatcttttgtgttttttttcaagccctgctgaatttgctagtcaactcaacaggaagtgaggccatggagggaggcagaagagccagggtggttattgaaagtaaaagaaacttcttcctgggagcctttcccacccccttccctgctga

SEQ ID NO: 8 - DNA, 1090 bp, Homo sapiens
ggatagtgtaagtgacccagagacttggccaatgtgtctctgttaaatacatccacttttaagaaagttagtactgccaggcacagtggctcacgcctgtaatcccagcactttgggaggccgaggcgggtggatcacaaggtcaggagttcaagaccagcctggccaagatgatgaaaacctgtctctactaaaaaatacaaaaattagctgggtgtggtggtgggcacttgtaatcccagctactcgggaggctgaggcagagaattgcttgaacccaggaggcggaggttgcagtgagccgagatcatggcactctactccagcctgagcaacagagcaagactctatctcaaaaaaaaaaaaaaaaaagaaagaaagttattacttaatcaaaggagcaaggaaaaaaaaaggaagggggaatttttctttagaccaacttccttttcttgaacctaattctaccccccttggtgccaacagatgaggttcacaatctcttccacaaaacatgcagttaaatatctgaggatattcagggacttggatttggtggcaggagatcaacataaaccaagacaaggaagaagtcaaagaaatgaatcaagtagattctctgggatataaggtagggggattggggggttggatagtgcagagtatggtactggcctaaggcactgaggatcatccttttcccacacccaccagagaaggcttaggctcccgagtcaacagggcattcaccgcctggggcgcctgagtcatcaggacactgccaggagacacagaaccctagatgccctgcagaatccttcctgttacggtccccctccctgaaacatccttcattgcaatatttccaggaaaggaagggggctggctcggaggaagagaggtggggaggtgatcagggttcacagaggagggaactgaatgacatcccaggattacataaactgtcagaggcagccgaagagttcacaagtgtgaagcctggaagccggcgggtgccgctgtgtaggaaagaagctaaagcacttccagagcctgtccggagctcagaggttcggaagacttatcgacc

SEQ ID NO: 9 - DNA, 960 bp, Homo sapiens
  misc_feature (680)..(680): n is a, c, g, or t
  misc_feature (723)..(723): n is a, c, g, or t
cctctagatgcatgctcgagcggccgccagtgtgatggatatctgcagaattcgcccttgttctcggatcccgatcagatccctgacctccagtccggccttcttagaggaccccgttcctcaatactcgccctccgaggccctcggccgtcccctagacacgaccctgaccccagccactgtacccggcttattattccgcggcggccgcagcggcagctacaacaaccgcgtcgctctccgctcaatttccaagagccagctttgaagccaagtgcgagcagtttcaaactcaccgcgctaaagggccccggattcaccaatcgggtagcccgtagactttcaaagcagccaatcagagcccagctacgctgggcaggccttcccgggtggctagagcgcgaaagaaagaggaaagggcggctagagaaaaagcaggagggcgggcgccaactgagtgcgagcgcaagcgctctcctccagtcgggagagtgtcgtcctactgtttctagtcagcggagcaggaagctactgttcgctccgttcttcttttaaattttttctcccagcattggcacagttcaaatttattatactcaaaatagctcatcaaaaaagtgatattgtgtttacatcgagattccattactttcacttctaatacttagggttaggagtgnatagttatgtttttctaaatgcgtgattcgcgggctggctccnaggagcacatttcagtgaccttaagaaggaaatggaaaactcaaaagaccgcctcaaaaatgtaaaggaaaatttattatttatatcgctgtgctttgtttctacctcatttttgaatttaatattaaattattttattatttacattttgtttattatacaattaaaaacatttgaaatgtattaaattttaaaatattttcacatcagaattttaaatatatagagagaggcatg

SEQ ID NO: 10 - DNA, 1102 bp, Homo sapiens
actcatattcccttccccctttataattacgaaaaatgcaaggtattttcagtaggaaagagaaatgtgagaagtgtgaaggagacaggacagtatttgaagctggtctttggatcactgtgcaactctgcttctagaacactgagcactttttctggtctaggaattatgactttgagaatggagtccgtccttccaatgactccctccccattttcctatctgcctacaggcagaattctcccccgtccgtattaaataaacctcatcttttcagagtctgctcttataccaggcaatgtacacgtctgagaaacccttgccccagacagccgttttacacgcaggaggggaaggggaggggaaggagagagcagtccgactctccaaaaggaatcctttgaactagggtttctgacttagtgaaccccgcgctcctgaaaatcaagggttgagggggtagggggacactttctagtcgtacaggtgatttcgattctcggtggggctctcacaactaggaaagaatagttttgctttttcttatgattaaaagaagaagccatactttccctatgacaccaaacaccccgattcaatttggcagttaggaaggttgtatcgcggaggaaggaaacggggcgggggcggatttctttttaacagagtgaacgcactcaaacacgcctttgctggcaggcgggggagcgcggctgggagcagggaggccggagggcggtgtggggggcaggtggggaggagcccagtcctccttccttgccaacgctggctctggcgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagc

SEQ ID NO: 11 - DNA, 1000 bp, Homo sapiens
acaaggaacacatcctgggccggtaattacgcaaagcattatctcctcttacctccttgcagatttttttttctctttcagtacgtgtcctaagatttctgtgccacccttggagttcactcacctaaacctgaaactaataaagcttggttcttttctccgacacgcaaaggaagcgctaaggtaaatgcatcagacccacactgccgcggaacttttcggctctctaaggctgtattttgatatacgaaaggcacattttccttcccttttcaaaatgcaccttgcaaacgtaacaggaacccgactaggatcatcgggaaaaggaggaggaggaggaaggcaggctccggggaagctggtggcagcgggtcctgggtctggcggaccctgacgcgaaggagggtctaggaagctctccggggagccggttctcccgccggtggcttcttctgtcctccagcgttgccaactggacctaaagagaggccgcgactgtcgcccacctgcgggatgggcctggtgctgggcggtaaggacacggacctggaaggagcgcgcgcgagggagggaggctgggagtcagaatcgggaaagggaggtgcggggcggcgagggagcgaaggaggagaggaggaaggagcgggaggggtgctggcgggggtgcgtagtgggtggagaaagccgctagagcaaatttggggccggaccaggcagcactcggcttttaacctgggcagtgaaggcgggggaaagagcaaaaggaaggggtggtgtgcggagtaggggtgggtggggggaattggaagcaaatgacatcacagcaggtcagagaaaaagggttgagcggcaggcacccagagtagtaggtctttggcattaggagcttgagcccagacggccctagcagggaccccagcgcccgagagaccatgcagaggtcgcctctggaaaaggccagcgttgtctccaaactttttttcaggtgagaaggtggcca

SEQ ID NO: 12 - DNA, 1000 bp, Homo sapiens
taaccatttaacaagaaagcagagtgatgttagattatagcaagatactgttgactgtagaaggctctgaggctagagagctgctttctataaaacagagtgatcatatattagaagaggtgttaaagacatgttcacaccaagctgagacttcctccttgataccaccaggaggatgggcagagactggaaaagacactaactttctccctatgggagtcagtattatttagcatcactttggcgggtcaccccaaaccatctgactacaagggtaccatatttgggttaacactcttttggtataatttatgttttagtccaatgtcttgggatgaaaatgacaggtgggccacttatgatctccagagaaattcagggcaatttggtgtgggagtaggcatggtagaggagagcagcatctaagaagtccccagcagaggctctcagcttgtcttgaggcatctgggcggagggctatgatactggccccatcctgcagaaggtggcagatattggcagctggcaccagtgcggttccattgtgatcatcatttctgaacgtcagactgttgaaggttcccccaacagactttctgtgcaactttctgtcttcaccaaattcagtccacagtaaggaagtgaaattaatttcagaggtgtggggagggcttaagggagtgtggtaaaattagagggtgttcagaaacagaaatctgaccgcttggggccaccttgcagggagagtttttttgatgatccctcacttgtttctttgcatgttggcttagcttggcgggctcccaactggtgactggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaa

SEQ ID NO: 13 - DNA, 1000 bp, Homo sapiens
taggaccagtattatgaggagaatttacctttcccgcctctctttccaagaaacaaggagggggtgaaggtacggagaacagtatttcttctgttgaaagcaacttagctacaaagataaattacagctatgtacactgaaggtagctatttcattccacaaaataagagttttttaaaaagctatgtatgtatgtgctgcatatagagcagatatacagcctattaagcgtcgtcactaaaacataaaacatgtcagcctttcttaaccttactcgccccagtctgtcccgacgtgacttcctcgaccctctaaagacgtacagaccagacacggcggcggcggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaatttgtggggggtacgaaaaggcggaaagaaacagtcatttcgtcacatgggcttggttttcagtcttataaaaaggaaggttctctcggttagcgaccaattgtcatacgacttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacaggtgagtacctggcg

SEQ ID NO: 14 - DNA, 1002 bp, Homo sapiens
cacgatggtttctgctcgaggatcacattctatccctccagagaagcaccccccttccttcctaatacccacctctccctccctcttcttcctctgcacacactctgcaggggggggcagaagggacgttgttctggtccctttaatcggggctttcgaaacagcttcgaagttatcaggaacacagacttcagggacatgacctttatctctgggtatgcgaggttgctattttctaaaatcaccccctcccttatttttcacttaagggacctatttctaaattgtctgaggtcaccccatcttcagataatctaccctacattcctggatcttaaatacaagggcaggaggattaggatccgttttgaagaagccaaagttggagggtcgtattttggcgtgctacacctacagaatgagtgaaattagagggcagaaataggagtcggtagttttttgtgggttgccctgtccggggcccctggcatgcagggctggatggagggagaggggtggggggtggcgggggaccgcgtttgaagttgggtcgggccagctgctgttctccttaataacgagaggggaaaaggagggagggagggagagattgaaaggaggaggggaggaccgggaggggaggaaaggggaggaggaaccagagcggggagcgcggggagagggaggagagctaactgcccagccagcttgcgtcaccgcttcagagcggagaagagcgagcaggggagagcgagaccagttttaaggggaggaccggtgcgagtgaggcagccccgaggctctgctcgcccaccacccaatcctcgcctcccttctgctccaccttctctctctgccctcacctctcccccgaaaaccccctatttagccaaaggaaggaggtcaggggaacgctctcccctccccttccaaaaaacaaaaacagaaaaacctttttccaggccggggaaagcaggagggagaggggccgccgggctggcc

SEQ ID NO: 15 - DNA, 1000 bp, Homo sapiens
ggactctaatgtgtattttacacttacagcacaattaatttgggactagctacatttcagctcaacaatagccaatagcatatgggatagcgcaaataaactctgcgtctctgttgcttctttgggtctcggagacctcaaccctttcttcagattgcaaaccttcttgccttcaagcctcggctccaacaccagtccggcagaggaacccagtctaatgaggtacgctcccttcctgccattctctattccattaacctgtttcgtggtaaacgtaggactgatcctccaaaattaccttattaattagcttacatatttattatctatctgtcccaccagaatgcaggtttccggaaggcagggatttaaaaaaatctgttttgttctatgtgattttcccataccaagcaccgtgcccggcacaagctgggatcccagtacacatctcgggacggaagaaccgtgtttccctagaacccagtcagagggcagcttagcaatgtgtcacaggtggggcgcccgcgttccgggcggacgcactggctccccggccggcgtgggtgtggggcgagtgggtgtgtgcggggtgtgcgcggtagagcgcgccagcgagcccggagcgcggagctgggaggagcagcgagcgccgcgcagaacccgcagcgccggcctggcagggcagctcggaggtgggtgggccgcgccgccagcccgcttgcagggtccccattggccgcctgccggccgccctccgcccaaaaggcggcaaggagccgagaggctgcttcggagtgtgaggaggacagccggaccgagccaacgccggggactttgttccctccgcggaggggactcggcaactcgcagcggcagggtctggggccggcgcctgggagggatctgcgccccccactcactccctagctgtgttcccgccgccgccccggctagtctccggcgctggcgcctatggtcggcctccgacagcgctccggag

SEQ ID NO: 16 - DNA, 1000 bp, Homo sapiens
atcatacgagggctttattttctgcttcaggaagaggccctatgttagcagccccagcctgcattcaggctgattgcagagtattttgctttttattttcatgtcttagtccctgtaccctcgccccttccccgcctctggtggtctccagagaacttcgtgtcccctcagcttctccctcctacatcctgcctacgtagagaagctcttgcttcattctgggaggttacgtgggctctcgcctacacaccgagagaaacaaacagtgtcaaacactcacagagagacgcgcagacacaaacggacccacacgggcaactcccgagacaaaacccacactcgatggatccacgcggccgtggaaacacctgccgccccagaaacactcaggtactcgcgacacacacagtacagtcacgcttaagggcaccaggattccgggtttgcgcgtatgcgcggtccctttggatgctcgtgcgcatagacacaacaccctacacgccccagacccacgaaactccctacggctcagccccagcccacccgggccgcccttccctcgaggcggcctcccgtctctcctcctctcgcttctcctcctcctccgcctaaagatgtacaaaacactcctcggaagcaaccccggcgttcagctcctccctccccgccccccggccgccgctcccccattcattttcggccgtcgccggctaagtccctcccccggcgtagcccggcctccgccgctccccgcccggagaccgcggcgcacttggacttccctctccattcgccagccgcctcgctcccggaccccacggctgcaaactgatctggcgcgcggggaggaggagagcgcaggcgagcgaacccgcgagagagggagagagcgagcgagcaacagcgagagcgagagcgagagagccgggaggcagagggagtagtgaccgccttccggagccgggattcatgcctgtcctcgggaccagcgaaggggact

SEQ ID NO: 17 - DNA, 1000 bp, Homo sapiens
ggagagtctcttgaacccggcaggcggaggttgcagtgagccgagatcgtgccactgcactccagcctgggcaagacagagcgagactccgtctcaaaaaatacaaacaaaacaaacaaacaaaaaattaggctgctagctcagtggctcatggctcacacctgaaatcctagcactttgggaggccaaggcaggaggatcgcttcagcccaggagttcgagaccaggctgggcaatacagggagacacagcgcccccactgcccctgtccgccccgacttgtctctctacaaaaaggcaaaagaaaaaaaaattagcctggcgtggtggtgtgcacctgtactcccagctactagagaggctggggccagaggaccgcttgagcccaggagttcgaggctgcagtgagctgtgatcgcaccactgcactccagcttgggtgaaagagtgagaccccatctccaaaacgaacaaacaaaaaatcccaaaaaacaaaagaactcagccaagtgtaaaagccctttctgatcccaggtcttagtgagccaccggcggggctgggattcgaacccagtggaatcagaaccgtgcaggtcccataacccacctagaccctagcaactccaggctagagggtcaccgcgtctatgcgaggccgggtgggcgggccgtcagctccgccctggggaggggtccgcgctgctgattggctgtggccggcaggtgaaccctcagccaatcagcggtacggggggcggtgcctccggggctcacctggctgcagccacgcaccccctctcagtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtaccccggatcccctgacttgcgaggg

SEQ ID NO: 18 - DNA, 1000 bp, Homo sapiens
ccgacaatgtaacataattgccaaagctttggttcgtgacctgaggttatgtttggtatgaaaaggtcacattttatattcagttttctgaagttttggttgcataaccaacctgtggaaggcatgaacacccatgtgcgccctaaccaaaggtttttctgaatcatccttcacatgagaattcctaatgggaccaagtacagtactgtggtccaacataaacacacaagtcaggctgagagaatctcagaaggttgtggaagggtctatctactttgggagcattttgcagaggaagaaactgaggtcctggcaggttgcattctcctgatggcaaaatgcagctcttcctatatgtataccctgaatctccgcccccttcccctcagatgccccctgtcagttcccccagctgctaaatatagctgtctgtggctggctgcgtatgcaaccgcacaccccattctatctgccctatctcggttacagtgtagtcctccccagggtcatcctatgtacacactacgtatttctagccaacgaggagggggaatcaaacagaaagagagacaaacagagatatatcggagtctggcacggggcacataaggcagcacattagagaaagccggcccctggatccgtctttcgcgtttattttaagcccagtcttccctgggccacctttagcagatcctcgtgcgcccccgccccctggccgtgaaactcagcctctatccagcagcgacgacaagtaaagtaaagttcagggaagctgctctttgggatcgctccaaatcgagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggactgcacttgctcccgtcgggtcgcccggcttcaccggacccg

SEQ ID NO: 19 - DNA, 1000 bp, Homo sapiens
gagaaagggagactaggggagaaaggtcactctagatttcgttcaattattgaaaatacggtgtatttactatgtgctgggcacttttctaggtgctagaaagactacagtgaccaaaacaaaaatccacatctgcagggatcttgcattctagtgagaaagtaagatggtaaaaaagataaatacgtaaattttatacaatgcttcgtaacgacaaatgctaaggagaaaaacagcacagaaaagacagaaaggaaaagagaaggggcgcatgtggtgcaattttgttaggatgccagggagggctgagcgtagtcgtaaatgaccacattatttgatggatcaagccagggactgcaagtctgtgtttctgagagacacataaagaaaagaaggcttaaggaatccagaaagatccagagtggggaaatgaaacgaaaagaaatccagccagtgggaagtcgtgaagggatagttaaacgcgttttgggaggaaagaaaaaagcaaaagtgcggtacagcctttcgttacacgtgaaaagaatcatgtttctttttctagttagaaaaagccaaagattgtgcgatttatgccccaaacccccttgtaaggggattctcacctcaacttgtcttctgtggtcagtgtttcccgcccctgaatcagggttactgtcactatggctttcaattggcccggcgtaggcgcatgctctgcgcgtattggcctccgctcctgtccccagacaagcggccatcttgggtcccgcccctaccgtggggtcttctgggaattgcagtccccgctctgctctgtccggtcacaggactttttgccctctgttcccgggtccctcaggcggccacccagtgggcacactcccaggcggcgctccggccccgcgctccctccctctgcctttcattcccagctgtcaacatcctggaaggtaggggcggggaggcaagcccaagtggaatactgtttctggggcgcggg

SEQ ID NO: 20 - DNA, 1000 bp, Homo sapiens
aatttaacgacctcgatagagcgcagtcaagtttggtgaacagaatatgtctctgaactagaggagtcctcacacaaggagtagggtcagaccccgcagtggaggaggagggaggagtagaaacagtccagctcgccgcccaagtaacctgggtcctgaatcggcccgccttggccagtgctccagaagcgcggagcaggaacgggctggggcccaaaaaagaggggggagcctgaacgtccgggggaagtttcggaggcggcggaacgcccacggatggaaccctgtctttggggaaaaggaccacacctgtcagcagagtccgtcagacgtgagaagggtgggagcggcggactgtgaacgctggtagggccccggcgctccgagaaagtcccagtttcgcggtcgcccttccctaccacgcttccggcttccggtgtcatagctgtgggatccggaagtaaaaacacaagccccgcccccgagaactcgggaagccggcgagaagtgtgaggccgcggtagggccgcatcccgctccggagagaagtctgagtccgccaggctctgcaggcccgcggaagctcggtaatgataagcacgccggccactttgcagggcgtcaccgcctacacgccccctcgtctctcggacggcggcgtctagcctcggggcgctcggccgccccgccctctccgggggaggaatcaagaagagactgcccaatagggccggcttgacccgcgaacaggcgagggttcccgggggagtggcgcggcagaaggccccgcccaggagccgagggacagcccagaggaggcgtggccacgctgccggcggaagtggagccctccgcgagcgcgcgaggccgccggggcaggcggggaaaccggacagtaggggcggggcctggccggcgatggggattcgggagcactacgcggagctgcacccgtgcccgccggaattggggatgcagagcagcggcagcgggtatggca

SEQ ID NO: 21 - DNA, 1000 bp, Homo sapiens
attgatttaaagaaaactgtccttgacttaccagtgtgtaagtccatgaaagcataattctgttgaaagcatatattgttaatgggtgttgggaaccgtgcactttccgctgctgtgggagcatgtccttggaggtacctttcatctgttttctcaactccaaacatcttaggaccatgggttgtgactggtaggactatgtatcttgctgctttcaagacggagtatattttcacgtggtgtcactctggctgtcctgtttccctaatactgtcacttcaccctctgcgattctgatgctacaaatgatagatatcgttttagcattttcttacgggtcctagcgattctattcatttttctttcagtctctttctctgacttgttcacattgaacaatttccttttgggataggttgctatttctgttttcgcaggtggtttacctgtcttcccagccagtcacagtggtccttgtccccatggtgggtccggggcaagagagggccctgggttgggggtggggttcagttgaagatggggtgagttttgaggggagcactacttgagtcccagaggcataggaaacagcagagggaggtgggattcccttatcctcaatgaggatgggcatggagggtttggggcgtggcgctgggaacggcagccctccccagcccacagccgcgcatgctccctgggctcccgcctcagtgcgcatgttcactgggcgtcttctgcccggccccttcgcccacgtgaagaacgccagggagctgtgaggcagtgctgtgtggttcctgccgtccggactctttttcctctactgagattcatctggtaggtgtgcaggccagtcatcccgggggctgaagtgtgagtgagggtggagagggcctcgggtgggtcaggcgggtcccgcttcctggtctgtggcctccgagggagaagggccacgaggtcgtcctccttcccttcacaggctgcgaggccaccggcg

SEQ ID NO: 22 - DNA, 915 bp, Homo sapiens
  misc_feature (431)..(432): n is a, c, g, or t
  misc_feature (754)..(754): n is a, c, g, or t
  misc_feature (836)..(836): n is a, c, g, or t
  misc_feature (855)..(855): n is a, c, g, or t
  misc_feature (868)..(868): n is a, c, g, or t
  misc_feature (890)..(890): n is a, c, g, or t
  misc_feature (892)..(892): n is a, c, g, or t
  misc_feature (900)..(901): n is a, c, g, or t
aattcgcccttggaagatctaccagtaccaacctgggtagcgaagagcagagaggaggaggaggcggcggcgtacgacctgctcggtcagattgcgttgctcgctctgtctcgctctccctccgtctctctctcttcttctctctctctctccctctctcagtatttttttttttttttacagggaatgcattctttctgaaagtatcaagacggcgccaggcagctcagtgttcgcagacagctgtggcgcgacgcaacttaagggggttctagtgtcatccgcgccgggggggaggagcctggcgctggcgagtaggggacaggatccccggcacaaggaaactgcaacccaaacccgctccaggacttctccccccgccccgcgcacccccgcccctcctcccgcccctccactgaccggaaaggggnnccgcagagggcggccgccggcggaggggcggcgggcagggtgggcgaggcccgcggggcttgggggcggacgggagggaacgcgcgctctggccctttaaatgtggccgcggctcctgccaattcattcgggtcgggtggacgattccgtcccggtgcagccagcctgccccattcatgaagttcatttcgatgggcagaattttctttttcagacttttaaaaaataggcacgcatggatcattattaggatccaatgcagggtgtttgggagagcgcatcgatgtggggaaacgtgcgcgttaaattgatcagaaaaacnaaatgtttcatgtcaaggtattttgagatttgcctctcgggccgacttcctaagagggtgagtcatcggataaaggggagangcctttgactggagcgtcngcgtcaattttgntgtcattgtcacctctttcccnanccttctgnnctctcaagccccca

SEQ ID NO: 23 - DNA, 1000 bp, Homo sapiens
gctccaggaaccaacctggggaatgtgtgtaggggaagggcgggatagacagtgcccggagcagggaggcgctgaaagacaggaccaagcagcccggccaccagacccgttgtgggaacggaatttcctggcccccagggccacactcgcgtgggaagcatgtcgcggactctttaaggcgtcatctccctgtctctccgcccccgcctgggacaggccgggacgcccgggacctgacatttggaggctcccaacgtgggagctaaaaatagcagccccgggttactttggggcattgctcctctcccaacccgcgcgccggctcgcgagccgtctcaggccgctggagtttccccggggcaagtacacctggcccgtcctctcctctcagaccccactgtccagacccgcagagtttaagatgcttctgcagcccgggatcctagctggtgggcggagtcctaacacgtgggtgggcggggccttttgttccagggactcttttctcaaaacttcccagtcggaggctggcgggaacccgagaggcgtgtctcgccagccacgcggaggggcgtggcctcattggcccgccccaccaactccagccaaactctaaaccccaggcggagggggcgtggccttctggggtgtgcgggctcctggccaatgggtgctgtgaagggcgtggcccgcgggggcaggagcgaggtggcgggggcttctcgcgtcttttcccccagccccgctccaccagatccgcgggagccccactgctctccgggtccttggcttgtggctgtgggtcccatcgggcccgccctcgcacgtcactccgggacccccgcggcctccgcaggttctgcgctccaggccggagtcagagactccaggatcggttctttcatcttcgccgcccctgcgcgtccagctcttctaagacgagatgccgtcgggcttccaacagataggctccgaagtaggattcatcatgagggggcgg

SEQ ID NO: 24 - DNA, 990 bp, Homo sapiens
  misc_feature (7)..(7): n is a, c, g, or t
  misc_feature (810)..(810): n is a, c, g, or t
  misc_feature (819)..(819): n is a, c, g, or t
  misc_feature (938)..(938): n is a, c, g, or t
  misc_feature (949)..(949): n is a, c, g, or t
  misc_feature (970)..(970): n is a, c, g, or t
ggcgaantgggccctctagatgcatgctcgagcggccgccagtgtgatggatatctgcagaattcgcccttggaagatcttcccggccatcctgcttcgcagggagctaggagagcgcgggagagtggcagccggagcgagagcagtcccaggactcggcaagcctggcagtggccctgaggagcaagagacgtgctgctacccagccgctgcaaaagtttcctcgcagctacctgggcgctgggcgagggcgggaaccgcttggcggcgcggggcagggcggggctgactggggtggggcggggcgaggagggacggggcggggcgaggcgagccgcgcggccagggggcggtggcggttgtgcggccggtagccggcggggtgcgggggcgcggcgtggagcgcggcgggggccactggggcaccgcggcgcggggaccgggcgaaggcagtgcgagaggagggtgcggagcccgcgcggtggctcccggcagccgagcccagctgcccgctcgcagccgctctacacagggcgctctggcataactactgcagaggggctgcaggctcgagcgcgctgattggcttcccagcagccgtccgctctgactggctctgggagaagttccccagcctcactcctccttcccgccgctcattggcctacagcctggagggcttttccctttaggatttttgtctccttttcatccttcctgggggcaggtgggggtccctgacttaggtcctcctccgcttcgccacaggccttctttcagcttgtgccagctctttcctggccaccaganccacacaangtgttccttcacacaaaatccacctcctcattctactctctgaggagcttcccgcgaaccgttttcctaacgcagctctgaccgggttctcaaagccaacctgcaaatagtcgcgttcngggacagccanggaccgctgggcttttcacancctgcctcacctttgaatat

SEQ ID NO: 25 - DNA, 1000 bp, Homo sapiens
agttgtttcaattcacagctttagaattttggtaaaagaccacatgccagtaagttatctttttgttgaactggttgtttaatagaagaaaatgtaaactgcagagtgagaggatctggatcatactttgtaggttggtactttacaatttagggcataaaaacaaaccccaaacctcttgggatgataccacacaacatttttgcaccccctatgctgcctacttggatgttctttcttgtcttataagcttgatcaccaaggaaagaatgagtgccttaatttttctgaaaccatagtggacttaaatttttacacagagcctctaagtggattcagaattaatgggaaaaataaatcggcctcttacaggctgaaagcctcaaaatacattcctacagaagttgccagtttgtctttttcaatatgtataggatgaagttgagcgtggcgtagcatggattttgttagctcttctttgtgaagagtaaagttattgtggagggaaggccaagggaagagagtgtcctaaatttacaaaaatgtcctaaaggagaaaggctaataaattctttacaaatttggcttaagaagtagtattgtttgtatatgtcatgtcttcgctgtgcttagttagaagaagaggtaggaatgagtaaagatatcgaaattatagaaagggaaatggagaaagactgataatctattggttgtcagattattttgggtgtaaaagaagacattaggttgtaacttttaactaaatgcttaatagtgtgtttgttgccttttctttttaggtattgcactctcagtctcgccatgttgaagtcagaatggcctgtattcactatcttcgagagaacagagagaaatttgaagcggtaacttgtaatttcaaacatgtaatggtgtcttgacttggttttacattttggcttttagaagtgttctagtagaatttcacaggctggatcttaatgcgggttatgaaaataac

SEQ ID NO: 26 - DNA, 1000 bp, Homo sapiens
tctcagcaacacctccatgcactggtatacaaagtccccctcaccccagccgcgacccttcaaggccaagaggcggcagagcccgaggcctgcacgagcagctctctcttcaggagtgaaggaggccacgggcaagtcgccctgacgcagacgctccaccagggccgcgcgctcgccgtccgccacataccgctcgtagtattcgtgctcagcctcgtagtggcgcctgacgtcgcgttcgcgggtagctacgatgaggcggcgacagaccaggcacagggccccatcgccctccggaggctccaccaccaaataacgctgggtccactcgggccggaaaactagagcctcgtcgacttccatcttgcttcttttgggcgtcatccacattctgcgggaggccacaagagcagggccaacgttagaaaggccgcaaggggagaggaggagcctgagaagcgccaagcacctcctccgctctgcgccagatcacctcagcagaggcacacaagcccggttccggcatctctgctcctattggctggatatttcgtattccccgagctcctaaaaacgaaccaataggaagagcggacagcgatctctaacgcgcaagcgcatatccttctaggtagcgggcagtagccgcttcagggagggacgaagagacccagcaacccacagagttgagaaatttgactggcattcaagctgtccaatcaatagctgccgctgaagggtggggctggatggcgtaagctacagctgaaggaagaacgtgagcacgaggcactgaggtgattggctgaaggcacttccgttgagcatctagacgtttccttggctcttctggcgccaaaatgtcgttcgtggcaggggttattcggcggctggacgagacagtggtgaaccgcatcgcggcgggggaagttatccagcggccagctaatgctatcaaagagatgattgagaactggtacggagggagtcgagccgggct

SEQ ID NO: 27 - DNA, 1000 bp, Homo sapiens
tgggcccggggcgcagactctgggctggacactgggaggggggcgagaggctgaggggagaaggggaggcggacagaagagagagggagggagaaagggggagaagaggaaaaagagggaaagggacagacaggaaggaaaacagaccgagagagatcagttttgagatccaggaactgcttttaggaaaagtgaaggaggaaaagggaaagaaaaggaagaccccttcccaaccaaaatctttcctttcttctctcttttctgtcttctctttctccatctctcaaactctctcttcttccctctctctttattctccctctctcatctcctctcttcctctgctcctttctcctgctttaacagaacttatgtggctgggacgcagggccctcgggtgtcaaaactttgaagattaatggattactttgttaatgactgcaggcgtcagactgaggtgcttaaatgatttgtgaggtgcgaggcgtcttcccgacagtcccaaacaatgcgcggagtgtgcgggggaggcagagggcagccaccggcgggaccgacagcagggcttacactcgcgcacattcacacacacacacacacactcccaggcacacacactagatagatccttgcagatcaggaggcacgcaggcaccctcgcccccacgtactccgggacatccccacccacaccaacatatatgtatttttgctctgaaaaaagtgtaaataaagcctcgctggcccccaatgaggcgttccttcccgacttttttggatcaatcaaacagacagtggcttcttttgattaaagcccaaattgtcattgggcagaagcaatcatgtgacagccaattcggtccaatttcaaccttgtctccatgaattcaatagtttaatagtagcgcggtccccatacggctgtaatcagtgaattagaaaaaaaacaccctagcagcgatattctatgatagattttttttcctctgcgctcgccttt

SEQ ID NO: 28 - DNA, 1000 bp, Homo sapiens
tgtggcaacttgtgggtacggtttaactggaccacgctgagcttctgcagcgttggaacctcaagtttggggggactgggcgggcagggtcgcctgccacgcaggcccgagaaagaggagagtggtggagggggcgttctcacgcctggccccagggcacacggctgcgcccgccgcccggaaccccaccggggctgcaagcgtcctcggggtgggttgcggtgggagtaggggagctggggtgcgtggtggtaggtggggtgcgcggccgctccacctgcgcggaagggcagccgggcaaccggaccccgcggccacccgggggcccccagctccgagcatcccgccttggtcccggcggatcccagcctttccccagcccgtagccccgggacctccgcggtgggcggcgccgcgctgccggcgcagggagggcctctggtgcaccggcaccgctgagtcgggttctctcgccggcctgttcccgggagagcccggggccctgctcggagatgccgccccgggcccccagacaccggctccctggccttcctcgagcaaccccgagctcggctccggtctccagccaagcccaaccccgagaggccgcggccctactggctccgcctcccgcgttgctcccggaagccccgcccgaccgcggctcctgacagacgggccgctcagccaaccggggtggggcggggcccgatggcgcgcagccaatggtaggccgcgcctggcagacggacgggcgcggggcggggcgtgcgcaggcccgcccgagtctccgccgcccgtgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtgagtgcgggcagggatcggccgggccgcgcgccctcctcgcccccaggcggcagcaatacgcg

SEQ ID NO: 29 - DNA, 1000 bp, Homo sapiens
cggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcg

SEQ ID NO: 30 - DNA, 1000 bp, Homo sapiens
ccctgggaatattctctacactgtatttcaaggatttaatatgacaaaaagaatgtcaaataccttattaacaatgtagtatattgatgcatactgaagtactatttgggatatattggtttaaatacaatatattttaaaattatatttaccttttaaaaaaacttttattaatgaggctactagatcatttaaatttacctgtgtggcttgtattgtatttctactgggcagtgctgatctagagcaatttgaaacttgtggtagatattttactaaccaactctgatgaaggacttcctcaccaaattgttcttttaaccgcattctttccttgctttctggtcatttgcaagaaaaattttaaaaggctgcccctttgtaaaggtttgagaggccctagaatttcgtttttcacttgttcccaaccacaagcaaatgatcaatgtgctttgtgaatgaagagtcaacattttaccagggcgaagtggggaggtacaaaaaaatttccagtccttgaatggtgtgaagtaaaagtgccttcaaagaatcccaccagaatggcacaggtgggcataatgggtctgtctcatcgtcaaaggacccaaggagtctaaaggaaactctaactacaacacccaaatgccacaaaaccttagttattaatacaaactatcatccctgcctatctgtcaccatctcatcttaaaaaacttgtgaaaatacgtaatcctcaggagacttcaattaggtataaataccagcagccagaggaggtgcagcacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagc

SEQ ID NO: 31 - DNA, 1000 bp, Homo sapiens
gcgggtacgactcctatagggcgattgggccctctagatgcatgctcgagcggccgccagtgtgatggatatctgcagaattcgcccttggaagatctaaccaatccccaatgactgctacccatatcatcttggttccaactgtctgattaaattgaaaacaaagtggaaaataaatgaaaaagatattcctggggtctccaacattggacataaaatttagaaaagtgtagtaagctcggtagtccttctgcaaatgctgaattatgagcactccattcctgtgaaggaaatccatcttgaaaaagaggcaattctaaacatagagcaattggagctgaagtgctctgattcccaccgtttttatactgtgcctttgtggcatgtcgagccattactgcaacatgtgatgctgaccatctgtggagagggcacaccagccctcctctgctgaatagctcatctatttatgattttaattggtggcaaagagtgaagtacatgctgatctgtggcaattcgagggggaaatttggatagaaacacaatgaatttcttatgcaacctcccttttgtgcgaacagttggatcatgtttgtttgaaattttttgtacagttcatttcctccaaggtcagacattagcaatttctatgtttggtgaaaagactttgcaaataattattgcatgtcaaatagcccataaagccctgcattttaatttaagataggctgtggctctctattttattgggtctttgaggaaaatggttgaataaatatctgggtatgaaaaatatatgatatgacagattatgttctgatcactgatttaaaataagaatagttcaattttctttatccaagagaatgatagaatatatatggaacaggggaaagaaatgtgttgtttttttgactataagacagaaaagcagaaatgaaagtcttttggataattgaaatgtgttaggatcaaatcgtatctttattactaaaga

SEQ ID NO: 32 - DNA, 1000 bp, Homo sapiens
attcaataaaaaacaagcagggcgggtggtggggcactgactaggagggctgatttgtaagttggtaagactgtagctctttttcctaattagctgaggatgtgtttaggttccattcaaaaagtgggcattcctggccaggcatggtggctcacacctgtaatctcagagctttgggagactgaggtaggaggatcacttgagcccaggaatttgagatgagcctaggcaacatagtgagactcttatctctatcaaaaaataaaaataaaaatgagccaggcatggtgcggtggcacgcacctactgctaggggggctgaggtgggaggatcacttgagcctgggaggttgaggctgcagtgatccctgatcacaacattgcatttcagcctgggtgacagagtgagaccctgtctcagaaaaaaaaaaaaaaaagtcattcctgaaacctcagaatagacctaccttgccaagggcttccttatgggtaaggaccttatggacctgctgggacccaaactaggcctcacctgatacgacctgtccttctcaaaacacctaaacttgggagaacattgtcccccagtgctggggtaggagagtctgcctgttattctgcctctatgcagagaaggagccccagatcagcttttccatgacaggacagtttccaagatgccacctgtacttggaagaagccaggttaaaatacttttcaagtaaaactttcttgatattactctatctttccccaggaggactgcattacaacaaattcggacacctgtggcctctcccttctatgcaaagcaaaaagccagcagcagccccaagctgataagattaatctaaagagcaaattatggtgtaatttcctatgctgaaactttgtagttaattttttaaaaaggtttcattttcctattggtctgatttcacaggaacattttacctgtttgtgaggcattttttctcctggaagagaggtgctgattggc

SEQ ID NO: 33 - DNA, 1000 bp, Homo sapiens
aggcctaggggtgagagacacattcccctcgctgctcccaaagccagagcccaggctgggcgcccatgcccagaaccatcaagggatcccttgcggcttgtcagcactttccctaatggaaatacaccattaattcctttccaaatgttttaattgtgagagtatctgatattcttgactgaacaatgtaaaaaacccaaagggggctgcgcacggcggctctcgcctaaatcccagcactttgggaggccgaggtgggcagatcacctgaggtcgggagttcgacaccagcctgaccaacatagagaaaccccgtctctactaaaaatacaaaattagccgggcgtggtggttcatgcctgtaatcccagctactcgggaggcttaggtaggagaatcacttgaacccgggaggcggaggttgtggtgggccaagattgtgccaccgcactccagcctgggtaacaaaagcgaaactccatctcaaaaaaagaaacgcaaacggtgcagctgcccctttttcgaggcacgtccacctcccattacccacttccttttttttttgagactgagtcttgctctgtcccctgggctgtagtggagtggctccatctcggctcactgcagccactcccaacgccctccactcctccctactccgcgctggccggggcggggttccgctggtcgcatccaataataagaacaggcggcgcgcgcccttcccggaaactcccgcctggccaccataaaagcgccggccctccgcttccccgcgagacgaaacttcccgtcccggcggctctggcacccaggtactggggaccccagacccacgcggtgcaggccgggagcgagagcctccgtgggggctccgtgaccccggaggggtagagccaagagctgggggagcctgagagatgagggtcgggcggggagggaggcggaggcggaggcggaggcggggttccgcggagctgagaaccggacggggtgggat

SEQ ID NO: 34 - DNA, 1000 bp, Homo sapiens
aatttctggcagacatgtctccatcttctacctggcatattttacctgcctcagtgtaccccaggccgcttactagctttctgcatatctagacttcccctaatgcctccttcccgcttacgggagagcctcagactctggactcagctcccatgagctcctggacccctactcatttcttgcaatttaatgggtcatgcagctccacccactcaccccttttgatctctcccctcctccgtcctgtgaaaattccagtcccgcatccttctgagcccgggacccccagtcaattcctgggtcaggtgtctccttaaccctcccgatttacagtgcttaaccctcatttctgctttttggggtctcccaatggattgtcagtcctcctacccctctcgtattctgggtacctcaggggtttcttcgcacatactgggaccctcaccccacttgctgcgtaccaggtcctggtatttgtcccagtggactccagggaaatcatcctcctccctgaaacccctcactcatgtgcctgggccccccagcacctccttccatgcgtaccccgaggtcctttgagcccctccccctgcagccccgccgagccacccggcccgtggccgctgtttacaaggacacgcgcttcctgacagtgacgcgagccgcctcctccccttccccacgctcgaggaggggggcgcgggggcccggctccggcgacggccaatcggagcgcacttccgtggctgactagcgcggtataaaggcgtgtggctcaggctgagcggctgggaccttgagagcggccaggccagcctcggagccagcagggagctgggagctgggggaaacgacgccaggaaagctatcgcgccagagagggcgacgggggctcgggaagcctgacagggcttttgcgcacagctgccggctggctgctacccgcccgcgccagcccccgagaacgcgcgaccaggcacccagtccggtcaccgcagcg

SEQ ID NO: 35 - DNA, 1000 bp, Homo sapiens
ttcttacaaactccagaaaggtaggtgtaaataagagacatttgtaagaatgacagcacattaaatgtgtagatttcaaccttcagttattgcaatattccagtatcaagttggaggatgttatcagtctgatattttttcctcaaatgagagagagaaagaaagacacacaaacaacacagggagaaaaaaagcacacgttacagagagacaaaaagggagacagggaactgtgaatttggactcttgtgtcataagacaaattctagataacacgaccagaccttcaattgacatattgtgtttttgctaataaggtggaattctatgatgcgaaataactatatagtcttttctactgggatttaaatcattttatctgtttctggcttaacaggaaaaatacaaccatggaaaattatgatgatttatttaatacgattgctctatagtgttaataaaacctattaggtattttgcatattacatatcaaggagagtttgaatctcaggtagaaacaaaaaaaaatacatcaaaagttcctcatgtgagtgcagaattcaatcgtcccgtgcaggggtaagtgagtctgagatgtgttttgagcctggccgttgcgcatgatgtgaagtgacaagtctagtctgcagttttcagaaaccctcattcctcccttgactgattcaccacttgaacctcatatgacgtagaagaagcctacctatgtccccttcacatgttgtggtcaatgtgtcaactgcacgatccgggcccctcaccacatcctctgcaccggtcagtcgagccgagtcactgcgtcctggcagcagaagctgcaccatgtccatgtcacccacggtcatcatcctggcatgtcttggtgagtcctggaagggaaggagcaccagggttacactatgggcctgcagattgggtgtctccccagcagagagccatgttctgaagcaagtgagtggtgaggatgagttaattttcagt

SEQ ID NO: 36 - DNA, 1000 bp, Homo sapiens
cttgtgatgggttcaaaatatcaagaaagatagcaaaatatcacaagcctcctgacccgagaagattagcgttgaaagggtctgtcgtgtttgtttgggcctggggctaaattcccagcccaagtgctgaggctgataataatcggggcggcgatcagacagccccggtgtgggaaatcgtccgcccggtctccctaagtccccgaagtcgcctcccacttttggtgactgcttgtttatttacatgcagtcaatgatagtaaatggatgcgcgccagtataggccgaccctgagggtggcggggtgctcttcgcagcttctctgtggagaccggtcagcggggcggcgtggccgctcgcggcgtctccctggtggcatccgcacagcccgccgcggtccggtcccgctccgggtcagaattggcggctgcggggacagccttgcggctaggcagggggcgggccgccgcgtgggtccggcagtccctcctcccgccaaggcgccgcccagacccgctctccagccggcccggctcgccaccctagaccgccccagccaccccttcctccgccggcccggcccccgctcctcccccgccggcccggcccggccccctccttctccccgccggcgctcgctgcctccccctcttccctcttcccacaccgccctcagccgctccctctcgtacgcccgtctgaagaagaatcgagcgcggaacgcatcgatagctctgccctctgcggccgcccggccccgaactcatcggtgtgctcggagctcgattttcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggtacggagcggaccacccctcctgggcccc

SEQ ID NO: 37 - DNA, 1000 bp, Homo sapiens
gcagggactgatactgccgaacccaggagccaggcccgacccagcctcaggtccagcaggtcccgcctgtccacctgggccaggcctagagcccgggagcccctggctggtgggaggccacccgcaacccaccccacacgcagctccagctcccccaccaggcggggcgactaggacagggacagaacccgttgaacccaggagtgagatccggccccgggtcccgctgggccctcccgtccaccttggctggacctggcgcctgggagaccttggctggcgcgaggccacgcccaccagacatgcagttccagctaccccaccagctgggcgaccaggacagggacggaggctgctgagcccagttagaggcctgccccccggggtctgtcctgggcgctcccccaaggacggacagggcaggcagggtccgggacgatggccgcacagtcccggccccgtgttcccaggcccgtcttgctcctcgatgtgagggagacccgggggatgggacaggctgggccccgcagtgcctgactccctgcagggctcccgggacaggggtccggcggacagccggctgctcacgggtgaggggtccaagctggcattgcggccaccttccggcccgggctctcttggggaggggcggggttggtgagaaccggtcacgtgctccggggctcactcggggtctcccagggccggaagtagggcccctgtgcgcaggcgccctgaggatcccgggctgcccatctcacgccagggggcggaacttcctgcagcctctctgcctccgcatcctcgtgggccctgaccttctctctgagagccgggcagaggctccggagccatgcaggccgaaggccagggcacagggggttcgacgggcgatgctgatggcccaggaggccctggcattcctgatggcccagggggcaatgctggcggcccaggagaggcgggtgccacgggcggcagaggtccccggggcgcaggg

SEQ ID NO: 38 - DNA, 1000 bp, Homo sapiens
ctgggaccacaggcatgcatcaccacactaggctattgttttacattttttgtagagatggggtctcaccatgttgcccaggttggtctcaaactcctgggctcaagcaatccgctcacgtcaacctccccaaatgctgggattacaggcgtgagccaccgcgccaggcctgagtaatcctaatcacaggattttaaaaagaaacttcctgcgccacccattaaacaatatctcctaccaatttggtagtaaatattttgctaatagtacctaatttttaggtaggcactgtgtttatacatatatccattccttcttttttgattgtctttctgtttaatgggcagctacctctcttggcatctagcagaatgagctgctgcagtttacacaaaaagaatggagatcagagtactttttgtgccaccaacgtgtctgagaaatttgtagtgttactatcatcacacattacttttatttcatcgaatatttcaccttccggtcctgcgtgggccgagaggattgccgtacgcatgtctgtacgtatgcatgtaactcacagccccttcctgcccgaacatgttggaggccttttggaagctgtgcagacaacagtaacttcagcctgaatcatttctttcaattgtggacaagctgccaagaggcttgagtaggagaggagtgccgccgaggcggggcggggcggggcgtggagctgggctggcagtgggcgtggcggtgctgcccaggtgagccaccgctgcttctgcccagacacggtcgcctccacatccaggtctttgtgctcctcgcttgcctgttccttttccacgcattttccaggataactgtgactccaggtaagcaaggtggggtagcagggctggtgacttccttttttcagggaaattcataaatatcgttatttgagctgatttgagatggtgaacaaaatggacttaggtccattttggggctgttttcaaagacgggctgttgg

SEQ ID NO: 39 - DNA, 980 bp, Homo sapiens
  misc_feature (24)..(24): n is a, c, g, or t
  misc_feature (29)..(29): n is a, c, g, or t
  misc_feature (800)..(800): n is a, c, g, or t
  misc_feature (958)..(959): n is a, c, g, or t
tagggcgattgggccctctagatngcatngctcgagcggccgccagtgtgatggatatctgcagaattcgcccttggaagatctagaaattcttaattctaattaaatttgattgcaaacttctagtcaagacaaatatattcataagattagatttgtaaaatacaaacaattagaaagagtatttgtaccttaccttttatctggttgcttcctgaagtgagtactcctaggagaatgagaaatgatctctaatctttaggaatctggagaatatctgaataaagtagatttcttcatgttctactcttcacaggtaaagagtaatgatagcctttaaaatggtaatacaagtgtttatcccagtaccagaggaggagctacatgaactaaggcaggcaggcttgaaagcactaatcagtgaaaacccaaggataagtttgggtggaggaagggtgggagtagagataaaataaattttgagtacatgactatggctccaaagcattgaagaaatatgtgtgatctttttgctaaggtgtaggacgccttaatgagcagttgaaaaaacaaacaaaaacctcgaagagttacatggcttagggattggggtataattgaaaagtagccagagttgagaagtttagccagaataggcagaatgaagattagaatctaagctaaaaaaaaaaaaaaaaaagagagagacttcttttggtaggttactgggaagaccttcaaatgagaagtgaagtaaaaattgaattaatttgttcaaatttttaatttctctttatccnctggctaaaaaataattagtaaatttcaatttaaaataccatatgatatttcaaacaaaattgaaaatgtaacaagaatttgaagtaataagtatggaaaatataaagataaattagctttatggaaattcatttgtttactttgcaattatatcagnntttaatttataatgaaaaagt

SEQ ID NO: 40 - DNA, 1000 bp, Homo sapiens
cattgtgaggtactgggagttaggactccaacatagcttctctggtggacacaattcaactcctaataacgtccacacaaccccaagcagggcctggcaccctgtgtgctctctggagagcggctgagtcaggctctggcagtgtctaggccatcggtgactgcagcccctggacggcatcgcccaccacaggccctggaggctgcccccacggccccctgacagggtctctgctggtctgggggtccctgactaggggagcggcaccaggaggggagagactcgcgctccgggctcagcgtagccgccccgagcaggaccgggattctcactaagcgggcgccgtcctacgacccccgcgcgctttcaggaccactcgggcacgtggcaggtcgcttgcacgcccgcggactatccctgtgacaggaaaaggtacgggccatttggcaaactaaggcacagagcctcaggcggaagctgggaaggcgccgcccggcttgtaccggccgaagggccatccgggtcaggcgcacagggcagcggcgctgccggaggaccagggccggcgtgccggcgtccagcgaggatgcgcagactgcctcaggcccggcgccgccgcacagggcatgcgccgacccggtcgggcgggaacaccccgcccctcccgggctccgccccagctccgcccccgcgcgccccggccccgcccccgcgcgctctcttgcttttctcaggtcctcggctccgccccgctctagaccccgccccacgccgccatccccgtgcccctcggccccgcccccgcgccccggatatgctgggacagcccgcgcccctagaacgctttgcgtcccgacgcccgcaggtcctcgcggtgcgcaccgtttgcgacttggtgagtgtctgggtcgcctcgctcccggaagagtgcggagctctccctcgggacggtggcagcctcgagtggtcctgcaggcgccctcacttcgccgtcgggtgt

SEQ ID NO: 41 - DNA, 1000 bp, Homo sapiens
ggcacaggcaggttacatagtcttctcaggatgtcagtggcagagctaggacgtctatctctgcagctcagttctgtgcgaagtccaggcagatggtgctgatcagtaaggggtgctggctgagcgctgatggccacctgcatctcaaggagaaacagtgtcactggctaatctgatggcttctctgggcaccagcacgtgggcaccatcaccctttctctgcagggggtttgtttagtgtatttggtagaacatcccccagcctactaggtgtggcatgctctatgccacaagctctgtatctcaggcagcattttgtactttgaaaaaacaagttgggaacagaaccctgatgaatgtgtttcatttcctgtcagagcaaatgaaacctgaaatattaatggcacgagatttcccttatcttcctacaaaatcttcctacattgaaaaatgtactccccacaagcttagcatgcagctctgctacctgtggcccgaaatcattagttgtccatactcactgacctttggaaataaacacgaaggttcacttgaagacttgggggagaatcacggtcaacttgtgacgcttggtttttcagatattcagctgctctggagagccttggagttccagctgctctagaggttctggggagggagctgttagcctcccatatgagcgtgtggcccatcgttgccatccacacctgcccctctgtgggtgaataagtggtttcctttctcagctggttgacgcttcatttgtttgtgttctttttctttacagtctcctgaatatttacgcgttgctgaatctcctgtggacaaaccaccaataggccaggactgtcctgtggacagacggggtgagcctcttcttgtgtctggagattctgagtgagtagaacccgttatgatccccactgcacttaatgtggcattcatgaatgagtctgggctgatgtgctaattgggggccgtaagaagagttatagcc

SEQ ID NO: 42 - DNA, 671 bp, Homo sapiens
ccggggcctctatcctggcgggaagggcaggccgacccggcagactgcggcctctcgggagggaagaaggtgtcagacgcgcggagcaaccataaatagcccccctttcccagaagacggcacggggttcaagactcaggcgccgcatactcagaatgagagcagagactcccgccaggaaaaaaaggcacttaggggatctgctcattagcatgaaatgcaaatgagcccgcccggcctcatttacacaactctgtgcatggattcggcgaaagggcaaccagggagacgacggcgcagcagccactctgccacttcccccatcccctccccccatcggccggggcgggaactgagacgaccccaaccctctgcggtggcgggaggtgcgcgggggctgcgtgggtggtgcagccttaggagagtgaacaacgcccaggggtgatggcctcagcaaagtgaggggtggtgatggaggtcatccgacccatcccgccgcctctccgcagtggcgcaagcgccccaaaatctccggagagggaactgactgacccactaggttccgccgtgtctacctctcgcagatgttggggaagtgcttcccggcgtctaatcctcgctgttcccccctccaccggcgcccagcacacccgcggcgctccgctcccggg

SEQ ID NO: 43 - DNA, 1000 bp, Homo sapiens
agtgttttgagctgcatttatgcgtacttgacacttacgcattttgatcgaggtgatttagtgggcattttcactgggacagggatgcttgtatgtgtaatcttactaaaagctaataaaaacttactaaaagctaataaaagcttactaaaagcttcttgcttgattgaaacgaagacaacagaacatcccatggtctggaacctgatgactttgctcaagttttaatgtgggttcatggtttaaggagctggtttttcagaaactttagtttgagcctttttacaatgtgcacaaagaacccgttgctgtagttgtcagggtgccagtgtctctgggcgacacacattactgtggtttttctctgcttggtgagcagagataaagggggcagcaggaccgggcccaccagccatccgggctgcccacgcaaaccacagggccgaatccggagccgcccaaggccacacagctaagccgagtgcgtgaatgcttatgtgaccgtgtgaaggaggttcccaccgtgtggctgtgggggatggaaaaaggctacttggaaagatgtagaagaccttcgagtaaacagttacgtttcagaaacagagcctgctcagaatgtgtacttggtgggattctattcttagggacgcttctttcttctgagagacccgagctctgtggcgagtggcacaggcagggccccttcctttcctagttgggttctgacagctccgaggcagtggtttacacaaccaacacgaaacatttctacgatccacccgattcctcccctcattgatattcaggaagcagctctccttcccctgccttcagctcaagtttgctgagcttttgtttcatttgtgaatacttcttgctggaagtccctcacccagagaccagtgctcccaacggcagagcagcgggggaggtaagtgctcagacattaagccgttgagtagaggcatgttttgcaatctctcgtttagctaccaattgg

SEQ ID NO: 44 - DNA, 1000 bp, Homo sapiens
ctaacacggattaatgttatgtagagtaataggaatatggaaggaaaaataaccctgtttcttgcattttaatttaatccggaatccgcatatcacctaaaatgatcccttttctgggagcattccacattttccaaactgtcatcctgtggtggggtgcccggctaggctatggggagacctggagagttttatgcaaaggaggacctgggcaaatgtgcccattcagcctctcaagagtggagaatgcaaggacgggggcagagccctgtgtctgttctgtccctagacataagagaaacgtggccaacagaccgaggtggggacggggacagggaccggcaatgcaggaaatccgagtgtcacatcctctgcctctcatttgcacactgctccctcgctatgctcaccgctcccgccgatccagggacgtgatccagggactctgggaaatgcaaagctacacacagtggagcgggggctgggggtgtgtagaccgccgggattccgagtttcccggcacgcctaggagagggagaggcaggcaatgtcagggaaattgggcaggcaagacgccagggacgccacgtactgccaggttctcaacgaggtggagccaaaggggcaggccccgcggtgcgcccggcgctgggctcacgggttgctgcacccggcccaggatcgcgggcggtgcagactcagcaggggcgggtgcaaggacgaggcggggcctctgcgcccggccctcttcccggactataaagagagccgccggcttctgggctccaccacgcttttcatctgtcccgctgcgtgttttcctcttgatcgggaactcctgcttctccttgcctcgaaatggaccccaactgctcctgctcgcctggtaagggacacctagctccgcgccttgggatgcccgtttcccagccacagtacagactcttcctgggtttgaagaagtcgcatttaaagttctgagctgaaggggctcctttat

SEQ ID NO: 45 - DNA, 1000 bp, Homo sapiens
ctggggagcctgggcaggctgtcacctcctcagctgtcaggcccgaggtcctcatgtggtccccaggagaaggggcagacggccacttccggccaccagccagctccctgtgtgcctgattccgtaacatgtcccctggctgggcatgtactccccaagttctaattacatgtaactgcagagaagggctcagcctgggaaaaggatgggcatagggggtggttgggggctggggcctctgacacagctccatgagcccggccaagagtcccacacaagtcagtggcccccccggaccctgaaggatcccacatcctccctgccctcggggaggcccctttctggggtcaggcctggaagctgccccagagcttgggccccaggaatgggttggtcctcccagcgtaacgtgagcctgatcaggcctggggacctgctcagcgggtgtctgggggcccatggcgggctaaggagcctgaccagacttgcttctggcaggacacccctcccccggccaccctgggctcgcccctctagtagctgcatgtgttccccgggtgtgtgttggcattcaggctacagggctgcctcatcctgaagaaggctgcgtttacccagggagccataaagagatgacctccgataacctgaatcaatatttccccattggggctcgggcccccgcagctgtcttcttgatcatctggcagatgccacacccacccttggccctcccctgccttcctgccctcctaccctcctgccaggacatataaggaccagacccctgcccccgggcgcaacccacaccgcccctgccagccaccatggggctgccactagcccgcctggcggctgtgtgcctggccctgtctttggcagggggctcggagctccagacaggtgagagagcagacacaggggtctggggcctggcagagtgtcctgggggcagggcgaggcgggcgggcaagtcgcgtctgggaggaggagctggtcc

SEQ ID NO: 46 - DNA, 926 bp, Homo sapiens
agggcgattgggccctctagatgcatgctcgagcggccgccagtgtgatggatatctgcagaattcgcccttgttctcggatcccgatcatatccgcactgcaggtgttctcggatcccgatcatatccacactgcaggtggagctcattggctcatgcctgtaatcccaacactttaggaggctgaggcataccgaccacttgcggtcaggaatcaagaccagcctggccaacatggcgaaacctcgtctctactagaaatacaaaaaataaaaataaaaataaattaaccaggcgtggtggcccacgcgcccctgtagtcgtagctactttggaggctgaggtgggagaatcacttgaactcgggaggcggaggtcgcagcgagcagagattgagccactgcactccaacctgggtgacacaagaaagaaagaaaatgaaggaaagaagaaggaaggaaagaaagaaggaaagaaggaaggaaggaagaaaggaaggaaggaaggaaaaaaatagctggacatgatggaggactagcatttctcaatttcaaaacgtactacaaaccacactaatcaaaacaatgtggtactggcataaggatagacatatagatcaatggagtagaattgagagtcagaaacccatacatctaaggtcaactgattttcaaagagatgtcaagaccatgcaattggaaaagaataatctcttcaacaaatggtgctggaatacttggatactcacatgcaaaagaatgaagctaggcccttacctcacgccatttacaaaaaataactcaaaatgaaccaaaggcctaaatataagagctaaaattgtaagcctcttagaaataaacagagggcgggtcgcgcgctcggtgggcgcgttgtgcgcgtgtgtggagtgccctgctgcccccagc

SEQ ID NO: 47 - DNA, 1000 bp, Homo sapiens
gtttggagagattggcgcgaagctttagcagcaatctccgattcctgtacaaccatagctgggtttctaagcgtctagggaagaaggactgggcccacgacctgctgagcaactcccaggtcggggactggcggaatatcagagcctctacgacccgtttgtctcgggctcgcccacttcaactctcggggtctctccgcctgttgttgcactcgtgcgtttctctgcccctgacgctctaagctttctgctttctgcgtgtctctcagcctctttcggtccctctttcacggtctcactcctcagctctgtgcccccaatgccttgcctctctccaaatctctcacgacctgatttctacagccgctctacccatgggtcccccacaaatcaggggacagaggagtattgaaagtcagctcagaggtgagcgcgcgcagccagcgtttcccgcggatacagcagtcgggtgttggagaggtttggaaagggcgtgccggagagccaagtgcagccgcctagggctgccggtcgctccctccctccctgcccggtaggggacctagcgcgcacgccagtgtggaggggcgggctggctggccagtctgcgggcccctgcggccaccccggggacccccccaagccccgccccgcagtgttcctattggcctcggactccccctcccccagctgcccgcctgggctccggggcgtttaggctactacggataaatagcccagggcgcctggcgagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggatatggagctactgtcgccaccgctccgcga

SEQ ID NO: 48 - DNA, 1000 bp, Homo sapiens
ttccctggcagggggtgcgggagaaggggcccttccccaagaacagaacttcctaaagcggatgtttgaacctcgcagttatacagaagacttgtaggaaggatggacaaacgttcttaagcccatgacggcccttaacctggtcgctcccttttctgatggagactcaggcaatagcgtgtgtgcgtgtgtgtgtgtgtgtgtgtgtgtgtgtgtatccgtgtgtcctaatatcagacatttgttcttgttttccaggcagcgtctctctagcttctttctgcaatgctgtagtactctctccagtatttcaggaggaggagcatttgctatttcaaaaacgaaaaacaaaaacctggccacatccatttttttcagcagccatgcgatttccatcattgctcacattttatggatgaggaaactgagtcttagaggaattcagtaagtgatacctctctcggatgtgttgagtaactgagactgcactccctcccaggctggaacgtcctggtactcccacccccacaggctcagttctgtgcattatctgcctttttcggggattgtgacccttcttcacagcctcctccctcagaaagccaccaccatcagatccgattctccatggtacagcttcttctttggttccactctccagcaccctggggaagcaggaacagaggctgctgccactctctgacctctaaggggttaaggcctgggtcccgcccctcttcccgcccgcctggcgggagtatgaatagcctcgctcccactcccgactctcagtcgctcaggctactcccaccccgccccgccccgtcattgtccccgtcggtctcttttctcttccgtcctaaaagctctgcgagccgctcccttctcccggtgccccgcgtctgtccatcctcagtgggtcagacgagcaggatggagggctgcatgggggaggagtcgtttcagatgtgggagctcaatcggcgcctggaggcctacc

SEQ ID NO: 49 - DNA, 1000 bp, Homo sapiens
atacctgcagtagtgccgcagtttcacgagtgtgtgtgtgtgtgtgtgtgtgtgtgcgcgcgcgcgcgcgcactcgcgcgcacattccctatgtgttaagcagctcattaaagaaaaagaaaaataatcaggagaaaggaagatgaattgcagaaagtgccagaaagctagaaagaaattaaaactcttctccatacatactgcatacacataacctagcctatttatttgtatctaaaattccctagccgcaccatcaccgtaaacaccaagggaaaaaattaaggaggttcctggtgggaaaagggcgagttggggggacagggtgtctgcgaggtgacgggatacagaaaactagggtgtcaaaagggagcaagaacctgttttgggggcaacttaaggatccaagtgtcacggggtctgggcaatgcaggacgggaggggctgcgtgagtgagtacagaagggaaatgagtgagggggcatgggatctcagagaaaatcagggccctctgagcaaagtggaaaggacgaccgccgcagctcctcgggccgtagctcgaccccgccttcccttttgcgcagaatcctcgccttggctgcagcagcgcgctgcccccactggccggcgtgccgtgatcgatcgcaggctgcgtcaggagcctcccggcgtataaataggggtggcagaacggcgccgagccgcacacagccatccatcctcccccttccctctctcccctgtcctctctctccgggctcccaccgccgccgcgggccggggagccaccggccgccaccatgagttccttcagctacgagccgtactactcgacctcctacaagcggcgctacgtggagacgccccgggtgcacatctccagcgtgcgcagcggctacagcaccgcacgctcagcttactccagctactcggcgccggtgtcttcctcgctgtccgtgcgccgcagctactcctccagctctggatcgttgatgcccag

SEQ ID NO: 50 - DNA, 1000 bp, Homo sapiens
ctggcacagggccaactctcagtgcatatctgcaaaggaaccaatgaatgaatgaatgaagtgacaaatgaataaaggaataaatgaatgaggcacttatcatgtaccaggctttcgttaccacgtcccatttattcctctgaggcagggtctattttatccttgttacagatggggaaactaaggcccagggaggagcaaagtcttccccaagtatgtacccactcagaacttgagctctgaatgtctcccacccagcttagcccaagagcggggttcagtgatgcccaccccctaaggctctagagaaagggggtaggcccacatgccagtttgggggtggtaaagccaggtaagttttctttatgggtcccctgaaaccctgaaagtgaaccccagtcctgcatgaaagtgagctccccatagctcaaggtattcaagcacaatacggctttgagtgctgaagcaggctgtgcaggcttggatagtgacatgccctctctgagcctcaatttccccacctgtcaacagcagacagtgacagctgtgatcaggggatcacagtgcatggggatgggtgggtgcatggggatggaggggcatttgggagccctccccgataccaccccctgcagccacccagatagcctgtcctggcctgtctgtcccagtccagggctgaaagggtgcgggtcctgcccgcccctaggtctggaggcggagtcgcggtgacccgggagcccaataaatctgcaacccacaatcacgagctgctcccgtaagccccaaggcgacctccagctgtcagcgctgagcacagcgcccagggagagggacagacagccggctgcatgggacagcggaacccagagtgagaggggaggtggcaggacagacagacagcaggggcggacgcagagacagacagcggggacagggaggccgacacggacatcgacagcccatagattcctaacccagggagccccggcccctctcg

SEQ ID NO: 51 - DNA, 1000 bp, Homo sapiens
actggaaaactcgaccgcactttagtgccaggtgggcagggatccccatgtcagggtgggagtggggcggctgattggggctggaaatgtaggtggggaggcggcagccagggagcagggcatcctgcgagaagagcatcccgctaaggagtctgaacgccatcctgtaggcgggggagtcatcaaggcagggcagaggcaggaccagatggccgtttgaggtgctgagcaaagctcccggtttgcgcggagaggtgagatcgaggccccttgggaggccgaggcttaaccagggctcaagcagaggggagggaaggctggatttcagaggtagggaggataaggaccgtgggtgcacgacggggagggagagccaagtcaaggttaatgccggtgctcgggcggatggtgaaagcagcagatggccttgaccggggtagagaactcgagcacaggagcaggttctgtgtgtgtgtgtgtgtgtgtgtgtgtgtaggagcttttggggtcacgggaagtactgagaggtgaggagtgggatttgggacgtgcgtagttgaactcataggacgtccaggtggagaaggaatcacttcctgtctctggatccgtctcgatctctgcctggcgagggcgcgccccggctgggcgtggacactgttctccggccgcgtcgggccgggcgggtggggcgttcctgcgggttgggcggctgggccctccggggtgtggccaccccgcgctccgccctgcgcccctcctccgccgccggctcccgggtgtggtggtcgcaccagctctctgctctcccagcgcagcgccgccgcccggcccctccagcttcccggtaaggcggtgggggcgcatcccctggcgactcctcccgttccctcttccgcttgcgctgccgcaggtgggcccggtctgtgggcgccccccgatttcccgcaggtcccgcgcggcgtcggagcgggagattcccttgcagcttgcgccccgc

SEQ ID NO: 52 - DNA, 1000 bp, Homo sapiens
tacatacaaagaggcttaaactgcccagaacctccgaatgacgaagaatcaccgccagtctcaactcgtaagctgggaggcaaaaccccaaagcttccctaccaagggaaaacctttggcctcaaaggtccttctgtccagcatagccgggtccaataaccctccatcccgcgtccgcgcttacccaatacaagccgggctacgtccgagggtaacaacatgatcaaaaccacagcaggaaccacaataaggaacaagactcaggttaaagcaaacacagcgacagctcctgcgccgcatctcctggttccagtggcggcactgaactcgcggcaatttgtcccgcctctttcgcttcacggcagccaatcgcttccgccagagaaagaaaggcgccgaaatgaaacccgcctccgttcgccttcggaactgtcgtcacttccgtcctcagacttggaggggcggggatgaggagggcggggaggacgacgagggcgaagagggtgggtgagagccccggagcccgagccgaagggcgagccgcaaacgctaagtcgctggccattggtggacatggcgcaggcgcgtttgctccgacgggccgaatgttttggggcagtgttttgagcgcggagaccgcgtgatactggatgcgcatgggcataccgtgctctgcggctgcttggcgttgcttcttcctccagaagtgggcgctgggcagtcacgcagggtttgaaccggaagcgggagtaggtagctgcgtggctaacggagaaaagaagccgtggccgcgggaggaggcgagaggagtcgggatctgcgctgcagccaccgccgcggttgatactactttgaccttccgagtgcagtggtaggggcgcggaggcaacgcagcggcttctgcgctgggaaattcagtcgtgtgcgacccagtctgtcctctccccagaccgccaatctcatgcacccctccagagtggcccttgactcctccctctcc

SEQ ID NO: 53 - DNA, 1170 bp, Homo sapiens
gggtaaccgactcctatagggcgaattgggccctctagatgcatgctcgagcggccgccagtgtgatggatatctgcagaattcgcccttctagctagcaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggagggaaggaagtgccctcctgcagcacgcgaggttccgggaccggctggcctgctggaactcggccaggctcagctggctcggcgctgggcagccaggagcctgggccccgggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggagtgagagagcgcggcagacaacaggggaccccgggccggcggcccagagccgagccaagcgtgcccgcgtgtgtccctgcttgtccggagatgcgtgtcccggtgtaaatcatcaaggcgatcagccacctggcagccgttatatggatccgactcggtaccaagctggcgtaatcagggt

SEQ ID NO: 54 - DNA, 1000 bp, Homo sapiens
caaagtttattaagggacttgagagactagagttttttgttttttttttttaatcttgagttcctttcttattttcattgagggagagcttgagttcatgataagtgccgcgtctactcctggctaatttctaaaagaaagacgttcgctttggcttcttccctaggcccccagcctccccagggatggcagaaacttctgggttaaggctgagcgaaccattgcccactgcctccaccagcccccagcaaaggcacgccggcgggggggcgcccagcccccccagcaaacgctccgcggcctcccccgcagaccacgaggtgggggccgctggggagggccgagctgggggcagctcgccaccccggctcctagcgagctgccggcgaccttcgcggtcctctggtccaggtcccggcttcccgggcgaggagcgggagggaggtcggggcttaggcgccgcggcgaacccgccaacgcagcgccgggccccgaacctcaggccccgccccaggttcccggccgtttggctagtttgtttgtcttaattttaatttctccgaggccagccagagcaggtttgttggcagcagtacccctccagcagtcacgcgaccagccaatctcccggcggcgctcggggaggcggcgcgctcgggaacgaggggaggtggcggaaccgcgccggggccaccttaaggccgcgctcgccagcctcggcggggcggctcccgccgccgcaaccaatggatctcctcctctgtttaaatagactcgccgtgtcaatcattttcttcttcgtcagcctcccttccaccgccatattgggccactaaaaaaagggggctcgtcttttcggggtgtttttctccccctcccctgtccccgcttgctcacggctctgcgactccgacgccggcaaggtttggagagcggctgggttcgcgggacccgcgggcttgcacccgcccagactcggacgggctttgccaccctctcc

SEQ ID NO: 55 - DNA, 1025 bp, Homo sapiens
aggacaagctgccccaagtcctagcgggcagctcgaagaagtgaaacttacacgttggtctcctgtttccttaccaagcttttaccatggtaacccctggtcccgttcagccaccaccaccccacccagcacacctccaacctcagccagacaaggttgttgacacaagagagccctcaggggcacagagagagtctggacacgtggggagtcagccgtgtatcatcggaggcggccgggcacatggcagggatgagggaaagaccaagagtcctctgttgggcccaagtcctagacagacaaaacctagacaatcacgtggctggctgcatgccctgtggctgttgggctgggcccaggaggagggaggggcgctctttcctggaggtggtccagagcaccgggtggacagccctgggggaaaacttccacgttttgatggaggttatctttgataactccacagtgacctggttcgccaaaggaaaagcaggcaacgtgagctgttttttttttctccaagctgaacactaggggtcctaggctttttgggtcacccggcatggcagacagtcaacctggcaggacatccgggagagacagacacaggcagagggcagaaaggtcaagggaggttctcaggccaaggctattggggtttgctcaattgttcctgaatgctcttacacacgtacacacacagagcagcacacacacacacacacacatgcctcagcaagtcccagagagggaggtgtcgagggggacccgctggctgttcagacggactcccagagccagtgagtgggtggggctggaacatgagttcatctatttcctgcccacatctggtataaaaggaggcagtggcccacagaggagcacagctgtgtttggctgcagggccaagagcgctgtcaagaagacccacacgcccccctccagcagctgaattcctgcagctcagcagccgccgccagagcaggacgaaccgccaatcgcaaggcacctctgagaacttcagg

SEQ ID NO: 56 - DNA, 1000 bp, Homo sapiens
ggagaaaggagagaagaaagggcggggagagcggggtggaggatttggacaggccctggaggcttgggctggggaggcctctggcctcgtttagttctcggcccggcaacctcctctcggcctaggcttcgccgcggcctccgcagctggaatggagctgccaggacccagtgacgctcccgcccctttcctcttcttccaaggggccaggtgggctggggtgcggccgccgctgtgctctgtgtcttggggccccggctgggatggggtgggggcgggcgggggcggggcggcaggccacgctgtcctggagttggcaagaaaggacagcacagaaacttgcaccctccgaggactgggagtcccgagtccagcttagggggagtgggggcgcgacccccaacccagaaaccttcacttgaccgctcaagttcgcggcagcagggcgggccgcgccgaatctcggcgtgcgcggagcggggagatgcaggcgagcgccagagcccgggctcgggggccctgcgccggggagaggagccgggacccaccggcggagccgaaaacaagtgtattcatattcaaacaaacggaccaattgcaccaggcggggagagggagcatccaatcggctggcgcgaggccccggcgctgctttgcataaagcaatattttgtgtgagagcgagcggtgcatttgcatgttgcggagtgattagtgggtttgaaaagggaaccgtggctcggcctcatttcccgctctggttcaggcgcaggaggaagtgttttgctggaggatgatgacagaggtcaggcttcgctaatgggccagtgaggagcggtggaggcgaggccgggcgccggcacacacacattaacacacttgagccatcaccaatcagcataggtgtgctggctgcagccacttccctcacccacactctttatctctcactctccagccgctgacagcccattttattgtcaatctctgtctccttccc

SEQ ID NO: 57 - DNA, 1000 bp, Homo sapiens
acccctggctgttgcattctcttggctgatcccagcgtgccccggggaggccgctgacagctggatgtttccccagcctccccttaccatttccagcttcgtccagcacctcctccttctttcccacagctccacgggctcgtgtatctggggtggaggctgtggcacagaaactgcctttctcctcactttagtcacagcattcttgaacacatggccacaggcgcgatgtatgtggcactttgcagtttatgaagcactttgctgctaagcctgagtgagcctcaggctggccctgggggaggggacctgcatggggatggaaccacgcaggggtcagtccaggaaggagctgtaatggccagtgctgggagagtcagggcaggcctgctggtggaggtggccttggagctgtccacgtcctggtcgtgctcggactaatctttcagcagacggcaggcagccgtgaggcagggctgggtggagggcctgccgaggcctctgaggtgccatctccaccagctgagctggcttccaggagggcgagtcccactgtcacgtgacgcgtctggcctcagcacacttcttccgggaaagagtgaagggccccactgccctttgccatccagcttcctctggctttgctaatggccctagggggcaggagaccaactgctggaatcccagagccctggaggtgtgcaagggcaggtcaaacagaatttggaggatctggtgcaagagccaggaagagagagagagagagagtgtgtgtgtgtgtgtgtgtgcgcatctgagagagagagagagagagactgactgagcaggaatggtgagatgtttatcatgggcctcgtaagtacctctccacgtcttgtcttcccctccccacattgaggagcctcttctgtgacaactcttcctatgttctggtttatttcattgtttattacctgctttctctactggagtgtcaaccccattagagagctttcctcct

SEQ ID NO: 58 - DNA, 1000 bp, Homo sapiens
ccccccaatgtgctgtgaataagcagtgaccacaaccagtaccacctatgactgagtcgggaggctgctctctaagaaccccagctgcgtgaccacggggacaaatcaggccacctggggctccttcacatctgtccattgctgtgttaaaagtacttttaaacaactttgtcgaaatgctcagcttgtaaagttttaatgtaggcccttgtcaatgcttcagaaataagcctctggcggcgcgacagagcaaaactccctcaggaaagaaaggaaagaaatggagaaagggagaaagggagaaagagaggaaaagaaagaaagaaagaaagaaagagagagagagagagaaagagagaaagagaaagaaagaaaagaaagaaagaaaaagaaagaaagggaaagaaagaaggaaaggaaggaaagaaaagaaaggaaagaaaggaaaagaaaacaaataagcctccaggtcattgcttagaaagaaaaagaaaaaagaaagaaagaaaagaaagaaaaagaaaagaaaagaaaatagcctcccggtcattgctcctctctctctctgcgggtccacccccatggcaccctcccccctccccatggtgcaaggttacaatggaaagtgcctcagctggaaaggtctcagaatgtggctcagggcagccacaatcttatcaggagcttctctgtttgggatcaggggaaccggtgactttcagaggccgataaggcgggacccaacttgtatataaggggcagctcatgctgctgctctgcaccttcctcccatcttgccttctccctcgagttgggacccgggaagaaccatgaagtggctgctgctgctgggtctggtggcgctctctgagtgcatcatgtacaagtgagtccgggtggtgtgggtgtgaagacgctgcctcccacatcacctttctttcctcccgtgtcttccttcttcccttttttttctctctctcttcagctgtctccatccccc

SEQ ID NO: 59 - DNA, 1000 bp, Homo sapiens
actaaagccaagccagaactccagggccaagggggatgttgaaaattgtctgagtccccagaccaccctgccagctcatggcaaagggagggatcagaggccacagggaaagcacttcagctgctcttcacagcatcaccctctccccatttaatggtttaggttaacaggactttttccttgaggcttgggacacggaagggagcctcccctaaaccaggcccttggagagcaggccccaggggagcagtgcaactcaccttcacacccacaagacggctcctgacttctgctccctcctcccctccccaaagtggaacagagagaatatgattccccacgacttccacatcacagtttccaaacaatggggaaatcggaggcctccccgtgtgcagacggtgatatttaccgccaaatgcgaaccaggcagatgccagccccagcacgcacgcaggtaacttcaccctcgcctcaacgacctcagaggctgcccggcctgccccacacgggggtgctaagcctcccgcccgttctaagcggagacccaacgccatccataattaagttcttcctgagggcgagcggccaggtgcgccttcggcaggacagtgctaattccagcccctttccagcgcgtctccccgcgctcgtcccccgtctggaagcccccctcccacgccccgcggccccccttcccctggcccggggagctgctccttgtgctgccgggaaggtcaaagtcccgcgcccaccaggagagctcggcaagtatataaggacagaggagcgcgggaccaagcggcggcgaaggaggggaagaagagccgcgaccgagagaggccgccgagcgtccccgccctcagagagcagcctcccgagacaggtaagggcgcagcgtgggggacccgtgctctttccccgggatcccctgtccccgtcctcgcgatgcagtcggccggctccggctccgaaggcggacctgggcgcctctggctct

SEQ ID NO: 60 - DNA, 1000 bp, Homo sapiens
gaggcgtgaagccagagtccgtccgactccgcccgcaccggacgcgctctcagggcagaggaggtcggcggagttgtgacgctgggactagaggaaggagaaggaaagccgagacgggccgggcagacgcgccgaggagcgcccagtgcacgctggcagccgcgggaggcgaggcgggcgcggtgagcagtcgcgccggaaccgagccgcgaatccgcgccgcctcgcgctcgcagccgccaggacccgcgggaatcctggtccgccggcagcggtactgaggagggaggggcgcggggctgagccgcttctcggagcccgagcgcctcccggagccggcaatccctgctgcccgggcgggatgcgggcgggaattcagctcgcgtggaatgtgggaccggccgggctcggagtctccagcgctgggggaaagcggggcccacacagccaggacgagagggggtgcggtcccagggccacccccgcgccactccccacgtggcggccgcgcccccggggcgtgagtgtgtacgcggacggtaggggggccgtgaatgaagccccagcggccaatcagcacggccggcgcgcgggaccccgggagcgacgcccaatggagagctctgggcggccgggcagggtggcgggcgggcgcgcggggcgggggccgggcaggggaggcgggaggcagctccgcgggcagccaatgggcggcgggcggggtggggctccggagcgccgagcgggtcggggctttaagccggcggagcgaggcggcggggcccgcagacggagcggagcggcggcggcggcgcggcgcagggcgcggggcggcatggccaccaccgcgcagtacctgccgcggggccccggtggcggagccgggggcaccgggccgcttatgcacccggacgccgcggcggcggcggcggcggcggcggccgcggagcgattgcatgcaggggccgcgtaccgcgaagtgcagaagctgatgcaccac

SEQ ID NO: 61 - DNA, 1000 bp, Homo sapiens
agatttaggcggaaatgtggaataactgctagtgggtattgagattttagagtcatactcatgttacaaaattaatagtgctgatggttgcacaactctgagtacatgaaaaatcaatgaactgatactttgagtgagctgtatgatactggaattacacctcaataaagcatggtaactgttttaagataggctggaaagagaaagcctgaaaacaacaataatgatattaataaattagtttacttctctagtctcatatacttctgtgcccacacttgctcctgttctattcataatggtccccttgcagttgccatattatatcctgccatttgatgcccggtgaacattctatacctgcttcccagaattctctttacctttcctctatctgcctaacttccacatatctaaaattaatcagagtaaactatttactagaacaaccaactccaaatcctagtaacctaacatgataaaggtttgtttctcactcatatagcccctccccagatgatcgaggggtccaggctccttacctctagtggctcccccaccttctggagtcttctgcattctttatacatggttgagataaactatgagtcattagcacagctagaccttgaggtcctacaagaaaatttgcaaatcattcactctgttttgaacaaggtatatttaagatgatgttaaaatacccaatggtcttgggtcaaatacagtttatgactgtgtatctaaaatatatattgcaatattcttccctttttctactgacttcatgaatttagcggggatccattttataagctcaaagataattacttttcagactaagaatatttagggtaaaaagtactgttcaacatctctactgaggatgttatgatgtagcacactgtataagctggagctaaaggaaactttccttaaagtgctatttactaaaaattggaacacattccttaagacaaatcgaagtgtggcacacaac

SEQ ID NO: 62 - DNA, 1000 bp, Homo sapiens
agaaagaaaaagaaaaaaaaggctgtttctggggattaaataagacaattatgtaaggtggccagcacagttcctggtacatagtaaatgtcaggcctgcctgacagacttctattcagcagctactgctcccctgaaaatcttcctcagacgtttccacggtgcttcccgttcttacaccactacaatcctttattacactactatccgttcattccccacagctccctcccttcctttccctaaccagtgatcccaaaaggccagcaagtgtctaacattttctatcttctaagtgactggtaaagttccgcacctatcagcgctccaagtttgtttttgttttggccgactttgcaaaacggattgggcgggatgagaggtggggggcgccgcccaaggagggagagtggcgctcccgccgagggtgcactagccagatattccctgcggggcccgagagtcttccctatcagaccccgggatagggatgaggcccacagtcacccaccagactctttgtatagccccgttaagtgcaccccggcctggagggggtggttctgggtagaagcacgtccgggccgcgccggatgcctcctggaaggcgcctggacccacgccaggtttcccagtttaattcctcatgacttagcgtcccagcccgcgcaccgaccagcgccccagttccccacagacgccggcgggcccgggagcctcgcggacgtgacgccgcgggcggaagtgacgttttcccgcggttggacgcggcgctcagttgccgggcgggggagggcgcgtccggtttttctcaggggacgttgaaattatttttgtaacgggagtcgggagaggacggggcgtgccccgacgtgcgcgcgcgtcgtcctccccggcgctcctccacagctcgctggctcccgccgcggaaaggcgtcatgccgcccaaaaccccccgaaaaacggccgccaccgccgccgctgccgccgcggaaccccc

SEQ ID NO: 63 - DNA, 914 bp, Homo sapiens
  misc_feature (908)..(908): n is a, c, g, or t
  misc_feature (912)..(912): n is a, c, g, or t
agggcgattgggccctctagatgcatgctcgagcggccgccagtgtgatggatatctgcagaattcgcccttgttctcggatcccgatcatgcagaaaaggtccaagggaacagcctctggttcttttgttacttaggcgtggaaagttggggttttcctttcaatttagttctaagaagtcacgtgaaacagccataggttccctgcctccagaccctattctcctgcctcatttactgcagtcttctctgcctgcctcttttagcgactagcatgagatgaggattcgtcttctaatatccgtcaccaatccttcccctctgtcatttagcgaaccactcactgggcactaggactttggggagagtcccaagaggcccctcttcgtccaggggctacttttttctcttccagcctccatctcctaactcaaggggtacagctcagattatgtttggcgcccagggacagtgacaaacccagggcccgtggatagaggaggcatctcactacgctgcacgaggccacctcgcagtaggcagcccagccctgccccaaaacccgagagcctaaccaggaggacagggggaggccgcgggcttcatctcccaagagatggactacacctcccagcaggctctgcgcgcgggctgaggatccctccgctctttttctgtcccgccggctgggccccccgcgaccagccaagggccaaggacaggtctttcagaatctgaggtacatcttcttatcacatttccggggagggactgctaggagctccggaggaaaaacggactttttttgaggagaaaagcggaggcagacggtggatgacaacacgtcccgcagctgcagattttcgcgcgctttggcgcaggtgggttgtgggtagcgcgcctgggangganaa

SEQ ID NO: 64 - DNA, 971 bp, Homo sapiens
  misc_feature (785)..(785): n is a, c, g, or t
  misc_feature (823)..(823): n is a, c, g, or t
  misc_feature (842)..(842): n is a, c, g, or t
  misc_feature (845)..(845): n is a, c, g, or t
  misc_feature (875)..(876): n is a, c, g, or t
  misc_feature (883)..(883): n is a, c, g, or t
  misc_feature (915)..(915): n is a, c, g, or t
  misc_feature (933)..(933): n is a, c, g, or t
  misc_feature (935)..(935): n is a, c, g, or t
  misc_feature (948)..(948): n is a, c, g, or t
agggcgattgggccctctagatgcatgctcgagcggccgccagtgtgatggatatctgcagaattcgcccttgtttcggatcccgatctcctaccagatccattcgggaatgaaggcagagacaagaacagagcagagaggtggcaggacgggcagcaggctccgccgaggagacaggcgggacacgggcgactggctgctgatgccggagtggaggtgacagatggcggcgacggcggcggccgcgtccggaactggatctctcctcttccgccctcttcgctaggacagtcgcttgcaattggccgcacgcccctagctcctccttaaggcacctttccccgcccccgggcgggctacttccggctgctgaccgccgggctcggagaagcaagcatcagctggctgtcgcttggggtcacgttgcctgtgtcgggcagggcagggcaagaactgggtgtggcttcctttggcccaggctctgccctgtccccgcactgccatctccttctttcctccttggcaccccaaaaattgccgctggatctaaactagattagactagtggattgtaaataaataaacaaactaggctctctctgttcattcattatttcctggagcagttctaaactgggatgacttgggagacagaaaacggcaggtttatagagggaaagggcctggaaaggacggtcggagtttgtggttgttgttgttgaagggcggggcgtggaatgcggaaaatgtgtaaaatgtgttacgtaacagtgaacaaaaataanactgcataataaaacttttgtttctgtattttgtaganattctaataaaatgaccanatnaaagataactaaacattgcatttcacttannatatcantgcactgttcaaatctttcattaactttttantcctcaaattaccgtgananctaaattctgtcnttatctctattttactgatattg

SEQ ID NO: 65 - DNA, 1000 bp, Homo sapiens
cagtagctgggaccccaggcacttgccaccacaccagactaatttttaaaaatattttttgagagagggtctcactatgttgtccaggctggtctcaaacttccagcctcaagcggtcctcctgcctcagaccccatttgctgggtttacaggcatgagccacagcacctgctaatttttcttaaatacataaatgaacataaaattctaacaatgcatgagtattttgaggaaggaactgacaaaatgttccactccctatgggaggcacgttatatgaagaattatgaaaaatggtcgaaatgactggagaggccaagcctggatgagactgggatggggacaggtgcgggacgaggggcaccaccctcacatctttcacaagtctgtcataggcaagagggcgtaggtttctcacagccccactggggagaatcggcaccattggtggcattacacgaagagaatgtgacctcctatgtaaaagaacaagcaactccacgcggtgctgtgaggctagtgctgcgagtccctgaggtgcgcaattcccgcacgaccgtgggtgggaaacaccgaagccaaaactccgctacagccctttagatgaaggcgtcgtctgattggtgatagtttggcgcgaacctgagcacgccgaacaaaggaagtgacggcagaagtcgcgcacttgacgagggtgggatcacacggcgctgcgtcgcggtagtattgttctgattggttgatttcttgcgataccgctctgccagccccttgcttccgctagtgcggagggttttgcccttcgtaaagatggccgcggaggcttttggagccaactgggagcgcagtacgcgttttctggagcatgggcagaggagacaggaacaagcgtagcatccgtgagcaccgattggctgaagcgagcaccccgggagctgactggctccgccattcgcgggaaggcgtttgtggtgccagagaaaagtagccagagcggcgc

SEQ ID NO: 66 - DNA, 1000 bp, Homo sapiens; identical to SEQ ID NO: 1

SEQ ID NO: 67 - DNA, 1000 bp, Homo sapiens
cgcgccgtgtgcactcaccgcgacttccccgaacccgggagcgcgcgggtctctcccgggagagtccctggaggcagcgacgcggaggcgcgcctgtgactccagggccgcggcggggtcggaggcaagattcgccgcccccgcccccgccgcggtccctcccccctcccgctcccccctccgggacccaggcggccagtgctccgcccgaaggcgggtctgccataaacaaacgcggctcggccgcacgtggacagcggaggtgctgcgcctagccacacatcgcgggctccggcgctgcgtctccaggcacagggagccgccaggaagggcaggagagcgcgcccgggccagggcccggccccagccgcctgcgactcgctcccctccgctgggctcccgctccatggctccgcggccaccgccgcccctgtcgccctccggtccggaggggccttgccgcagccggttcgagcactcgacgaaggagtaagcagcgcctccgcctccgcgccggccgcccccaccccccaggaaggccgaggcaggagaggcaggagggaggaaacaggagcgagcaggaacggggctccggttgctgcaggacggtccagcccggaggaggctgcgctccgggcagcggcgggcggcgccgccgggttgctcggagctcaggcccggcggctgcggggaggcgtctcggaaccccgggaggccccccgcacctgcccgcggcccactccgcggactcacctggctcccggctcccccttccccatccccgccgccgcagcccgagcggggctccgcgggcctggagcacggccgggtctaatatgcccggagccgaggcgcgatgaaggagaagtccaagaatgcggccaagaccaggagggagaaggaaaatggcgagttttacgagcttgccaagctgctcccgctgccgtcggccatcacttcgcagctggacaaagcgtccatcatccgcctcaccacgagc

SEQ ID NO: 68 - DNA, 1000 bp, Homo sapiens
atcaaagcaaagaccagtgcctagtctaacgcttttaaggattttaaaagaggtgaaggtgtcctgcttatcctccaagcttgggtgctggggccggggcggctgagatttaccagtgaaacccaaagaaagagagggcagaaaactagagaaaagaaaccagataatgctacccaagaggacgaaataaagaagcaggaaacgaagcctgaggctaaaccctggagatgactattaggaaaacaccagaggatgccccgcccgccagcccacaatgagcagcctgtccaagtcacaaagcggggcctcgggccttgacagttcgcgatctgtaagcagaatgttccagggcctccctgtcgcctgcatccagcctgggggcaatcttcactggtgtgggaggccgaaagtggacggcgacggaggcccctctggttatctctttgccgtgccaacacagtctctgcgcccactaagatgcatgaaataaaaatttccgtgactcgccctttgcagtggagaactgaaacaggcacaccagggaattggagcggaggagggtaactcaaactcagagtgagagggtttgcagggggccgatttggggccaacaggcttcccagcaggcccccggcgcgggacagcggaaggcgaaacgctttcaagagaccccgctgccaacatccccacgccctcgcgccctcccgccgccccagaaggccaactccgcctgcctgagtcacagctggagctggggaggagccagggaaaggaggcccctgaccgtagtgcggccagcagttgcaggcagacggagcagagcggtcagggatcatgagggagagtgcgttggagcgggggcctgtgcccgaggcgccggcggggggtcccgtgcacgccgtgacggtggtgaccctgctggagaagctggcctccatgctggagactctgcgggagcggcagggaggcctggctcgaaggcagggaggcctggcagggt

SEQ ID NO: 69 - DNA, 833 bp, Homo sapiens
  misc_feature (691)..(691): n is a, c, g, or t
  misc_feature (794)..(794): n is a, c, g, or t
  misc_feature (808)..(808): n is a, c, g, or t
gggcgattgggccctctagatgcatgctcgagcggccgccagtgtgatggatatctgcagaattcgcccttgttctcggatcccgatcggttctgaacatagtttgtagagctcactgcacatacaagtggagaggcaagtgggagttgtaggtgtgaagcccagaggagaggtgtggacgggataagcatttaagactcctccatctagaaggaaactgaagctgtgggtaaggtcatcacagcacagcgtttaggagaagcccaggtaaagaagctgacgaatgtctggaccctgacaaccttaacatataatggtttgatagtggaggtggaggcaatgtagaaagaatgccagaggcaggaaaaagcaaggaggatgtgttatcatcatgaccaaggaagaaacgtgtttcaagaacaaaggcgtcaactctgccccatgcttccgagctgtcaagtaaagtgagaaaaacagaaaagcgttccctgggtttagcaacacggaggtcagttgctaaagggagcttctagaatgacgacgtcgccaaatctgtcctctgcctggattctcggcgatgaaactactacagagacctccaagtttgggcttctgcaaacacagcacgtccttctgatcgttctctaagatatgtaaacagaacgccagttcccagcgtggcaacacgggnactgggctgcagctcacccagccggcggcccccgccggaagccggcggaaataccccagtgcgtgggcggagcagcggcccgcagagggaggcggtggcgccncacggaacagcccncgtctaattggctgagcgcggaggc

SEQ ID NO: 70 - DNA, 937 bp, Homo sapiens
  misc_feature (775)..(775): n is a, c, g, or t
  misc_feature (779)..(779): n is a, c, g, or t
  misc_feature (823)..(823): n is a, c, g, or t
  misc_feature (919)..(919): n is a, c, g, or t
  misc_feature (935)..(935): n is a, c, g, or t
agggcgattgggccctctagatgcatgctcgagcggccgccagtgtgatggatatctgcagaattcgcccttgttctcggatcccgatccctgcctgaagggaactgctggagggcacaggtgccaagtgggacccacccaaatgtggcaatgggtttgtatccagccaccgacaggctgcatgacggtggcaaagtcacttcccctctctggcctttgtttttccacttgtaaaatcatctttatggtcacttccagctgtggcacttggctttcattccagttgaccccctagctctgtgtctgaccctcccctgccaaatccattgcccagagtgggaaaggagaggagagggactatacttcctcctccctggggccccctgcagagcatctgggaagcaaggcttccctacatcctccatgcacccccttagagttttcaattcctttcctcgtgatcctgccaactaagacactgtgaccacacagagaaggtggggagaacgcagacattttggcttctgcagctttgaagttctttttttttcctctgaagttaaaagaatgaaactgggagaggtagtaaggggcaagaaaggagagtggaaatggagagaaaagggcagctctgagaagcggctggggagggaggcagatgagaatgcaccccccccaacagaacatgcagtcttggcccagctgtgctgtgagtgggcagctgggctggcccctcctctggtgctgccaacccgctgccaggcagaggggaggnccanaggagagggaagctgggcaaaggggatggaaggcgtccagcccnaccttaccaaaccccttgggcctcgtgggaaggggcctcttggagaggggactgaggctctagacaggatattcactgctgcggcaaggcctgtanagagtttcgaagttanga

SEQ ID NO: 71 - DNA, 1000 bp, Homo sapiens
tgcgaagggaaaggaggagtttgccctgagcacaggcccccaccctccactgggctttccccagctcccttgtcttcttatcacggtagtggcccagtccctggcccctgactccagaaggtggccctcctggaaacccaggtcgtgcagtcaacgatgtactcgccgggacagcgatgtctgctgcactccatccctcccctgttcatttgtccttcatgcccgtctggagtagatgctttttgcagaggtggcaccctgtaaagctctcctgtctgactttttttttttttttagactgagttttgctcttgttgcctaggctggagtgcaatggcacaatctcagctcactgcaccctctgcctcccgggttcaagcgattctcctgcctcagcctcccgagtagttgggattacaggcatgcaccaccacgcccagctaatttttgtatttttagtagagacaaggtttcaccgtgatggccaggctggtcttgaactccaggactcaagtgatgctcctgcctaggcctctcaaagtgttgggattacaggcgtgagccactgcacccggcctgcacgcgttctttgaaagcagtcgagggggcgctaggtgtgggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcgggtgagactgcccggcctcctggggtcccccacgcccgcct

SEQ ID NO: 72 - DNA, 1000 bp, Homo sapiens
aaaagtatctatttgttttagcaacactgttgagaattctgtctgtaaaggagaggtgagagaaagaccactagcttatctgtgtttggtctgtgtttgatgagggggcttggggtatggggttaagaaaggtgactttggaatgttttagatgagagaaattttgacagcctttaagtcctgatagtaaagagcgagttagcagagagccgttgaggagtcatgcaacggaagggttcatcagaggagcttgactctgagtcggcaacagggaatagagatggaagagggctggcttagatcaaaggagagtagtcgtttattattattattattgcaaaaagaataggagaaaggattggtgaggggtacaagaaaattagaaaatttcatggcgaaagtagaggcagttcctgtcagatgaattctattttgtctgtgaggaaacgggcgacgctgcctactgagactaagcaggagagacggggcaagcttggctcttcatttatgccgcctactcattgctggtagattctttatctagcctgcatcctctcattttcctggatccctatacggcatttgacgctgtttaccacaagagctgtcgaacgaacgtgaaacactcagtgatactccaaccggaactactactcccagaatgcagtacggctcctgggaagtgcggggggctgggaacgcagcaggcctagccgtgtcgcctgctgccattggaggagcgctcccactcccaagaggccacgcgtagacggggcgcttcatgcggaagtcagcggcgtccggtcccagcctcctctgggagcgggcagttggcgaccctgcactgacccgcgtccctccgtcccgagcccgcgcgccctcagagggtgcccggacaggtaaatggagtggggtgcgcctgcgggaggcggggagagaactgcggagggagggcggaggtgtcgatggaaaggtgctggggtggagcgaggaggcagtg

SEQ ID NO: 73 - DNA, 1000 bp, Homo sapiens
cggagacaacgtacagatgttctctctttccctctttattttttttaagacagggtctctgttgcccaggctggagtgcagtggcgcgaccacagctcactacagcctcaacctcctgggctcaacacgatcctcctgcctcagcctccagagcggctgggactacaagcgcgcaccactgcacagggattattattattattttattattttgtagagaaacgggtgggagtggtctcgctatgttgcccaggctggtctcaaactcagctcaagagatcctcccgcctcggcgtcccaaagtgttgggattacaggcgcctgccaccgcgcccggacgcagatattttctatgggcatctggaatggcgtccccaaagcttggcgccgtgctatggtcaagccgggtcgggggctcgggccagccttcaacaccgttggcagcaatcggaacgatcaactgtaccctcagtaccgcgacctcgcccggtcctgccaatggccggcccctagccggtcctgaggcctcgcgagagctcccgtggctacgccttccccggcctcggaacggccccatccttcctctttccccgcctcccagcggcgctccactctcggattggctgattgatccgagtcagtttttttcctcgccagaaagcggttcgacaattggtccttcttttggcccctcctgcgatgcccgcggattggacggctgagtctggctacgcgggcctccgcgggagcgcgaccgggccaatcaagagcttggcgtattttacaaactgagaaagtagctccagcagcacccgagagggtcaggagaaaagcggaggaagctgggtaggccctgaggggcctcggtaaggtaaggcacgggggtcttgaagggaacgaaggctgctgggttcatagggaggagggcagtttggggcccgagggcgaaagagtaggctcggggtgtctggagatagcacccataagagcggtcttgcag

SEQ ID NO: 74 - DNA, 1000 bp, Homo sapiens
cccccagcccctcccagaaggagacttaatctgtcgctcaggctggagtgcagtagggtgatctcgactcactgcaacctccgcctcccaggttcaagtgattctcctgacttaacctccagagtagctaggattacaggcacccgccaccatgcctggctaatttttgtattttttttttttgtagagacggggtttcgccatgttggccaggctagtctcaaactcctgactttaagtgatccgcctgctttggcctcccaaagtgttgggattacaggcgtgagccactgcgccaggcctacaatttcattattaaaacaattccactgtaaaagaattagcttaggcctagacggaatgggcttcatgagctccttcccttccccctgcaaggtcacggtggccaccccgtgagccactgttgtcacggccaagcctttttccggccatctctcactatgaatcacttctgcagtgagtacagtatttaccctggcgggagggcctctcagatatgagtaggacctggattaaggtcaggttggaggagactcccatgggaaagagggactttctgaatctcagatccctcagccaagatgacctcaccacatgtcgtctctgtctatcagcaaatccttccatgtagcttgaccatgtctaggaaacacctttgataaaaatcagtggagattattgtctcagaggatccccgggcctccttaggcaaatgttatctaacgctctttaagcaaacagagcctgccctataaaatccggggctcgggcggcctctcatccctgactcggggtcgcctttggagcagagaggaggcaatggccaccatggagaacaaggtgatctgcgccctggtcctggtgtccatgctggccctcggcaccctggccgaggcccagacaggtaaggcgtgcttcttcctgctctgtggggccacagccagctctggcagcctccgccaggagccactgttttaca

SEQ ID NO: 75 - DNA, 1000 bp, Homo sapiens
aattcgagtagaaagcagctgtcctccccgggccccttgatgagaatacgcacaccgcccccaagcggccggccgagggagcgccgcggcagcgggagaggcgtctctgtgggccccctggcagccgcggcaggaaagggcccgaaggcagcgaaggcgaacgcggcgcaccaacctgccggccccgccgacgccgcgctcacctccctccggggcgggcgtggggccagctcaggacaggcgctcgggggacgcgtgtcctcaccccacggggacggtggaggagagtcagcgagggcccgaggggcaggtactttaacgaatggctctcttggtgtcccctgcgccccgtcggcccatttttctttttacaaaacgggcccagtctctagtatccacctctcgccatcaaccaggcattccgggagatcagctcgcccgaaagcccctgcgccaccccgcgggccctcctaggtggtctccccagccccgtcccttttcgggatgcttgctgatcaccccgagcccgcgtggcgcaagagtacgagcgccgagcccgtgcgcgccaaggctgcgtgggcgggcaccgacttttctgagaagttctagtgctcccaagccccgacccccgcccccttcactttctagctggaaagttgcgcgccaggcagcggggggcggagagaggagcccagactggcccccacctcccgcttcctgcccggccgccgcccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggtaagtcgcccccggcggccgccgaggaccaaagctgcccgggacatcca

SEQ ID NO: 76 - DNA, 1000 bp, Homo sapiens
caccttagagcagcagcttcccctttccactgtataccctgacctgggagaagcagcccctccgcatccatcgtccaccctgacctctgagaagcggtgccccccacccccatgcagagtgcaccctgattgcgggtgatgcctgaggtgtgggaggggcgggggttagctgctgccactgcttctcgttctctcgagtccttgctctgtgcctgcacgtcaggttgttcctgtgatggggccacgtgcaagtgtgcaccaaggggacttggccgggtactgtacgtccactgggacacacccttctacgggtattgcacgtccactgggagacgtccttctaggggatcctcactgagcaaatgaagcagaatttgggtaaaaatgaattttcccaaagctgcagtacagcttttcagtcctctaactgcctgagataaatgttggcaacttccttttatattaaatttcatttttgtcacataatacacttgattattgaccataataactttattaatatacagactgattattgatactcaccgatgtatttcatgtgttattgagagtcactcatttggtttagaaagaccaatatcacattgagtaattcgaaacatatttaaggcatagaacttgcatttttttctcttaagcaaaatgaggagttctagccaatcttgctagtgttatttatagcatcttatttcctgagagaagacaggaaaagtgagtccctgccttccctctctccgtctggctcctcccaggcctgtctggcaggggccggggtgcaggaggaggagacggcatccagtacagaggggctggacttggacccctgcagcaggtactcggagcaaatggtgagatcagaagggggatgatgtcattccttcgaaggaatgaattaaacgtgcttcctcgtgtgtctgattgacagccctgcacaggagaagcggcatataaagccgcgctgcccgggagccgctcggcc

SEQ ID NO: 77 - DNA, 337 bp, Homo sapiens
gggcgattgggccctctagatgcatgctcgagcggccgccagtgtgatggatatctgcagaattcgcccttagaggaggagaagccgtctgagcgcccgccgcctgcctgctgcccgctctgcgccgctgcctgggcggccgagtgatatagcgctgggcccccggggaccccgcctcgggctgttggggcccgccccctcagaccaatggcagagccgcattacctcatcggccctccaaaaagggggcggggccgggggcaaggggtaacggggcggggccgcccccggatcgttcagatccttatagggaataatgccgccgtgggcacgcgag

SEQ ID NO: 78 - DNA, 1000 bp, Homo sapiens
tgactacaaggaacagtgattgttacaacccagatgagagggaaaaataaaggattccaaatatcccccttgggaagtagagtcaggattcaaacaaagaactgtatggcttcaagttcatggtctttaatctcctggaggctgtctctctttcttttttcttttttttaatcagtgttgggatcaaattctggctcccctaggaagcatctggcaaggtttcgggagccatcgggttggccatgttatgctggaatatttataagcaccggagggttatccccatgtcgtagaaaatgaaactgaagctcagagagatttgcactctctgcccttttgtacaactcatttttccccagtatgtggaattgagggagcttcacgcttctagctgtcatgattccaagattctacgacatgtgggagaggatcctaaggttcggggaaccgcggaggtttcggggttctagaaatccgaggttctaagcctaggtgctccaataaacccagtgagagccagcccaggtttccggtctgtacccgctggtgcaagcccagagacaagcaggcgccacccatgagcccctctgcggccccctcccgggtcccacctcgcaggccagctggagggcgcgatcctggcgtcccccgacggcctggggccccaatccagaggcctgggtgggaggggaccaagggtgtagtaaggaagcgccttttgctggagggcaacggaccggggcggggagtcgggagaccagagtgggaggaaggcggggagtccaggttccgccccggagccgacttcctcctggtcggcggctgcagcggggtgagcggcggcagcggccggggatcctggagccatggggcgcgcgcgcgacgccatcctggatgcgctggagaacctgaccgccgaggagctcaagaagttcaagctgaagctgctgtcggtgccgctgcgcgagggctacgggcgcatcccgcggggcgcgctgct

SEQ ID NO: 79 - DNA, 1000 bp, Homo sapiens
cccgggagtgttcgcgtcctgggtgacccctggaaggacgtggggcccaaactccggctggggttgggagagcagcccccagaggctctccgcgggatcctctgccgggcgggaccgtggctccacaggagaagtgggtggcaagccctgcttggcggaaagcagccgttcccctcctcctgggcctggggcggcgcccctcacccctgttccccgcccctcacccctgttccccgccggccacatcccctgccccttggattccaagcgccccgcgcgccgaggagcccagcgctagtggcggcggccaggagagacccgggtgtcaggaaagatgggccgtctgggggacagcagggagtccgggggaaacgcaggcgtcgggcacagagtcggcaccggcgtccccagctctgccgaagatcgcggtcgggtctggcccgcgggaggggccctggcgccggacctgcttcggccctgcgtgggcggcctcgccgggctctgcaggagcgacgcgcgccaaaaggcggcgggaaggaggcggggcagagcgcgcccgggaccccgacttggacgcggccagctggagaggcggagcgccgggaggagaccttggccccgccgcgactcggtggcccgcgctgccttcccgcgcgccgggctaaaaaggcgctaacgcccgcggccgcctactccccgcggcgcctcccctccccgcgcccatataacccgcctaggggccgggcagcccgccctgcctccccgcccgcgcacccgcccggaggctcgcgcgcccgcgaaggggacgcagcgaaaccggggcccgcgccaggccagccgggacggacgccgatgcccggggctgcgacggctgcaggtaggaggcccagggccggggggcggttcggctccgcgggcgggggctggagcgcagcgctgggcaggcacctgggctcgcagctccgaagctgggaggtgaggggagagcgatcggggacga

SEQ ID NO: 80 - DNA, 1000 bp, Homo sapiens; identical to SEQ ID NO: 75

SEQ ID NO: 81 - DNA, 775 bp, Homo sapiens
tgatgattgggtgttcccgtgtgagatgcgccaccctcgaaccttgttacgacgtcggcacattgcgcgtctgacatgaagaaaaaaaaaattcagttagtccaccaggcacagtggctaaggcctgtaatccctgcactttgagaggccaaggcaggaggatcacttgaacccaggagttcgagaccagcctaggcaacatagcgagactccgtttcaaacaacaaataaaaataattagtcgggcatggtggtgcgcgcctacagtaccaactactcgggaggctgaggcgagacgatcgcttgagccagggaggtcaaggctgcagtgagccaagctcgcgccactgcactccagcccgggcgacagagtgagaccctgtctccaaaaaaaaaaaaaaacaccaaaccttagaggggtgaaaaaaaattttatagtggaaatacagtaacgagttggcctagcctcgcctccgttacaacagcctacggtgctggaggatccttctgcgcacgcgcacagcctccggccggctatttccgcgagcgcgttccatcctctaccgagcgcgcgcgaagactacggaggtcgactcgggagcgcgcacgcagctccgccccgcgtccgacccgcggatcccgcggcgtccggcccgggtggtctggatcgcggagggaatgccccggagggcggagaactgggacgaggccgaggtaggcgcggaggaggcaggcgtcgaagagtacggccctgaagaagacggcggggaggagt

SEQ ID NO: 82 - DNA, 1000 bp, Homo sapiens
ctgttttcccggcttaaccgtagaagaattagatattcctcactggaaagggaaactaagtgctgctgactccaattttaggtaggcggcaaccgccttccgcctggcgcaaacctcaccaagtaaacaactactagccgatcgaaatacgcccggcttataactggtgcaactcccggccacccaactgagggacgttcgctttcagtcccgacctctggaacccacaaagggccacctctttccccagtgaccccaagatcatggccactcccctacccgacagttctagaagcaagagccagactcaagggtgcaaagcaagggtatacgcttctttgaagcttgactgagttctttctgcgctttcctgaagttcccgccctcttggagcctacctgcccctccctccaaaccactcttttagattaacaaccccatctctactcccaccgcattcgaccctgcccggactcactgcttacctgaacggactctccagtgagacgaggctcccacactggcgaaggccaagaaggggaggtggggggagggttgtgccacaccggccagctgagagcgcgtgttgggttgaagaggagggtgtctccgagagggacgctccctcggacccgccctcaccccagctgcgagggcgcccccaaggagcagcgcgcgctgcctggccgggcttgggctgctgagtgaatggagcggccgagcctcctggctcctcctcttccccgcgccgccggcccctcttatttgagctttgggaagctgagggcagccaggcagctggggtaaggagttcaaggcagcgcccacacccgggggctctccgcaacccgaccgcctgtccgctcccccacttcccgccctccctcccacctactcattcacccacccacccacccagagccgggacggcagcccaggcgcccgggccccgccgtctcctcgccgcgatcctggacttcctcttgctgcaggacccggc

SEQ ID NO: 83 - DNA, 24 bp, Artificial
  synthetic oligonucleotide linker
aggcaactgtgctatccgagggat

SEQ ID NO: 84 - DNA, 12 bp, Artificial
  synthetic oligonucleotide linker
taatccctcgga

SEQ ID NO: 85 - DNA, 387 bp, Homo sapiens
ataagcgtgatgattgggtgttcccgtgtgagatgcgccaccctcgaaccttgttacgacgtcggcacattgcgcgtctgacatgaagaaaaaaaaaattcagttagtccaccaggcacagtggctaaggcctgtaatccctgcactttgagaggccaaggcaggaggatcacttgaacccaggagttcgagaccagcctaggcaacatagcgagactccgtttcaaacaacaaataaaaataattagtcgggcatggtggtgcgcgcctacagtaccaactactcgggaggctgaggcgagacgatcgcttgagccagggaggtcaaggctgcagtgagccaagctcgcgccactgcactccagcccgggcgacagagtgagaccc

SEQ ID NO: 86 - DNA, 385 bp, Homo sapiens
gggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtacgggcccggcgcttaggcccgacccagcagggacgatagcacggtctgaagc

SEQ ID NO: 87 - DNA, 402 bp, Homo sapiens
ggagtagatgctttttgcagaggtggcaccctgtaaagctctcctgtctgactttttttttttttttagactgagttttgctcttgttgcctaggctggagtgcaatggcacaatctcagctcactgcaccctctgcctcccgggttcaagcgattctcctgcctcagcctcccgagtagttgggattacaggcatgcaccaccacgcccagctaatttttgtatttttagtagagacaaggtttcaccgtgatggccaggctggtcttgaactccaggactcaagtgatgctcctgcctaggcctctcaaagtgttgggattacaggcgtgagccactgcacccggcctgcacgcgttctttgaaagcagtcgagggggcgctaggtgtgggcagggacga

SEQ ID NO: 88 - DNA, 378 bp, Homo sapiens
ctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcgggtgagactgcccggcctcctggggtcccccacgcccgccttgccctgtccctagcgaggccac

SEQ ID NO: 89 - DNA, 337 bp, Homo sapiens
gctggcggaagccccacggcggtgaggtccatcctgaccaaggagcggcggccggagggcgggtacaaggctgtctggtttggcgaggacatcgggacggaggcagacgtggtcgttctcaacgcgcccaccctggacgtggatggcgccagtgactccggcagcggcgatgagggcgagggcgcggggaggggtgggggtccctacgatgcgcccggtggtgatgactcctacatctaagtggcccctccaccctctcccccagccgcacgggcactggaggtctcgctcccccagcctccgacccgaggcagaataaagcaaggctcccgaaacc

SEQ ID NO: 90 - DNA, 353 bp, Homo sapiens
tgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagaggtaagagcccggcccaccctctccagatgccagtccccgagcgccctgcagccggccctgactctccgcggccgggcacccgcagggcagccccacgcgtgctgttcggagagtggctccttggagagatcagcagcggctgctatgagggg

SEQ ID NO: 91 - DNA, 392 bp, Homo sapiens
ttagtgtgacgtgaccccacccctagctaacccaggctgcttccttaccagcttcccgccccctggggaggcggcaatgcaaagaccgtccgctgccagctctgccgctatctctgtggggtgaatctaacatggcggacaaagacagtaactagtcccgtttctccgcgttttcgccaagaagattggctcttaccacttgtccctcaaaacgaccaccccattgactggtggcgattgcgtcgacggagacggggcaaaagcaagctgaacccgaaaaataacaaacactggggctgaggggtggaactacgagtgcgcagacatgggccagagcgcatttcccctgccccaggcaaattcggcgctcactgcgtccccgcaggccactg

SEQ ID NO: 92 - DNA, 349 bp, Homo sapiens
taaattaaaactgcgactgcgcggcgtgagctcgctgagacttcctggacgggggacaggctgtggggtttctcagataactgggcccctgcgctcaggaggccttcaccctctgctctgggtaaaggtagtagagtcccgggaaagggacagggggcccaagtgatgctctggggtactggcgtgggagagtggatttccgaagctgacagatgggtattctttgacggggggtaggggcggaacctgagaggcgtaaggcgttgtgaaccctggggaggggggcagtttgtaggtcgcgagggaagcgctgaggatcaggaagggggcactgagtgtccgtggggga

SEQ ID NO: 93 - DNA, 46 bp, Artificial
  synthetic oligonucleotide probe
ctgaattttttttttcttcatgtcatttttctcttggaaagaaagt

SEQ ID NO: 94 - DNA, 43 bp, Artificial
  synthetic oligonucleotide probe
gtgcagggattacaggccttagtttttctcttggaaagaaagt

SEQ ID NO: 95 - DNA, 41 bp, Artificial
  synthetic oligonucleotide probe
gttgcctaggctggtctcgatttttctcttggaaagaaagt

SEQ ID NO: 96 - DNA, 44 bp, Artificial
  synthetic oligonucleotide probe
tgtttgaaacggagtctcgctattttttctcttggaaagaaagt

SEQ ID NO: 97 - DNA, 40 bp, Artificial
  synthetic oligonucleotide probe
gccttgacctccctggctctttttctcttggaaagaaagt

SEQ ID NO: 98 - DNA, 39 bp, Artificial
  synthetic oligonucleotide probe
gggctggagtgcagtggctttttctcttggaaagaaagt

SEQ ID NO: 99 - DNA, 47 bp, Artificial
  synthetic oligonucleotide probe
gaacacccaatcatcacgcttattttttaggcataggacccgtgtct

SEQ ID NO: 100 - DNA, 41 bp, Artificial
  synthetic oligonucleotide probe
tggcgcatctcacacggtttttaggcataggacccgtgtct

SEQ ID NO: 101 - DNA, 45 bp, Artificial
  synthetic oligonucleotide probe
cgtcgtaacaaggttcgagggtttttaggcataggacccgtgtct

SEQ ID NO: 102 - DNA, 41 bp, Artificial
  synthetic oligonucleotide probe
gacgcgcaatgtgccgatttttaggcataggacccgtgtct

SEQ ID NO: 103 - DNA, 45 bp, Artificial
  synthetic oligonucleotide probe
ccactgtgcctggtggactaatttttaggcataggacccgtgtct

SEQ ID NO: 104 - DNA, 43 bp, Artificial
  synthetic oligonucleotide probe
cctgccttggcctctcaaatttttaggcataggacccgtgtct

SEQ ID NO: 105 - DNA, 49 bp, Artificial
  synthetic oligonucleotide probe
tgcccgactaattatttttatttgttttttaggcataggacccgtgtct

SEQ ID NO: 106 - DNA, 46 bp, Artificial
  synthetic oligonucleotide probe
gcctcccgagtagttggtactgtttttaggcataggacccgtgtct

SEQ ID NO: 107 - DNA, 43 bp, Artificial
  synthetic oligonucleotide probe
aagcgatcgtctcgcctcatttttaggcataggacccgtgtct

SEQ ID NO: 108 - DNA, 43 bp, Artificial
  synthetic oligonucleotide probe
gcgagcttggctcactgcatttttaggcataggacccgtgtct

SEQ ID NO: 109 - DNA, 43 bp, Artificial
  synthetic oligonucleotide probe
gggtctcactctgtcgccctttttaggcataggacccgtgtct

SEQ ID NO: 110 - DNA, 22 bp, Artificial
  synthetic oligonucleotide probe
actcctgggttcaagtgatcct

SEQ ID NO: 111 - DNA, 16 bp, Artificial
  synthetic oligonucleotide probe
taggcgcgcaccacca

SEQ ID NO: 112 - DNA, 37 bp, Artificial
  synthetic oligonucleotide probe
gccggactcctcggcgtttttctcttggaaagaaagt

SEQ ID NO: 113 - DNA, 39 bp, Artificial
  synthetic oligonucleotide probe
cgtcccagttctccgccctttttctcttggaaagaaagt

SEQ ID NO: 114 - DNA, 38 bp, Artificial
  synthetic oligonucleotide probe
cgcgcctacctcggccttttttctcttggaaagaaagt

SEQ ID NO: 115 - DNA, 38 bp, Artificial
  synthetic oligonucleotide probe
gggccggactcttccggtttttctcttggaaagaaagt

SEQ ID NO: 116 - DNA, 42 bp, Artificial
  synthetic oligonucleotide probe
tgcgattgcagaagatgaccttttttctcttggaaagaaagt

SEQ ID NO: 117 - DNA, 38 bp, Artificial
  synthetic oligonucleotide probe
gcggcagcgttgggtagtttttctcttggaaagaaagt

SEQ ID NO: 118 - DNA, 39 bp, Artificial
  synthetic oligonucleotide probe
cctgctgggtcgggcctatttttctcttggaaagaaagt

SEQ ID NO: 119 - DNA, 43 bp, Artificial
  synthetic oligonucleotide probe
cttcgacgcctgcctcctctttttaggcataggacccgtgtct

SEQ ID NO: 120 - DNA, 45 bp, Artificial
  synthetic oligonucleotide probe
cgtcttcttcagggccgtacttttttaggcataggacccgtgtct

SEQ ID NO: 121 - DNA, 41 bp, Artificial
  synthetic oligonucleotide probe
cggcgcccagttcctcctttttaggcataggacccgtgtct

SEQ ID NO: 122 - DNA, 43 bp, Artificial
  synthetic oligonucleotide probe
ccggcctccatctcctccttttttaggcataggacccgtgtct

SEQ ID NO: 123 - DNA, 42 bp, Artificial
  synthetic oligonucleotide probe
gttcaccgagcgcagcactttttaggcataggacccgtgtct

SEQ ID NO: 124 - DNA, 40 bp, Artificial
  synthetic oligonucleotide probe
gggagggctcgcgcgatttttaggcataggacccgtgtct

SEQ ID NO: 125 - DNA, 40 bp, Artificial
  synthetic oligonucleotide probe
agcacgacgcgcggactttttaggcataggacccgtgtct

SEQ ID NO: 126 - DNA, 44 bp, Artificial
  synthetic oligonucleotide probe
cgaagttgagccatacgggctttttaggcataggacccgtgtct

SEQ ID NO: 127 - DNA, 40 bp, Artificial
  synthetic oligonucleotide probe
ggctgcggctcgccgttttttaggcataggacccgtgtct

SEQ ID NO: 128 - DNA, 44 bp, Artificial
  synthetic oligonucleotide probe
acctcggtagctgtggatgctttttaggcataggacccgtgtct

SEQ ID NO: 129 - DNA, 45 bp, Artificial
  synthetic oligonucleotide probe
gcttcagaccgtgctatcgtctttttaggcataggacccgtgtct

SEQ ID NO: 130 - DNA, 16 bp, Artificial
  synthetic oligonucleotide probe
cccgactcctccccgc

SEQ ID NO: 131 - DNA, 13 bp, Artificial
  synthetic oligonucleotide probe
gggccgcggccgc

SEQ ID NO: 132 - DNA, 15 bp, Artificial
  synthetic oligonucleotide probe
ggcggcccgtgccag

SEQ ID NO: 133 - DNA, 14 bp, Artificial
  synthetic oligonucleotide probe
agcgccgggcccgt

SEQ ID NO: 134 - DNA, 42 bp, Artificial
  synthetic oligonucleotide probe
ggagagctttacagggtgccatttttctcttggaaagaaagt

SEQ ID NO: 135 - DNA, 41 bp, Artificial
  synthetic oligonucleotide probe
ttgtgccattgcactccagctttttctcttggaaagaaagt

SEQ ID NO: 136 - DNA, 43 bp, Artificial
  synthetic oligonucleotide probe
cagagggtgcagtgagctgagatttttctcttggaaagaaagt

SEQ ID NO: 137 - DNA, 39 bp, Artificial
  synthetic oligonucleotide probe
tcgcttgaacccgggaggtttttctcttggaaagaaagt

SEQ ID NO: 138 - DNA, 39 bp, Artificial
  synthetic oligonucleotide probe
ccgggtgcagtggctcactttttctcttggaaagaaagt

SEQ ID NO: 139 - DNA, 40 bp, Artificial
  synthetic oligonucleotide probe
ttcaaagaacgcgtgcaggtttttctcttggaaagaaagt

SEQ ID NO: 140 - DNA, 47 bp, Artificial
  synthetic oligonucleotide probe
cctctgcaaaaagcatctactcctttttaggcataggacccgtgtct

SEQ ID NO: 141 - DNA, 47 bp, Artificial
  synthetic oligonucleotide probe
ctaggcaacaagagcaaaactcatttttaggcataggacccgtgtct

SEQ ID NO: 142 - DNA, 43 bp, Artificial
  synthetic oligonucleotide probe
ggaggctgaggcaggagaatttttaggcataggacccgtgtct

SEQ ID NO: 143 - DNA, 44 bp, Artificial
  synthetic oligonucleotide probe
ggcctaggcaggagcatcactttttaggcataggacccgtgtct

SEQ ID NO: 144 - DNA, 46 bp, Artificial
  synthetic oligonucleotide probe
tgcctgtaatcccaactactcgtttttaggcataggacccgtgtct

SEQ ID NO: 145 - DNA, 41 bp, Artificial
  synthetic oligonucleotide probe
ctgggcgtggtggtgcatttttaggcataggacccgtgtct

SEQ ID NO: 146 - DNA, 54 bp, Artificial
  synthetic oligonucleotide probe
ccttgtctctactaaaaatacaaaaattagtttttaggcataggacccgtgtct

SEQ ID NO: 147 - DNA, 48 bp, Artificial
  synthetic oligonucleotide probe
ttgagtcctggagttcaagaccagtttttaggcataggacccgtgtct

SEQ ID NO: 148 - DNA, 48 bp, Artificial
  synthetic oligonucleotide probe
gcctgtaatcccaacactttgagatttttaggcataggacccgtgtct

SEQ ID NO: 149 - DNA, 41 bp, Artificial
  synthetic oligonucleotide probe
gcgccccctcgactgcttttttaggcataggacccgtgtct

SEQ ID NO: 150 - DNA, 43 bp, Artificial
  synthetic oligonucleotide probe
tcgtccctgcccacacctatttttaggcataggacccgtgtct

SEQ ID NO: 151 - DNA, 27 bp, Artificial
  synthetic oligonucleotide probe
gtctaaaaaaaaaaaaaaagtcagaca

SEQ ID NO: 152 - DNA, 19 bp, Artificial
  synthetic oligonucleotide probe
cctggccatcacggtgaaa

SEQ ID NO: 153 - DNA, 41 bp, Artificial
  synthetic oligonucleotide probe
ggagttgtagtcctcccgcctttttctcttggaaagaaagt

SEQ ID NO: 154 - DNA, 38 bp, Artificial
  synthetic oligonucleotide probe
gagtagaggcggggcggtttttctcttggaaagaaagt

SEQ ID NO: 155 - DNA, 41 bp, Artificial
  synthetic oligonucleotide probe
caaatctggcggttaatggctttttctcttggaaagaaagt

SEQ ID NO: 156 - DNA, 40 bp, Artificial
  synthetic oligonucleotide probe
tccttgagaaagggctgcctttttctcttggaaagaaagt

SEQ ID NO: 157 - DNA, 37 bp, Artificial
  synthetic oligonucleotide probe
cggggtgcaggcgcagtttttctcttggaaagaaagt

SEQ ID NO: 158 - DNA, 40 bp, Artificial
  synthetic oligonucleotide probe
gtggcctcgctagggacagtttttctcttggaaagaaagt

SEQ ID NO: 159 - DNA, 41 bp, Artificial
  synthetic oligonucleotide probe
ggtcgcggtgcacccagtttttaggcataggacccgtgtct

SEQ ID NO: 160 - DNA, 40 bp, Artificial
  synthetic oligonucleotide probe
gcgtggctctgcccgttttttaggcataggacccgtgtct

SEQ ID NO: 161 - DNA, 43 bp, Artificial
  synthetic oligonucleotide probe
cctcttaggcggtccaccctttttaggcataggacccgtgtct

SEQ ID NO: 162 - DNA, 40 bp, Artificial
  synthetic oligonucleotide probe
tgtcgggagcgcacgctttttaggcataggacccgtgtct

SEQ ID NO: 163 - DNA, 41 bp, Artificial
  synthetic oligonucleotide probe
caacgggtcccgcgatttttttaggcataggacccgtgtct

SEQ ID NO: 164 - DNA, 46 bp, Artificial
  synthetic oligonucleotide probe
cttgaatgtagagatgcggtggtttttaggcataggacccgtgtct

SEQ ID NO: 165 - DNA, 44 bp, Artificial
  synthetic oligonucleotide probe
ccctccaagaagggccagtttttttaggcataggacccgtgtct

SEQ ID NO: 166 - DNA, 42 bp, Artificial
  synthetic oligonucleotide probe
gggcagtctcacccgctctttttaggcataggacccgtgtct

SEQ ID NO: 167 - DNA, 41 bp, Artificial
  synthetic oligonucleotide probe
ggggaccccaggaggcctttttaggcataggacccgtgtct

SEQ ID NO: 168 - DNA, 14 bp, Artificial
  synthetic oligonucleotide probe
cgcggggtgtgccg

SEQ ID NO: 169 - DNA, 15 bp, Artificial
  synthetic oligonucleotide probe
cccgcggccttctgg

SEQ ID NO: 170 - DNA, 13 bp, Artificial
  synthetic oligonucleotide probe
gcgccgcggggca

SEQ ID NO: 171 - DNA, 16 bp, Artificial
  synthetic oligonucleotide probe
ccgccgccacctctgc

SEQ ID NO: 172 - DNA, 15 bp, Artificial
  synthetic oligonucleotide probe
ggggcacccatgccg

SEQ ID NO: 173 - DNA, 17 bp, Artificial
  synthetic oligonucleotide probe
aggcagggggcaacgtc

SEQ ID NO: 174 - DNA, 15 bp, Artificial
  synthetic oligonucleotide probe
ggcaaggcgggcgtg

SEQ ID NO: 175 - DNA, 37 bp, Artificial
  synthetic oligonucleotide probe
tggggcttccgccagctttttctcttggaaagaaagt

SEQ ID NO: 176 - DNA, 38 bp, Artificial
  synthetic oligonucleotide probe
gatggacctcaccgccgtttttctcttggaaagaaagt

SEQ ID NO: 177 - DNA, 38 bp, Artificial
  synthetic oligonucleotide probe
cgccgctccttggtcagtttttctcttggaaagaaagt

SEQ ID NO: 178 - DNA, 38 bp, Artificial
  synthetic oligonucleotide probe
cacgtccagggtgggcgtttttctcttggaaagaaagt

SEQ ID NO: 179 - DNA, 40 bp, Artificial
  synthetic oligonucleotide probe
ttattctgcctcgggtcggtttttctcttggaaagaaagt

SEQ ID NO: 180 - DNA, 39 bp, Artificial
  synthetic oligonucleotide probe
ggtttcgggagccttgcttttttctcttggaaagaaagt

SEQ ID NO: 181 - DNA, 40 bp, Artificial
  synthetic oligonucleotide probe
gtacccgccctccggctttttaggcataggacccgtgtct

SEQ ID NO: 182 - DNA, 43 bp, Artificial
  synthetic oligonucleotide probe
cgccaaaccagacagcctttttttaggcataggacccgtgtct

SEQ ID NO: 183 - DNA, 42 bp, Artificial
  synthetic oligonucleotide probe
cctccgtcccgatgtccttttttaggcataggacccgtgtct

SEQ ID NO: 184 - DNA, 45 bp, Artificial
  synthetic oligonucleotide probe
cgttgagaacgaccacgtctgtttttaggcataggacccgtgtct

SEQ ID NO: 185 - DNA, 43 bp, Artificial
  synthetic oligonucleotide probe
cggagtcactggcgccatctttttaggcataggacccgtgtct

SEQ ID NO: 186 - DNA, 40 bp, Artificial
  synthetic oligonucleotide probe
ccctcatcgccgctgctttttaggcataggacccgtgtct

SEQ ID NO: 187 - DNA, 40 bp, Artificial
  synthetic oligonucleotide probe
tcaccaccgggcgcattttttaggcataggacccgtgtct

SEQ ID NO: 188 - DNA, 47 bp, Artificial
  synthetic oligonucleotide probe
gggccacttagatgtaggagtcatttttaggcataggacccgtgtct

SEQ ID NO: 189 - DNA, 41 bp, Artificial
  synthetic oligonucleotide probe
cctccagtgcccgtgcgtttttaggcataggacccgtgtct

SEQ ID NO: 190 - DNA, 14 bp, Artificial
  synthetic oligonucleotide probe
tccccgcgccctcg

SEQ ID NO: 191 - DNA, 18 bp, Artificial
  synthetic oligonucleotide probe
cgtagggacccccacccc

SEQ ID NO: 192 - DNA, 19 bp, Artificial
  synthetic oligonucleotide probe
gctgggggagagggtggag

SEQ ID NO: 193 - DNA, 17 bp, Artificial
  synthetic oligonucleotide probe
aggctgggggagcgaga

SEQ ID NO: 194 - DNA, 42 bp, Artificial
  synthetic oligonucleotide probe
ctcggtatggatctcttggcatttttctcttggaaagaaagt

SEQ ID NO: 195 - DNA, 38 bp, Artificial
  synthetic oligonucleotide probe
gcagcagacgccaggcctttttctcttggaaagaaagt

SEQ ID NO: 196 - DNA, 41 bp, Artificial
  synthetic oligonucleotide probe
cttctgcaggggcagttaggtttttctcttggaaagaaagt

SEQ ID NO: 197 - DNA, 40 bp, Artificial
  synthetic oligonucleotide probe
gccgggctcttacctctcatttttctcttggaaagaaagt

SEQ ID NO: 198 - DNA, 37 bp, Artificial
  synthetic oligonucleotide probe
cagggcgctcggggactttttctcttggaaagaaagt

SEQ ID NO: 199 - DNA, 39 bp, Artificial
  synthetic oligonucleotide probe
tctccgaacagcacgcgttttttctcttggaaagaaagt

SEQ ID NO: 200 - DNA, 42 bp, Artificial
  synthetic oligonucleotide probe
tgtagccaccgacgctgctttttaggcataggacccgtgtct

SEQ ID NO: 201 - DNA, 45 bp, Artificial
  synthetic oligonucleotide probe
cacaggtgtggactgagggcttttttaggcataggacccgtgtct

SEQ ID NO: 202 - DNA, 44 bp, Artificial
  synthetic oligonucleotide probe
ggccaggtgtcacaggtgtctttttaggcataggacccgtgtct

SEQ ID NO: 203 - DNA, 41 bp, Artificial
  synthetic oligonucleotide probe
gcggccacaggtcgtgttttttaggcataggacccgtgtct

SEQ ID NO: 204 - DNA, 44 bp, Artificial
  synthetic oligonucleotide probe
gggaggtaagggctcctgtctttttaggcataggacccgtgtct

SEQ ID NO: 205 - DNA, 46 bp, Artificial
  synthetic oligonucleotide probe
tggcggtcaggtgttataacagtttttaggcataggacccgtgtct

SEQ ID NO: 206 - DNA, 43 bp, Artificial
  synthetic oligonucleotide probe
ggagccaaggccattgctctttttaggcataggacccgtgtct

SEQ ID NO: 207 - DNA, 43 bp, Artificial
  synthetic oligonucleotide probe
tggcatctggagagggtggtttttaggcataggacccgtgtct

SEQ ID NO: 208 - DNA, 42 bp, Artificial
  synthetic oligonucleotide probe
gagagtcagggccggctgtttttaggcataggacccgtgtct

SEQ ID NO: 209 - DNA, 46 bp, Artificial
  synthetic oligonucleotide probe
gctgatctctccaaggagccactttttaggcataggacccgtgtct

SEQ ID NO: 210 - DNA, 42 bp, Artificial
  synthetic oligonucleotide probe
cccctcatagcagccgcttttttaggcataggacccgtgtct

SEQ ID NO: 211 - DNA, 13 bp, Artificial
  synthetic oligonucleotide probe
gtgcccggccgcg

SEQ ID NO: 212 - DNA, 15 bp, Artificial
  synthetic oligonucleotide probe
ggggctgccctgcgg

SEQ ID NO: 213 - DNA, 41 bp, Artificial
  synthetic oligonucleotide probe
agcagcctgggttagctaggtttttctcttggaaagaaagt

SEQ ID NO: 214 - DNA, 40 bp, Artificial
  synthetic oligonucleotide probe
ggcgggaagctggtaaggatttttctcttggaaagaaagt

SEQ ID NO: 215 - DNA, 40 bp, Artificial
  synthetic oligonucleotide probe
agcggacggtctttgcatttttttctcttggaaagaaagt

SEQ ID NO: 216 - DNA, 40 bp, Artificial
  synthetic oligonucleotide probe
agatagcggcagagctggctttttctcttggaaagaaagt

SEQ ID NO: 217 - DNA, 41 bp, Artificial
  synthetic oligonucleotide probe
ttcgggttcagcttgctttttttttctcttggaaagaaagt

SEQ ID NO: 218 - DNA, 40 bp, Artificial
  synthetic oligonucleotide probe
gcagtgagcgccgaatttgtttttctcttggaaagaaagt

SEQ ID NO: 219 - DNA, 45 bp, Artificial
  synthetic oligonucleotide probe
ggtggggtcacgtcacactaatttttaggcataggacccgtgtct

SEQ ID NO: 220 - DNA, 46 bp, Artificial
  synthetic oligonucleotide probe
ccatgttagattcaccccacagtttttaggcataggacccgtgtct

SEQ ID NO: 221 - DNA, 48 bp, Artificial
  synthetic oligonucleotide probe
gggactagttactgtctttgtccgtttttaggcataggacccgtgtct

SEQ ID NO: 222 - DNA, 43 bp, Artificial
  synthetic oligonucleotide probe
gggtggtcgttttgagggatttttaggcataggacccgtgtct

SEQ ID NO: 223 - DNA, 44 bp, Artificial
  synthetic oligonucleotide probe
gcaatcgccaccagtcaatgtttttaggcataggacccgtgtct

SEQ ID NO: 224 - DNA, 41 bp, Artificial
  synthetic oligonucleotide probe
gccccgtctccgtcgactttttaggcataggacccgtgtct

SEQ ID NO: 225 - DNA, 46 bp, Artificial
  synthetic oligonucleotide probe
tcagccccagtgtttgttattttttttaggcataggacccgtgtct

SEQ ID NO: 226 - DNA, 44 bp, Artificial
  synthetic oligonucleotide probe
cgcactcgtagttccacccctttttaggcataggacccgtgtct

SEQ ID NO: 227 - DNA, 42 bp, Artificial
  synthetic oligonucleotide probe
cgctctggcccatgtctgtttttaggcataggacccgtgtct

SEQ ID NO: 228 - DNA, 42 bp, Artificial
  synthetic oligonucleotide probe
cctggggcaggggaaatgtttttaggcataggacccgtgtct

SEQ ID NO: 229 - DNA, 41 bp, Artificial
  synthetic oligonucleotide probe
cagtggcctgcggggactttttaggcataggacccgtgtct

SEQ ID NO: 230 - DNA, 15 bp, Artificial
  synthetic oligonucleotide probe
gccgcctccccaggg

SEQ ID NO: 231 - DNA, 19 bp, Artificial
  synthetic oligonucleotide probe
ggcgaaaacgcggagaaac

SEQ ID NO: 232 - DNA, 24 bp, Artificial
  synthetic oligonucleotide probe
caagtggtaagagccaatcttctt

SEQ ID NO: 233 - DNA, 39 bp, Artificial
  synthetic oligonucleotide probe
tctcagcgagctcacgcctttttctcttggaaagaaagt

SEQ ID NO: 234 - DNA, 37 bp, Artificial
  synthetic oligonucleotide probe
agcgcaggggcccagttttttctcttggaaagaaagt

SEQ ID NO: 235 - DNA, 41 bp, Artificial
  synthetic oligonucleotide probe
cagagggtgaaggcctcctgtttttctcttggaaagaaagt

SEQ ID NO: 236 - DNA, 39 bp, Artificial
  synthetic oligonucleotide probe
gccccctgtccctttccctttttctcttggaaagaaagt

SEQ ID NO: 237 - DNA, 39 bp, Artificial
  synthetic oligonucleotide probe
cgcctctcaggttccgcctttttctcttggaaagaaagt

SEQ ID NO: 238 - DNA, 40 bp, Artificial
  synthetic oligonucleotide probe
gcccccttcctgatcctcatttttctcttggaaagaaagt

SEQ ID NO: 239 - DNA, 46 bp, Artificial
  synthetic oligonucleotide probe
gcgcagtcgcagttttaatttatttttaggcataggacccgtgtct

SEQ ID NO: 240 - DNA, 42 bp, Artificial
  synthetic oligonucleotide probe
tgtcccccgtccaggaagtttttaggcataggacccgtgtct

SEQ ID NO: 241 - DNA, 45 bp, Artificial
  synthetic oligonucleotide probe
tatctgagaaaccccacagcctttttaggcataggacccgtgtct

SEQ ID NO: 242 - DNA, 49 bp, Artificial
  synthetic oligonucleotide probe
gggactctactacctttacccagagtttttaggcataggacccgtgtct

SEQ ID NO: 243 - DNA, 45 bp, Artificial
  synthetic oligonucleotide probe
gtaccccagagcatcacttggtttttaggcataggacccgtgtct

SEQ ID NO: 244 - DNA, 43 bp, Artificial
  synthetic oligonucleotide probe
aatccactctcccacgccatttttaggcataggacccgtgtct

SEQ ID NO: 245 - DNA, 44 bp, Artificial
  synthetic oligonucleotide probe
acccatctgtcagcttcggatttttaggcataggacccgtgtct

SEQ ID NO: 246 - DNA, 44 bp, Artificial
  synthetic oligonucleotide probe
ccagggttcacaacgccttatttttaggcataggacccgtgtct

SEQ ID NO: 247 - DNA, 40 bp, Artificial
  synthetic oligonucleotide probe
gcgcttccctcgcgactttttaggcataggacccgtgtct

SEQ ID NO: 248 - DNA, 43 bp, Artificial
  synthetic oligonucleotide probe
tcccccacggacactcagttttttaggcataggacccgtgtct

SEQ ID NO: 249 - DNA, 20 bp, Artificial
  synthetic oligonucleotide probe
cctaccccccgtcaaagaat

SEQ ID NO: 250 - DNA, 19 bp, Artificial
  synthetic oligonucleotide probe
ctacaaactgcccccctcc